(19) United States
(12) Patent Application Publication
(10) Pub. No.: US 2025/0262193 A1
(43) Pub. Date: Aug. 21, 2025

(54) Therapeutic Tyrosine Kinase Inhibitors For Multiple Sclerosis and Myasthenia Gravis

(71) Applicant: Principia Biopharma Inc., Bridgewater, NJ (US)

(72) Inventor: Sana Syed, Chestnut Hill, MA (US)

(73) Assignee: Principia Biopharma Inc., Bridgewater, NJ (US)

(21) Appl. No.: 18/987,395

(22) Filed: Dec. 19, 2024

Related U.S. Application Data
(63) Continuation of application No. PCT/US23/26526, filed on Jun. 29, 2023.
(60) Provisional application No. 63/357,465, filed on Jun. 30, 2022.
(60) Provisional application No. 63/433,866, filed on Dec. 20, 2022.

(51) Int. Cl. A61K 31/437; G01N 33/84

(52) U.S. Cl. CPC A61K 31/437; G01N 33/84; G01N 2800/52

(57) ABSTRACT

This disclosure relates to the field of therapeutic tyrosine kinase inhibitors, in particular, Bruton tyrosine kinase (“BTK”) inhibitors, for treatment of patients with MG or MS.

### CROSS-REFERENCE TO RELATED APPLICATIONS

This application claims priority to U.S. Provisional Application No. 63/357,465, filed on Jun. 30, 2022, and U.S. Provisional Application No. 63/433,866, filed on Dec. 20, 2022, which are incorporated by reference herein in their entirety for any purpose.

### INTRODUCTION

This disclosure relates to the field of therapeutic tyrosine kinase inhibitors, in particular, Bruton tyrosine kinase (“BTK”) inhibitors, for treating multiple sclerosis (MS) and myasthenia gravis (MG). Multiple Sclerosis (MS) is a neurological disease affecting more than 1 million people worldwide. It is the most common cause of neurological disability in young and middle-aged adults and has a major physical, psychological, social and financial impact on subjects and their families. MS involves an immune-mediated process in which an abnormal response of the body's immune system is directed against the central nervous system (CNS). In the course of the disease, scleroses, i.e., lesions or scars, appear in the myelin sheath of nerve cells, disrupting transmission of electrical signals. Scleroses accumulate over time and result in the debilitating symptoms experienced by MS patients.

MS patients generally experience one of four clinical courses of disease, each of which might be mild, moderate, or severe: clinically isolated syndrome, relapsing remitting, secondary progressive (nonrelapsing secondary progressive multiple sclerosis (NRSPMS)) and primary progressive (PPMS). About 85% of MS patients have the relapsing remitting form of the disease, in which they experience clearly defined relapses (also called flare-ups or exacerbations), which are episodes of acute worsening of neurologic function, followed by partial or complete recovery periods (remissions) that are free of disease progression. Within the scope of the present disclosure, “relapsing multiple sclerosis,” “relapsing MS,” or “RMS” may include clinically isolated syndrome (“CIS”), relapsing remitting multiple sclerosis (“RRMS”), and relapsing secondary progressive multiple sclerosis (“R-SPMS.”) See, e.g., Lublin et al., Defining the clinical course of multiple sclerosis; the 2013 revisions, Neurology 2014; 83:278-286. Immunomodulatory drugs have been the mainstay of MS therapy. Recent results from clinical studies have demonstrated efficacy of agents that target B lymphocytes, especially B-cell-depleting agents like ocrelizumab (anti-CD20) (Hauser et al., N Engl J Med. 2017; 376(3):221-34).

Targeting B-cells represents a departure from the prevailing dogma based on animal models that demonstrated therapeutic benefits from modulating T-cell activity and positions the B cell as the centerpiece of current MS drug development (Lehmann-Horn K et al., Int J Mol Sci. 2017; 18(10):2048). The importance of immune cells residing in the CNS is also well known and needs to be considered in MS pathogenesis (Hemmer B et al, Nat Clin Pract Neurol. 2006; 2(4):201-11).

MG is a chronic autoimmune, neuromuscular disease that causes weakness in the skeletal muscles that worsens after periods of activity and improves after periods of rest. These muscles are responsible for functions involving breathing and moving parts of the body, including the arms and legs. The hallmark of myasthenia gravis is muscle weakness that worsens after periods of activity and improves after periods of rest. Certain muscles such as those that control eye and eyelid movement, facial expression, chewing, talking, and swallowing are often (but not always) involved in the disorder. The onset of the disorder may be sudden, and symptoms often are not immediately recognized as myasthenia gravis. The degree of muscle weakness involved in myasthenia gravis varies greatly among individuals. People with myasthenia gravis may experience the following symptoms: weakness of the eye muscles (called ocular myasthenia) drooping of one or both eyelids (ptosis), blurred or double vision (diplopia), a change in facial expression, difficulty swallowing, shortness of breath, impaired speech (dysarthria), weakness in the arms, hands, fingers, legs, and neck.

MG is an autoimmune disease, which means the immune system-which normally protects the body from foreign organisms-mistakenly attacks itself. Myasthenia gravis is caused by an error in the transmission of nerve impulses to muscles. It occurs when normal communication between the nerve and muscle is interrupted at the neuromuscular junction—the place where nerve cells connect with the muscles they control. Neurotransmitters are chemicals that neurons, or brain cells, use to communicate information. Normally when electrical signals or impulses travel down a motor nerve, the nerve endings release a neurotransmitter called acetylcholine that binds to sites called acetylcholine receptors on the muscle. The binding of acetylcholine to its receptor activates the muscle and causes a muscle contraction. In myasthenia gravis, antibodies block, alter, or destroy the receptors for acetylcholine at the neuromuscular junction, which prevents the muscle from contracting. This is most often caused by antibodies to the acetylcholine receptor itself, but antibodies to other proteins, such as MuSK (Muscle-Specific Kinase) protein, also can impair transmission at the neuromuscular junction.

Tolebrutinib is a BTK inhibitor that is being studied for both MG and MS. The Bruton's tyrosine kinase pathway is critical to signaling in B lymphocytes and myeloid cells including CNS microglia. Each of these cell types has been implicated in the pathophysiology of multiple sclerosis. Further, as BTK signaling is vital for maturation of B cells into antibody-secreting plasma cells, BTK inhibition can modulate both cellular and humoral immunity.

Accordingly, an inhibitor of BTK signaling represents a dual mechanism targeting both aspects of the immune system.

Accordingly, compounds that inhibit BTK that are able to both inhibit antigen-induced B-cell activation responsible for neuroinflammation and modulate maladaptive microglial cells linked to neuroinflammation in the brain and spinal cord may be useful in treating MS and MG with superior benefits when compared to currently available therapies.

Drug-induced liver injury has been identified in the ongoing tolebrutinib Phase 3 trials. The reported events occurred between months 2 to 3 after the start of tolebrutinib administration, and the elevation of liver enzymes appears reversible after tolebrutinib discontinuation. Thus, there is a need to mitigate the risk of hepatic injury and provide safe treatment for patients with MS or MG.

### SUMMARY

The present disclosure relates to methods of treating MS in a patient in need thereof comprising determining whether the patient has elevated transferrin or elevated ferritin levels and when the patient is found to not have elevated transferrin or elevated ferritin levels, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

The present disclosure relates to methods of treating MS comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have elevated transferrin or elevated ferritin levels.

The present disclosure relates to methods of treating MG in a patient in need thereof comprising determining whether the patient has elevated transferrin or elevated ferritin levels and when the patient is found to not have elevated transferrin or elevated ferritin levels, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

The present disclosure relates to methods of treating MG comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one the wherein the patient does not have elevated transferrin or elevated ferritin levels.

The present disclosure relates to methods of treating MS in a patient in need thereof comprising determining the patient's iron panel, and when the patient is found to have a suitable iron panel, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

The present disclosure relates to methods of treating MS comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient has a suitable iron panel.

The present disclosure relates to methods of treating MG in a patient in need thereof comprising determining the patient's iron panel, and when the patient is found to have a suitable iron panel, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

The present disclosure relates to methods of treating MG comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient has a suitable iron panel.

The present disclosure relates to methods of treating MS in a patient in need thereof comprising determining the patient's alcohol consumption, and when the patient is found to have a suitable alcohol consumption, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

The present disclosure relates to methods of treating MS comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient has a suitable alcohol level.

The present disclosure relates to methods of treating MG in a patient in need thereof comprising determining the patient's alcohol consumption, and when the patient is found to have a suitable alcohol consumption, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

The present disclosure relates to methods of treating MG comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient has a suitable alcohol consumption.

The present disclosure relates to methods of treating MS in a patient in need thereof comprising determining if the patient has inducers of CYP3A or inhibitor of CYP3C8 in the patient's system, and when the patient is found to not have an inducer of CYP3A or an inhibitor of CYP3C8, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

The present disclosure relates to methods of treating MS comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have an inducer of CYP3A or an inhibitor of CYP3C8 in the patient's system.

The present disclosure relates to methods of treating MG in a patient in need thereof comprising determining if the patient has inducers of CYP3A or inhibitor of CYP3C8 in the patient's system, and when the patient is found to not have an inducer of CYP3A or an inhibitor of CYP3C8, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

The present disclosure relates to methods of treating MG comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have an inducer of CYP3A or an inhibitor of CYP3C8 in the patient's system.

The present disclosure relates to methods of treating MS in a patient in need thereof comprising determining if the patient has elevated ALT enzymes, and when the patient is found to not have elevated ALT enzymes, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

The present disclosure relates to methods of treating MS comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have elevated ALT enzymes

The present disclosure relates to methods of treating MG in a patient in need thereof comprising determining if the patient has elevated ALT enzymes, and when the patient is found to not have elevated ALT enzymes, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

The present disclosure relates to methods of treating MG comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have elevated ALT enzymes.

In some embodiments, a dose of about 5 mg to about 60 mg of the BTK inhibitor is administered.

In some embodiments, the dose is 5 mg.

In some embodiments, the dose is 15 mg.

In some embodiments, the dose is 30 mg.

In some embodiments, the dose is 60 mg.

In some embodiments, the dose is once daily.

In some embodiments, the dose is administered once daily with food.

In some embodiments, the dose is 60 mg and is administered once daily with food.

In some embodiments, the BTK inhibitor compound is administered as monotherapy.

In some embodiments, the subject or patient is a human.

In some embodiments, the subject or patient is a human subject or patient ranging in age from 12 to 55 years old.

The present disclosure also relates to a BTK inhibitor comprising (R)-1-(1-acryloylpiperidin-3-yl)-4-amino-3-(4-phenoxyphenyl)-1H-imidazo[4,5-c]pyridin-2(3H)-one for use in a method for treating MG or MS in a patient in need thereof.

The present disclosure also relates to methods of treating MS, comprising the steps of:
(a) performing an iron panel test in a patient's blood or serum;
(b) detecting levels of the iron panel test that are within normal ranges; and
(c) administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the iron panel test measures any one or more of levels of iron, ferritin, transferrin saturation, and total iron-binding capacity (TIBC) in a patient's blood or serum and wherein the normal ranges of the iron panel test include one or more of (i) an iron level of 60 to 170 μg/dL, (ii) a ferritin level of ≤500 μg/L (iii) a transferrin saturation level ≤50% in a male patient or ≤40% in a female patient, and (iv) a TIBC of 240 to 450 μg/dL.

The present disclosure also relates to methods of treating MS, comprising the steps of:
(a) detecting a level of transferrin saturation in a patient's blood or serum that is within normal range; and
(b) administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the transferrin saturation level that is within normal range in the blood or serum of a male patient is a transferrin saturation of ≤50%, and the transferrin saturation level that is within normal range in the blood or serum of a female patient is a transferrin saturation of ≤40%.

The present disclosure also relates to methods of treating MS, comprising the steps of:
(a) detecting a level of ferritin in a patient's blood or serum that is within normal range; and
(b) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the ferritin level that is within normal range in the blood or serum of the patient is ≤500 μg/L.

The present disclosure also relates to methods of treating MS, comprising the steps of:
(a) performing liver function tests in a patient;
(b) detecting suitable liver function in the patient; and
(c) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the liver function tests measure one or more of the levels of aspartate transaminase (AST), alanine transaminase (ALT), albumin, alkaline phosphatase, total and direct bilirubin, and total protein in a patient's blood, and wherein the patient having a suitable liver function has one or more of ALT ≤1.5× upper limit of normal (ULN), AST levels of ≤1.5×ULN, alkaline phosphatase ≤2×ULN (unless caused by non-liver related disorder or explained by a stable chronic liver disorder) and total bilirubin ≤1.5×ULN (unless due to Gilbert syndrome or non-liver-related disorder).

The present disclosure also relates to methods of treating MS, comprising the steps of:
(a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
(b) measuring the level of alanine aminotransferase (ALT) in the patient;
(c) detecting a level of ALT of >8× upper limit of normal (ULN);
(d) ceasing administration of the Compound to the patient; and optionally
(e) monitoring the level of ALT in the patient; and
(f) resuming administration of a therapeutically effective amount of the Compound to the patient when the patient's level of ALT is determined to be <1.5×ULN.

The present disclosure also relates to methods of treating MS, comprising the steps of:
(a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
(b) measuring the level of alanine aminotransferase (ALT) in the patient;
(c) detecting a level of ALT of >5× upper limit of normal (ULN) during a period of at least two weeks;
(d) ceasing administration of the Compound to the patient; and optionally
(e) monitoring the level of ALT in the patient; and
(f) resuming administration of a therapeutically effective amount of the Compound to the patient when the patient's level of ALT is determined to be <1.5×ULN.

The present disclosure also relates to methods of treating MS, comprising the steps of:
(a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
(b) measuring the level of alanine aminotransferase (ALT) in the patient;
(c) detecting a level of ALT of >3× upper limit of normal (ULN);
(d) measuring one or more of total bilirubin and international normalized ratio (INR) in a patient;
(e) detecting one or more of total bilirubin >2×ULN and INR >1.5;
(f) ceasing administration of the Compound to the patient; and optionally
(g) monitoring the level of ALT in the patient; and
(h) resuming administration of a therapeutically effective amount of the Compound to the patient when the patient's level of ALT is determined to be <1.5×ULN.

The present disclosure also relates to methods of treating MS, comprising the steps of:
(a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
(b) measuring the level of alanine aminotransferase (ALT) in the patient;
(c) detecting a level of ALT of >3× upper limit of normal (ULN);
(d) ceasing administration of the Compound to the patient if the patient experiences one or more of fatigue, nausea, vomiting, right upper quadrant pain or tenderness, fever, rash, and eosinophilia >5%; and optionally
(e) monitoring the level of ALT in the patient; and
(f) resuming administration of a therapeutically effective amount of the Compound to the patient when the patient's level of ALT is determined to be <1.5×ULN.

In some embodiments, the level of ALT in step (b) is determined at least monthly.

In some embodiments, the level of ALT in step (d) is monitored at least weekly.

In some embodiments, the level of ALT in step (d) is monitored every 2 to 3 days.

The present disclosure also relates to methods of treating MS in a patient in need thereof, comprising administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one to the patient, wherein the patient is not receiving potent and moderate inducers of cytochrome P450 3A (CYP3A) or potent inhibitors of CYP2C8 hepatic enzymes.

The present disclosure also relates to methods of treating MS in a patient in need thereof, comprising the steps of:
(a) advising the patient to limit alcohol consumption during treatment; and
(b) administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the patient is female and is advised to limit alcohol consumption to 14 grams/day or less, or the patient is male and is advised to limit alcohol consumption to 28 grams/day or less.

The present disclosure also relates to methods of treating MG, comprising the steps of:
(a) performing an iron panel test in a patient's blood or serum;
(b) detecting levels of the iron panel test that are within normal ranges; and
(c) administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the iron panel test measures any one or more of levels of iron, ferritin, transferrin saturation, and total iron-binding capacity (TIBC) in a patient's blood or serum and wherein the normal ranges of the iron panel test include one or more of (i) an iron level of 60 to 170 μg/dL, (ii) a ferritin level of ≤500 μg/L (iii) a transferrin saturation level ≤50% in a male patient or ≤40% in a female patient, and (iv) a TIBC of 240 to 450 μg/dL.

The present disclosure also relates to methods of treating MG, comprising the steps of:
(a) detecting a level of transferrin saturation in a patient's blood or serum that is within normal range; and
(b) administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the transferrin saturation level that is within normal range in the blood or serum of a male patient is a transferrin saturation of ≤50%, and the transferrin saturation level that is within normal range in the blood or serum of a female patient is a transferrin saturation of ≤40%.

The present disclosure also relates to methods of treating MG, comprising the steps of:
(a) detecting a level of ferritin in a patient's blood or serum that is within normal range; and
(b) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the ferritin level that is within normal range in the blood or serum of the patient is ≤500 μg/L.

The present disclosure also relates to methods of treating MG, comprising the steps of:
(a) performing liver function tests in a patient;
(b) detecting suitable liver function in the patient; and
(c) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the liver function tests measure one or more of the levels of aspartate transaminase (AST), alanine transaminase (ALT), albumin, alkaline phosphatase, total and direct bilirubin, and total protein in a patient's blood, and wherein the patient having a suitable liver function has one or more of ALT ≤1.5× upper limit of normal (ULN), AST levels of ≤1.5×ULN, alkaline phosphatase ≤2×ULN (unless caused by non-liver related disorder or explained by a stable chronic liver disorder) and total bilirubin ≤1.5×ULN (unless due to Gilbert syndrome or non-liver-related disorder).

The present disclosure also relates to methods of treating MG, comprising the steps of:
(a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
(b) measuring the level of alanine aminotransferase (ALT) in the patient;
(c) detecting a level of ALT of >8× upper limit of normal (ULN);
(d) ceasing administration of the Compound to the patient; and optionally
(e) monitoring the level of ALT in the patient; and
(f) resuming administration of a therapeutically effective amount of the Compound to the patient when the patient's level of ALT is determined to be <1.5×ULN.

The present disclosure also relates to methods of treating MG, comprising the steps of:
(a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof;
(b) measuring the level of alanine aminotransferase (ALT) in the patient;
(c) detecting a level of ALT of >5× upper limit of normal (ULN) during a period of at least two weeks;
(d) ceasing administration of the Compound to the patient; and optionally
(e) monitoring the level of ALT in the patient; and
(f) resuming administration of a therapeutically effective amount of the Compound to the patient when the patient's level of ALT is determined to be <1.5×ULN.

The present disclosure also relates to methods of treating MG, comprising the steps of:
(a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof;
(b) measuring the level of alanine aminotransferase (ALT) in the patient;
(c) detecting a level of ALT of >3× upper limit of normal (ULN);
(d) measuring one or more of total bilirubin and international normalized ratio (INR) in a patient;
(e) detecting one or more of total bilirubin >2×ULN and INR >1.5;
(f) ceasing administration of the Compound to the patient; and optionally
(g) monitoring the level of ALT in the patient; and
(h) resuming administration of a therapeutically effective amount of the Compound to the patient when the patient's level of ALT is determined to be <1.5×ULN.

The present disclosure also relates to methods of treating MG, comprising the steps of:
(a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
(b) measuring the level of alanine aminotransferase (ALT) in the patient;
(c) detecting a level of ALT of >3× upper limit of normal (ULN);
(d) ceasing administration of the Compound to the patient if the patient experiences one or more of fatigue, nausea, vomiting, right upper quadrant pain or tenderness, fever, rash, and eosinophilia >5%; and optionally
(e) monitoring the level of ALT in the patient; and
(f) resuming administration of a therapeutically effective amount of the Compound to the patient when the patient's level of ALT is determined to be <1.5×ULN.

In some embodiments, the level of ALT in step (b) is determined at least monthly.

In some embodiments, the level of ALT in step (d) is monitored at least weekly.

In some embodiments, the level of ALT in step (d) is monitored every 2 to 3 days.

The present disclosure also relates to methods of treating MG in a patient in need thereof, comprising administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one to the patient, wherein the patient is not receiving potent and moderate inducers of cytochrome P450 3A (CYP3A) or potent inhibitors of CYP2C8 hepatic enzymes.

The present disclosure also relates to methods of treating MG in a patient in need thereof, comprising the steps of:
(a) advising the patient to limit alcohol consumption during treatment; and
(b) administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the patient is female and is advised to limit alcohol consumption to 14 grams/day or less, or the patient is male and is advised to limit alcohol consumption to 28 grams/day or less.

### BRIEF DESCRIPTION OF DRAWINGS

FIG. 1A provides the study design of Example 1.

FIG. 1B provides the study design of Example 2.

FIG. 1C provides the study design of Example 3.

FIG. 1D provides the study design of Example 4.

FIG. 2 provides suggested actions and follow-up assessments in the event of neutropenia.

FIG. 3 provides suggested actions and follow-up assessments in the event of thrombocytopenia.

FIG. 4A provides suggested actions and follow-up assessments in the event of increased alanine aminotransferase (ALT) algorithm.

FIG. 4B provides suggested actions and follow-up assessments in the event of increased alanine aminotransferase (ALT) algorithm.

FIG. 5 provides suggested actions and follow-up assessments in the event of serum creatinine.

FIG. 6 provides suggested actions and follow-up assessments when progressive multifocal leukoencephalopathy (PML) is suspected.

FIG. 7 provides a description of the expanded disability status scale score (EDSS) in view of the level of disability.

FIG. 8 provides suggested actions and follow-up assessments in the event of increase in CPK of non-cardiac origin and not related to intensive physical activity.

### DETAILED DESCRIPTION

Reference will now be made in detail to certain embodiments, examples of which are illustrated in the accompanying drawings. While the disclosure provides illustrated embodiments, it will be understood that they are not intended to limit the invention to those embodiments. On the contrary, the invention is intended to cover all alternatives, modifications, and equivalents, which may be included within the disclosure as defined by the appended claims.

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless otherwise stated, the following terms used in the specification and claims are defined for the purposes of this disclosure and have the following meaning.

As used herein, “the BTK inhibitor,” “the BTK inhibitor compound,” and “the compound”, refers to (R)-1-(1-acryloylpiperidin-3-yl)-4-amino-3-(4-phenoxyphenyl)-1H-imidazo[4,5-c]pyridin-2(3H)-one having the following structure:

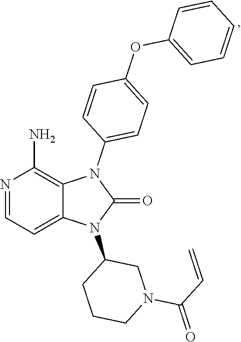 [SMILES: C=CC(=O)N1CCC[C@@H](n2c(=O)n(-c3ccc(Oc4ccccc4)cc3)c3c(N)nccc32)C1]

which is also known as “tolebrutinib,” and 4-amino-3-(4-phenoxyphenyl)-1-[(3R)-1-(prop-2-enoyl)piperidin-3-yl]-1,3-dihydro-2H-imidazo[4,5-c]pyridin-2-one having the following structure:

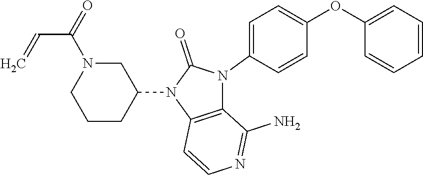 [SMILES: C=CC(=O)N1CCC[C@@H](n2c(=O)n(-c3ccc(Oc4ccccc4)cc3)c3c(N)nccc32)C1]

or a pharmaceutically acceptable salt thereof.

A “pharmaceutically acceptable carrier” or a “pharmaceutically acceptable excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Treating” or “treatment” of a disease includes:
(1) inhibiting the disease, e.g., arresting or reducing the development of the disease or its clinical symptoms; or
(2) relieving the disease, e.g., causing regression of the disease or its clinical symptoms.

“Optional” or “optionally” means that the subsequently described event or circumstance may but need not occur, and that the description includes instances where the event or circumstance occurs and instances in which it does not.

A “therapeutically effective amount” means the amount of the BTK inhibitor compound, that, when administered to a mammal for treating a disease, is sufficient to affect such treatment for the disease. The “therapeutically effective amount” will vary depending on the compound, the disease and its severity and the age, weight, etc., of the mammal to be treated.

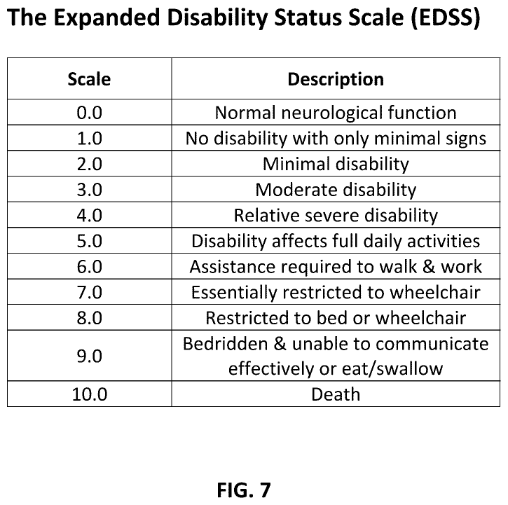

“Expanded disability status scale (EDSS) score” is a method of quantifying disability in multiple sclerosis and monitoring changes in the level of disability over time. The EDSS scale ranges from 0 to 10 in 0.5 unit increments that represent higher levels of disability. EDSS steps 1.0 to 4.5 refer to people with MS who are able to walk without any aid and is based on measures of impairment in eight functional systems (FS): pyramidal—muscle weakness or difficulty moving limbs; cerebellar—ataxia, loss of balance, coordination or tremor; brainstem—problems with speech, swallowing and nystagmus; sensory—numbness or loss of sensations; bowel and bladder function; visual function—problems with sight; cerebral functions—problems with thinking and memory. EDSS steps 5.0 to 9.5 are defined by the impairment to walking. See, e.g., FIG. 7. Information about this score is found at Kurtzke et al. Neurology 1983, 33, 1444-1452

Before describing the present teachings in detail, it is to be understood that the disclosure is not limited to specific compositions or process steps, as such may vary.

It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims.)

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “or,” unless the context requires otherwise.

“Ceasing” or “cessation” when used regarding administration of a BTK inhibitor compound means that the a BTK inhibitor compound is no longer being administered to the patient on either a temporary or permanent basis.

“Monitoring” with reference to assessment of the level of ALT in a patient means checking, and/or detecting the level of ALT in a patient over at least two points in time; in some embodiments, over a period of time; in some embodiments, monthly; in some embodiments, at least monthly; in some embodiments, weekly; in some embodiments, at least weekly; in some embodiments, every 5 days; in some embodiments, every 3 days; in some embodiments, every 2 to 3 days; in some embodiments, every 2 days; in some embodiments, daily.

### II. Administered BTK Inhibitor Compound

In some embodiments, a BTK inhibitor compound, (R)-1-(1-acryloylpiperidin-3-yl)-4-amino-3-(4-phenoxyphenyl)-1H-imidazo[4,5-c]pyridin-2(3H)-one is administered for treating MS or MG in a patient in need thereof. In some embodiments, the BTK inhibitor compound is a pharmaceutically acceptable salt of (R)-1-(1-acryloylpiperidin-3-yl)-4-amino-3-(4-phenoxyphenyl)-1H-imidazo[4,5-c]pyridin-2(3H)-one. In some embodiments, a therapeutically effective amount of the BTK inhibitor compound is administered. In some embodiments, a dose of 5 to 60 mg of the BTK inhibitor compound is administered. In some embodiments, a dose of 60 mg of the BTK inhibitor compound is administered. In some embodiments, a dose of 60 mg once daily of the BTK inhibitor compound is administered.

In some embodiments, a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one is provided for use in a method for treating MS or MG in a patient in need thereof.

In some embodiments, a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one is provided for use in a method for reducing the frequency of MS or MG relapse in a patient in need thereof.

The BTK inhibitor compound can be prepared according to the methods and schemes described in, e.g., U.S. Pat. No. 9,688,676 B2, in particular the content of column 62, line 8 to column 65 line 32, and column 67, line 28 to column 69, which is incorporated herein by reference.

The following preparation of the compound of (R)-1-(1-acryloylpiperidin-3-yl)-4-amino-3-(4-phenoxyphenyl)-1H-imidazo[4,5-c]pyridin-2(3H)-one, is given to enable those skilled in the art to prepare the BTK inhibitor compound. The synthetic route should not be considered as limiting the scope of the disclosure, but merely as being illustrative and representative thereof.

### Exemplary Synthesis of (R)-1-(1-acryloylpiperidin-3-yl)-4-amino-3-(4-phenoxyphenyl)-1H-imidazo[4,5-c]pyridin-2(3H)-one

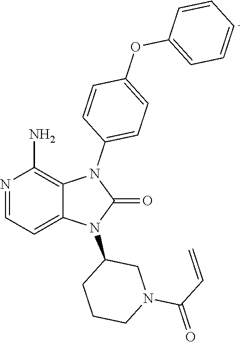 [SMILES: C=CC(=O)N1CCC[C@@H](n2c(=O)n(-c3ccc(Oc4ccccc4)cc3)c3c(N)nccc32)C1]

Into a 100 mL round-bottom flask was placed (R)-4-amino-3-(4-phenoxyphenyl)-1-(piperidin-3-yl)-1H-imidazo[4,5-c]pyridin-2(3H)-one (150 mg, 0.37 mmol, 1.00 equiv), DCM-CH3OH (6 mL), TEA (113 mg, 1.12 mmol, 3.00 equiv). This was followed by the addition of prop-2-enoyl chloride (40.1 mg, 0.44 mmol, 1.20 equiv) dropwise with stirring at 0° C. in 5 min. The resulting solution was stirred for 2 h at 0° C. The resulting mixture was concentrated under vacuum. The residue was applied onto a silica gel column with dichloromethane/methanol (30:1). The crude product (100 mg) was purified by Prep-HPLC under the following conditions (Column, XBridge Prep C18 OBD Column, 5 μm, 19*150 mm; mobile phase, water with 0.05% TFA and ACN (25.0% ACN up to 45.0% in 8 min). 54.5 mg product of (R)-1-(1-acryloylpiperidin-3-yl)-4-amino-3-(4-phenoxyphenyl)-1H-imidazo[4,5-c]pyridin-2(3H)-one was obtained as a white solid. LC-MS m/z: 465.2 (M+1).

### III. Therapeutic Methods to Mitigate Risk of Hepatic Injury

Provided herein are methods of treating MS, including RMS, NRSPMS, and PPMS, or MG comprising administering to a patient in need thereof a therapeutically effective amount of (R)-1-(1-acryloylpiperidin-3-yl)-4-amino-3-(4-phenoxyphenyl)-1H-imidazo[4,5-c]pyridin-2(3H)-one, or a pharmaceutically acceptable salt thereof. However, the administration of the compound will (in certain embodiments) only occur if certain pre-conditions are met.

In one embodiment, the patient's levels of transferrin or ferritin are measured. If it is found that the patient's levels of transferrin or ferritin are not elevated, then the patient will receive the BTK inhibitor compound. However, if is found that the patient's transferrin or ferritin are elevated, the patient will not receive the compound. Elevated transferrin levels are determined by examining the saturation level of transferrin. Particularly an elevated transferrin level is where the patient's transferrin saturation level is >50% in males and >40% in females. Stated differently, if the patient's transferrin saturation level is >50% in males and >40% in females, the patient will not receive the BTK compound, whereas if the patient's transferrin saturation level is measured and it is found that the saturation is less than or equal to 50% in males or less than or equal to 40% in females, then the patient can receive study drug. This measurement of transferrin saturation may be done for either or both the MS and MG patients.

In some embodiments, the patient's ferritin levels are measured. If it is found that the patient's levels of ferritin are not elevated, then the patient will receive the BTK inhibitor compound. Elevated ferritin levels means a level of >500 μg/L. In some embodiments, for both treating MS and MG, the patient's level of ferritin is measured, and if it is found to be higher than 500 μg/L, the patient is not administered the BTK compound. But if it is found to be less than or equal to 500 μg/L, then the patient will receive the BTK compound.

In some embodiments, for both treatment of MG and MS, the patient's iron panel will be measured, which includes both iron levels in blood and serum, ferritin levels and transferrin saturation. If either the ferritin level or the transferrin level is above the thresholds listed above, the patient will not receive the BTK inhibitor. But if both the ferritin level and the transferrin level are below the thresholds listed above, the patient will receive the BTK inhibitor.

In some embodiments, there are methods of treating MS and MG comprising determining the patient's alcohol consumption, and when the patient is found to have a suitable alcohol consumption, administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one. In some embodiments, suitable alcohol consumption means that patient does not have an active alcohol use disorder or a history of alcohol or drug abuse within 1 year prior to the first visit. In other embodiments, suitable alcohol consumption means that the patient has a current alcohol intake of >2 drinks per day for men and >1 drink per day for women (1 drink=approximately 14 grams of alcohol=350 mL beer=140 mL wine=40 mL of spirits). Thus, the patient needs to have an alcohol consumption less than 2 drinks/day for men and 1 drink/day for women in order for the BTK compound to be administered.

In some embodiments, there are methods for treating MS and MG in which it is measured whether the patient is concomitantly taking inducers of the CYP3A enzyme. If the patient has taken such an inhibitor, the patient will not receive the BTK compound. Likewise, the patient will not be given the BTK compound if patient is concomitantly taking an inhibitor of CYP3C8.

In some embodiments, for both treating MS and MG, it will be determined if the patient has elevated ALT enzymes, and if so, the patient will not receive the BTK inhibitor. This refers to the alanine aminotransferase (ALT) enzyme that has been increased and/or has a starting value that is >3× upper limit of normal (ULN). Such patients will not receive the BTK compound.

In one embodiment, the patient's levels of transferrin or ferritin are measured. If it is found that the patient's levels of transferrin or ferritin are not elevated, then the patient will continue to receive the BTK inhibitor compound. However, if is found that the patient's transferrin or ferritin are elevated, the patient discontinues receiving the compound (or the compound is no longer administered to the patient). Specifically, in some embodiments if the patient's transferrin saturation level is >50% in males and >40% in females, the patient will discontinue receiving the compound. Such discontinuance of the therapy based upon transferrin levels may be done for either or both the MS and MG patients.

In some embodiments, the patient's ferritin levels are measured. If it is found that the patient's levels of ferritin are not elevated, then the patient will continue to receive the BTK inhibitor compound. However, if is found that the patient's ferritin is elevated, the patient discontinues receiving the compound (or the compound is no longer administered to the patient). Specifically, for both treating MS and MG, the patient's level of ferritin is measured, and if it is found to be higher than >500 μg/L, the patient is not administered the BTK compound and/or the patient stops receiving the compound. But if it is found to be less than or equal to 500 μg/L, then the patient will continue to receive the BTK compound.

In some embodiments, for both treatment of MG and MS, the patient's iron panel will be measured, which includes both iron levels in blood and serum, ferritin levels and transferrin saturation. If either the ferritin level or the transferrin level is above the thresholds listed above, the patient will not receive the BTK inhibitor or will stop receiving the compound. If both the ferritin level or the transferrin level is above the thresholds listed above, the patient will also not receive the BTK inhibitor or will stop receiving the compound.

In some embodiments, there are methods of treating MS and MG comprising determining the patient's alcohol consumption, and when the patient is found to have a suitable alcohol consumption, administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one. In some embodiments, suitable alcohol consumption means that patient does not have an active alcohol use disorder or a history of alcohol or drug abuse within 1 year prior to the first visit. In other embodiments, suitable alcohol consumption means that the patient has a current alcohol intake of >2 drinks per day for men and >1 drink per day for women (1 drink=approximately 14 grams of alcohol=350 mL beer=140 mL wine=40 mL of spirits). Thus, the patient needs to have an alcohol consumption less than 2 drinks/day for men and 1 drink/day for women in order for the BTK compound to be administered.

In some embodiments, there are methods of treating MS and MG comprising determining the patient's alcohol consumption, and when the patient is found to have a higher than suitable alcohol consumption, the patient is not administered the compound or the administration of the compound is discontinued. In some embodiments, higher than suitable alcohol consumption means that patient has an active alcohol use disorder or a history of alcohol or drug abuse within 1 year prior to the first visit. In other embodiments, higher than suitable alcohol consumption means that the patient has a current alcohol intake of >2 drinks per day for men and >1 drink per day for women (1 drink=approximately 14 grams of alcohol=350 mL beer=140 mL wine=40 mL of spirits). Thus, the patient needs to have an alcohol consumption less than 2 drinks/day for men and 1 drink/day for women in order for the BTK compound to be administered or for the patient to continue to receive the compound.

In some embodiments, there are methods for treating MS and MG in which it is measured whether the patient is concomitantly taking inducers of the CYP3A enzyme. If the patient has taken such an inhibitor, the patient will not receive the BTK compound and/or the administration of the compound will be discontinued. Likewise, the patient will not be given the BTK compound (or the administration of the compound will be discontinued) if patient is concomitantly taking an inhibitor of CYP3C8.

In some embodiments, for both treating MS and MG, it will be determined if the patient has elevated ALT enzymes, and if so, the patient will not receive the BTK inhibitor or will discontinue receiving the compound. This refers to the alanine aminotransferase (ALT) enzyme that has been increased and/or has a starting value that is >3× upper limit of normal (ULN). Such patients will not receive the BTK compound or the administration of the compound will cease.

In one embodiment, for either MG or MS patients, the patient will be classified as having mild, moderate or severe hepatic impairment as measured by the Child-Pugh class scale. In some embodiments, if the patient is determined to have severe hepatic impairment, the patient will not receive the compound or the administration of the compound to said patient will cease. In some embodiments, if the patient is determined to have moderate hepatic impairment, the patient will not receive the compound or the administration of the compound to said patient will cease. In some embodiments, if the patient is determined to have mild hepatic impairment, the patient will not receive the compound or the administration of the compound to said patient will cease.

In some embodiments, for both MS and MG treatment, the patient will be screened to determine if the ALT >1.5×ULN OR AST >1.5×ULN OR alkaline phosphatase >2×ULN (unless caused by non-liver-related disorder or explained by a stable chronic liver disorder) OR total bilirubin >1.5×ULN (unless due to Gilbert syndrome or non-liver-related disorder). Such patient's that have enzymes above these levels will not be administered the compound or if they have already received the compound, will discontinue receiving the compound.

In some embodiments, the dose is once daily. In some embodiments, the dose is administered once daily with food. In some embodiments, the dose is 60 mg and is administered once daily with food. In some embodiments, the BTK inhibitor compound is administered as monotherapy.

In some embodiments, the patient is a mammal. In some embodiments, the mammal is a human. In some embodiments, the patient is a human subject ranging in age from 12 to 55 years old.

In some embodiments the therapeutically effective amount is about 5 to about 60 mg. In some embodiments, the patient is a mammal. In some embodiments, the mammal is a human. In some embodiments, the patient is a human patient ranging in age from 12 to 55 years old.

In some embodiments the therapeutically effective amount is about 5 to about 60 mg. In some embodiments, the patient is a mammal. In some embodiments, the mammal is a human. In some embodiments, the patient is a human patient ranging in age from 12 to 55 years old.

In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered as monotherapy. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered as monotherapy in 60 mg doses. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered as monotherapy in 60 mg doses once daily. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered as monotherapy in 60 mg doses once daily with food.

In some embodiments, a dose of about 5-10 mg, 10-15 mg, 15-20 mg, 20-25 mg, 25-30 mg, 30-35 mg, 35-40 mg, 40-45 mg, 45-50 mg, 50-55 mg, or 55-60 mg is administered. In some embodiments, the dose is 5 mg, 10 mg, 15 mg, 20 mg, 25 mg, 30 mg, 35 mg, 40 mg, 45 mg, 50 mg, 55 mg, or 60 mg. In some embodiments, the dose is 5 mg. In some embodiments, the dose is 15 mg. In some embodiments, the dose is 30 mg. In some embodiments, the dose is 60 mg.

In some embodiments, the dose is administered daily. The daily dose can be delivered as a single dose or split into multiple parts. For example, in some embodiments, the dose is administered once a day (e.g., about every 24 hours). In some embodiments, the dose is administered twice daily. In some embodiments, the dose is subdivided in two parts to be administered twice per day (e.g., about every 12 hours). In some embodiments, the dose is subdivided in three parts to be administered three times per day (e.g., about every 8 hours). In some embodiments, the dose is subdivided in four parts to be administered four times per day (e.g., about every 6 hours).

In some embodiments, the dose is administered orally. In some embodiments, the dose is administered in a form of tablets. In some embodiments, the dose is administered in the form of pills, capsules, semisolids, powders, sustained release formulations, solutions, suspensions, elixirs, aerosols, or any other appropriate compositions.

In some embodiments, the patient is administered the BTK inhibitor compound for a period of about 1, 2, 3, 4, 5, 6, 7, 8, 9, 10, 11, or 12 months, or for life. In some embodiments, the patient is administered the BTK inhibitor compound for a period of about 12 months. In some embodiments, the dose is once daily. In one embodiment, a method of treating MS or MG is provided, the method comprising administering to a patient in need thereof 60 mg BTK inhibitor comprising (R)-1-(1-acryloylpiperidin-3-yl)-4-amino-3-(4-phenoxyphenyl)-1H-imidazo[4,5-c]pyridin-2(3H)-one.

In some embodiments, the BTK inhibitor compound is administered as monotherapy. In some embodiments, the method comprises administering the BTK inhibitor compound and at least one additional therapeutic agent. The additional therapeutic agent may be administered concurrently or sequentially with the BTK inhibitor compound.

Determination of the frequency of administration can be made by persons skilled in the art, such as an attending physician based on considerations of the condition being treated, age of the subject being treated, severity of the condition being treated, general state of health of the subject being treated and the like. In some embodiments, BTK inhibitor compounds are administered in a therapeutically effective amount for treatment of RMS. The therapeutically effective amount is typically dependent on the weight of the subject being treated, his or her physical or health condition, the extensiveness of the condition to be treated, or the age of the subject being treated, pharmaceutical formulation methods, or administration methods (e.g., administration time and administration route).

In some embodiments, a method of treating MS, including RMS, NRSPMS, and PPMS, or MG is provided, comprising the steps of performing an iron panel test using a patient's blood or serum, and if the patient has a suitable iron panel, administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments the iron panel test measures any one or more of levels of iron, ferritin, transferrin saturation, and total iron-binding capacity (TIBC) in a patient's blood or serum. In some embodiments, a suitable iron panel includes one or more of the following: (i) an iron level of 60 to 170 μg/dL, (ii) a ferritin level of ≤500 μg/L (iii) a transferrin saturation level ≤50% in a male patient or ≤40% in a female patient, and (iv) a TIBC of 240 to 450 μg/dL.

In some embodiments, a method of treating MS, including RMS, NRSPMS, and PPMS, or MG is provided, comprising the steps of performing an iron panel test in a patient's blood or serum, detecting levels of the iron panel test that are within normal ranges, and administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments the iron panel test measures any one or more of levels of iron, ferritin, transferrin saturation, and total iron-binding capacity (TIBC) in a patient's blood or serum. In some embodiments, the normal ranges of the iron panel test include one or more of (i) an iron level of 60 to 170 μg/dL, (ii) a ferritin level of ≤500 μg/L (iii) a transferrin saturation level ≤50% in a male patient or ≤40% in a female patient, and (iv) a TIBC of 240 to 450 μg/dL.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating MS, including RMS, NRSPMS, and PPMS, or MG, comprising the steps of performing an iron panel test in a patient's blood or serum, detecting levels of the iron panel test that are within normal ranges, and administering a therapeutically acceptable amount of Compound to the patient. In some embodiments the iron panel test measures any one or more of levels of iron, ferritin, transferrin saturation, and total iron-binding capacity (TIBC) in a patient's blood or serum. In some embodiments, the normal ranges of the iron panel test include one or more of (i) an iron level of 60 to 170 μg/dL, (ii) a ferritin level of ≤500 μg/L (iii) a transferrin saturation level ≤50% in a male patient or ≤40% in a female patient, and (iv) a TIBC of 240 to 450 μg/dL.

In some embodiments, a method of treating MS, including RMS, NRSPMS, and PPMS, or MG is provided, comprising the steps of determining the level of transferrin saturation in a patient's blood or serum, and if the level of transferrin saturation is suitable, administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, a suitable transferrin saturation level in the blood or serum of a male patient is a transferrin saturation of ≤50%. In some embodiments, a suitable transferrin saturation level in the blood or serum of a female patient is a transferrin saturation of ≤40%.

In some embodiments, a method of treating MS, including RMS, NRSPMS, and PPMS, or MG is provided, comprising the steps of detecting a level of transferrin saturation in a patient's blood or serum that is within normal range, and administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, a transferrin saturation level that is within normal range in the blood or serum of a male patient is a transferrin saturation of ≤50%. In some embodiments, a transferrin saturation level that is within normal range in the blood or serum of a female patient is a transferrin saturation of ≤40%.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating MS, including RMS, NRSPMS, and PPMS, or MG, comprising the steps of detecting a level of transferrin saturation in a patient's blood or serum that is within normal range, and administering a therapeutically effective amount of Compound to the patient. In some embodiments, a transferrin saturation level that is within normal range in the blood or serum of a male patient is a transferrin saturation of ≤50%. In some embodiments, a transferrin saturation level that is within normal range in the blood or serum of a female patient is a transferrin saturation of ≤40%.

In some embodiments, a method of treating MS, including RMS, NRSPMS, and PPMS, or MG is provided, comprising the steps of determining the level of ferritin in a patient's blood or serum, and if the level of ferritin is suitable, administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, a suitable ferritin level in the blood or serum of a patient is ≤500 μg/L.

In some embodiments, a method of treating MS, including RMS, NRSPMS, and PPMS, or MG is provided, comprising the steps of detecting a level of ferritin in a patient's blood or serum that is within normal range, and administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, a ferritin level that is within normal range in the blood or serum of a patient is ≤500 μg/L.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating MS, including RMS, NRSPMS, and PPMS, or MG, comprising the steps of detecting a level of ferritin in a patient's blood or serum that is within normal range, and administering a therapeutically acceptable amount of Compound to the patient. In some embodiments, a ferritin level that is within normal range in the blood or serum of a patient is ≤500 μg/L.

In some embodiments, a method of treating MS, including RMS, NRSPMS, and PPMS, or MG is provided, comprising the steps of performing liver function tests in a patient, and if the patient has suitable liver function, administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, the liver function tests measure one or more of the levels of aspartate transaminase (AST), alanine transaminase (ALT), albumin, alkaline phosphatase, total and direct bilirubin, and total protein in a patient's blood. In some embodiments a patient having a suitable liver function has one or more of ALT levels of ≤1.5× upper limit of normal (ULN), AST levels of ≤1.5×ULN, and alkaline phosphatase ≤2×ULN (unless caused by non-liver related disorder or explained by a stable chronic liver disorder) and total bilirubin ≤1.5×ULN (unless due to Gilbert syndrome or non-liver-related disorder).

In some embodiments, a method of treating MS, including RMS, NRSPMS, and PPMS, or MG is provided, comprising the steps of performing liver function tests in a patient, detecting suitable liver function, and administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, the liver function tests measure one or more of the levels of aspartate transaminase (AST), alanine transaminase (ALT), albumin, alkaline phosphatase, total and direct bilirubin, and total protein in a patient's blood. In some embodiments a patient having a suitable liver function has one or more of ALT levels of ≤1.5× upper limit of normal (ULN), AST levels of ≤1.5×ULN, and alkaline phosphatase ≤2×ULN (unless caused by non-liver related disorder or explained by a stable chronic liver disorder) and total bilirubin ≤1.5×ULN (unless due to Gilbert syndrome or non-liver-related disorder).

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating MS, including RMS, NRSPMS, and PPMS, or MG, comprising the steps of performing liver function tests in a patient, detecting suitable liver function, and administering a therapeutically acceptable amount of Compound to the patient. In some embodiments, the liver function tests measure one or more of the levels of aspartate transaminase (AST), alanine transaminase (ALT), albumin, alkaline phosphatase, total and direct bilirubin, and total protein in a patient's blood. In some embodiments a patient having a suitable liver function has one or more of ALT levels of ≤1.5× upper limit of normal (ULN), AST levels of ≤1.5×ULN, and alkaline phosphatase ≤2×ULN (unless caused by non-liver related disorder or explained by a stable chronic liver disorder) and total bilirubin ≤1.5×ULN (unless due to Gilbert syndrome or non-liver-related disorder).

In some embodiments, the liver function tests are performed at least about every 6 months, at least about every 5 months, at least about every 4 months, at least about every 3 months, at least about every 2 months, or at least about monthly. In some embodiments, the liver function tests are performed at least about every 12 weeks, at least about every 11 weeks, at least about every 10 weeks, at least about every 9 weeks, at least about every 8 weeks, at least about every 7 weeks, at least about every 6 weeks, at least about every 5 weeks, at least about every 4 weeks, at least about every 3 weeks, at least about every 2 weeks, or at least about weekly.

In some embodiments, a method of treating MS, including RMS, NRSPMS, and PPMS, or MG is provided, comprising the steps of:
a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
b) measuring the level of alanine aminotransferase (ALT) in the patient;
c) detecting a level of ALT of >8× upper limit of normal (ULN);
d) ceasing administration of the Compound to the patient; and optionally
e) monitoring the level of ALT in the patient; and
f) resuming administration of a therapeutically effective amount of the Compound to the patient when the patient's level of ALT is determined to be <1.5×ULN.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating MS, including RMS, NRSPMS, and PPMS, or MG, comprising the steps of:
a) administering a therapeutically effective amount of Compound to a patient in need thereof;
b) measuring the level of alanine aminotransferase (ALT) in the patient;
c) detecting a level of ALT of >8× upper limit of normal (ULN);
d) ceasing administration of the Compound to the patient; and optionally
e) monitoring the level of ALT in the patient; and
f) resuming administration of a therapeutically effective amount of the Compound to the patient when the patient's level of ALT is determined to be <1.5×ULN.

In some embodiments, a method of treating MS, including RMS, NRSPMS, and PPMS, or MG is provided, comprising the steps of:
a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
b) measuring the level of alanine aminotransferase (ALT) in the patient;
c) detecting a level of ALT of >5× upper limit of normal (ULN) during a period of at least two weeks;
d) ceasing administration of the Compound to the patient; and optionally
e) monitoring the level of ALT in the patient; and
f) resuming administration of a therapeutically effective amount of the Compound to the patient when the patient's level of ALT is determined to be <1.5×ULN.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating MS, including RMS, NRSPMS, and PPMS, or MG, comprising the steps of:
a) administering a therapeutically effective amount of Compound to a patient in need thereof;
b) measuring the level of alanine aminotransferase (ALT) in the patient;
c) detecting a level of ALT of >5× upper limit of normal (ULN) during a period of at least two weeks;
d) ceasing administration of the Compound to the patient; and optionally
e) monitoring the level of ALT in the patient; and
f) resuming administration of a therapeutically effective amount of the Compound to the patient when the patient's level of ALT is determined to be <1.5×ULN.

In some embodiments, a method of treating MS, including RMS, NRSPMS, and PPMS, or MG is provided, comprising the steps of.
a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof;
b) measuring the level of alanine aminotransferase (ALT) in the patient;
c) detecting a level of ALT of >3× upper limit of normal (ULN);
d) measuring one or more of total bilirubin and international normalized ratio (INR) in a patient;
e) detecting one or more of total bilirubin >2×ULN and INR >1.5;
f) ceasing administration of the Compound to the patient; and optionally
g) monitoring the level of ALT in the patient; and
h) resuming administration of a therapeutically effective amount of the Compound to the patient when the patient's level of ALT is determined to be <1.5×ULN.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating MS, including RMS, NRSPMS, and PPMS, or MG, comprising the steps of.
a) administering a therapeutically effective amount of Compound to a patient in need thereof;
b) measuring the level of alanine aminotransferase (ALT) in the patient;
c) detecting a level of ALT of >3× upper limit of normal (ULN);
d) measuring one or more of total bilirubin and international normalized ratio (INR) in a patient;
e) detecting one or more of total bilirubin >2×ULN and INR >1.5;
f) ceasing administration of the Compound to the patient; and optionally
g) monitoring the level of ALT in the patient; and
h) resuming administration of a therapeutically effective amount of the Compound to the patient when the patient's level of ALT is determined to be <1.5×ULN.

In some embodiments, a method of treating MS, including RMS, NRSPMS, and PPMS, or MG is provided, comprising the steps of:
a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
b) measuring the level of alanine aminotransferase (ALT) in the patient;
c) detecting a level of ALT of >3× upper limit of normal (ULN);
d) ceasing administration of the Compound to the patient if the patient experiences one or more of fatigue, nausea, vomiting, right upper quadrant pain or tenderness, fever, rash, and eosinophilia >5%; and optionally
e) monitoring the level of ALT in the patient; and
f) resuming administration of a therapeutically effective amount of the Compound to the patient when the patient's level of ALT is determined to be <1.5×ULN.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating MS, including RMS, NRSPMS, and PPMS, or MG, comprising the steps of:
a) administering a therapeutically effective amount of Compound to a patient in need thereof;
b) measuring the level of alanine aminotransferase (ALT) in the patient;
c) detecting a level of ALT of >3× upper limit of normal (ULN);
d) ceasing administration of the Compound to the patient if the patient experiences one or more of fatigue, nausea, vomiting, right upper quadrant pain or tenderness, fever, rash, and eosinophilia >5%; and optionally
e) monitoring the level of ALT in the patient; and
f) resuming administration of a therapeutically effective amount of the Compound to the patient when the patient's level of ALT is determined to be <1.5×ULN.

In some embodiments, the ALT level in a patient is measured at least about every 6 months, at least about every 5 months, at least about every 4 months, at least about every 3 months, at least about every 2 months, or at least about monthly. In some embodiments, the ALT level in a patient is measured at least about every 12 weeks, at least about every 11 weeks, at least about every 10 weeks, at least about every 9 weeks, at least about every 8 weeks, at least about every 7 weeks, at least about every 6 weeks, at least about every 5 weeks, at least about every 4 weeks, at least about every 3 weeks, at least about every 2 weeks, or at least about weekly.

In some embodiments, after ceasing administration of the compound, the ALT level in a patient is monitored about every 2 to 3 days, about every 3 days, about every 2 days, or about daily.

In some embodiments, a method of treating MS, including RMS, NRSPMS, and PPMS, or MG is provided, comprising administering to a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to a patient, wherein the patient is not receiving potent and moderate inducers of cytochrome P450 3A (CYP3A) or potent inhibitors of CYP2C8 hepatic enzymes. In some embodiments the potent CYP3A inducers are selected from rifampin, carbamazepine, phenobarbital, St John's Wort extract, avasimibe, lumacaftor, rifapentine, rifabutin, and phenytoin. In some embodiments the moderate CYP3A inducers are selected from semagacestat, asunaprevir, beclabuvir, daclatasvir, cenobamate, nafcillin, lesinurad, modafinil, bosentan, telotristat ethyl, thioridazine, elagolix and rifabutin. In some embodiments, potent CYP2C8 inhibitors are selected from Gemfibrozil and Clopidogrel.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating MS, including RMS, NRSPMS, and PPMS, or MG, comprising administering to a therapeutically acceptable amount of Compound to a patient, wherein the patient is not receiving potent and moderate inducers of cytochrome P450 3A (CYP3A) or potent inhibitors of CYP2C8 hepatic enzymes. In some embodiments the potent CYP3A inducers are selected from rifampin, carbamazepine, phenobarbital, St John's Wort extract, avasimibe, lumacaftor, rifapentine, rifabutin, and phenytoin. In some embodiments the moderate CYP3A inducers are selected from semagacestat, asunaprevir, beclabuvir, daclatasvir, cenobamate, nafcillin, lesinurad, modafinil, bosentan, telotristat ethyl, thioridazine, elagolix and rifabutin. In some embodiments, potent CYP2C8 inhibitors are selected from Gemfibrozil and Clopidogrel.

In some embodiments, a method of treating MS, including RMS, NRSPMS, and PPMS, or MG is provided, comprising the steps of advising the patient to limit alcohol consumption during treatment, and administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, the patient is female and is advised to limit alcohol consumption to 1 drink per day or less. In some embodiments, 1 drink is approximately 14 grams of alcohol (e.g., 350 mL beer, 140 mL wine, or 40 mL spirits). In some embodiments, the patient is male and is advised to limit alcohol consumption to 2 drinks per day or less. In some embodiments, 2 drinks is approximately 28 grams of alcohol.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating MS, including RMS, NRSPMS, and PPMS, or MG, comprising the steps of advising the patient to limit alcohol consumption during treatment, and administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, the patient is female and is advised to limit alcohol consumption to 1 drink per day or less. In some embodiments, 1 drink is approximately 14 grams of alcohol (e.g., 350 mL beer, 140 mL wine, or 40 mL spirits). In some embodiments, the patient is male and is advised to limit alcohol consumption to 2 drinks per day or less. In some embodiments, 2 drinks is approximately 28 grams of alcohol.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability. Bioavailability of drugs that decompose at stomach pH can be increased by administration of such drugs in a formulation that releases the drug intraduodenally.

The compositions are comprised of in general, the BTK inhibitor compound or a pharmaceutically acceptable salt thereof in combination with a pharmaceutically acceptable excipient such as binders, surfactants, diluents, buffering agents, antiadherents, glidants, hydrophilic or hydrophobic polymers, retardants, stabilizing agents or stabilizers, disintegrants or superdisintegrants, antioxidants, antifoaming agents, fillers, flavors, colors, lubricants, sorbents, preservatives, plasticizers, or sweeteners, or mixtures thereof, which facilitate processing of the BTK inhibitor compound or a pharmaceutically acceptable salt thereof into preparations which can be used pharmaceutically. Any of the well-known techniques and excipients may be used as suitable and as understood in the art, see for example, Remington: The Science and Practice of Pharmacy, Twenty-first Ed., (Pharmaceutical Press, 2005); Liberman, H. A., Lachman, L., and Schwartz, J. B. Eds., Pharmaceutical Dosage Forms, Vol. 1-2 Taylor & Francis 1990; and R. I. Mahato, Ansel's Pharmaceutical Dosage Forms and Drug Delivery Systems, Second Ed. (Taylor & Francis, 2012).

In certain embodiments, the formulations may include one or more pH adjusting agents or buffering agents, for example, acids such as acetic, boric, citric, fumaric, maleic, tartaric, malic, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate, ammonium chloride, and the like. Such buffers used as bases may have other counterions than sodium, for example, potassium, magnesium, calcium, ammonium, or other counterions. Such acids, bases and buffers are included in an amount required to maintain pH of the composition in an acceptable range.

In certain embodiments, the formulations may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite and ammonium sulfate.

In certain embodiments, the formulations may also include one or more antifoaming agents to reduce foaming during processing which can result in coagulation of aqueous dispersions, bubbles in the finished film, or generally impair processing. Exemplary anti-foaming agents include silicon emulsions or sorbitan sesquioleate.

In certain embodiments, the formulations may also include one or more antioxidants, such as non-thiol antioxidants, for example, butylated hydroxytoluene (BHT), sodium ascorbate, ascorbic acid or its derivative, and tocopherol or its derivatives. In certain embodiments, antioxidants enhance chemical stability where required. Other agents such as citric acid or citrate salts or EDTA may also be added to slow oxidation.

In certain embodiments, the formulations may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide, and cetylpyridinium chloride.

In certain embodiments, the formulations may also include one or more binders. Binders impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinyl-pyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum mucilage of isapol husks, polyvinylpyrrolidone (e.g., Polyvidone® CL, Kollidon® CL, Polyplasdone® XL-10), larch arabogalactan, Veegum®, polyethylene glycol, polyethylene oxide, waxes, sodium alginate, and the like.

In certain embodiments, the formulations may also include dispersing agents or viscosity modulating agents. Dispersing agents or viscosity modulating agents include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween®60 or 80, PEG, polyvinylpyrrolidone (PVP; commercially known as Plasdone®), and the carbohydrate-based dispersing agents such as, for example, hydroxypropyl celluloses (e.g., HPC, H-PC-SL, and HPC-L), hydroxypropyl methylcelluloses (e.g., HPMC K100, RPMC K4M, HPMC K15M, and HPMC K100M), carboxymethylcellulose sodium, methylcellulose, hydroxyethyl-cellulose, hydroxypropylcellulose, hydroxypropylmethylcellulose phthalate, hydroxypropyl-methylcellulose acetate stearate (HPMCAS), noncrystalline cellulose, polyethylene oxides, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F10©8, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate. In general, binder levels of about 10 to about 70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 90% and more typically up to 70% in tablet formulations is common.

In certain embodiments, the formulations may also include one or more diluents which refer to chemical compounds that are used to dilute the compound of interest prior to delivery.

Diluents can also be used to stabilize compounds because they can provide a more stable environment Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); hydroxypropyl-methylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In certain embodiments, the formulations may also include one or more disintegrants which includes both the dissolution and dispersion of the dosage form when contacted with gastrointestinal fluid. Disintegration agents or disintegrants facilitate the breakup or disintegration of a substance. Examples of disintegration agents include a starch, e.g., e.g., a natural starch such as corn starch or potato starch, a pregelatinized starch such as National 1551 or sodium starch glycolate such as Promogel® or Explotab®, a cellulose such as a wood product, methylcrystalline cellulose, e.g., e.g., Avicel®, Avicel® PH101, Avicel® PH 102, Avicel® PH105, Elceme® P100, Emcocel®, Vivacel®, and Solka-Floc®, methylcellulose, croscarmellose, or a cross-linked cellulose, such as cross-linked sodium carboxymethyl-cellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In certain embodiments, the formulations may also include erosion facilitators. Erosion facilitators include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

In certain embodiments, the formulations may also include one or more filling agents which include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

In certain embodiments, the formulations may also include one or more flavoring agents or sweeteners, e.g., acacia syrup, acesulfame K, alitame, anise, apple, aspartame, banana, Bavarian cream berry, black currant, butterscotch, calcium citrate, camphor, caramel, cherry, cherry cream chocolate, cinnamon, bubble gum, citrus, citrus punch, citrus cream, cotton candy, cocoa, cola, cool cherry, cool citrus, cyclamate, cyclamate, dextrose, eucalyptus, eugenol, fructose, fruit punch, ginger, glycyrrhizinate, glycyrrhiza (licorice) syrup, grape, grapefruit, honey, isomalt, lemon, lime, lemon cream, monoammonium glyrrhizinate, maltol, mannitol, maple, marshmallow, menthol, mint cream, mixed berry, neohesperidine DC, neotame, orange, pear, peach, peppermint, peppermint cream, Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, xylitol, sucralose, sorbitol, Swiss cream, tagatose, tangerine, thaumatin, tutti frutti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

In certain embodiments, the formulations may also include one or more lubricants and glidants which are compounds that prevent, reduce or inhibit adhesion or friction of materials.

Exemplary lubricants include, e.g., stearic acid, calcium hydroxide, talc, sodium stearyl lumerate, a hydrocarbon such as mineral oil, or hydrogenated vegetable oil such as hydrogenated soybean oil, higher fatty acids and their alkali-metal and alkaline earth metal salts, such as aluminum, calcium, magnesium, zinc, stearic acid, sodium stearates, glycerol, talc, waxes, boric acid, sodium benzoate, sodium acetate, sodium chloride, leucine, a polyethylene glycol (e.g., PEG4000) or a methoxypolyethylene glycol such as Carbowax®, sodium oleate, sodium benzoate, glyceryl behenate, polyethylene glycol, magnesium or sodium lauryl sulfate, colloidal silica such as Syloid®, Cab-O-Sil®, a starch such as corn starch, silicone oil, a surfactant, and the like.

In certain embodiments, the formulations may also include one or more plasticizers which are compounds used to soften the enteric or delayed release coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl citrate, dibutyl sebacate, triethyl cellulose and triacetin. In some embodiments, plasticizers can also function as dispersing agents or wetting agents.

In certain embodiments, the formulations may also include one or more solubilizers which include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins for example Captisol®, ethanol, n-butanol, isopropyl alcohol, cholesterol, bile salts, polyethylene glycol 200-600, glycofurol, transcutol, propylene glycol, and dimethyl isosorbide and the like. In one embodiment, the solubilizer is vitamin E TPGS or Captisol® or 8-hydroxypropylcyclodextrin.

In certain embodiments, the formulations may also include one or more suspending agents which include compounds such as polyvinylpyrrolidone, e.g., polyvinylpyrrolidone K112, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monoleate, povidone and the like.

In certain embodiments, the formulations may also include one or more surfactants which include compounds such as sodium lauryl sulfate, sodium docusate, Tween 20, 60 or 80, triacetin, vitamin E TPGS, sorbitan monooleate, polyoxyethylene sorbitan monooleate, polyoxyethylene sorbitan monolaurate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

In certain embodiments, the formulations may also include one or more viscosity enhancing agents which include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol alginates, acacia, chitosans and combinations thereof.

In certain embodiments, the formulations may also include one or more wetting agents which include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

Pharmaceutical preparations disclosed herein can be obtained by mixing one or more solid excipient such as carrier, binder, filling agent, suspending agent, flavoring agent, sweetening agent, disintegrating agent, dispersing agent, surfactant, lubricant, colorant diluent, solubilizer, moistening agent, plasticizer, stabilizer, penetration enhancer, wetting agent, anti-foaming agent, antioxidant, preservative, or one or more combination thereof with one or more of the compounds described herein, optionally grinding the resulting mixture, and processing the mixture of granules, after adding suitable excipients, if desired, to obtain tablets.

Pharmaceutical preparations disclosed herein also include capsules made of gelatin, as well as soft, sealed capsules made of gelatin and a plasticizer, such as glycerol or sorbitol. Capsules may also be made of polymers such as hypromellose. The capsules can contain the active ingredients in admixture with filler such as lactose, binders such as starches, or lubricants such as talc or magnesium stearate and, optionally, stabilizers. In soft capsules, the active compounds may be dissolved or suspended in suitable liquids, such as fatty oils, liquid paraffin, lipids, solubilizers, or liquid polyethylene glycols. In addition, stabilizers may be added. All formulations for oral administration should be in dosages suitable for such administration.

These formulations can be manufactured by conventional pharmacological techniques. Conventional pharmacological techniques include, e.g., one or a combination of methods: (1) dry mixing, (2) direct compression, (3) milling, (4) dry or non-aqueous granulation, (5) wet granulation, (6) fusion, or (7) extrusion. See, e.g., Lachman et al., The Theory and Practice of Industrial Pharmacy, 3^{rd} ed. (1986). Other methods include, e.g., spray drying, pan coating, melt granulation, granulation, fluidized bed spray drying or coating (e.g., wurster coating), tangential coating, top spraying, tableting, extruding, extrusion/spheronization, and the like.

It should be appreciated that there is considerable overlap between excipients used in the solid dosage forms described herein. Thus, the above-listed additives should be taken as merely exemplary, and not limiting, of the types of excipients that can be included in solid dosage forms described herein. The type and amounts of such excipient can be readily determined by one skilled in the art, according to the particular properties desired.

In some embodiments, the solid dosage forms described herein are enteric coated oral dosage forms, i.e., as an oral dosage form of a pharmaceutical composition as described herein which utilizes an enteric coating to affect the release of the compound in the intestine of the gastrointestinal tract. An “enterically coated” drug or tablet refers to a drug or tablet that is coated with a substance that remains intact in the stomach but dissolves and releases the drug once the intestine (in one embodiment small intestine) is reached. As used herein “enteric coating”, is a material, such as a polymer material or materials which encase the therapeutically active agent core either as a dosage form or as particles. Typically, a substantial amount or all of the enteric coating material is dissolved before the therapeutically active agent is released from the dosage form, so as to achieve delayed dissolution of the therapeutically active agent core or particles in the small or large intestine. Enteric coatings are discussed, for example, Loyd, V. Allen, Remington: The Science and Practice of Pharmacy, Twenty-first Ed., (Pharmaceutical Press, 2005; and P. J. Tarcha, Polymers for Controlled Drug Delivery, Chapter 3, CRC Press, 1991. Methods for applying enteric coatings to pharmaceutical compositions are well known in the art, and include for example, U.S. Patent Publication No. 2006/0045822.

The enteric coated dosage form may be a compressed or molded or extruded tablet (coated or uncoated) containing granules, powder, pellets, beads, or particles of the BTK inhibitor compound or a pharmaceutically acceptable salt thereof or other excipients, which are themselves coated or uncoated provided at least the tablet or the BTK inhibitor compound is coated. The enteric coated oral dosage form may also be a capsule (coated or uncoated) containing pellets, beads, or granules of the BTK inhibitor compound or a pharmaceutically acceptable salt thereof or other excipients, which are themselves coated or uncoated provided at least one of them is coated. Some examples of coatings that were originally used as enteric coatings are beeswax and glyceryl monostearate; beeswax, shellac, and cellulose; and cetyl alcohol, mastic, and shellac as well as shellac and stearic acid (U.S. Pat. No. 2,809,918); polyvinylacetate and ethyl cellulose (U.S. Pat. No. 3,835,221). More recently, the coatings used are neutral copolymers of polymethacrylic acid esters (Eudragit L3OD). (F. W. Goodhart et al, Pharm. Tech., p. 64-71, April 1984); copolymers of methacrylic acid and methacrylic acid methyl ester (Eudragit S), or a neutral copolymer of polymethacrylic acid esters containing metallic stearates (Mehta et al U.S. Pat. Nos. 4,728,512 and 4,794,001), cellulose acetate succinate, and hypromellose phthalate.

Any anionic polymer exhibiting a pH-dependent solubility profile can be used as an enteric coating in the methods and compositions described herein to achieve delivery to the intestine. In one embodiment, delivery can be to the small intestine. In another embodiment, delivery can be to the duodenum. In some embodiments the polymers described herein are anionic carboxylic polymers. In other embodiments, the polymers, and compatible mixtures thereof, and some of their properties, include, but are not limited to the following.

Shellac: Also called purified lac, it is a refined product obtained from the resinous secretion of an insect. This coating dissolves in media of pH>7.

Acrylic polymers: The performance of acrylic polymers (primarily their solubility in biological fluids) can vary based on the degree and type of substitution. Examples of suitable acrylic polymers include methacrylic acid copolymers and ammonium methacrylate copolymers. The Eudragit series L, S, and RS (manufactured Rohm Pharma and known as Evonik®) are available as solubilized in organic solvent, aqueous dispersion, or dry powders. The Eudragit series RL, NE, and RS are insoluble in the gastrointestinal tract but are permeable and are used primarily for colonic targeting. The Eudragit series L, L-30D and S are insoluble in stomach and dissolve in the intestine and may be selected and formulated to dissolve at a value of pH greater than 5.5 or as low as greater than 5 or as high as greater than 7.

Cellulose Derivatives: Examples of suitable cellulose derivatives are ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP pseudolatex with particles <1 μm. Other components in Aquateric can include pluronics, Tweens, and acetylated monoglycerides. Other suitable cellulose derivatives include cellulose acetate tritnellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (HPMCAS e.g., AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions.

Poly Vinyl Acetate Phthalate (PVAP): PVAP dissolves in pH>5, and it is much less permeable to water vapor and gastric fluids. Detailed description of above polymers and their pH-dependent solubility can be found at in the article titled “Enteric coated hard gelatin capsules” by Professor Karl Thoma and Karoline Bechtold at http://pop.www.capsugel.com/media/library/enteric-coated-hard-gelatin-capsules.pdf. In some embodiments, the coating can, and usually does, contain a plasticizer and possibly other coating excipients such as colorants, talc, or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually contain 10-25% by weight of a plasticizer, especially dibutyl phthalate, polyethylene glycol, triethyl citrate and triacetin. Conventional coating techniques such as fluid bed or Wurster coaters, or spray or pan coating are employed to apply coatings. The coating thickness must be sufficient to ensure that the oral dosage form remains intact until the desired site of topical delivery in the intestinal tract is reached.

Colorants, surfactants, anti-adhesion agents, antifoaming agents, lubricants (e.g., carnauba wax or PEG) and other additives may be added to the coatings besides plasticizers to solubilize or disperse the coating material, and to improve coating performance and the coated product.

To accelerate the dissolution of the enteric coat, a half-thickness, double coat of enteric polymer (for instance, Eudragit L30 D-55) may be applied, and the inner enteric coat may have a buffer up to pH 6.0 in the presence of 10% citric acid, followed by a final layer of standard Eudragit L 30 D-55. Applying two layers of enteric coat, each half the thickness of a typical enteric coat, Liu and Basit were able to accelerate enteric coating dissolution compared to a similar coating system applied, unbuffered, as a single layer (Liu, F. and Basit, A. Journal of Controlled Release. 147 (2010) 242-245.)

The intactness of the enteric coating may be measured, for example, by the degradation of the drug within the micropellets. The enteric coated dosage forms or pellets may be tested in dissolution testing first in gastric fluid and separately in intestinal fluid as described in USP to determine its function.

The enteric coated tablets and capsules formulation containing the disclosed compounds can be made by methods well known in the art. For example, tablets containing a compound disclosed herein can be enterically coated with a coating solution containing Eudragit®, diethylphthalate, isopropyl alcohol, talc, and water using a side vented coating pan (Freund Hi-Coater).

Alternatively, a multi-unit dosage form comprising enteric-coated pellets that can be incorporated into a tablet or into a capsule can be prepared as follows.

Core material: The core material for the individually enteric coating layered pellets can be constituted according to different principles. Seeds layered with the active agent (i.e., the BTK inhibitor compound or a pharmaceutically acceptable sale thereof), optionally mixed with alkaline substances or buffer, can be used as the core material for the further processing. The seeds which are to be layered with the active agent can be water insoluble seeds comprising different oxides, celluloses, organic polymers, and other materials, alone or in mixtures or water-soluble seeds comprising different inorganic salts, sugars, non-pareils and other materials, alone or in mixtures.

Further, the seeds may comprise the active agent in the form of crystals, agglomerates, compacts etc. The size of the seeds is not essential for the present disclosure but may range in size approximately from 0.1 to 2 mm. The seeds layered with the active agent are produced either by powder or solution/suspension layering using for instance granulation or spray coating layering equipment. Before the seeds are layered, active agent may be mixed with further components.

Such components can be binders, surfactants, fillers, disintegrating agents, alkaline additives or other or pharmaceutically acceptable ingredients alone or in mixtures. The binders are for example polymers such as hydroxypropyl methylcellulose (HPMC), hydroxypropylcellulose (HPC), carboxymethylcellulose sodium, polyvinyl pyrrolidone (PVP), or sugars, starches, or other pharmaceutically acceptable substances with cohesive properties. Suitable surfactants are found in the groups of pharmaceutically acceptable non-ionic or ionic surfactants such as for instance sodium lauryl sulfate.

Alternatively, the active agent optionally mixed with suitable constituents can be formulated into a core material. Said core material may be produced by extrusion/spheronization, balling or compression utilizing conventional process equipment. The size of the formulated core material is approximately from 0.1 to 4 mm, and for example, from 0.1 to 2 mm. The manufactured core material can further be layered with additional ingredients comprising the active agent or be used for further processing.

The active agent is mixed with pharmaceutical constituents to obtain preferred handling and processing properties and a suitable concentration of the active agent in the final preparation. Pharmaceutical constituents such as fillers, binders, lubricants, disintegrating agents, surfactants, and other pharmaceutically acceptable additives may be used.

Alternatively, the aforementioned core material can be prepared by using spray drying or spray congealing technique.

Enteric Coating Layer(s): Before applying the enteric coating layer(s) onto the core material in the form of individual pellets, the pellets may optionally be covered with one or more separating layer(s) comprising pharmaceutical excipients optionally including alkaline compounds such as pH-buffering compounds. This/these separating layer(s), separate(s) the core material from the outer layers being enteric coating layer(s). This/these separating layer(s) protecting the core material of active agent should be water soluble or rapidly disintegrating in water.

A separating layer(s) can be optionally applied to the core material by coating or layering procedures in suitable equipment such as coating pan, coating granulator or in a fluidized bed apparatus using water or organic solvents for the coating process. As an alternative the separating layer(s) can be applied to the core material by using powder coating technique. The materials for the separating layers are pharmaceutically acceptable compounds such as, for instance, sugar, polyethylene glycol, polyvinylpyrrolidone, polyvinyl alcohol, polyvinyl acetate, hydroxypropyl cellulose, methylcellulose, ethylcellulose, hydroxypropyl methyl cellulose, carboxymethylcellulose sodium, water soluble salts of enteric coating polymers and others, used alone or in mixtures. Additives such as plasticizers, colorants, pigments, fillers anti-tacking and anti-static agents, such as for instance magnesium stearate, titanium dioxide, talc and other additives may also be included into the separating layer(s).

When the optional separating layer is applied to the core material it may constitute a variable thickness. The maximum thickness of the separating layer(s) is normally only limited by processing conditions. The separating layer may serve as a diffusion barrier and may act as a pH-buffering zone. The optionally applied separating layer(s) is not essential for the embodiments of the present disclosure. However, the separating layer(s) may improve the chemical stability of the active substance or the physical properties of the novel multiple unit tableted dosage form.

Alternatively, the separating layer may be formed in situ by a reaction between an enteric coating polymer layer applied on the core material and an alkaline reacting compound in the core material. Thus, the separating layer formed comprises a water-soluble salt formed between the enteric coating layer polymer(s) and an alkaline reacting compound which is in the position to form a salt.

One or more enteric coating layers are applied onto the core material or onto the core material covered with separating layer(s) by using a suitable coating technique. The enteric coating layer material may be dispersed or dissolved in either water or in suitable organic solvents. As enteric coating layer polymers, one or more, separately or in combination, of the following can be used, e.g., solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating polymer(s).

The enteric coating layers contain pharmaceutically acceptable plasticizers to obtain the desired mechanical properties, such as flexibility and hardness of the enteric coating layers. Such plasticizers are for instance, but not restricted to triacetin, citric acid esters, phthalic acid esters, dibutyl sebacate, cetyl alcohol, polyethylene glycols, polysorbates or other plasticizers.

The amount of plasticizer is optimized for each enteric coating layer formula, in relation to the selected enteric coating layer polymer(s), selected plasticizer(s) and the applied amount of said polymer(s), in such a way that the mechanical properties, i.e., flexibility and hardness of the enteric coating layer(s), for instance exemplified as Vickers hardness, are adjusted so that if a tablet is desired the acid resistance of the pellets covered with enteric coating layer(s) does not decrease significantly during compression of pellets into tablets. The amount of plasticizer is usually above 5% by weight of the enteric coating layer polymer(s), such as 15-50% and further such as 20-50%. Additives such as dispersants, colorants, pigments polymers e.g., poly(ethylacrylate, methylmethacrylate), anti-tacking and anti-foaming agents may also be included into the enteric coating layer(s). Other compounds may be added to increase film thickness and to decrease diffusion of acidic gastric juices into the acid susceptible material. The maximum thickness of the applied enteric coating is normally only limited by processing conditions and the desired dissolution profile.

Over-Coating Layer: Pellets covered with enteric coating layer(s) may optionally further be covered with one or more over-coating layer(s). The over-coating layer(s) should be water soluble or rapidly disintegrating in water. The over-coating layer(s) can be applied to the enteric coating layered pellets by coating or layering procedures in suitable equipment such as coating pan, coating granulator or in a fluidized bed apparatus using water or organic solvents for the coating or layering process. The materials for over-coating layers are chosen among pharmaceutically acceptable compounds such as, for instance sugar, polyethylene glycol, polyvinylpyrrolidone, polyvinyl alcohol, polyvinyl acetate, hydroxypropyl cellulose, methylcellulose, ethylcellulose, hydroxypropyl methyl cellulose, carboxymethylcellulose sodium and others, used alone or in mixtures. Additives such as plasticizers, colorants, pigments, fillers, anti-tacking, and anti-static agents, such for instance magnesium stearate, titanium dioxide, talc and other additives may also be included into the over-coating layer(s). The over-coating layer may further prevent potential agglomeration of enteric coating layered pellets, further it may protect the enteric coating layer towards cracking during the compaction process and enhance the tableting process. The maximum thickness of the applied over-coating layer(s) is normally limited by processing conditions and the desired dissolution profile. The over-coating layer may also be used as a tablet film coating layer.

Enteric coating of soft gelatin capsules may contain an emulsion, oil, microemulsion, self-emulsifying system, lipid, triglycerides, polyethylene glycol, surfactants, other solubilizers and the like, and combinations thereof, to solubilize the active agent. The flexibility of the soft gelatin capsule is maintained by residual water and plasticizer. Moreover, for gelatin capsules the gelatin may be dissolved in water so that spraying must be accomplished at a rate with relatively low relative humidity such as can be accomplished in a fluid bed or Wurster. In addition, drying should be accomplished without removing the residual water or plasticizer causing cracking of the capsule shell. Commercially available blends optimized for enteric coating of soft gelatin capsules such as Instamodel EPD (Enteric Polymeric Dispersion), available from Ideal Cures, Pvt. Ltd. (Mumbai, India). On a laboratory scale enteric coated capsules may be prepared by: a) rotating capsules in a flask or dipping capsules in a solution of the gently heated enteric coating material with plasticizer at the lowest possible temperature or b) in a lab scale sprayer/fluid bed and then drying.

For aqueous active agents, it can be especially desirable to incorporate the drug in the water phase of an emulsion. Such “water-in-oil” emulsion provides a suitable biophysical environment for the drug and can provide an oil-water interface that can protect the drug from adverse effects of pH or enzymes that can degrade the drug. Additionally, such water-in-oil formulations can provide a lipid layer, which can interact favorably with lipids in cells of the body, and can increase the partition of the formulation onto the membranes of cells. Such partition can increase the absorption of drugs in such formulations into the circulation and therefore can increase the bioavailability of the drug.

In some embodiments the water-in-oil emulsion contains an oily phase composed of medium or long chain carboxylic acids or esters or alcohols thereof, a surfactant or a surface-active agent, and an aqueous phase containing primarily water and the active agent.

Medium and long chain carboxylic acids are those ranging from C8 to C22 with up to three unsaturated bonds (also branching). Examples of saturated straight chain acids are n-dodecanoic acid, n-tetradecanoic acid, n-hexadecanoic acid, caproic acid, caprylic acid, capric acid, lauric acid, myristic acid, palmitic acid, stearic acid, arachidic acid, behenic acid, montanic acid and melissic acid. Also useful are unsaturated monoolefinic straight chain monocarboxylic acids.

Examples of these are oleic acid, gadoleic acid and erucic acid. Also useful are unsaturated (polyolefinic) straight chain monocarboxylic acids. Examples of these are linoleic acid, ricinoleic acid, linolenic acid, arachidonic acid and behenolic acid. Useful branched acids include, for example, diacetyl tartaric acid. Unsaturated olefinic chains may also be hydroxylated or ethoxylated to prevent oxidation or to alter the surface properties.

Examples of long chain carboxylic acid esters include, but are not limited to, those from the group of: glyceryl monostearates; glyceryl monopalmitates; mixtures of glyceryl monostearate and glyceryl monopalmitate; glyceryl monolinoleate; glyceryl monooleate; mixtures of glyceryl monopalmitate, glyceryl monostearate, glyceryl monooleate and glyceryl monolinoleate; glyceryl monolinolenate; glyceryl monogadoleate; mixtures of glyceryl monopalmitate, glyceryl monostearate, glyceryl monooleate, glyceryl monolinoleate, glyceryl monolinolenate and glyceryl monogadoleate; acetylated glycerides such as distilled acetylated monoglycerides; mixtures of propylene glycol monoesters, distilled monoglycerides, sodium steroyl lactylate and silicon dioxide; d-alpha tocopherol polyethylene glycol 1000 succinate; mixtures of mono- and di-glyceride esters such as Atmul; calcium stearoyl lactylate; ethoxylated mono- and di-glycerides; lactated mono- and di-glycerides; lactylate carboxylic acid ester of glycerol and propylene glycol; lactylic esters of long chain carboxylic acids; polyglycerol esters of long chain carboxylic acids, propylene glycol mono- and di-esters of long chain carboxylic acids; sodium stearoyl lactylate; sorbitan monostearate; sorbitan monooleate; other sorbitan esters of long chain carboxylic acids; succinylated monoglycerides; stearyl monoglyceryl citrate; stearyl heptanoate; cetyl esters of waxes; stearyl octanoate; C8-C30 cholesterol/lavosterol esters; and sucrose long chain carboxylic acid esters.

Examples of the self-emulsifying long chain carboxylic acid esters include those from the groups of stearates, palmitates, ricinoleates, oleates, behenates, ricinolenates, myristates, laurates, caprylates, and caproates. In some embodiments the oily phase may comprise a combination of 2 or more of the long chain carboxylic acids or esters or alcohols thereof. In some embodiments medium chain surfactants may be used and the oil phase may comprise a mixture of caprylic/capric triglyceride and C8/C10 mono-/di-glycerides of caprylic acid, glyceryl caprylate or propylene glycol monocaprylate or their mixtures.

The alcohols that can be used are exemplified by the hydroxyl forms of the carboxylic acids exemplified above and also stearyl alcohol.

Surface active agents or surfactants are long chain molecules that can accumulate at hydrophilic/hydrophobic (water/oil) interfaces and lower the surface tension at the interface. As a result, they can stabilize an emulsion. In some embodiments, the surfactant may comprise: Tween® (polyoxyethylene sorbate) family of surfactants, Span® (sorbitan long chain carboxylic acid esters) family of surfactants, Pluronic® (ethylene or propylene oxide block copolymers) family of surfactants, Labrasol®, Labrafil® and Labrafac® (each polyglycolyzed glycerides) families of surfactants, sorbitan esters of oleate, stearate, laurate or other long chain carboxylic acids, poloxamers (polyethylene-polypropylene glycol block copolymers or Pluronic®), other sorbitan or sucrose long chain carboxylic acid esters, mono and diglycerides, PEG derivatives of caprylic/capric triglycerides and mixtures thereof or mixture of two or more of the above. In some embodiments the surfactant phase may comprise a mixture of Polyoxyethylene (20) sorbitan monooleate (Tween 80®) and sorbitan monooleate (Span 80®).

The aqueous phase may optionally comprise the active agent suspended in water and a buffer.

In some embodiments, such emulsions are coarse emulsions, microemulsions and liquid crystal emulsions. In other embodiments such emulsion may optionally comprise a permeation enhancer. In other embodiments, spray-dried dispersions or microparticles or nanoparticles containing encapsulated microemulsion, coarse emulsion or liquid crystal can be used.

In some embodiments, the solid dosage forms described herein are non-enteric time-delayed release dosage forms. The term “non-enteric time-delayed release” as used herein refers to the delivery so that the release of the drug can be accomplished at some generally predictable location in the intestinal tract more distal to that which would have been accomplished if there had been no delayed release alterations. In some embodiments the method for delay of release is a coating that becomes permeable, dissolves, ruptures, or is no longer intact after a designed duration. The coating in the time-delayed release dosage forms can have a fixed time to erode after which the drug is released (suitable coating include polymeric coating such as HPMC, PEO, and the like) or has a core comprised of a superdisintegrant(s) or osmotic agent(s) or water attractant such as a salt, hydrophilic polymer, typically polyethylene oxide or an alkylcellulose, salts such as sodium chloride, magnesium chloride, sodium acetate, sodium citrate, sugar, such as glucose, lactose, or sucrose, or the like, which draw water through a semi-permeable membrane or a gas generating agent such as citric acid and sodium bicarbonate with or without an acid such as citric acid or any of the aforementioned acids incorporated in dosage forms. The semi-permeable membrane, while mostly not permeable to the drug nor the osmotic agent, is permeable to water that permeates at a near constant rate to enter the dosage form to increase the pressure and ruptures after the swelling pressure exceeds a certain threshold over a desired delay time. The permeability through this membrane of the drug should be less than 1/10 than water and in one embodiment less than 1/100 the water permeability. Alternatively, a membrane could become porous by leaching an aqueous extractable over a desired delay time.

Osmotic dosage forms have been described in Theeuwes U.S. Pat. No. 3,760,984, and an osmotic bursting dosage form is described in Baker U.S. Pat. No. 3,952,741. This osmotic bursting dosage form can provide a single pulse of release or multiple pulses if different devices with different timings are employed. The timing of the osmotic burst may be controlled by the choice of polymer and the thickness or the area of the semipermeable membrane surrounding the core that contains both the drug and the osmotic agent or attractant. As the pressure in the dosage form increase with additional permeated water, the membrane elongates until its breaking point, and then the drug is released. Alternatively, specific areas of rupture can be created in the membrane by having a thinner, weaker area in the membrane or by adding a weaker material to an area of the coating membrane. Some preferred polymers with high water permeabilities that may be used as semipermeable membranes are cellulose acetate, cellulose acetate butyrate, cellulose nitrate, crosslinked polyvinyl, alcohol, polyurethanes, nylon 6, nylon 6.6, and aromatic nylon. Cellulose acetate is an especially preferred polymer.

In another embodiment, the time-delayed coating that begins its delay to releasing drug after the enteric coating is at least partially dissolved is comprised of hydrophilic, erodible polymers that upon contact with water begin to gradually erode over time. Examples of such polymers include cellulose polymers and their derivatives including, but not limited to, hydroxyalkyl celluloses, hydroxymethyl cellulose, hydroxyethyl cellulose, hydroxypropyl cellulose, hydroxypropylmethyl cellulose, carboxymethylcellulose, microcrystalline cellulose; polysaccharides and their derivatives; polyalkylene oxides, such as polyethylene oxide or polyethylene glycols, particularly high molecular weight polyethylene glycols; chitosan; poly(vinyl alcohol); xanthan gum; maleic anhydride copolymers; poly(vinyl pyrrolidone); starch and starch-based polymers; maltodextrins; poly (2-ethyl-2-oxazoline); poly(ethyleneimine); polyurethane; hydrogels; crosslinked polyacrylic acids; and combinations or blends of any of the foregoing.

Some preferred erodible hydrophilic polymers suitable for forming the erodible coating are poly(ethylene oxide), hydroxypropyl methyl cellulose, and combinations of poly(ethylene oxide) and hydroxypropyl methyl cellulose. Poly(ethylene oxide) is used herein to refer to a linear polymer of unsubstituted ethylene oxide. The molecular weight of the poly(ethylene oxide) polymers can range from about 10⁵ Daltons to about 10⁷ Daltons. A preferred molecular weight range of poly(ethylene oxide) polymers is from about 2×10⁵ to 2×10⁶ Daltons and is commercially available from The Dow Chemical Company (Midland, Mich.) referred to as SENTRYR POLYOX™ water-soluble resins, NF (National Formulary) grade. When higher molecular weights of polyethylene oxide are used, other hydrophilic agents, such as salts or sugars, like glucose, sucrose, or lactose, that promote erosion or disintegration of this coating, are also included.

The time-delayed dosage form can be a mechanical pill such as an Enterion® capsule or pH sensitive capsule which can release the drug after a pre-programmed time or when it receives a signal which can be transmitted or once it leaves the stomach.

The amount of the compound of the disclosure in a formulation can vary within the full range employed by those skilled in the art. Typically, the formulation will contain, on a weight percent (wt %) basis, from about 0.01-99.99 wt % of the BTK inhibitor compound based on the total formulation, with the balance being one or more suitable pharmaceutical excipients. In one embodiment, the compound is present at a level of about 1-80 wt %.

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled. Additionally, the attached examples provide exemplary study protocols showing how a clinical study may be implemented.

### EXAMPLES

The following examples are provided to illustrate certain disclosed embodiments and are not to be construed as limiting the scope of this disclosure in any way. In the Examples discussed below, the BTK inhibitor, as defined above, may be also referred as “the compound” or “the drug” interchangeably.

### Example 1—A Phase 3, Randomized, Double-Blind, Placebo-Controlled, Parallel-Group Study to Evaluate the Efficacy and Safety of Tolebrutinib (SAR442168) in Adults with Generalized Myasthenia Gravis

The purpose of this study is to evaluate the efficacy and safety of tolebrutinib 60 mg daily compared with placebo in adult participants with moderate-to-severe generalized myasthenia gravis (gMG) receiving the SoC.

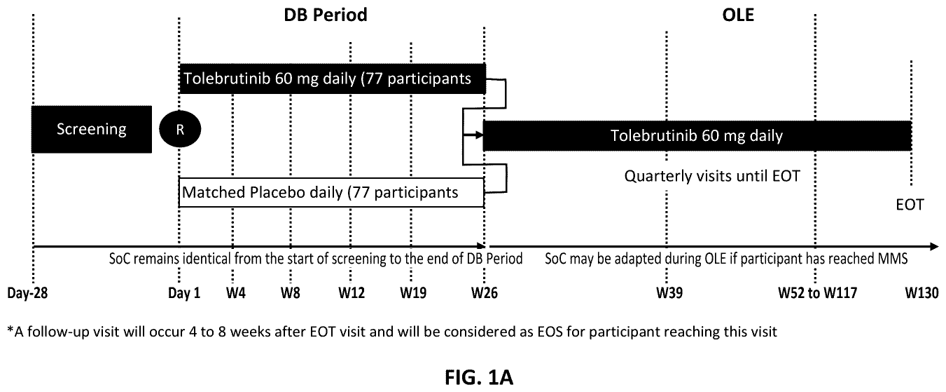

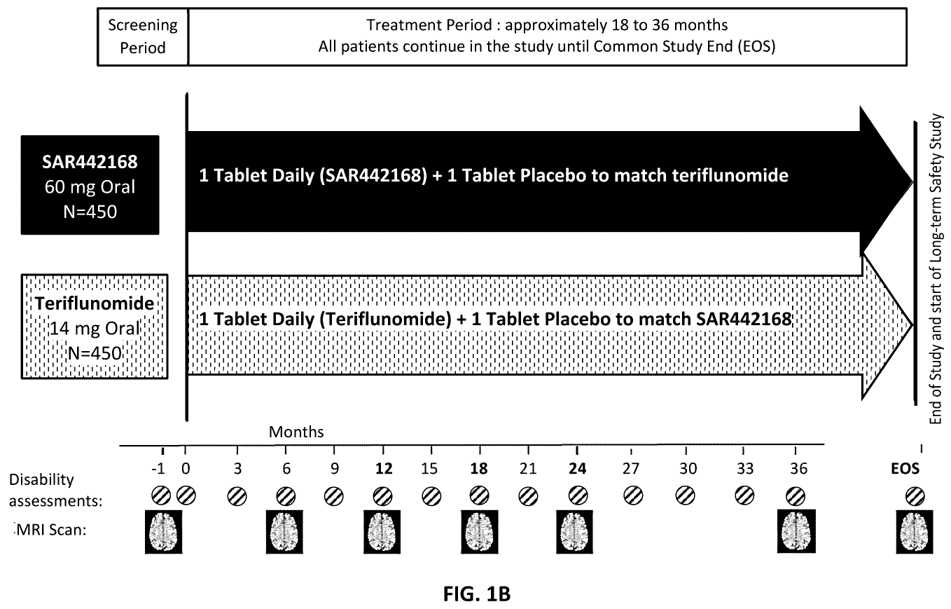

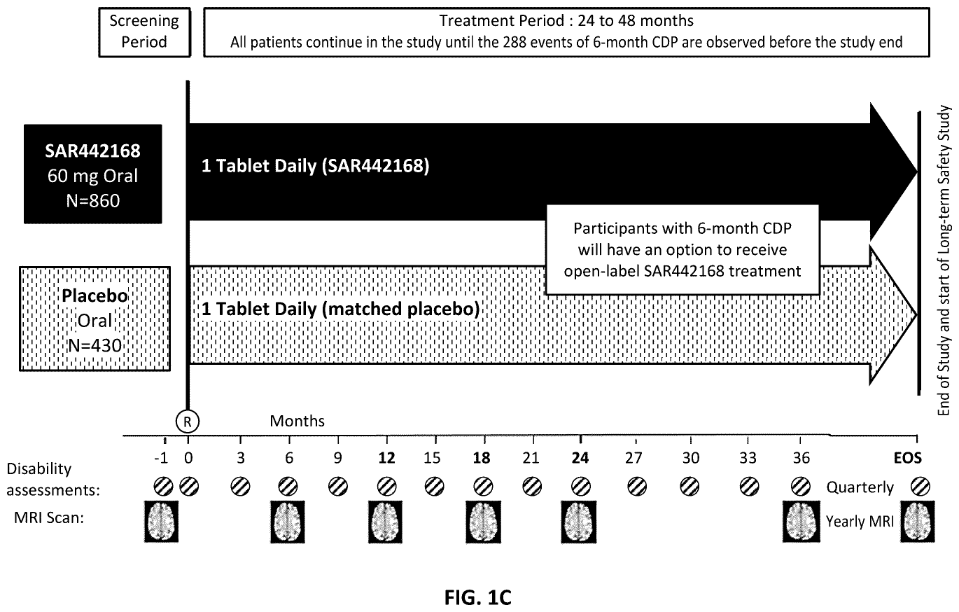

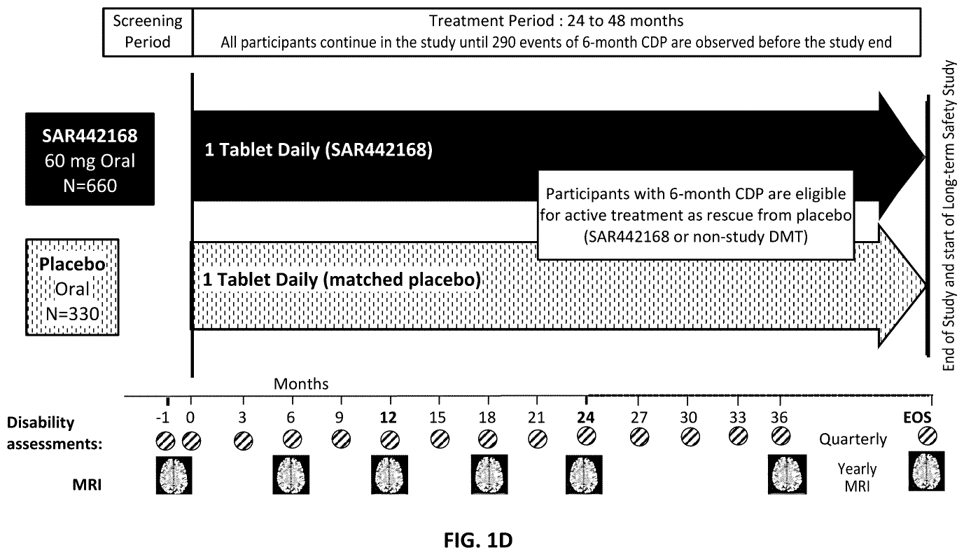

A graphical scheme of the study design is shown in FIG. 1A. Abbreviations used in FIG. 1A: DB: double-blind; EOT: end of treatment; MMS: minimal manifestation status; OLE: open-label extension; R: randomization; SoC: standard of care; W: week. Tables 1A1 and 1A2 shown below describe the schedule of activities during the course of study. Table 1B that follows describes the objective and endpoints of the overall study.

TABLE 1A1
Schedule of Activities (SOA)- Screening and DB Period

| | Screening | | | | | | | | |
|---|---|---|---|---|---|---|---|---|---|
| | (up to 28 days | DB period^{b} | | | | | | | |
| | before D 1)^{a} | D 1 | W 4 | W 6 | W 8 | W 10 | W 12 | W 19 | W 26^{c} |
| | Visit (a window of ±7 days is allowed for all visits after D 1) | | | | | | | | |
| | V1 | 2 | 3 | | 4 | | 5 | 6 | 7 |
| | Place of the visit (S site; H: Home)^{g} | | | | | | | | |
| Procedure | S | S | S | S | S | S | S | H | S |
| Informed consent | X | | | | | | | | |
| Inclusion and exclusion | X | X | | | | | | | |
| criteria | | | | | | | | | |
| Demography | X | | | | | | | | |
| Medical and surgical | X | | | | | | | | |
| history | | | | | | | | | |
| Prior/concomitant | <=====================================================================> | | | | | | | | |
| medications^{h} | | | | | | | | | |
| Randomization | | X | | | | | | | |
| IRT contact | X | X | | | | | X | | X |
| IMP dispensation | | X | | | | | X | | Xⁱ |
| IMP compliance | | | | | | | X | | X |
| Diary dispensation and | | X | | | | | X | | X |
| collection^{j} | | | | | | | | | |

Safety

| | | | | | | | | | |
|---|---|---|---|---|---|---|---|---|---|
| Physical examination^{k} | X | X | | | | | X | | X |
| Vital signs^{l} | X | X | X | | X | | X | | X |
| Body height | X | | | | | | | | |
| Body weight | X | X | | | | | X | | X |
| Pregnancy test | X | X | X | | X | | X | | X |
| (WOCBP only) ^{m} | | | | | | | | | |
| Serum FSHⁿ | X | | | | | | | | |
| HIV, hepatitis B and C | X | | | | | | | | |
| screening | | | | | | | | | |
| Tuberculosis testing^{o} | X | | | | | | | | |
| Laboratory tests (include | X | X | X | | X | | X | X | X |
| hematology and clinical | | | | | | | | | |
| chemistry)^{p} | | | | | | | | | |
| Liver function tests^{q} | | | | X | | X | | | |
| Iron panel (serum): iron, | X | | | | | | | | |
| ferritin, transferrin | | | | | | | | | |
| saturation, TIBC; to be | | | | | | | | | |
| repeated during the study, | | | | | | | | | |
| if needed | | | | | | | | | |
| Urinalysis | X | | | | | | | | |
| Coagulation: PT/INR, | X | | | | | | | | |
| aPTT (to be repeated | | | | | | | | | |
| during the study, if | | | | | | | | | |
| needed) | | | | | | | | | |
| 12-lead ECG | X | | X | | | | X | | |
| Suicidality assessment by | X | X | | | | | X | | |
| C-SSRS | | | | | | | | | |

| | |
|---|---|
| AE/SAE review | <=====================================================================> |

Efficacy^{t}

| | | | | | |
|---|---|---|---|---|---|
| MG-ADL | X | X | X | X | X |
| QMG | | X | X | X | X |
| MGII | | X | X | X | X |
| MG-QoL15 | | X | | | |
| HCRU-MG | | X | | | |
| MGFA-PIS | | X | | | |
| EQ-5D-5L | | X | X | X | X |
| PGIS | | X | X | X | X |

Pharmacokinetics

| | | |
|---|---|---|
| Plasma samples for | X^{v} | X^{w} |
| tolebrutinib and active M2 | | |
| metabolite^{u} | | |

Pharmacogenetics

| | |
|---|---|
| Archival DNA sample^{x} | X |

Pharmacodyamics/Biomarkers

| | | |
|---|---|---|
| Blood sample for | X | |
| archiving^{y} | | |
| Exploratory biomarkers: | X | |
| serum auto-antibodies | | |
| (anti-AChR and/or anti- | | |
| MuSK) | | |
| Exploratory biomarkers: | | X |
| serum IgM and IgG | | |

| | | |
|---|---|---|
| Digital tool for activity/ | X | <================================================> |
| function^{z} | | |

TABLE 1A2
Schedule of Activities (SOA)- OLE to Follow Up

| | OLE^{b} | | | | |
|---|---|---|---|---|---|
| | | Quarterly visits | | | |
| | | W 39, W 52, W 65, | | | Follow up^{f} |
| | | W 78 (ie, 1 year | | | (4 to 8 |
| | W 30 W 32 | in OLE), W 91, | EOT^{d} | | weeks after |
| | W 34 W 36 | W 104, W 117 | W 130 | pEOT^{e} | pEOT/EOT) |
| | Visit (a window of ±7 days is allowed for all visits after D 1) | | | | |
| | | 8, 9, 10, 11, | | | |
| | | 12, 13, 14 | 15 | | |
| | Place of the visit (S site; H: Home)^{g} | | | | |
| Procedure | S | S | S | S | H |

Informed consent
Inclusion and
exclusion criteria
Demography
Medical and
surgical history

| Prior/concomitant | <=====================================================================> | | | | |
|---|---|---|---|---|---|
| medications^{h} | | | | | |
| Randomization | | | | | |
| IRT contact | | X | X | X | X |
| IMP dispensation | | X | | | |
| IMP compliance | | X | X | X | |
| Diary dispensation | | X | X | X | |
| and collection^{j} | | | | | |

Safety

| | | | | | |
|---|---|---|---|---|---|
| Physical | | X | X | X | X |
| examination^{k} | | | | | |
| Vital signs^{l} | | X | X | X | X |
| Body height | | | | | |
| Body weight | | X | X | X | X |
| Pregnancy test | | X | X | X | X |
| (WOCBP only) ^{m} | | | | | |
| Serum FSHⁿ | | | | | |
| HIV, hepatitis B | | | | | |
| and C screening | | | | | |
| Tuberculosis testing^{o} | | | | | |
| Laboratory tests | | X | X | X | X |
| (include | | | | | |
| hematology and | | | | | |
| clinical chemistry)^{p} | | | | | |
| Liver function tests^{q} | X | | | | |
| Iron panel (serum): | | | | | |
| iron, ferritin, | | | | | |
| transferrin saturation, | | | | | |
| TIBC; to be repeated | | | | | |
| during the study, if | | | | | |
| needed | | | | | |
| Urinalysis | | X^{r} | X | X | |
| Coagulation: PT/INR, | | | | | |
| aPTT (to be repeated | | | | | |
| during the study, if | | | | | |
| needed) | | | | | |
| 12-lead ECG | | X^{s} | X | X | X |
| Suicidality | | X | X | X | X |
| assessment by | | | | | |
| CSSRS | | | | | |

| | |
|---|---|
| AE/SAE review | ←=================================================================→ |

Efficacy^{t}

| | | | |
|---|---|---|---|
| MG-ADL | X | X | X |
| QMG | X | X | X |
| MGII | X | X | X |
| MG-QoL15 | X | X | X |
| HCRU-MG | | X | X |
| MGFA-PIS | X | X | X |
| EQ-5D-5L | X | X | X |
| PGIS | X | X | X |

Pharmacokinetics

Plasma samples
for tolebrutinib
and active M2
metabolite^{u}

Pharmacogenetics

Archival DNA
sample^{x}

Pharmacodynamics/Biomarkers

| | |
|---|---|
| Blood sample | |
| for archiving^{y} | |
| Exploratory | X |
| biomarkers: serum | |
| auto-antibodies | |
| (anti-AChR and/or | |
| anti-MuSK) | |
| Exploratory | X |
| biomarkers: serum | |
| IgM and IgG | |
| Digital tool for | |
| activity/function^{z} | |

AChR: acetylcholine receptor; AE: adverse event; ALT: alanine aminotransferase; aPTT: activated partial thromboplastin time; AST: aspartate aminotransferase; BUN: blood urea nitrogen; AChEI: acetylcholinesterase inhibitor; C-SSRS: Columbia-Suicide Severity Rating Scale; D: day; eCRF: electronic case report form; DB: double-blind; EOS: End of Study; EQ-5D-5L: EuroQoL 5 Dimensions 5 Levels; EU: European Union; ECG: electrocardiogram; EOT: end of treatment; FU: follow-up; FSH: follicle-stimulating hormone; h: hour; HCRU-MG: healthcare resource utilization myasthenia gravis; HIV: human immunodeficiency virus; ICF: informed consent form; IgG: immunoglobulin G; IgM: immunoglobulin M; IMP: investigational medicinal product; INR: international normalized ratio; IRT: interactive response technology; MG: myasthenia gravis; MG-ADL: Myasthenia Gravis-Activities of Daily Living; MGFA-PIS: Myasthenia Gravis Foundation of America Post-intervention Status; MGII: Myasthenia Gravis Impairment Index; MG-QoL15: Myasthenia Gravis Quality of Life 15-item Scale; MuSK: muscle-specific kinase; OLE: open-label extension; pEOT: premature end of treatment; PGIS: Patient Global Impression of Severity; PK: pharmacokinetic(s); PT: prothrombin time; QMG: Quantitative Myasthenia Gravis; SAE: serious adverse event; TB: tuberculosis; TIBC: total iron-binding capacity; V: visit; W: week; WOCBP: woman of childbearing potential; β-HCG: β-human chorionic gonadotropin.
a The screening period can range from D −28 to D −1. The randomization visit can be performed only once IMPs are available onsite. The interval between screening and randomization visits can range from 11 days (minimum) to 28 days (maximum). However, if required, the randomization visit can be performed earlier than 11 days upon IMP receipt at the site, assuming the participant is eligible for randomization.
^{b}From D 1 to the EOT, unscheduled visits may be performed at any time by the Investigator (eg, for a suspected MG crisis or evaluation of an AE). Assessments may be performed as needed to evaluate the participant in accordance with the Investigator's best judgment and in line with the study protocol. At a minimum, a physical examination should be performed, and body temperature and vital signs should be measured. In case of a suspected MG crisis suspicion, at least the MG-ADL assessment should be performed (see Rescue therapy).
c The Week 26 Visit will also be Day 1 for the OLE. Participants will begin the OLE treatment (open label tolebrutinib once daily 60 mg) on the next day.
^{d}The EOS visit will be the FU visit, 4 to 8 weeks after last dose of study intervention.
^{e}During the DB period, if a participant prematurely and permanently discontinues treatment with the IMP, he/she will undergo a pEOT visit as soon as possible. Participants will then be asked to continue in the study with all study procedures/visits, except those associated with IMP administration. In the OLE period, participants with pEOT will be encouraged to attend the safety FU visit (4 to 8 weeks after pEOT), which will be considered as the EOS.
^{f}A safety FU visit needs to be performed 4 to 8 weeks after pEOT for participants who discontinue the DB IMP prematurely and who do not wish to continue the study, or 4 to 8 weeks after EOT. This visit will be considered as the EOS. In the case the FU visit is conducted as a home visit and some assessments are not possible during the visit (eg, neurological examination part of the physical examination), these parts of the assessment may be skipped.
^{g}Home visits can be replaced by site visits with the same assessments and procedures, depending on the Investigator's assessment and/or local regulatory requirement(s).
^{h}Any MG medication taken at any time prior to signing the informed consent needs to be reported in the eCRF; other prio medications will be reported for the period of 6 months prior to signing the ICF.
i Investigational medicinal product dispensation for participants who will continue in the OLE period.
^{j}Diaries will be collected at EOS for participants completing their study and at pEOT for participants who prematurely discontinue the study.
^{k}Complete physical examination due at Screening, D 1, Visit 7 (Week 26), Visit 11 (Week 78), and EOT. Brief physical examination is sufficient for the rest of the visits (complete and brief physical examination will include neurological examination).
^{l}Vital signs, including systolic and diastolic blood pressure (mmHg), pulse rate (beats per minute), and body temperature (° C.) will be measured.
^{m}Serum β-HCG at central laboratory at Screening and urine pregnancy tests within 24 hours before the first dose of IMP and at scheduled times during the study. In addition to scheduled visits, pregnancy tests will be performed monthly for all participants who are WOCBP. Additional serum or urine pregnancy tests may be performed, as deemed necessary by the Investigator or required by local regulations, to establish the absence of pregnancy at any time during the participant's participation in the study.
ⁿOnly in female participants, if needed to establish menopausal status.
^{o}To be performed at Screening for all participants. To be repeated based on clinical judgment, borderline results, or clinical suspicion of TB infection. Refer to the exclusion criteria (Table 1D).
^{p}Clinical chemistry (blood urea nitrogen [BUN], creatinine, glucose, potassium, sodium, chloride, bicarbonate, calcium, liver function tests [AST, ALT, albumin, alkaline phosphatase, total and direct bilirubin, total protein; creatine phosphokinase], hematology (platelet count, red blood cell count, hemoglobin, hematocrit, mean corpuscular volume, mean corpuscular hemoglobin, reticulocytes, white blood cell count with differential: neutrophils, lymphocytes, monocytes, eosinophils, basophils). Lipase will be tested at the Screening Visit only. Week 19 will include hematology and full liver panel only (see Table 1G).
^{q}At intermediate timepoints W 6, W 10, W 30, W 32, W 34, and W 36, only liver function tests will be collected (AST, ALT, albumin, alkaline phosphatase, total and direct bilirubin, total protein, and creatine phosphokinase); these can be central or local labs.
^{r}During OLE, urinalysis will be conducted at Visits 9, 11, and 13.
^{s}During OLE, ECG will be performed at Visits 8, 9, and 13.
^{t}For all visits with QMG or MGII assessments: to reduce fluctuations in performance on functional tests due to temporary symptomatic effects associated with AChEI use, AChEIs should not be taken for at least 12 hours prior to efficacy endpoint testing at each visit. It is important that participants complete the outcome assessments after the ICF is signed and prior to any treatment- or study-related activities including administration of study intervention, laboratory work, radiological assessments, discussion with participants regarding their treatment or health status, and similar activities. This ensures the objectivity of the data. Detailed site training on clinical outcome assessments will be provided. These clinical outcome assessments will be administered and documented electronically by both the Investigator and participants.
^{u}Pharmacokinetic analysis will be performed in a subset of participants ≥50 years of age (approximately 40 participants). On days of PK sampling, the IMP needs to be taken at the study site with a meal.
v Two plasma samples at Visit 2 between 0.5 to 1.5 hours and 3 to 4 hours post-dose.
w One plasma sample at Visits 5 and 7 between 0.5 to 1.5 hours post-dose.
^{x}The DNA sampling may be done, if locally applicable, at any time after signature of consent (in case it is not done for some reason at Screening). For participants in China, this DNA sample will not be collected.
^{y}At Screening, the archiving blood sample will be collected and stored for use if any unexpected safety issue occurs to ensure that a pre-dose baseline value is available for previously unassessed parameters (eg, serology) and for biomarker research, if agreed. For participants in China, this archiving sample will not be collected.
^{z}A noninvasive digital tool to monitor participant activity or function may be optionally implemented by the Sponsor in a subset of participants during the study, if results from pilot assessment demonstrate feasibility.
Baseline assessment will be done at Screening.

Objectives and endpoints for the treatment are shown in Table 1B.

TABLE 1B
Objectives and endpoints

| Objectives | Endpoints |
|---|---|
| Primary | |
| DB period | Change from baseline in MG-ADL total |
| To evaluate the efficacy of tolebrutinib 60 mg | score at Week 26 |
| daily compared to placebo as measured by | |
| MG-ADL score in participants with gMG | |
| who are receiving SoC | |
| OLE | AEs, serious AEs, AEs leading to permanent |
| To evaluate the long-term safety and | study intervention discontinuation, AESIs, |
| tolerability of tolebrutinib 60 mg daily in | potentially clinically significant abnormalities |
| participants with gMG who are receiving SoC | in laboratory tests, ECG, and vital signs during |
| | the treatment period |
| Secondary | |
| DB period | Change from baseline in QMG total score at |
| To evaluate the efficacy of tolebrutinib 60 mg | Week 26 |
| daily compared to placebo on additional | Change from baseline in QMG total score at |
| efficacy measurements: QMG, MGII, | Week 12 (for the interim analysis only) |
| MG-QoL15, MG-ADL in participants with | Change from baseline in MGII total score at |
| gMG who are receiving SoC | Week 26 Change from baseline in MG-QoL15 |
| | total score at Week 26 |
| | Proportion of participants with ≥2-point |
| | improvement (reduction) in MG-ADL total |
| | score at Week 26 |
| | Proportion of participants with ≥3-point |
| | improvement (reduction) in QMG total score |
| | at Week 26 |
| To evaluate the safety and tolerability of | AEs, serious AEs, AEs leading to permanent |
| tolebrutinib 60 mg daily compared to placebo | study intervention discontinuation, AESIs, |
| in participants with gMG who are | potentially clinically significant abnormalities |
| receiving SoC | in laboratory tests, ECG, and vital signs during |
| | the treatment period |
| OLE | Change from baseline in MG-ADL total score |
| To evaluate the long-term efficacy of | over time Change from baseline in QMG total |
| tolebrutinib 60 mg daily in participants | score over time Change from baseline in MGII |
| with gMG who are receiving SoC | total score over time Change from |
| | baseline in MG-QoL15 total score over time |
| | Proportion of participants with ≥2-point |
| | improvement (reduction) in MG-ADL total score |
| | at EOT (timeframe: baseline, up to Week 130) |
| | Proportion of participants with ≥3-point |
| | clinical improvement (reduction) in QMG total |
| | score at EOT (timeframe: baseline, up to Week 130) |
| | Proportion of participants achieving any reduction |
| | from baseline of daily dose of OCS over time |

Abbreviations: AChR: acetylcholine receptor; AE: adverse event; AESI: adverse event of special interest; DB: double-blind; ECG: electrocardiogram; EOS: end of study; EOT: end of treatment; gMG: generalized MG; HCRU-MG: health care resource utilization MG; IgG: immunoglobulin G; IgM: immunoglobulin M; MG-ADL: Myasthenia Gravis Activities of Daily Living; MGFA-PIS: Myasthenia Gravis Foundation of America Post-intervention status; MG-QoL15: Myasthenia Gravis Quality of Life 15-item scale; MGII: Myasthenia Gravis Impairment Index; MuSK: muscle-specific kinase; OCS: oral corticosteroid; OLE: open-label extension; PD: pharmacodynamic; PK: pharmacokinetic; QMG: Quantitative Myasthenia Gravis; QoL: quality of life; SoC: standard of care.

### BRIEF SUMMARY

This is a multicenter, randomized, DB, placebo-controlled, Phase 3 study to evaluate the efficacy and safety of tolebrutinib 60 mg daily compared with placebo in adult participants aged 18 to 85 years with moderate-to-severe gMG who are receiving the SoC. The DB treatment period of 26 weeks will comprise 7 site visits followed by a 2-year OLE period with quarterly visits.

The efficacy of tolebrutinib versus placebo during the DB period will be assessed by clinical evaluations, which include scales based on the physician's examination or direct participant feedback, ie, clinical outcome assessments. These evaluations will continue during the OLE in order to measure long-term efficacy and safety.

### Number of Participants

Approximately 192 people will be screened to achieve 154 participants randomized to the study intervention at a randomization ratio of 1:1 (assuming a screen failure rate of 20%).

### Intervention Groups and Duration

The DB period will include a screening period (up to 28 days), after which eligible participants will be randomized to a treatment group, 60 mg oral, daily tolebrutinib or matching placebo.

The duration of the treatment period will be 26 weeks.

The OLE will include all eligible participants who have completed the DB period on treatment. Participants will receive 60 mg of oral, daily tolebrutinib for a duration of up to 2 years.

Post-trial access may be considered, if required, and approved by local regulations.

### Study Intervention(s)

### Investigational Medicinal Product

Formulation: tolebrutinib film-coated tablet
Route of administration: oral
Dose regimen: 60 mg once daily taken with a meal

### Investigational Medicinal Product

Formulation: placebo to match tolebrutinib film-coated tablet
Route of administration: oral
Dose regimen: once daily taken with a meal

The modified intention-to-treat (mITT) population will include all randomized and treated participants with a baseline value and at least 1 post-baseline value for any efficacy assessment. Participants will be analyzed as randomized. This will be the primary efficacy population.

### Study Population

### Inclusion Criteria

Participants are eligible to be included in the study only if all of the following criteria apply as shown in Table 1C.

TABLE 1C
Inclusion Criteria

| Category | | Criteria |
|---|---|---|
| Age | I 01. | The participant must be 18 to 85 years of age inclusive, at the time |
| | | of signing the informed consent. |
| Type of | I 02. | Participants with a diagnosis of gMG at Screening with generalized |
| participant | | muscle weakness meeting the clinical criteria for diagnosis of |
| and disease | | MG, as defined by the MGFA Clinical Classification Class II, III, |
| characteristics | | or IV, and likely not in need of a respirator for the duration of the |
| | | study, as judged by the Investigator. |
| | I 03. | a) Positive serologic testing for anti-AChR or anti-MuSK |
| | | autoantibody at Screening OR |
| | | b) Seronegative for both anti-AChR and anti-MuSK autoantibodies |
| | | and with prior diagnosis supported by ≥1 of the following 3 tests: |
| | | History of abnormal neuromuscular transmission demonstrated by |
| | | single-fiber electromyography or repetitive nerve stimulation |
| | | History of positive edrophonium chloride test |
| | | Participant has demonstrated improvement in gMG signs on oral |
| | | acetylcholinesterase inhibitors as assessed by the treating physician. |
| | | The initial 40 participants randomized in the study will include only |
| | | those participants positive for anti-AChR or anti-MuSK |
| | | autoantibodies. Seronegative participants will be recruited after the |
| | | IA, depending on the outcome. |
| | I 04. | The participant must have a score ≥6 on MG-ADL scale at |
| | | Screening and Day 1 visits with greater than half of the score |
| | | attributed to non-ocular items. |
| | I 05. | Participants are allowed to use a stable dose of one or more of the |
| | | following gMG treatments prior to randomization: AChEIs, OCS |
| | | or IST as described thereafter. Allowed ISTs include azathioprine, |
| | | mycophenolate mofetil, tacrolimus or methotrexate, and only one |
| | | can be used at any time, during the study. Participants must be on a |
| | | stable dose of their treatments prior to Screening visit, as |
| | | applicable and according to the following requirements: |
| | | a) Stable dose of AChEIs for at least 2 weeks. |
| | | b) Stable dose of OCS ≤20 mg/daily for at least 1 month. |
| | | c) Azathioprine, mycophenolate mofetil, tacrolimus or methotrexate |
| | | should have been initiated at least 6 months prior to the Screening |
| | | visit and continued on a stable dose for at least 3 months. |
| Weight | I 06. | Not Applicable. |
| Sex | I 07. | Male or Female. |
| | | The methods of contraception should be consistent with local |
| | | regulations of participating sites. |
| | a) | Male participants |
| | | Not applicable. |
| | b) | Female participants |
| | | A female participant is eligible to participate if she is not pregnant |
| | | or breastfeeding, and at least one of the following conditions applies: |
| | | Is not a woman of childbearing potential (WOCBP); or |
| | | Is a WOCBP and agrees to use an acceptable contraceptive method |
| | | during the intervention period (at a minimum until after the last |
| | | dose of study intervention). |
| | | A WOCBP must have a negative highly sensitive pregnancy test |
| | | (urine or serum as required by local regulations) at screening before |
| | | the first dose of study intervention. If a urine test cannot be |
| | | confirmed as negative (eg, an ambiguous result), a serum pregnancy |
| | | test is required. In such cases, the participant must be excluded |
| | | from participation if the serum pregnancy result is positive. |
| | | Additional requirements for pregnancy testing during and after |
| | | study intervention are located in the schedule of activities (SoA), |
| | | Table 1A (Tables 1A1 and 1A2). |
| | | The Investigator is responsible for review of medical history, |
| | | menstrual history, and recent sexual activity to decrease the risk for |
| | | inclusion of a woman with an early undetected pregnancy, if |
| | | allowed by local regulations. |
| Informed | I 09. | Capable of giving signed informed consent which includes |
| Consent | | compliance with the requirements and restrictions listed in the |
| | | informed consent form (ICF). |

### Exclusion Criteria

Participants are excluded from the study if any of the following criteria apply as shown in Table 1D.

TABLE 1D
Exclusion Criteria

| Category | | Criteria |
|---|---|---|
| Medical conditions | E 01. | MGFA Class I (ocular MG) or Class V. |
| | E 02. | History of thymectomy within 6 months from Screening or |
| | | planned for a thymectomy during the study period. |
| | E 03. | The participant has a history of infection or may be at risk for |
| | | infection: |
| | a) | A history or a current diagnosis of active or untreated latent |
| | | tuberculosis (TB), or currently undergoing treatment for latent TB. |
| | | In case of confirmed active or latent TB, the patient can be |
| | | re-screened after full completion and written documentation of |
| | | anti-tuberculosis treatment. |
| | | In the cases where latent TB is suspected or being treated based on |
| | | the screening TB testing and an infectious disease expert is starting |
| | | TB treatment, this specialist can decide and provide written |
| | | documentation that the patient has completed treatment for latent |
| | | TB, even if it is shorter than standard treatment timelines. |
| | b) | Participants with existing household contacts with active TB, with |
| | | the exception of those for whom prophylaxis treatment has been |
| | | completed for both the participant and household contact. |
| | c) | A positive TB test at Screening or during the study, with the |
| | | exception of patients for whom latent TB treatment has been |
| | | completed per local guidelines. TB testing should be performed at |
| | | Screening and again during the study, if clinically indicated, and |
| | | may be repeated based on clinical judgment, borderline results, or |
| | | clinical suspicion of TB infection. QuantiFERON-TB Gold blood |
| | | test is preferred; tuberculin skin testing with ancillary testing is |
| | | allowable if blood testing is not available. For an indeterminate |
| | | QuantiFERON-TB Gold or blood test result, results may be repeated |
| | | once and will be considered positive if retest results are positive. |
| | d) | If repeat QuantiFERON-TB Gold continues to be indeterminate, |
| | | T-SPOT.TB testing is preferred as the next appropriate test. |
| | | Screening tests for TB are described in Table 1G. |
| | | The Investigator may also consult with an infectious disease expert |
| | | if required, eg, test results are unclear or there is a suspicion of |
| | | false positive test results. If the infectious disease expert considers |
| | | the test results false positive and not clinically relevant and |
| | | confirms that the participant does not have TB, the Investigator |
| | | must document this in source data and may then randomize the |
| | | participant provided other recruitment criteria are met. |
| | e) | Participants at risk of developing or having reactivation of |
| | | hepatitis, ie, results at Screening for serological markers for |
| | | hepatitis B and C indicating acute or chronic infection. Serology |
| | | tests will include hepatitis B virus surface antigen, anti-hepatitis B |
| | | core antigen immunoglobulin M (IgM) and total immunoglobulins |
| | | (Igs), anti-hepatitis B surface antigen Igs and anti-hepatitis C virus |
| | | Igs; in case these results are inconclusive (eg, anti-hepatitis B |
| | | surface antigen negative and anti-hepatitis B core positive or |
| | | anti-hepatitis C virus immunoglobulin G [IgG] positive), hepatitis |
| | | B virus- DNA and/or hepatitis C virus-RNA testing, respectively, |
| | | should be performed for confirmation. |
| | f) | Persistent chronic or active recurring infection requiring treatment |
| | | with antibiotics, antivirals, or antifungals. |
| | g) | Fever within 4 weeks of the Screening visit (≥38° C.; however, if |
| | | due to brief and mild ear, nose, throat viral infection participant |
| | | may be included based on the Investigator's judgment). |
| | h) | A history of infection with human immunodeficiency virus (HIV) |
| | | (eg, any known positive HIV test or information from |
| | | participant interview). |
| | i) | A history of T-lymphocyte or T-lymphocyte-receptor vaccination, |
| | | transplantation (including solid organ, stem cell, and bone marrow |
| | | transplantation) and/or antirejection therapy. |
| | j) | The participant has a lymphocyte count less than the lower limit of |
| | | normal at the Screening visit. |
| | k) | Any other active infections that would adversely affect |
| | | participation or IMP administration in this study, as judged by |
| | | the Investigator. |
| | E 04. | Any malignancy within 5 years prior to the Screening Visit |
| | | (except for effectively treated carcinoma in situ of the cervix, |
| | | adequately treated non-metastatic squamous or basal cell |
| | | carcinoma of the skin and malignant thymoma that have been |
| | | resected or are considered as cured by any treatment with no |
| | | evidence of metastatic disease for ≥3 years) will be exclusionary. |
| | E 05. | History of autoimmune disease other than gMG (eg, thyroiditis, |
| | | rheumatoid arthritis, etc.) that would interfere with an accurate |
| | | assessment of clinical symptoms and gMG diagnosis. |
| | E 06. | Conditions that may predispose the participant to excessive |
| | | bleeding: |
| | | A bleeding disorder or known platelet dysfunction at any time |
| | | prior to the screening visit. |
| | | A platelet count <150 000/μL at the screening visit. |
| | | The participant has had major surgery within 4 weeks prior to the |
| | | screening visit, which could affect the participant's safety (as |
| | | judged by the Investigator) or has planned any elective major |
| | | surgery during the study. |
| | | A history of significant bleeding event within 6 months prior to |
| | | screening, according to the Investigator's judgment such as, but |
| | | not limited to cerebral or gastrointestinal bleeding. |
| | E 07. | Confirmed screening ALT >1.5 × ULN OR AST >1.5 × ULN |
| | | OR alkaline phosphatase >2 × ULN (unless caused by |
| | | non-liver-related disorder or explained by a stable chronic liver |
| | | disorder) OR total bilirubin >1.5 × ULN (unless due to Gilbert |
| | | syndrome or non-liver-related disorder). |
| | E 08. | A history or presence of significant other concomitant illness |
| | | according to the Investigator's judgment such as, but not limited to |
| | | cardiovascular (including Stage III or IV cardiac failure according |
| | | to New York Heart Association classification), or renal (ie, |
| | | undergoing dialysis), neurological, endocrine, gastrointestinal, |
| | | metabolic, pulmonary, or lymphatic disease that would adversely |
| | | affect participation in this study. |
| | E 09. | A history or presence of psychiatric disturbance or substance |
| | | abuse, as evidenced by: |
| | | a) A history of any psychiatric disease, behavioral condition, or |
| | | depression requiring hospitalization within 2 years prior to the |
| | | Screening Visit. |
| | | b) A documented history of attempted suicide or suicidal ideation |
| | | of category 4 or 5 according to the Columbia-Suicide Severity |
| | | Rating Scale (C-SSRS) baseline/screening version over the 6 |
| | | months prior to the Screening Visit, OR if in the Investigator's |
| | | judgment, the participant is at risk for a suicide attempt. |
| | | c) Active alcohol use disorder or a history of alcohol or drug abuse |
| | | within 1 year prior to the Screening Visit. |
| | | d) Current alcohol intake >2 drinks per day for men and >1 drink |
| | | per day for women (1 drink = approximately 14 grams of |
| | | alcohol = 350 mL beer = 140 mL wine = 40 mL of spirits). |
| | E 10. | The following findings obtained during the Screening Visit |
| | | considered in the Investigator's judgment to be clinically |
| | | significant: |
| | | a) Any Screening laboratory values outside normal limits. |
| | | b) Abnormal ECG. |
| | | Note: a one-time retest at Screening may be performed if an |
| | | abnormal laboratory test value is considered temporary. |
| Prior/concomitant | E 11. | The participant has received any live (attenuated) vaccine |
| therapy | | (including but not limited to varicella zoster, oral polio, and |
| | | nasal influenza) within 2 months before the first treatment visit. |
| | E 12. | The participant is receiving potent and moderate inducers of |
| | | cytochrome P450 3A (CYP3A) or potent inhibitors of CYP2C8 |
| | | hepatic enzymes. |
| | E 13. | The participant is receiving anticoagulant/antiplatelet |
| | | therapies, including: |
| | | Acetylsalicylic acid (aspirin) >81 mg/day, |
| | | Antiplatelet drugs (eg, clopidogrel), |
| | | Warfarin (vitamin K antagonist), |
| | | Heparin, including low molecular weight heparin |
| | | (antithrombin agents), |
| | | Dabigatran (direct thrombin inhibitor), |
| | | Apixaban, edoxaban, rivaroxaban (direct factor Xa inhibitors). |
| | | Note: All the above-mentioned drugs must be stopped at least 5 |
| | | half-lives before study intervention administration except for |
| | | aspirin, which must be stopped at least 8 days before. These |
| | | washout periods are only applicable in the case that the Investigator |
| | | deems it clinically appropriate to discontinue the listed medications |
| | | or there is a recent history of use of these medications (as in the |
| | | case when short term treatment with anticoagulants is clinically |
| | | recommended for certain thrombotic events), and therefore these |
| | | washout periods will need to be followed prior to enrollment. |
| | | If, however, the participant has a chronic underlying medical |
| | | condition (stroke, coronary or carotid artery disease, heart valvular |
| | | disease etc.) requiring continued use of these medications, the |
| | | participant cannot be enrolled in the study. |
| | E 14. | Treatments prior to randomization: |
| | | a) Intravenous immunoglobulin (IVIg) or plasma exchange |
| | | within 4 weeks. |
| | | b) Oral or IV cyclophosphamide or cyclosporine treatment |
| | | within 3 months. |
| | | c) Intravenous CS bolus (dose higher than 1 mg/kg) |
| | | within 1 month. |
| | | d) Rituximab and other B-cell-depleting therapies (anti-CD20 or |
| | | anti-CD19) used within 6 months. |
| | | Eculizumab, and other complement pathway targeting drugs or |
| | | anti-neonatal Fc receptor (FcRn) targeting drugs used |
| | | within 3 months. |
| Other exclusion | E 15. | At screening, elevated transferrin saturation (>50% in males and >40% |
| criteria | | in females) and/or with elevated ferritin levels >500 μg/L. |
| | E 16. | Acute liver disease, cirrhosis, chronic liver disease (unless |
| | | considered stable for >6 months). |
| Other exclusions | E 17. | Individuals accommodated in an institution because of regulatory or |
| | | legal order; prisoners or participants who are legally institutionalized |
| | E 18. | Participant not suitable for participation, whatever the reason, as |
| | | judged by the Investigator, including medical or clinical |
| | | conditions, or participants potentially at risk of noncompliance |
| | | to study procedures |
| | E 19. | Participants are employees of the clinical study site or other |
| | | individuals directly involved in the conduct of the study, or |
| | | immediate family members of such individuals (in conjunction |
| | | with section 1.61 of the ICH-GCP Ordinance E6) |
| | E 20. | Any specific situation during study implementation/course that |
| | | may raise ethics considerations |
| | E 21. | Sensitivity to any of the study interventions, or components thereof, |
| | | or drug or other allergy that, in the opinion of the Investigator, |
| | | contraindicates participation in the study |
| | | Note: a one-time retest at screening may be performed if an |
| | | abnormal laboratory test value is considered temporary |

### Lifestyle Considerations

### Meals and Dietary Restrictions

Tolebrutinib shall be taken with a regular meal. When possible, the meal with which tolebrutinib is taken (eg, breakfast, lunch, or dinner) should be consistent throughout the study. The typical meal with which the study intervention is taken will be recorded at each visit. In case the mealtime for study intervention administration needs to be changed, a gap of a minimum of 12 hours between 2 doses should be maintained.

### Caffeine, Alcohol, and Tobacco

For each visit with PK/PD assessment, participants will abstain from ingesting caffeine- or xanthine-containing products (eg, coffee, tea, cola drinks, and chocolate) for 2 hours before the start of treatment until after collection of the final PK and/or PD sample later that day.

For each visit with PK/PD assessment, participants will abstain from alcohol for 24 hours before the start of treatment until after collection of the final PK and/or PD sample later that day.

During the entire study, participants should be warned not to consume substantial quantities of alcohol, defined as >14 grams (1 standard drink) per day in female participants or >28 grams (2 standard drinks) per day in male participants on a regular basis.

Individuals who do not meet the criteria for participation in this study may be rescreened up to 2 times. Rescreened participants should be assigned a new participant number. There is no requirement for a waiting period between the screen failure date and the rescreen.

If a participant does not meet the inclusion criteria for certain dynamic laboratory tests at Screening (Visit 1), these laboratory assessments may be repeated, at the discretion of the Investigator, if the parameter result is judged to be likely to return to acceptable range for study inclusion within the screening period prior to Baseline/Randomization (Visit 2). There is no need to screen fail such participants if the test finally meets the inclusion criteria.

### Study Intervention(s) and Concomitant Therapy

Study intervention is defined as any investigational intervention(s), marketed product(s), placebo, or medical device(s) intended to be administered to a study participant according to the study protocol.

TABLE 1E
Overview of study interventions administered

| | | |
|---|---|---|
| Intervention label | tolebrutinib 60 mg | Placebo |
| Intervention name | tolebrutinib 60 mg | Placebo |
| Type | Drug | Drug |
| Dose formulation | film-coated tablet | film-coated tablet |
| Unit dose strength(s) | 60 mg | 0 mg |
| Dosage level(s) | Once daily | Once daily |
| Route of administration | Oral taken with a meal | Oral taken with a meal |
| Use | Investigative | Placebo comparator |
| IMP and NIMP | IMP | IMP |
| Packaging and labeling | Study intervention will be | Study intervention will be |
| | provided in wallet blister | provided in wallet blister |
| | packaging. The content of | packaging. The content of the |
| | the labeling is in accordance | labeling is in |
| | with the local regulatory | accordance with the local |
| | specifications and | regulatory specifications |
| | requirements. | and requirements. |
| [Current/Former name(s) or | SAR442168-Tolebrutininb | Not applicable |
| alias(es)] | | |

TABLE 1F
Arms and associated interventions

| | | |
|---|---|---|
| Arm name | tolebrutinib | Placebo |
| Associated interventions | tolebrutinib −60 mg | Placebo |
| (intervention label[s]) | | |

### Dose Modification

Dose reduction is not foreseen in this study. Treatment may need to be interrupted or permanently discontinued if deemed necessary due to an AE.

### Concomitant Therapy

### Prohibited Medications

Any medication (including over-the-counter or prescription medicines, recreational drugs, vitamins, and/or herbal supplements), vaccine or medical procedure that the participant is receiving at the time of enrollment or receives during the study must be recorded along with:
Reason for use
Dates of administration including start and end dates
Dosage information including dose and frequency
   The Sponsor should be contacted if there are any questions regarding concomitant or prior therapy.

Participants must abstain from taking prescription or nonprescription herbal medications containing Saint John's Wort extract within 14 days before the start of study intervention until completion of the last visit.

Live (attenuated) vaccines should not be administered during the study.

For some prohibited concomitant medications (eg, aspirin for headache), if use is not chronic, temporary discontinuation of IMP can be considered prior to a decision to permanently stop the IMP.

Prohibited treatments during the study will also include:
IV CS and OCS >20 mg/daily except if used as rescue treatment
Cyclosporine and cyclophosphamide.
Rituximab and other B-cell-depleting therapies (anti-CD20 or anti-CD19), eculizumab and other complement pathway targeting drugs, anti-FcRn, or any monoclonal antibodies.

Anticoagulant/antiplatelet therapies are not permitted to be taken concomitantly with the IMP, including:
Acetylsalicylic acid (aspirin) >81 mg/day
Antiplatelet drugs (eg, clopidogrel)
Warfarin (vitamin K antagonist)
Heparin, including low molecular weight heparin (antithrombin agents)
Dabigatran (direct thrombin inhibitor)
Apixaban, edoxaban, rivaroxaban (direct factor Xa inhibitors)
CYP inhibitors/inducers: Potent and moderate inducers of CYP3A or potent inhibitors of CYP2C8 hepatic enzymes are not permitted throughout the study.

Tolebrutinib is a substrate of the CYP3A4 and CYP2C8 isoenzymes. In healthy participants, potent CYP3A4 inhibitor (itraconazole 200 mg once daily for 4 days) increased tolebrutinib (area under the curve [AUC]) exposure by 1.8-fold and potent CYP2C8 inhibitor (gemfibrozil 600 mg twice daily for 6 days) increased tolebrutinib (AUC) exposures by 8.4-fold. Based on a satisfactory safety and tolerability profile and on the observed exposure in healthy participants who received tolebrutinib at a dose of up to 240 mg once daily for 14 days under fed conditions, drugs that strongly inhibit CYP3A4 are allowed and drugs that strongly inhibit CYP2C8 are not permitted. In healthy participants, the potent CYP3A4 and moderate CYP2C8 induction by rifampicin (600 mg once daily for 8 days) decreased tolebrutinib exposure by 6-fold. Therefore, potent and also moderate (based on prediction) CYP3A inducers are not permitted due to their potential to decrease tolebrutinib exposure and efficacy.

Other Drugs Permitted with Some Restrictions:

### Anticoagulant/Antiplatelet

Acetylsalicylic acid (aspirin)≤81 mg/day
Paracetamol/acetaminophen, at doses of ≤3 grams/day, is permitted for use at any time during the study. Short courses (up to 5 days) of nonsteroidal anti-inflammatory drugs (NSAIDs) (other than acetylsalicylic acid) at the recommended dose may be given during the study if clinically necessary for the treatment of an existing medical condition or a new event. The Investigator must record the use of NSAIDs (and any other comedication) in the eCRF. The Investigator should assess for signs of bleeding events for a participant taking NSAIDs with IMP. Nonsteroidal anti-inflammatory drugs should be interrupted for Grade 2 and above bleeding events.

### Rescue Therapy

The use of rescue therapy for gMG worsening will be allowed at any time during both the DB and OLE parts of the study at discretion of the Investigator in case of at least a 2-point increase of individual non-ocular MG-ADL items compared to the Day 1 MG-ADL value or new or worsening of respiratory/bulbar symptoms. Rescue therapy can include IVIg, plasma exchange, change in the SoC OCS dose or any use of new CS. If rescue therapy is required, the Sponsor should be informed, preferably prior to administration of treatment, where possible, without compromising the participant's safety. The date and time of rescue therapy administration as well as the name and dosage regimen must be recorded. In case of use of rescue therapy during the DB period, the study intervention should be permanently discontinued.

The supply of rescue therapy will be specified at the country level. Use of rescue therapy must be recorded in the eCRF.

### Liver Chemistry Stopping Criteria

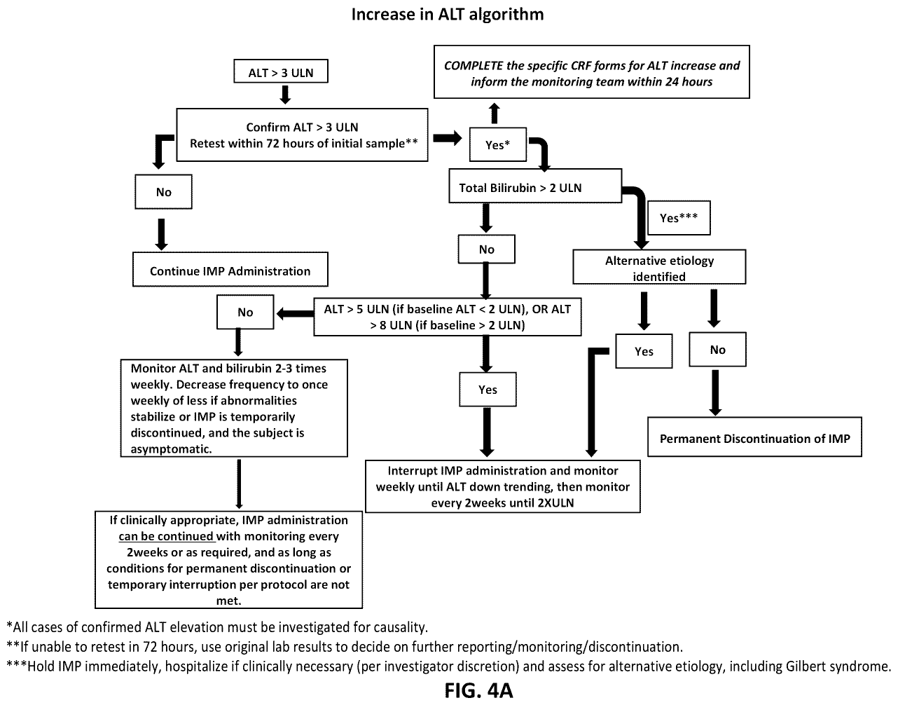

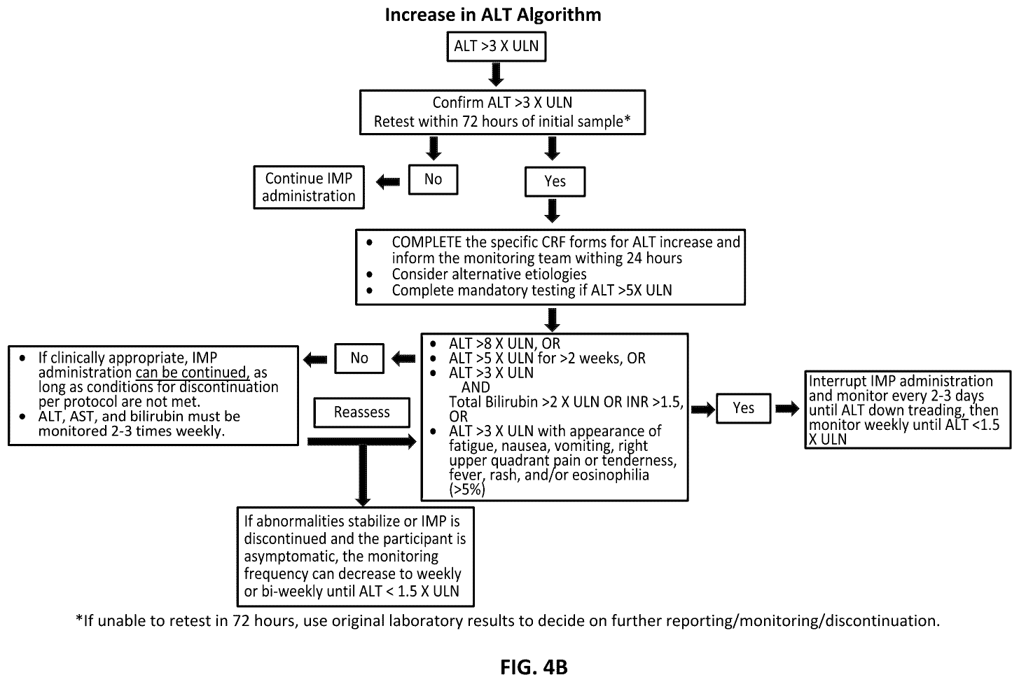

Discontinuation of study intervention for abnormal liver function tests is required when a participant meets one of the conditions outlined in the algorithm (See FIGS. 4A and 4B). Discontinuation of study intervention in the presence of abnormal liver chemistries not meeting protocol-specified stopping rules may also be required if the Investigator believes that it is in best interest of the participant.

### QTc Stopping Criteria

If a clinically significant finding is identified in the ECG (including, but not limited to changes from baseline in QT interval corrected using Fridericia's formula [QTcF]) after enrollment, the Investigator or qualified designee will determine if the participant can continue to receive study intervention and if any change in participant management is needed. Review of ECG findings by a cardiologist may be considered for a decision of a definitive discontinuation of study intervention because of ECG changes. This review of the ECG printed at the time of collection must be documented. Any new clinically relevant finding should be reported as an AE.

### Temporary Discontinuation

Temporary intervention discontinuation may be considered by the Investigator because of suspected AEs or disruption of the clinical study due to a regional or national emergency declared by a governmental agency. For all temporary intervention discontinuations, duration should be recorded by the Investigator in the appropriate pages of the eCRF.

If surgery is needed during the study, the benefit-risk of withholding the IMP for at least 3 to 7 days pre- and post-surgery and the risk of bleeding should be considered.

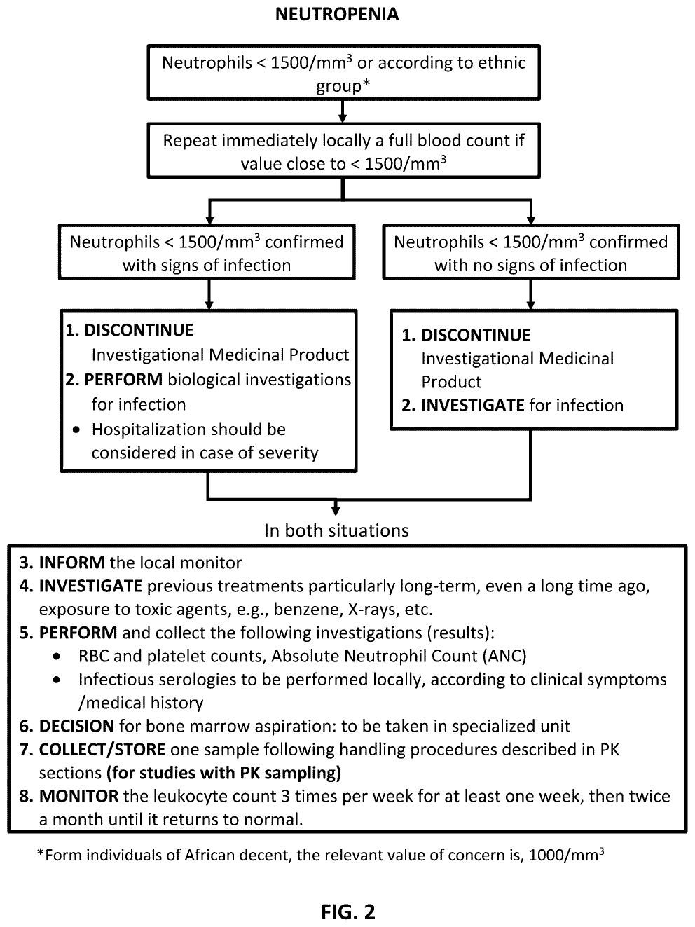

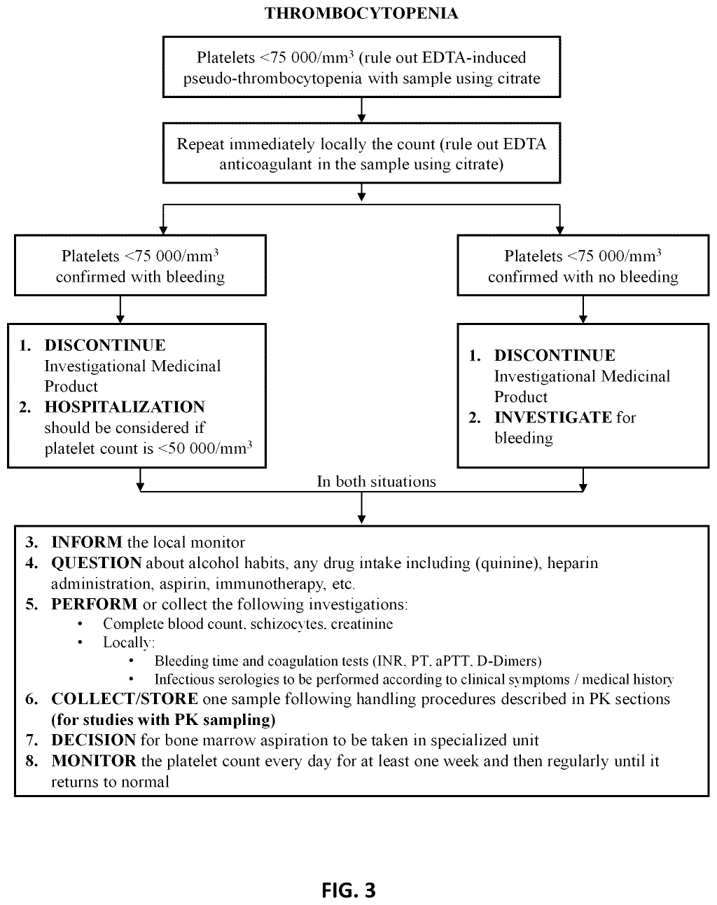

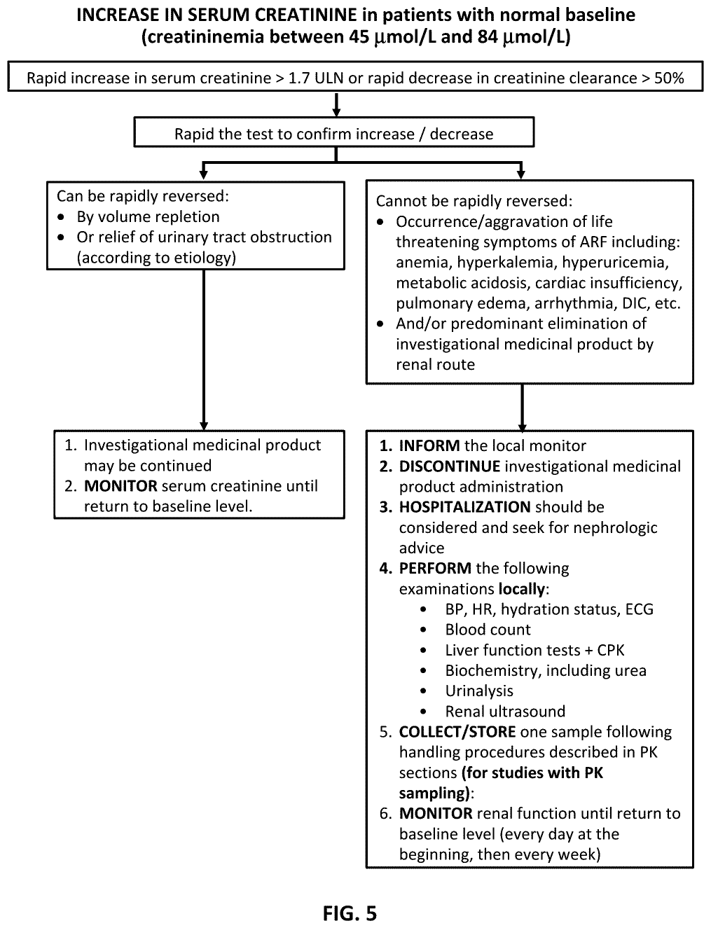

The following shall lead to temporary treatment discontinuation:
Cytopenias: the Sponsor's algorithms for neutropenia and thrombocytopenia as per FIGS. 2 and 3 should be followed.
Serum creatinine, creatine phosphokinase (CPK) and liver enzyme increase: follow corresponding algorithms as per FIG. 5.
Cardiac arrhythmia (atrial fibrillation): Any Grade 3 event (symptomatic, urgent intervention indicated, device [eg, pacemaker], ablation, new onset).
Suicidal risk as per C-SSRS: if a participant scores “yes” on items 4 or 5 of the Suicidal Ideation Section, or “yes” on any item of the Suicidal Behavior Section.

If needed, temporary treatment discontinuation can be considered by the Investigator or by the participant for any other reason, including due to any safety concerns because of disruption of the study due to a regional or national emergency declared by a governmental agency such as COVID-19, or another illness or if there is a need for a prohibited concomitant medication. Treatment can be resumed later when it is considered safe and appropriate.

If participants no longer wish to take the IMP, they will be encouraged to remain in the study.

Investigators should discuss with participants key visits to attend. The value of all their study data collected during their continued involvement will be emphasized as important to the public health value of the study.

Participants who withdraw from the study intervention should be explicitly asked about the contribution of possible AEs to their decision, and any AE information elicited must be documented.

All study withdrawals should be recorded by the Investigator in the appropriate screens of the CRF or eCRF and in the participant's medical records. In the medical record, at least the date of the withdrawal and the reason should be documented.

In addition, a participant may withdraw his/her consent to stop participating in the study. Withdrawal of consent for intervention should be distinguished from withdrawal of consent for FU visits and from withdrawal of consent for non-participant contact FU, eg, medical record checks. The site should document any case of withdrawal of consent.

Participants who have withdrawn from the study cannot be rerandomized/reallocated (treated) in the study. Their inclusion and intervention numbers must not be reused.

TABLE 1G
Protocol-required laboratory assessments

| Laboratory | | | |
|---|---|---|---|
| assessments | Parameters | | |
| Hematology | Platelet count | RBC indices: | WBC count with |
| | | | differential: |
| | RBC count | MCV | Neutrophils |
| | Hemoglobin | MCH | Lymphocytes |
| | Hematocrit | % Reticulocytes | Monocytes |
| | | | Eosinophils |
| | | | Basophils |
| Clinical | BUN | Sodium | AST |
| chemistry | Creatinine | Calcium | ALT |
| | Glucose | Total and direct bilirubin | Alkaline phosphatase |
| | Potassium | Total protein | Albumin |
| | | Chloride | Creatine phosphokinase |
| | | Bicarbonate | Lipase (screening only) |

| | |
|---|---|
| Routine | Specific gravity |
| urinalysis | pH, glucose, protein, blood, ketones, bilirubin, urobilinogen, nitrite, |
| | leukocyte esterase by dipstick |
| | Microscopic examination (if blood or protein is abnormal and for signs of |
| | infection) |
| Other | FSH and estradiol (if needed, only in female participants to confirm |
| screening | postmenopausal state) |
| tests | Highly sensitive serum or urine β-hCG pregnancy test (as needed for |
| | women of childbearing potential)^{c} |
| | Iron panel (serum): iron, ferritin, transferrin saturation TIBC. |
| | Coagulation: PT/INR, aPTT |
| | Serology tests for HIV and other infectious diseases, if locally required |
| | Hepatitis serologic testing at Screening (Visit 1): hepatitis B surface antigen |
| | (HBsAg), hepatitis B surface antibody (HBsAb), hepatitis B core antibody |
| | (HBcAb IgM and Total), and hepatitis C virus antibodies (HCVAb). In case |
| | of results showing HBsAg (negative) and HBcAb Total (positive), HBV |
| | DNA testing will be performed prior to randomization to rule out a false |
| | positivity to clarify the serological status. In case of results showing HCV Ab |
| | (positive), HCV RNA testing will be performed to rule out a false positivity.^{d} |
| | Tuberculosis test: Blood testing (eg, QuantiFERON ® TB Gold test) is |
| | preferred; skin testing (eg, tuberculin skin test) with ancillary testing will be |
| | allowed if blood testing is not available. T-SPOT can also be performed, if |
| | available. |

AChR: acetylcholine receptor; AESI: adverse event of special interest; aPTT: activated thromboplastin time; DNA: deoxyribonucleic acid; HBV: hepatitis B virus; HIV: human immunodeficiency virus; IEC: Institutional Ethics Committee; IgM: immunoglobulin M; INR: international normalized ratio; IRB: Institutional Review Board; MCV: mean corpuscular volume; MCH: mean corpuscular hemoglobin; MuSK: muscle- specific kinase, PT: prothrombin time, RNA: ribonucleic acid; SAE: serious adverse event; TIBC: total iron-binding capacity; ULN: upper limit of normal.
NOTES:
^{a} Details of liver chemistry stopping criteria and required actions and follow-up after observations of ALT >3 × ULN are given under headings Liver chemistry stopping criteria and Liver and other safety. All events of ALT >3 × ULN which may indicate liver injury must be reported to the Sponsor in an expedited manner. Clinical laboratory findings of ALT >3 ULN and bilirubin ≥2 × ULN (>35% direct bilirubin) or ALT >3 × ULN and INR >1.5, if INR measured, that may suggest severe liver injury (possible Hy's Law) must be reported as an SAE.
^{b} Other renal function parameters, creatinine clearance (CrCl) will be calculated.
^{c}With the mentioned exception of the Screening Visit when a serum test is required, local monthly urine testing will be standard for the protocol unless serum testing is required by local regulation or IRB/IEC.
^{d}See E 03 for further details.

### Liver and Other Safety: Actions and Follow-Up Assessments

These actions described in Table 1H and FIGS. 2-8 are required for ALT increase and thrombocytopenia events ONLY. For all other safety events described, these are suggested per the Investigator's medical judgement.

Neutropenia is to be recorded as an AE only if at least 1 of the criteria listed in the general guidelines for reporting AEs is met.

Thrombocytopenia is to be recorded as an AE only if at least 1 of the criteria listed in the general guidelines for reporting AEs in Appendix 3 (Section 10.3) is met.

Abbreviations in FIG. 4A: ALT: alanine aminotransferase; ANCA: antineutrophil cytoplasmic antibodies; AST: aspartate aminotransferase; CMV: cytomegalovirus; CRF: case report form; DNA: deoxyribonucleic acid; dsDNA: double stranded DNA; EBV: Epstein-Barr Virus; GGT: Gamma-glutamyl transferase; HAV: Hepatitis A virus; HBc: Hepatitis B core; HBV: Hepatitis B virus; HCV: Hepatitis C virus; HEV: Hepatitis E virus; IgM: immunoglobulin M; IMP: investigational medicinal product, INR international normalized ratio; lab: laboratory; LFT: liver function test; PT: prothrombin time, RNA: ribonucleic acid; LKM: liver kidney microsome; ULN: upper limit of normal.

Note: “Baseline” refers to ALT sampled at baseline visit; or if baseline value unavailable, to the latest ALT sampled before the baseline visit. The algorithm does not apply to the instances of increase in ALT during Screening.

TABLE 1H
Actions for cases of confirmed ALT elevation
In ANY CONFIRMED CASE, FOLLOW the instructions listed below:

INFORM the Site Monitor, who will forward the information to the Study Manager.
INVESTIGATE specifically for malaise with or without loss of consciousness,
dizziness, and/or hypotension and/or episode of arrhythmia in the previous 72
hours; rule out muscular injury.
PERFORM the following tests:
LFTs: AST, ALT, alkaline phosphatase, total and conjugated bilirubin and
prothrombin time/INR;
CPK, serum creatinine, complete blood count;
Anti-HAV IgM, anti-HBc IgM, (HBV-DNA if clinically indicated),
anti-HCV and HCV RNA, anti-CMV IgM and anti- HEV IgM
antibodies;
Depending on the clinical context, check for recent infection with EBV, herpes
viruses, and toxoplasma;
Consider iron, ferritin, transferrin saturation;
Consider hepatobiliary ultrasonography (or other imaging investigations if
needed);
CONSIDER auto-antibodies: antinuclear, anti-DNA, anti-smooth muscle, anti-LKM,
anti-mitochondrial;
CONSIDER DNA test for Gilbert's disease if clinically indicated;
CONSIDER consulting with a hepatologist;
CONSIDER patient hospitalization if INR >2 (or PT <50%) and/or central
nervous system disturbances suggesting hepatic encephalopathy;
MONITOR LFTs after permanent discontinuation of IMP:
Monitor as closely as possible (every 48 hours to every week)
until ALT is down-trending, then every 2 weeks until 1.5 ×
ULN, and then every scheduled visit;
Rechallenge Re-initiation of the study drug can only be considered after
discussion with the Sponsor's Medical Monitor once the ALT/AST decreases
below 1.5 × ULN, and there is no clinical contraindication. In case it is agreed
to re-start the study drug, it is recommended that ALT/AST be assessed
weekly for the first month and then monthly for the second and third months.
The occurrence of new elevation above 3 × ULN for the ALT/AST values will
lead to permanent discontinuation of the study drug; No rechallenge will be
considered for participants with >3 × ULN ALT and >2 × ULN bilirubin
increase
FREEZE serum sample (5 ml × 2).
COLLECT/STORE one PK sample following the instructions in the central
laboratory manual

Increase in serum creatinine is to be recorded as an AE only if at least 1 of the criteria listed in the general guidelines for reporting adverse events in Appendix 3 (Section 10.3) is met.

Increase in CPK is to be recorded as an AL only if at least 1 of the criteria in the general guidelines for reporting adverse events is met.

Examples of Drugs with a Potential to Change Tolebrutinib Metabolism

The following drugs should not be taken during the study concomitantly with the IMP due to their potential to change tolebrutinib kinetics due to interaction with P450-mediated metabolism, being potent/moderate inducers of CYP3A or potent inhibitors of CYP2C8 liver enzymes (per the lists of the Drug Interaction Database Program of the University of Washington).

Please note that the lists provided are not exhaustive and that the product information of drugs intended for concomitant use should be consulted.

TABLE 1J
CYP3A Inducers and CYP2C8 Inhibitors

| | |
|---|---|
| Potent CYP3A Inducers: | |
| rifampin | carbamazepine |
| phenobarbital | St John's wort extract |
| avasimibe | lumacaftor |
| rifapentine | rifabutin |
| phenytoin | |
| Potent CYP2C8 Inhibitors: | |
| gemfibrozil | |
| clopidogrel | |
| Moderate CYP3A Inducers: | |
| semagacestat | rifabutin |
| cenobamate | nafcillin |
| lesinurad | asunaprevir/beclabuvir/daclatasvir |
| bosentan | modafinil |
| thioridazine | telotristat ethyl |
| | elagolix |

### Abbreviations

AChEI: acetylcholinesterase inhibitor
AChR: acetylcholine receptor
ADL: activities of daily living
AE: adverse event
AESI: adverse event of special interest
ALT: alanine aminotransferase
ANCOVA: analysis of covariance
AUC: area under the curve
BCR: B-cell receptor
BTK: Bruton's tyrosine kinase
BUN: blood urea nitrogen
CFR: Code of Federal Regulations
CI: confidence interval
CIOMS: Council for International Organizations of Medical Sciences
COVID-19: coronavirus disease 2019
CPK: creatine phosphokinase
CRF: case report form
CS: corticosteroid(s)
CSICF: core study informed consent form
C-SSRS: Columbia Suicide Severity Rating Scale
CTCAE: common terminology criteria for adverse event(s)
CYP: cytochrome
DB: double-blind
DILI: drug-induced liver injury
DTP: direct-to-patient
ECG: electrocardiogram
eCRF: electronic case report form
EOS: end of study
EOT: end of treatment
EQ: EuroQoL
EQ-5D: EuroQoL 5 Dimensions
EQ-5D-5L: EuroQoL 5 Dimensions 5 Levels
EQ-VAS: EuroQoL-visual analogue scale
EU: European Union
FcRn: neonatal Fc receptor
FcγR: Fc-gamma receptor
FcεR: Fc-epsilon receptor
FSH: follicle-stimulating hormone
FU: follow-up
GCP: good clinical practice
GDPR: General Data Protection Regulation
gMG: generalized myasthenia gravis
HCRU-MG: healthcare resource utilization myasthenia gravis
HIV: human immunodeficiency virus
HRT: hormone replacement therapy
IA: interim analysis
IB: Investigator's Brochure
ICE: intercurrent event
ICF: informed consent form
ICH: International Council for Harmonisation
IDMC: Independent Data Monitoring Committee
IEC: Independent Ethics Committee
Ig: immunoglobulin
IgG: immunoglobulin G
IgM: immunoglobulin M
IMP: investigational medicinal product
IMP: investigational medicinal product
IRB: Institutional Review Board
IST: immunosuppressive treatment
ITT: intention-to-treat
IUD: intrauterine device
IUS: intrauterine hormone-releasing system
IVIg: intravenous immunoglobulin
MG: myasthenia gravis
MG-ADL: myasthenia gravis-activities of daily living
MGFA: myasthenia gravis foundation of America
MGFA-PIS: myasthenia gravis foundation of America-post-intervention status
MGII: myasthenia gravis impairment index
MG-QoL15: myasthenia gravis-quality of life 15-item scale
mITT: modified intention-to-treat
MRM: mixed-effect model with repeated measures
MS: multiple sclerosis
MuSK: muscle-specific kinase
NCI: National Cancer Institute
NSAIDs: nonsteroidal anti-inflammatory drugs
OCS: oral corticosteroid(s)
OLE: open-label extension
PCSA: potentially clinically significant abnormalities
PD: pharmacodynamic(s)
pEOT: premature end of treatment
PGIS: Patient Global Impression of Severity
PK: pharmacokinetic(s)
QMG: quantitative myasthenia gravis
QoL: quality of life
QTcF: QT interval corrected using Fridericia's formula
RMS: relapsing multiple sclerosis
SAE: serious adverse event
SAP: statistical analysis plan
SD: standard deviation
SoA: schedule of activities
SoC: standard of care
SUSAR: suspected unexpected serious adverse reaction
TE: treatment-emergent
TEAE: treatment-emergent adverse event
TLR: toll-like receptor
ULN: upper limit of normal
US: United States
VAS: visual analog scale
WOCBP: woman of childbearing potential

### Example 2—A Phase 3, Randomized, Double-Blind Efficacy and Safety Study Comparing SAR442168 to Teriflunomide (Aubagio®) in Participants with Relapsing Forms of Multiple Sclerosis (Gemini 2)

The goal of this Phase 3 study is to assess SAR442168 in the RMS population. Efficacy will be assessed by adjudicated relapse rate, disability progression, and MRI findings of disease activity (Gd-enhancing lesions and new/enlarging T2-hyperintense lesions). Together with evaluation of other secondary and exploratory endpoints, this study will provide a comprehensive evaluation of the efficacy and safety of SAR442168 in the RMS population.

A graphical scheme of the study design is shown in FIG. 1B. Abbreviations used in FIG. 1B: EOS, end of study; MRI, magnetic resonance imaging; R, randomization. ‘Month-1 (D-28-D-1)’ refers to screening period as “Day-28 to Day-1”; ‘Month 0 (D1)’ refers to randomization on Day 1.

Table 2A1 and 2A2 shown below describe the schedule of activities during the course of study. Table 2B that follows describes the objective and endpoints of the overall study.

TABLE 2A1
Schedule of Activities (SOA)- Screening and Year 1

| | | Randomization/ | | | | | | | | | |
|---|---|---|---|---|---|---|---|---|---|---|---|
| | Screening^{a} | Start of IMP | Year 1 (M12)^{b} | | | | | | | | |
| | Visit (a window of ±7 days is | | | | | | | | | | |
| | allowed for all visits after D 1) | | | | | | | | | | |
| | D-28 to | | | | | | | M 4, | | | |
| | D-1 | D 1 | M 1^{d} | M 1.5 | M 2^{d} | M 2.5 | M 3 | M 5^{d} | M 6 | M 9 | M 12 |
| | Visit number | | | | | | | | | | |
| | | | | | | | | V6, | | | |
| Procedure | V1 | V2 | V3 | | V4 | | V5 | V7 | V8 | V9 | V10 |
| Informed consent | X | | | | | | | | | | |
| Demography | X | | | | | | | | | | |
| Inclusion/ | X | X | | | | | | | | | |
| exclusion criteria | | | | | | | | | | | |
| Medical/ | X | | | | | | | | | | |
| surgical history | | | | | | | | | | | |
| Prior/ | <=================================================================================> | | | | | | | | | | |
| concomitant | | | | | | | | | | | |
| medications^{g} | | | | | | | | | | | |
| Randomization | | X | | | | | | | | | |
| IRT contact | X | X | | | | | X | | X | X | X |
| IMP | | X | | | | | X | | X | X | X |
| dispensation^{e} | | | | | | | | | | | |
| IMP compliance | | | | | | | X | | X | X | X |
| Paper diary | | X | | | | | X | | X | X | X |
| dispensation/ | | | | | | | | | | | |
| collection | | | | | | | | | | | |

Safety^{h}

| Physical | X | X | | | | | X | | X | X | X |
|---|---|---|---|---|---|---|---|---|---|---|---|
| examinationⁱ | | | | | | | | | | | |
| and vital signs | | | | | | | | | | | |
| Height | X | | | | | | | | | | |
| Body weight | X | X | | | | | X | | X | | |
| Serology tests | X | | | | | | | | | | |
| for hepatitis B, | | | | | | | | | | | |
| C (HIV and | | | | | | | | | | | |
| other infectious | | | | | | | | | | | |
| diseases, if | | | | | | | | | | | |
| required locally) | | | | | | | | | | | |
| TB/QuantiFERON ® | X | | | | | | | | | | |
| TB Gold | | | | | | | | | | | |
| test or | | | | | | | | | | | |
| equivalent^{j} | | | | | | | | | | | |
| Body | X | X | | | | | X | | X | X | X |
| temperature | | | | | | | | | | | |
| 12-lead ECG | X | | | | | | X | | X | X | X |
| Hematology, | X | X^{w} | X | | X | | X | X | X | X | X |
| biochemistry^{k} | | | | | | | | | | | |
| Liver | | | | X | | X | | | | | |
| function | | | | | | | | | | | |
| tests^{l} | | | | | | | | | | | |
| Iron panel | X | | | | | | | | | | |
| (serum): iron, | | | | | | | | | | | |
| ferritin, | | | | | | | | | | | |
| transferrin | | | | | | | | | | | |
| saturation, | | | | | | | | | | | |
| TIBC; to be | | | | | | | | | | | |
| repeated during | | | | | | | | | | | |
| the study if | | | | | | | | | | | |
| needed | | | | | | | | | | | |
| Coagulation: | X | | | | | | | | | | |
| PT/INR, aPTT | | | | | | | | | | | |
| (to be repeated | | | | | | | | | | | |
| during the study, | | | | | | | | | | | |
| if needed) | | | | | | | | | | | |
| Urinalysis | X | | | | | | X | | | | X |
| Pregnancy test | X | X | Xⁿ | | | | X | Xⁿ | X | X | X |
| (if applicable)^{m,n} | | | | | | | | | | | |
| Serum FSH^{o} | X | | | | | | | | | | |
| Suicidality | X | X | | | | | X | | X | X | X |
| assessment by | | | | | | | | | | | |
| C-SSRS | | | | | | | | | | | |
| Adverse event | <=================================================================================> | | | | | | | | | | |
| collection | | | | | | | | | | | |
| Efficacy | | | | | | | | | | | |
| EDSS | X | X | | | | | X | | X | X | X |
| Timed 25-foot | | X | | | | | X | | X | X | X |
| walk test | | | | | | | | | | | |
| 9-hole peg test | | X | | | | | X | | X | X | X |
| SDMT and | | X | | | | | X | | X | X | X |
| CVLT-II, | | | | | | | | | | | |
| where available^{p} | | | | | | | | | | | |
| Basic or | X^{q} | | | | | | | | X | | X |
| expanded MRI | | | | | | | | | | | |
| scan^{q} | | | | | | | | | | | |
| Clinical outcome assessment^{r} | | | | | | | | | | | |
| MSQoL-54 | | X | | | | | | | X | | X |
| EQ-5D-5L | | X | | | | | | | X | | X |
| Pharmacogenetics^{s} | | | | | | | | | | | |
| DNA sample^{u} | | X | | | | | | | | | |
| Pharmacodynamics/biomarkers^{s} | | | | | | | | | | | |
| Blood sample for | X | | | | | | | | | | |
| archiving^{v} | | | | | | | | | | | |
| Plasma samples | | X | | | | | X | | X | | X |
| (NfL), serum | | | | | | | | | | | |
| samples | | | | | | | | | | | |
| (Chi3L1)^{t} | | | | | | | | | | | |
| Serum | | X | | | | | | | X | | X |
| samples (Ig | | | | | | | | | | | |
| levels)^{t} | | | | | | | | | | | |

TABLE 2A2
Schedule of Activities (SOA)- From M15 to Follow-up Visit

| | | | Only for | | Only for participants |
|---|---|---|---|---|---|
| | | | participants who | | who completed |
| | | | prematurely | For all | treatment to EOS but |
| | From M 15 to EOS^{b} | | discontinue IMP | participants | do not enter LTS^{c} |
| | Visit (a window of ±7 days is allowed for all visits after D 1) | | | | |
| | Quarterly visits | Semi-annual | | EOS^{f} | |
| | (M 15, 18, 21, 24, | visits (M 18, M 24, | | “Common study | FU visit (4 to 8 weeks |
| | 27, 30, 33, M 36 . . . ) | M 30, M 36 . . . ) | pEOT^{e} | end date” visit | after EOS) |
| | Visit number | | | | |
| | V11, V12, V13, | | | | |
| | V14, V15, V16, | V12, V14, V16, | | | |
| Procedure | V17, V18 . . . | V18 . . . | pEOT^{e} | EOS | FUV |

Informed consent
Demography
Inclusion/exclusion
criteria
Medical/surgical
history

| Prior/concomitant | <===================================================================================> | | | | |
|---|---|---|---|---|---|
| medications^{g} | | | | | |
| Randomization | | | | | |
| IRT contact | X | X | X | X | X |
| IMP dispensation^{e} | X | X | | | |
| IMP compliance | X | X | X^{e} | X | |
| Paper diary | X | X | X | X | |
| dispensation/ | | | | | |
| collection | | | | | |

Safety^{h}

| Physical | X | X | X | X | X |
|---|---|---|---|---|---|
| examinationⁱ | | | | | |
| and vital signs | | | | | |
| Height | | | | | |
| Body weight | | X | X | X | X |
| Serology tests | | | | | |
| for hepatitis B, | | | | | |
| C (HIV and | | | | | |
| other infectious | | | | | |
| diseases, if | | | | | |
| required locally) | | | | | |
| TB/QuantiFERON ® | | | | | |
| TB Gold test or | | | | | |
| equivalent^{j} | | | | | |
| Body temperature | X | X | X | X | X |
| 12-lead ECG | | yearly | X | X | |
| Hematology, | X | X | X | X | X |
| biochemistry^{k} | | | | | |
| Liver function | | | | | |
| tests^{l} | | | | | |
| Iron panel (serum): | | | | | |
| iron, ferritin, | | | | | |
| transferrin | | | | | |
| saturation, TIBC; | | | | | |
| to be repeated | | | | | |
| during the study | | | | | |
| if needed | | | | | |
| Coagulation: | | | | | |
| PT/INR, aPTT | | | | | |
| (to be repeated | | | | | |
| during the study, | | | | | |
| if needed) | | | | | |
| Urinalysis | | X | X | X | |
| Pregnancy test | X | Xⁿ | X | X | X |
| (if applicable)^{m, n} | | | | | |
| Serum FSH^{o} | | | | | |
| Suicidality | X | X | X | X | X |
| assessment by | | | | | |
| C-SSRS | | | | | |
| Adverse event | <=================================================================================> | | | | |
| collection | | | | | |

Efficacy

| | | | | |
|---|---|---|---|---|
| EDSS | X | X | X | X |
| Timed 25-foot | X | X | X | X |
| walk test | | | | |
| 9-hole peg test | X | X | X | X |
| SDMT and CVLT-II, | X | X | X | X |
| where available^{p} | | | | |
| Basic or expanded | | M 18, M 24, M 36 | X | X |
| MRI scan^{q} | | | | |

Clinical outcome assessment^{r}

| | | | |
|---|---|---|---|
| MSQoL-54 | X | X | X |
| EQ-5D-5L | X | X | X |

Pharmacogenetics^{s}

DNA sample^{u}

Pharmacodynamics/biomarkers^{s}

| | | | |
|---|---|---|---|
| Blood sample | | | |
| for archiving^{v} | | | |
| Plasma samples | yearly | X | X |
| (NfL), serum | | | |
| samples (Chi3L1)^{t} | | | |
| Serum samples | yearly | X | X |
| (Ig levels)^{t} | | | |

aPTT: activated partial thromboplastin time; β-HCG: β-human chorionic gonadotropin; AST: aspartate aminotransferase; ALT: alanine aminotransferase; Chi3L1: chitinase-3 like protein-1; CRF: case report form; C-SSRS: Columbia Suicide Severity Rating Scale; D: day; DNA: deoxyribonucleic acid; ECG: electrocardiogram; EDSS: Expanded Disability Status Scale; EOS: end of study; EOT: end of treatment; EQ-5D-5L: EuroQol 5-dimension 5-level questionnaire; FSH: follicle-stimulating hormone; FU: follow-up; Ig: immunoglobulin; HIV: human immunodeficiency virus; ICF: informed consent form; IMP: investigational medicinal product; INR: international normalized ratio; IRT: interactive response technology; LTS: long-term safety study; M: month (28 days); MRI: magnetic resonance imaging; MS: multiple sclerosis; MSQoL-54: Multiple Sclerosis Quality of Life-54; NfL: neurofilament light chain; pEOT: premature end of treatment; PT: prothrombin time; SDMT: Symbol Digit Modalities Test; SWI: susceptibility-weighted imaging; TB: tuberculosis; TIBC: total iron-binding capacity; V: visit; WBC: white blood cell.
Note:
All assessments shall be done as designated in this SoA unless not permitted according to local regulations. All visit assessments should be performed during the visit window unless otherwise specified in this protocol.
^{a} Screening period can range from D −28 to D −1; Randomization visit can be performed only once IMPs are available at site. The interval between screening and randomization visits can range from 11 days (minimum) to 28 days (maximum). However, if required, the randomization visit can be performed earlier than 11 days upon IMP receipt at the site, assuming the participant is eligible for randomization. In case the screening MRI must be rescheduled (eg, technical issues) or repeated, an additional 1 week (7 days) is allowed.
^{b}From D 1 to EOS, unscheduled visits may be performed at any time by the Investigator (eg, for evaluation of an adverse event). Assessments may be done as needed to evaluate the participant in accordance with the Investigator's best judgment and in line with the study protocol. At a minimum, a physical examination should be performed, and, body temperature and vital signs should be measured.
^{c}At the EOS, the participants who have completed the study and remain on IMP will be offered participation in a long-term safety study. Follow-up visit assessment only performed for those participants who are not willing to take part in the long-term safety study.
^{d} These visits may be done as home health visits (where applicable) or onsite visits (preferable that tests are performed at the central laboratory). In any situations where this is not possible (to be documented in source documents), the tests for these visits may be performed at a local laboratory.
^{e}If a participant prematurely permanently discontinues treatment with IMP, the participant will undergo a premature EOT visit as soon as possible. Participants will then be asked to continue with the study visits as scheduled, until global EOS visit is reached. During these visits, all study procedures/assessments will be performed except IMP administration and blood sampling for biomarkers (NfL, Chi3L1, and Ig levels). MRI scans for these participants will only be performed annually (using the next annual visit as the starting point).
^{f}Common EOS visit will be done when the prespecified number of events for 6-month CDW is expected to be reached. The timing and window of this visit will be communicated to sites.
^{g}Any disease-modifying therapy for MS taken at any time prior to signing the informed consent need to be reported in the CRF; other prior medications will be reported for the period of 6 months prior to signing the ICF.
^{h}Additional safety assessments can be performed if required by local regulations; such testing may be performed locally. Additional visits may be added if required by local regulations.
ⁱComplete physical examination due at screening, baseline, yearly and EOS. Brief physical examination is sufficient for the rest of the visits (complete and brief physical examination will include neurological examination and collection of the following vital signs: arterial blood pressure, heart rate, and temperature).
^{j}To be performed at screening for all participants. To be repeated based on clinical judgment, borderline results, or clinical suspicion of TB infection. Screening tests for TB are described in Table 2F.
^{k}Hematology (platelet count, red blood cell count, hemoglobin, hematocrit, mean corpuscular volume, mean corpuscular hemoglobin, reticulocytes, white blood cell count with differential: neutrophils, lymphocytes, monocytes, eosinophils, basophils).
Biochemistry (blood urea nitrogen [BUN], creatinine, glucose, potassium, sodium, chloride, bicarbonate, calcium; liver function tests [AST, ALT, albumin, alkaline phosphatase, total and direct bilirubin, total protein; creatine phosphokinase], lipase at Screening Visit, then quarterly). Monthly visits (M 1, M 2, M 4, M 5) will include hematology and full liver panel only. Additional safety assessments can be performed if required by local regulations. Such testing shall be performed at local laboratories. Note: a one-time retest at screening may be performed if an abnormal laboratory test value is considered temporary. Additional visits may be added if required by local regulations.
^{l}At intermediate timepoints M 1.5 and M .2.5, only liver function tests will be collected (AST, ALT, albumin, alkaline phosphatase, total and direct bilirubin, total protein, and creatine phosphokinase); these can be central or local labs.
^{m}Serum β-HCG pregnancy test at central laboratory at screening and urine pregnancy tests within 24 hours before the first dose of IMP and at scheduled times during study. Additional serum or urine pregnancy tests may be performed, as determined necessary by the Investigator or required by local regulation, to establish the absence of pregnancy at any time during the participant's participation in the study. Communication by phone of the result of a pregnancy test performed at home to the site is allowed.
ⁿPregnancy tests will be performed monthly in all concerned EU countries.
^{o}Only in female participants, if needed to establish menopausal status.
^{p}SDMT and CVLT-II will be performed in all participants. If for some reason, CVLT-II is not available at a given site due to reasons such as lack of translation, local certification, etc, then only the SDMT will be assessed for that participant.
^{q}A subset of sites that have 3T MRI capacity will perform additional sequences (eg, SWI). Further details will be defined in a separate central MRI manual. For systemic corticosteroids and adrenocorticotropic hormone, 1 month washout is required prior to the MRI scans. A visit window of ±21 days is acceptable for MRIs performed after D 1.The screening MRI scan should be performed as close as possible before the start of IMP. As much as possible, the MRI scan should be performed during the screening period after it has been established that the participant meets all inclusion and no exclusion criteria.
^{r}When available, clinical outcome assessments are to be completed by the participant prior to discussing their health status and prior to study treatment administration or other study-related procedures.
^{s}Where available per local regulations.
^{t}Pharmacodynamics/biomarkers samples collected are not timed samples.
^{u}The DNA testing may be done, if locally applicable, at any time after signature of consent (in case it could not be done for some reason at Day 1).
^{v}This sample will be collected and stored for use if any unexpected safety issue occurs to ensure that a pre-dose baseline value is available for previously not assessed parameters (eg, serology) and for biomarkers research, if agreed.
^{w} Samples for hematology and biochemistry tests on Day 1 (randomization) must be collected prior to administration of the first IMP dose.

Objectives and endpoints for the treatment are shown in Table 2B.

TABLE 2B
Objectives and endpoints

| | |
|---|---|
| Objectives | Endpoints |

Primary

| | |
|---|---|
| To assess efficacy of daily tolebrutinib | ARR during the study period assessed by |
| compared to a daily dose of 14 mg | confirmed protocol-defined adjudicated |
| teriflunomide (Aubagio) measured by | relapses |
| annualized adjudicated relapse rate (ARR) in | |
| participants with relapsing forms of MS | |

Secondary

| | |
|---|---|
| To assess efficacy of tolebrutinib compared | Time to onset of confirmed disability |
| to teriflunomide (Aubagio) on disability | worsening (CDW), confirmed over at least |
| progression, magnetic resonance imaging | 6 months, defined as follows: |
| (MRI) lesions, cognitive performance and | increase of ≥1.5 points from the |
| quality of life | baseline Expanded Disability Status |
| | Scale (EDSS) score when the baseline |
| | score is 0, OR |
| | increase of >1.0 point from the |
| | baseline EDSS score when the baseline |
| | score is 0.5 to ≤5.5, OR |
| | increase of ≥0.5 point from the |
| | baseline EDSS score when the baseline |
| | score is >5.5 |
| | Time to onset of CDW, assessed by the |
| | EDSS score and confirmed over at least 3 |
| | months |
| | Total number of new and/or enlarging T2- |
| | hyperintense lesions as detected by MRI, |
| | defined as the sum of the individual |
| | number of new and/or enlarging T2 |
| | lesions at all scheduled visits starting after |
| | baseline up to and including the End-of- |
| | Study (EOS) visit and number of new |
| | and/or enlarging |
| | T2-hyperintense lesions by visit over time |
| | Total number of new gadolinium- (Gd—) |
| | enhancing |
| | T1-hyperintense lesions as detected by |
| | MRI, defined as the sum of the individual |
| | number of new Gd— enhancing |
| | T1-hyperintense lesions at all scheduled |
| | visits starting after baseline up to and |
| | including the EOS visit |
| | Time to confirmed disability improvement |
| | (CDI), defined as a ≥1.0 point decrease on |
| | the EDSS from the baseline EDSS_score |
| | confirmed over at least 6 months |
| | Percent change in brain volume loss as |
| | detected by brain MRI scans at the EOS |
| | compared to Month 6 |
| | Change in cognitive function at the EOS |
| | compared to baseline as assessed by the |
| | Symbol Digit Modalities Test (SDMT) |
| | Change in cognitive function at the EOS |
| | compared to baseline as assessed by the |
| | CVLT-II, where available |
| | Change in Multiple Sclerosis Quality of |
| | Life 54 (MSQoL-54) questionnaire score at |
| | the EOS compared to baseline |
| To evaluate the safety and tolerability of | Adverse events (AEs), serious AEs, AEs |
| daily tolebrutinib | leading to permanent study intervention |
| | discontinuation, AEs of special interest, and |
| | potentially clinically significant |
| | abnormalities in laboratory tests, safety |
| | scales, ECG, and vital signs during the |
| | study period |
| To evaluate pharmacodynamics (PD) of | Change in plasma neurofilament light chain |
| tolebrutinib | (NfL) levels at the EOS compared to baseline, |
| | where available |
| | Changes in serum immunoglobulin level at the |
| | EOS compared to baseline |
| | Change in serum Chi3L1 levels at the EOS |
| | compared to baseline |
| Tertiary/exploratory | EDSS score change from baseline at |
| To evaluate the efficacy of tolebrutinib on | scheduled visits starting after baseline and |
| disease activity as measured by additional | including the EOS visit |
| clinical, brain MRI, and composite | Proportion of adjudicated relapse-free |
| measurements | participants from randomization until the |
| | EOS visit |
| | Time to onset of 20% worsening in the 9- |
| | hole peg test (9-HPT) confirmed over at |
| | least 3 and 6 months |
| | Time to onset of 20% worsening in the |
| | timed 25-foot walk (T25-FW) test |
| | confirmed over at least 3 and 6 months |
| | Time to onset of 4-point decrease in Symbol |
| | Digit Modalities Test (SDMT) confirmed |
| | over at least 3 and 6 months |
| | Change from baseline of total volume of |
| | T2-hyperintense lesions as detected by brain |
| | MRI at Months 18, 24, and the EOS |
| | Magnetization transfer ratio recovery at the |
| | EOS in new magnetization transfer ratio |
| | lesions detected at Months 6 and 12 |
| | Change in number of phase rim lesions in |
| | susceptibility weighted imaging (SWI) MRI |
| | from baseline by visit over time (subset of |
| | centers with capacity of 3T MRI) |
| | Proportion of participants with no evidence |
| | of disease activity (NEDA-3) at Months 18, |
| | 24, and the EOS |
| | Change from baseline to Months 12, 18, and |
| | 24 and to the EOS in modified Multiple |
| | Sclerosis Functional |
| | Composite 3 (MSFC-3), assessed as the |
| | composite of the T25-FW test, 9-HPT, and |
| | SDMT |
| | Change from baseline by visit over time in |
| | volume of T1-hypointense lesions, and |
| | cumulative number of new T1-hypointense |
| | lesions |
| | Number and volume of slowly evolving |
| | lesions (SELs) |
| | Normalized T1 intensity evolution in SELs |
| To evaluate the treatment effect of | Change in EuroQol 5-dimension 5-level |
| SAR442168 via changes in participants' | questionnaire (EQ-5D-5L) from baseline by |
| health-related quality of life (HRQoL), and | visit over time |
| working capacity | |

### Overall Design:

This is a Phase 3, randomized, double-blind, double-dummy, 2-arm, active-controlled, parallel group, multicenter, event-driven (6-month confirmed disability worsening [CDW]) trial with a variable treatment duration ranging from approximately 18 to 36 months.

### Disclosure Statement:

This is a parallel treatment study with 2 arms that is blinded/masked for participants, any Investigator, site staff, and the Sponsor.

### Number of Participants:

Approximately 1200 people will be screened to achieve approximately 900 (±10%) participants randomly assigned to the study intervention with a total sample size of at least 1800 patients across the 2 RMS studies of identical design (EFC16033 and EFC16034).

### Intervention Groups and Duration:

Participants will be randomly assigned at a 1:1 ratio to receive the 60 mg selected dose (established from dose-finding Study DRI15928) of oral SAR442168 daily as well as a placebo to match the teriflunomide tablet or 14 mg oral teriflunomide as well as a placebo to match the SAR442168 tablet daily. Randomization will be stratified by the Expanded Disability Status Scale (EDSS) score at screening (<4 versus ≥4) and geographic region (US versus non-US).

### Study Intervention(s)

Investigational medicinal product
Formulation: tolebrutinib film-coated tablet
Route(s) of administration: oral
Dose regimen: 60 mg once daily

### Investigational Medicinal Product

Formulation: teriflunomide tablet
Route of administration: oral
Dose regimen: 14 mg once daily

### Investigational Medicinal Product

Formulation: placebo to match SAR442168 film-coated tablet
Route of administration: oral
Dose regimen: once daily

### Investigational Medicinal Product

Formulation: placebo to match teriflunomide tablet
Route of administration: oral
Dose regimen: once daily

### Noninvestigational Medicinal Products

Formulation: MRI contrast-enhancing preparations
Route(s) of administration: intravenous (IV)
Dose regimen: as per respective label

### Noninvestigational Medicinal Products

Formulation: cholestyramine
Route(s) of administration: oral
Dose regimen: 8 g 3 times daily for 11 days for accelerated elimination procedure
   (4 g 3 times daily for 11 days in case of intolerance). The teriflunomide local label should be followed.

### Temporary Investigational Medicinal Product (IMP) Interruption Due to Surgery

If surgery is needed during the study, consider the benefit/risk of withholding the IMP for at least 3 to 7 days pre- and post-surgery depending upon the type of surgery and the risk of bleeding.

The goal of this Phase 3 study is to assess SAR442168 in the RMS population. Efficacy will be assessed by adjudicated relapse rate, disability progression, and MRI findings of disease activity (Gd-enhancing lesions and new/enlarging T2-hyperintense lesions). Together with evaluation of other secondary and exploratory endpoints, this study will provide a comprehensive evaluation of the efficacy and safety of SAR442168 in the RMS population.

In the ongoing Phase 3 and LTS studies, tolebrutinib has been generally well tolerated to date. An identified risk for tolebrutinib has been identified as follows:
Treatment-emergent SAEs of drug-induced liver injury (DILI) were reported in the ongoing Phase 3 trials; however, all cases occurred between Months 2 to 3 and appear reversible after treatment discontinuation, with potential confounders identified for some of the cases.

### Study Population

### Inclusion Criteria

Participants are eligible to be included in the study only if all of the following criteria apply as shown in Table 2C.

TABLE 2C
Inclusion Criteria

| Category | Criteria | |
|---|---|---|
| Age | I 01. | The participant must be 18 to 85 years of age inclusive, |
| | | at the time of signing the informed consent. |
| Type of | I 02. | The participant must have been diagnosed with RMS according to |
| participant | | the 2017 revision of the McDonald diagnostic criteria (Thompson |
| and disease | | et al. Lancet Neurol. 2018, 17, 162). |
| characteristics | I 03. | The participant has an EDSS score ≤5.5 at the first visit |
| | | (Screening Visit) |
| | I 04. | The participant must have at least 1 of the following |
| | | prior to screening: |
| | | ≥1 documented relapse within the previous year OR |
| | | >2 documented relapses within the previous 2 years, |
| | | OR |
| | | ≥1 documented Gd-enhancing lesion on an MRI scan |
| | | within the previous year |
| | | Note: The initial clinical demyelinating episode of MS should |
| | | be counted as a relapse for the first 2 criteria. |
| Weight | I 05. | Not Applicable. |
| Sex | I 06. | Male or Female. |
| | | Contraceptive use by men or women should be consistent with local |
| | | regulations regarding the methods of contraception for those participating |
| | | in clinical studies. |
| | | Male participants wishing to conceive a child and female participants |
| | | becoming pregnant or wishing to become pregnant must permanently |
| | | discontinue the study intervention and follow the local teriflunomide label |
| | | recommendation. |
| | | a) Male participants |
| | | Male participants are eligible to participate if they agree to the following |
| | | during the intervention period and until the accelerated elimination |
| | | procedure is performed. |
| | | Refrain from donating sperm Plus either: |
| | | Be abstinent from heterosexual intercourse as their preferred and |
| | | usual lifestyle (abstinent on a long-term and persistent basis) and |
| | | agree to remain abstinent |
| | | OR |
| | | Must agree to use contraception/barrier method as detailed below |
| | | Agree to use a male condom and should also be advised of |
| | | the benefit for a female partner to use a highly effective |
| | | method of contraception as a condom may break or leak |
| | | when having sexual intercourse with a woman of |
| | | childbearing potential (WOCBP) who is not currently |
| | | pregnant |
| | | b) Female participants |
| | | A female participant is eligible to participate if she is not |
| | | pregnant or breastfeeding, and at least one of the following |
| | | conditions applies: |
| | | Is not a woman of childbearing potential (WOCBP); or |
| | | Is a WOCBP and agrees to use a contraceptive method that is |
| | | highly effective (with a failure rate of <1% per year), |
| | | preferably with low user dependency, during the intervention |
| | | period and until the accelerated elimination procedure is |
| | | completed after the last dose of study intervention |
| | | A WOCBP must have a negative highly sensitive pregnancy |
| | | test at screening and within 24 hours before the first dose of |
| | | study intervention. If a urine test cannot be confirmed as |
| | | negative (eg, an ambiguous result), a serum pregnancy test is |
| | | required. In such cases, the participant must be excluded from |
| | | participation if the serum pregnancy result is positive. |
| | | Additional requirements for pregnancy testing during and after study |
| | | intervention are located in the schedule of activities (SoA); Tables |
| | | 2A1 and 2A2). |
| | | The Investigator is responsible for review of medical history, |
| | | menstrual history, and recent sexual activity to decrease the |
| | | risk for inclusion of a woman with an early undetected |
| | | pregnancy, if allowed by local regulations. |

### Exclusion Criteria

Participants are excluded from the study if any of the following criteria apply as shown in Table 2D.

TABLE 2D
Exclusion Criteria

| Category | Criteria | |
|---|---|---|
| Medical conditions | E 01. | The participant has been diagnosed with PPMS according to |
| | | the 2017 revision of the McDonald diagnostic criteria |
| | | (Thompson et al. Lancet Neurol. 2018, 17, 162) or with |
| | | nonrelapsing SPMS (Lublin et al. Neurology 2014, 83, 278-286). |
| | E 02. | The participant has a history of infection or may be at risk for |
| | | infection: |
| | | A history of T-lymphocyte or T-lymphocyte-receptor |
| | | vaccination, transplantation (including solid organ, |
| | | stem cell, and bone marrow transplantation) and/or |
| | | antirejection therapy. |
| | | The participant has received any live (attenuated) |
| | | vaccine (including but not limited to varicella zoster, |
| | | oral polio, and nasal influenza) within 2 months |
| | | before the first treatment visit. |
| | | The participant has a lymphocyte count less than the |
| | | lower limit of normal (LLN) at the Screening Visit. |
| | | A history of diagnosis of progressive multifocal |
| | | leukoencephalopathy (PML) or evidence of findings |
| | | suggestive of PML on the screening MRI. |
| | | A history of infection with human immunodeficiency |
| | | virus (HIV) (eg, any known positive HIV test or |
| | | information from participant interview). |
| | | A history of active or latent tuberculosis (TB); TB |
| | | testing should be performed at screening and again |
| | | during the study, if clinically indicated, and may be |
| | | repeated based on clinical judgment, borderline |
| | | results, or clinical suspicion of TB infection. |
| | | Screening tests for TB are described in Table 2F. |
| | | NOTE: The Investigator may consult with an |
| | | infectious disease expert if required, eg, test results |
| | | are unclear or there is a suspicion of false positive test |
| | | results. If the infectious disease expert considers the |
| | | test results as false positive and not clinically relevant |
| | | and confirms that the participant can be enrolled in |
| | | the trial, the Investigator must document this in |
| | | source data and may then randomize the participant. |
| | | Persistent chronic or active recurring infection |
| | | requiring treatment with antibiotics, antivirals, or |
| | | antifungals. |
| | | Fever within 4 weeks of the Screening Visit (≥38° C.; |
| | | however, if due to brief and mild ear, nose, throat |
| | | viral infection participant may be included based on |
| | | the Investigator's judgment). |
| | | Participants at risk of developing or having |
| | | reactivation of hepatitis, ie, results at screening for |
| | | serological markers for hepatitis B and C viruses |
| | | indicating acute or chronic infection. See the Study |
| | | Manual for further details. |
| | | Any other active infections that would adversely |
| | | affect participation or IMP administration in this |
| | | study, as judged by the Investigator. |
| | E 03. | The presence of psychiatric disturbance or substance abuse as |
| | | evidenced by: |
| | | A history of any psychiatric disease, behavioral |
| | | condition, or depression requiring hospitalization |
| | | within 2 years prior to the Screening Visit. |
| | | A documented history of attempted suicide or suicidal |
| | | ideation of category 4 or 5 according to the Columbia |
| | | Suicide Severity Rating Scale (C-SSRS) |
| | | baseline/screening version over the 6 months prior to |
| | | the Screening Visit, OR if in the Investigator's |
| | | judgment, the participant is at risk for a suicide attempt. |
| | | Active alcohol use disorder or a history of alcohol or |
| | | drug abuse within 1 year prior to the Screening Visit. |
| | | Current alcohol intake >2 drinks per day for men and >1 drink |
| | | per day for women (1 drink = approximately 14 grams of |
| | | alcohol = 350 mL beer = 140 mL wine = 40 mL of spirits). |
| | E 04. | The following findings obtained during the Screening Visit |
| | | considered in the Investigator's judgment to be clinically |
| | | significant in the context of this clinical trial: |
| | | Any screening laboratory values outside normal limits. |
| | | Abnormal ECG. |
| | E 05. | Conditions that may predispose the participant to excessive |
| | | bleeding: |
| | | A bleeding disorder or known platelet dysfunction at |
| | | any time prior to the screening visit. |
| | | A platelet count <150 000/μL at the screening visit. |
| | | The participant has had major surgery within 4 weeks |
| | | prior to the screening visit, which could affect the |
| | | participant's safety (as judged by the Investigator) or |
| | | has planned any elective major surgery during the study. |
| | | A history of significant bleeding event within 6 |
| | | months prior to screening, according to the |
| | | Investigator's judgment such as, but not limited to |
| | | cerebral or gastrointestinal bleeding. |
| | E 06. | Conditions that would adversely affect participation in the |
| | | study or make the primary efficacy endpoint non-evaluable: |
| | | Sensitivity to any of the study interventions, |
| | | or components thereof, or has a drug or other |
| | | allergy that, in the opinion of the |
| | | Investigator, contraindicates participation in the study. |
| | | A short life expectancy due to pre-existing |
| | | health condition(s) as determined by their |
| | | treating neurologist. |
| | | A history or presence of significant other |
| | | concomitant illness according to the |
| | | Investigator's judgment such as, but not |
| | | limited to cardiovascular (including Stage III |
| | | or IV cardiac failure according to New York |
| | | Heart Association [NYHA] classification), or |
| | | renal (ie, undergoing dialysis), neurological, |
| | | endocrine, gastrointestinal, metabolic, |
| | | pulmonary (eg, interstitial pneumonia or |
| | | pulmonary fibrosis), or lymphatic disease |
| | | that would adversely affect participation in |
| | | this study. |
| | | Acute liver disease, cirrhosis, chronic liver |
| | | disease (unless considered stable for >6 months). |
| | | Confirmed screening ALT >1.5 × ULN OR AST >1.5 × ULN |
| | | OR alkaline phosphatase >2 × ULN (unless caused by non- |
| | | liver-related disorder or explained by a stable chronic |
| | | liver disorder) OR total bilirubin >1.5 × ULN (unless |
| | | due to Gilbert syndrome or non-liver-related disorder). |
| | | At screening, elevated transferrin saturation (>50% in |
| | | males and >40% in females) and/or with elevated |
| | | ferritin levels >500 μg/L. |
| | | Any malignancy within 5 years prior to the |
| | | Screening Visit (except for effectively |
| | | treated carcinoma in situ of the cervix or |
| | | adequately treated non-metastatic squamous |
| | | or basal cell carcinoma of the skin) will also |
| | | be exclusionary. |
| | | Any other medical condition(s) or |
| | | concomitant disease(s) making them non- |
| | | evaluable for the primary efficacy endpoint |
| | | or that would adversely affect participation |
| | | in this study, as judged by the Investigator. |
| Prior/concomitant | E 07. | The participant has received any of the following |
| therapy | | medications/treatments within the specified time frame before |
| | | any baseline assessment (no washout is required for dimethyl |
| | | fumarate, interferon beta, or glatiramer acetate treatments): |

| | Exclusionary if used/used within |
|---|---|
| Medication | required washout period |
| Systemic corticosteroids, | 1 month prior to screening MRI scan |
| adrenocorticotropic hormone | |
| Siponimod, ponesimod | 1 week before randomization with |
| | MRI and clinical assessment for |
| | PML prior to randomization |
| Plasma exchange | 1 month prior to randomization |
| IV immunoglobulin | 1 month prior to randomization |
| Fingolimod, ozanimod | 6 weeks before randomization with |
| | MRI and clinical assessment for PML |
| Mildly to moderately | 3 months prior to randomization |
| immunosuppressive/chemotherapeutic | |
| medications such as azathioprine and | |
| methotrexate, mycophenolate | |
| Lymphoid irradiation, bone marrow | A participant who has received any |
| transplantation, mitoxantrone (with | of these treatments at any time is not |
| evidence of cardiotoxicity following | eligible. |
| treatment, or cumulative lifetime | |
| dose >120 mg/m²), other strongly | |
| immunosuppressive treatments with | |
| very long-lasting effects | |
| Teriflunomide | |
| <3 months treatment | 3 months prior to randomization* |
| ≥3 months treatment | A participant who has received this |
| | treatment at any time is not eligible. |
| Natalizumab | 2 months before randomization with |
| | MRI and clinical assessments for PML |
| B-cell-depleting therapies such as | 6 months prior |
| ocrelizumab and rituximab | |
| Ofatumumab | 4 months prior |
| Highly | 2 years prior to randomization |
| immunosuppressive/chemotherapeutic | |
| medications: mitoxantrone up to 120 | |
| mg/m2 body surface area, | |
| cyclophosphamide, cladribine, | |
| cyclosporine | |
| Alemtuzumab | 4 years prior to randomization |
| Other MS-disease modifying | 5 half-lives or until end of pharmacodynamics |
| treatments | activity, whichever is longer |

Abbreviations: MRI: magnetic resonance imaging, MS:
multiple sclerosis, PML: progressive multifocal leukoencephalopathy
*No time restriction if accelerated elimination procedure is done

| Category | Criteria | |
|---|---|---|
| | E 08. | The participant is receiving potent and moderate inducers of |
| | | cytochrome P450 3A (CYP3A) or potent inhibitors of |
| | | CYP2C8 hepatic enzymes. |
| | E 09. | The participant is receiving anticoagulant/antiplatelet |
| | | therapies, including: |
| | | Acetylsalicylic acid (aspirin) >81 mg/day, |
| | | Antiplatelet drugs (eg, clopidogrel), |
| | | Warfarin (vitamin K antagonist), |
| | | Heparin, including low molecular weight heparin |
| | | (antithrombin agents), |
| | | Dabigatran (direct thrombin inhibitor), |
| | | Apixaban, edoxaban, rivaroxaban (direct factor Xa |
| | | inhibitors). |
| | | Note: All the above-mentioned drugs must be stopped at least 5 half- |
| | | lives before study drug administration except for aspirin, which must |
| | | be stopped at least 8 days before. If this is not clinically appropriate, |
| | | the participant cannot be included. These washout periods are only |
| | | applicable in the case that the Investigator deems it clinically |
| | | appropriate to discontinue the listed medications or there is a recent |
| | | history of use of these medications (as in the case when short term |
| | | treatment with anticoagulants is clinically recommended for certain |
| | | thrombotic events), and therefore these washout periods will need to |
| | | be followed prior to randomization. |
| | | If the participant has a chronic underlying medical condition (stroke, |
| | | coronary or carotid artery disease, heart valvular disease etc.) |
| | | requiring continued use of these medications, the participant cannot |
| | | be enrolled in the study. |
| | E 10. | A history of a hypersensitivity reaction to teriflunomide, |
| | | leflunomide, or to any of the inactive ingredients in Aubagio |
| | | (this includes anaphylaxis, angioedema, and serious skin |
| | | reactions). |
| Prior/concurrent | E 11. | The participant was previously exposed to any BTK inhibitor, |
| clinical study | | including tolebrutinib. |
| experience | E 12. | The participant has taken other investigational drugs within 3 |
| | | months or 5 half-lives, whichever is longer, before the |
| | | screening visit. |
| Diagnostic | E 13. | The participant has had a relapse in the 30 days prior to |
| assessments | | randomization. |
| | E 14. | The participant has a contraindication for MRI, ie, presence of |
| | | pacemaker, metallic implants in high-risk areas (ie, artificial |
| | | heart valves, aneurysm/vessel clips), presence of metallic |
| | | material (eg, shrapnel) in high risk areas, known history of |
| | | allergy to any contrast medium, or history of claustrophobia |
| | | that would prevent completion of all protocol scheduled MRI. |
| | | Note: People with a contraindication to Gd can be enrolled |
| | | into the study but cannot receive Gd contrast dyes during their |
| | | MRI scan. |
| Other exclusions | E 15. | Individuals accommodated in an institution because of |
| | | regulatory or legal order; prisoners or participants who are |
| | | legally institutionalized. |
| | E 16. | Participant not suitable for participation, whatever the reason, |
| | | as judged by the Investigator, including medical or clinical |
| | | conditions, or participants potentially at risk of noncompliance |
| | | to study procedures. |
| | E 17. | Participants are employees of the clinical study site or other |
| | | individuals directly involved in the conduct of the study, or |
| | | immediate family members of such individuals. |
| | E 18. | Any other situation during study implementation/course that |
| | | may raise ethics considerations |
| | | Note: a one-time retest at screening may be performed if an |
| | | abnormal laboratory test value is considered temporary |

### Lifestyle Considerations

Meals and dietary restrictions: tolebrutinib (IMP) shall be taken with a regular meal. When possible, the meal with which IMP is taken (eg, breakfast, lunch, or dinner) should be consistent throughout the study. The typical meal with which the IMP is taken will be recorded at each visit. In case the mealtime for IMP administration needs to be changed, a gap of a minimum of 12 hours between 2 doses should be maintained.

Caffeine, alcohol, and tobacco: During the entire study, participants should be warned not to consume substantial quantities of alcohol, defined as >14 grams (1 standard drink) per day in female participants or >28 grams (2 standard drinks) per day in male participants on a regular basis.

### Study Intervention

Study intervention is defined as any investigational intervention(s), marketed product(s), or placebo intended to be administered to a study participant according to the study protocol.

TABLE 2E
Overview of study interventions administered

| ARM name | tolebrutinib | Teriflunomide |
|---|---|---|
| Intervention name | tolebrutinib 60 mg | Teriflunomide 14 mg |
| | Placebo matched to | Placebo matched to |
| | teriflunomide | tolebrutinib |

| Type | Drug | Drug |
|---|---|---|
| Dose formulation | tolebrutinib: | Teriflunomide: |
| | Film-coated tablet | Tablet |
| | Placebo matched to | Placebo matched to |
| | teriflunomide: | tolebrutinib: |
| | Tablet | Film-coated |
| | | tablet |
| Unit dose strength(s) | 60 mg | 14 mg |
| Dosage level(s) | Once daily | Once daily |
| Route of administration | Oral | Oral |
| IMP and NIMP | IMP | IMP |
| Packaging and | Study intervention will | Study intervention will |
| labeling | be provided in wallet | be provided in wallet |
| | blister packaging. The | blister packaging. The |
| | content of the labeling | content of the labeling |
| | is in accordance with | is in accordance with |
| | the local regulatory | the local regulatory |
| | specifications and | specifications and |
| | requirements. | requirements. |
| Current/Former | Not applicable | Aubagio |
| name(s) or alias(es) | | |

IMP: investigational medicinal product,
NIMP: noninvestigational medicinal product

Between the protocol-scheduled, onsite visits, interim visits may be required for IMP dispensing. As an alternative to these visits or to replace onsite IMP dispensation, if needed, IMP may be supplied from the site to the participant via a Sponsor-approved courier company (direct-to-patient shipment) where allowed by local regulations and approved by the Sponsor.

### Noninvestigational Medicinal Product

### MRI Contrast-Enhancing Preparations

Route(s) of administration: IV
Dose regimen: as per respective label

### Cholestyramine

Route(s) of administration: oral
Dose regimen: 8 g 3 times daily for 11 days for accelerated elimination procedure (4 g 3 times daily for 11 days in case of intolerance). The teriflunomide local label should be followed.

### Concomitant Therapy

Any medication or vaccine (including over-the-counter or prescription medicines, vitamins, and/or herbal supplements) that the participant is receiving at the time of enrollment or receives during the study must be recorded along with:
Reason for use
Dates of administration including start and end dates
Dosage information including dose and frequency

Participants must abstain from taking prescription or nonprescription drugs (including vitamins and dietary or herbal supplements) within 7 days (or 14 days if the drug is a potential enzyme inducer) or 5 half-lives (whichever is longer) before the start of study intervention until completion of the follow-up visit, unless, in the opinion of the Investigator, the medication will not interfere with the study.

Live (attenuated) vaccines should not be administered during the intervention period.

Therapies for MS noted in the exclusion criterion E07 are not permitted after randomization while the participant is on study treatment. Short term use (3 to 5 days) of glucocorticoids (eg, for MS relapse treatment or an acute illness) and local corticosteroids (eg, topical, nasal, ocular, otic, intra-articular) are allowed.

The Medical Monitor should be contacted if there are any questions regarding concomitant or prior therapy.

For some prohibited concomitant medications (eg, aspirin >81 mg/day for headache), if use is not chronic, temporary discontinuation of IMP can be considered prior to a decision to permanently stop the IMP.

Medications for treatment of MS symptoms (eg, walking impairment, fatigue, spasticity, incontinence, pain) should be maintained at a stable dose prior to screening and for the duration of the treatment period, if clinically feasible.

Anticoagulant/antiplatelet therapies are not permitted to be taken concomitantly with the IMP, including:
Acetylsalicylic acid (aspirin) >81 mg/day
Antiplatelet drugs (eg, clopidogrel)
Warfarin (vitamin K antagonist)
Heparin, including low molecular weight heparin (antithrombin agents)
Dabigatran (direct thrombin inhibitor)
Apixaban, edoxaban, rivaroxaban (direct factor Xa inhibitors)

Paracetamol/acetaminophen, at doses of ≤3 grams/day, is permitted for use at any time during the study. A short course (up to 5 days) of nonsteroidal anti-inflammatory drugs (NSAIDs) (other than acetylsalicylic acid), preferably selective cyclooxygenase-2 inhibitors at the lowest effective dose, may be given during the study if clinically necessary for the treatment of an existing medical condition or a new event. The Investigator must record the use of NSAIDs (and any other comedication) in the eCRF.

### CYP Inhibitor/Inducer:

Potent and moderate inducers of CYP3A or potent inhibitors of CYP2C8 hepatic enzymes are not permitted throughout the study (see Appendix 8A [Section 10.8]).
Tolebrutinib: Tolebrutinib is a substrate of the CYP3A4 and CYP2C8 isoenzymes. In healthy participants, potent CYP3A4 inhibitor (itraconazole 200 mg once daily for 4 days) increased Tolebrutinib area under the curve (AUC) exposure 1.8-fold (INT16385 study) and potent CYP2C8 inhibitor (gemfibrozil 600 mg twice daily for 6 days) increased Tolebrutinib (AUC) exposure 8.4-fold (INT16726 study). Based on a satisfactory safety and tolerability profile and on the observed exposure in healthy participants who received Tolebrutinib at a dose of up to 240 mg once daily for 14 days under fed conditions (TDR16862 study), drugs that strongly inhibit CYP3A4 are allowed and drugs that strongly inhibit CYP2C8 are not permitted. In healthy participants, potent CYP3A4 and moderate CYP2C8 induction by rifampicin (600 mg once daily for 8 days) decreased Tolebrutinib exposure 6-fold (INT16726 study). Therefore, potent and also moderate (based on prediction) CYP3A inducers are not permitted due to their potential to decrease Tolebrutinib exposure and efficacy. See Table 2H for the list of drugs not to be used.
Teriflunomide: Other potent CYP and transporter inducers should be avoided due to their potential to decrease teriflunomide exposure. Rifampicin and other known potent CYP and transporter inducers such as carbamazepine, phenobarbital, phenytoin, avasimibe, lumacaftor, rifapentine, rifabutin, and St John's wort, should be avoided. Leflunomide is prohibited, because teriflunomide is a metabolite of leflunomide.

Cholestyramine (and other bile acid sequestrants) and activated charcoal use should be avoided because these may lead to significant decrease in plasma concentration of teriflunomide.

Cholestyramine can be used only when an accelerated elimination procedure is needed. In exceptional situations (eg, when cholestyramine is not tolerated, or cholestyramine is not available) use of activated charcoal (as per the current Aubagio SmPC) can be considered.

Breast cancer resistance protein (BCRP) inhibitors (eg, eltrombopag and gefitinib) should be avoided. Based on in vitro studies, teriflunomide is a substrate of the efflux transporter BCRP, so these drugs may increase exposure of teriflunomide (Appendix 8B [Section 10.9]).

A number of drugs need to be used with caution as their exposure may be altered by teriflunomide. Adequate arrangements need to be made to monitor their effects and to ensure timely decision for change of medication.
Medicinal products metabolized by CYP2C8 (eg, repaglinide, pioglitazone, and rosiglitazone) should be used with caution during treatment with the IMP due to potential of increase of concentrations of these drugs following CYP2C8 inhibition by teriflunomide.
Increase in ethinylestradiol and levonorgestrel exposure following repeated doses of teriflunomide has been observed (Aubagio (teriflunomide) [package insert]. Genzyme Corporation. Cambridge, MA 02142; 2021).
While this interaction of teriflunomide is not expected to adversely impact the efficacy of oral contraceptives, it should be considered when selecting or adjusting oral contraceptive treatment used during this trial.
Medicinal products metabolized by CYP1A2 (eg, duloxetine, alosetron, theophylline, and tizanidine) should be used with caution during treatment with IMP due to the capacity of teriflunomide to induce CYP1A2 and to reduce efficacy of these products.
Administration of substrates of organic anion transporter 3 (OAT3) (eg, cefaclor, benzylpenicillin, ciprofloxacin, indomethacin, ketoprofen, furosemide, and cimetidine) should be performed with caution during the study due to the potential of teriflunomide to increase exposure to these medications through inhibition of OAT3.
For substrates of BCRP (eg, sulfasalazine) and the organic anion transporter polypeptide (OATP) family (eg, rosuvastatin, simvastatin, atorvastatin, pravastatin, nateglinide, repaglinide, and rifampicin), concomitant administration with teriflunomide should be used with caution during the study due to the potential of teriflunomide to increase exposure to these products. Participants should be closely monitored for signs and symptoms of excessive exposure to the medicinal products, and reduction of the dose should be considered. If used together with the IMP, the dose of rosuvastatin should not exceed 10 mg once daily.

### Dose Modification

Dose reduction is not foreseen in this study. Treatment may need to be interrupted or permanently discontinued if deemed necessary due to an AE (Section 7 and Section 8.3).

### Discontinuation of Study Intervention

### Definitive Discontinuation

The study intervention should be continued whenever possible.

Permanent intervention discontinuation is any treatment discontinuation associated with the definitive decision from the Investigator or the participant not to re-expose the participant to the study intervention at any time.

In rare instances, it may be necessary for a participant to permanently discontinue the study intervention. If study intervention is permanently discontinued, the participant shall be asked to remain in the study to be evaluated until the EOS visit. This will be important to continue to evaluate for safety. See the SoA (Tables 2A1 and 2A2) for data to be collected at the time of discontinuation of the study intervention. In the case that the study intervention is permanently discontinued, the participant should be treated for MS according to local clinical practice and the best judgment of the Investigator.

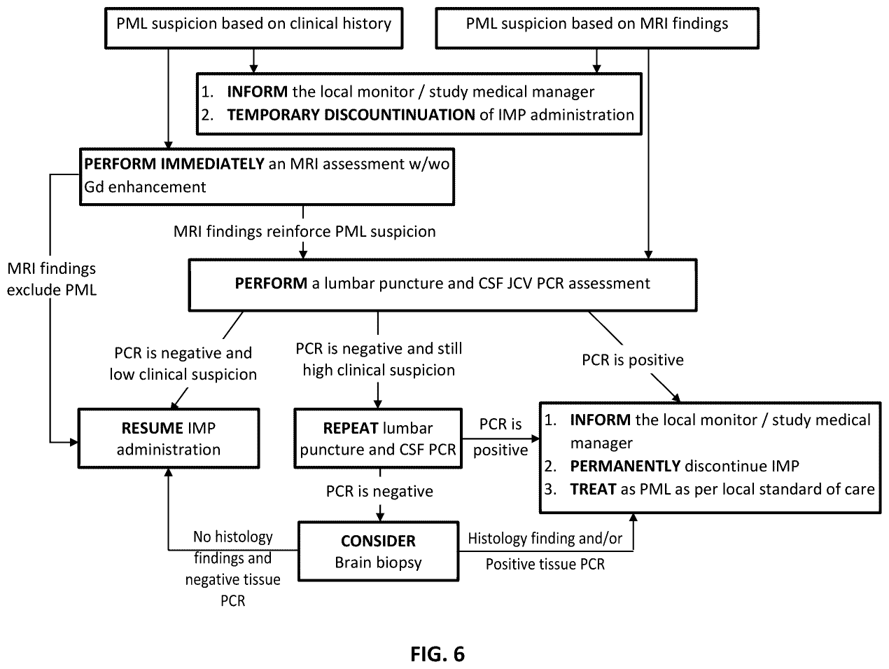

The following may be justifiable reasons for the Investigator or Sponsor to discontinue a participant from study treatment:
Adverse events that endanger the safety of the participant, or if discontinuation of study intervention is desired or considered necessary by the Investigator and/or participant.
If IMP discontinuation criteria are met as per guidance for the follow up of laboratory abnormalities in FIG. 2-8.
The participant is no longer deriving a therapeutic/clinical benefit in the opinion of the Investigator.
At participant's request, ie, withdrawal of the consent for treatment.
If a female participant becomes pregnant or wishes to become pregnant during the study.
If a male participant wishes to conceive a child during the study.
Any serious opportunistic infections (eg, PML [see FIG. 6], HIV).
Continued need for/chronic use of a prohibited concomitant medication (see CONCOMITANT THERAPY in this example and Table 2H).

Discontinuation of the study intervention for abnormal liver function should be considered by the Investigator when a participant meets one of the conditions outlined in the algorithm (FIGS. 4A and 4B) or if the Investigator believes that it is in the best interest of the participant.

Any clinically significant abnormal laboratory value or ECG parameter will be immediately rechecked for confirmation after 24 hours before making a decision of definitive discontinuation of the IMP for the concerned participant.

If a clinically significant finding is identified in the ECG (including, but not limited to changes from baseline in QT interval corrected (QTc) using Fridericia's formula [QTcF] after enrollment), the Investigator or a qualified designee will determine if the participant can continue in the study and if any change in participant management is needed. Review of ECG findings by a cardiologist may be considered for a decision of a definitive discontinuation of study intervention because of ECG changes. This review of the ECG findings recorded at the time of collection must be documented. Any new clinically relevant ECG finding should be reported as an AE.

See the SoA (Tables 2A1 and 2A2) for data to be collected at the time of intervention discontinuation and follow up and for any further evaluations that need to be completed.

Additional tests may be performed at any time during the study as determined necessary by the Investigator or required by local regulations.

TABLE 2F
Protocol-required laboratory assessments

| Laboratory | | | |
|---|---|---|---|
| assessments | Parameters | | |
| Hematology | Platelet count | RBC indices: | WBC count with differential: |
| | RBC count | MCV | Neutrophils |
| | Hemoglobin | MCH | Lymphocytes |
| | Hematocrit | % Reticulocytes | Monocytes |
| | | | Eosinophils |
| | | | Basophils |
| Clinical | BUN | Sodium | AST |
| chemistry | Creatinine | Calcium | ALT |
| | Glucose | Total and direct bilirubin | Alkaline phosphatase |
| | Potassium | Total protein | Albumin |
| | | Chloride | Creatine phosphokinase |
| | | Bicarbonate | Lipase (screening only) |

| | |
|---|---|
| Routine | Specific gravity |
| urinalysis | pH, glucose, protein, blood, ketones, bilirubin, urobilinogen, nitrite, |
| | leukocyte esterase by dipstick |
| | Microscopic examination (if blood or protein is abnormal and for signs of |
| | infection) |
| Other | FSH and estradiol (if needed, only in female participants to confirm |
| screening | postmenopausal state) |
| tests | Highly sensitive serum or urine β-hCG pregnancy test (as needed for |
| | women of childbearing potential)^{c} |
| | Coagulation: PT/INR, aPTT |
| | Serology tests for hepatitis B (HBs Ag, anti-HBc IGM and total, anti-HBs) |
| | and C virus (anti-HCV); in case these results are inconclusive (eg anti-HBs |
| | negative and anti-HBc positive or anti-HC IgG positive), HBV-DNA or |
| | HCV-RNA testing, respectively, should be performed for confirmation. HIV |
| | and other infectious diseases, if locally required. |
| | Tuberculosis test: Blood testing (eg, QuantiFERON ® TB Gold test) is |
| | preferred; skin testing (eg, tuberculin skin test) with ancillary testing will be |
| | allowed if blood testing is not available. T-SPOT can also be performed, if |
| | available. |
| | Iron panel (serum): iron, ferritin, transferrin saturation TIBC. |

ALT: alanine aminotransferase;
anti-HBc; antibody to hepatitis B core antigen;
anti-HBs: hepatitis B surface antibody;
aPTT: activated partial thromboplastin time;
AST: aspartate aminotransferase;
BUN: blood urea nitrogen;
β-hCG: human chorionic gonadotropin;
FSH; follicle-stimulating hormone;
IEC: independent ethics committee;
INR: international normalized ratio;
HBsAg: hepatitis B surface antigen;
HBV: hepatitis B virus;
HCV: hepatitis C virus;
HIV: human immunodeficiency virus;
Ig: immunoglobulin;
IRB: institutional review board;
MCH: mean corpuscular hemoglobin;
MCV: mean corpuscular volume;
PT: prothrombin time;
RBC: red blood cell;
SGOT: serum glutamic-oxaloacetic transaminase;
SGPT: serum glutamic-pyruvic transaminase;
TB: tuberculosis;
ULN: upper limit of normal;
WBC: white blood cell
a Details of liver chemistry stopping criteria and required actions and follow-up assessments after observations of ALT >3 × ULN are given in FIG. 4A and 4B. Clinical laboratory findings of ALT >3 × ULN and bilirubin ≥2 × ULN (>35% direct bilirubin), or ALT >3 × ULN and international normalized ratio (INR) >1.5, if INR measured, that may suggest severe liver injury and must be reported as an SAE.
b Other renal function parameters, reatinine clearance (CrCl) will be calculated.
^{c}Local urine testing for pregnancy will be standard for the protocol (except for the Screening Visit, when a serum pregnancy test is required) unless serum testing is required by local regulation or IRB/IEC.

### Liver and Other Safety: Actions and Follow-Up Assessments

These actions described in Table 2G-A and FIGS. 2-8 are required for ALT increase and thrombocytopenia events ONLY. For all other safety events described, these are suggested per the Investigator's medical judgement.

Neutropenia is to be recorded as an AE only if at least 1 of the criteria listed in the general guidelines for reporting AEs in Appendix 3 (Section 10.3) is met.

Thrombocytopenia is to be recorded as an AE only if at least 1 of the criteria listed in the general guidelines for reporting AEs in Appendix 3 (Section 10.3) is met.

Abbreviations in FIG. 4A: ALT: alanine aminotransferase; ANCA: antineutrophil cytoplasmic antibodies; AST: aspartate aminotransferase; CMV: cytomegalovirus; CRF: case report form; DNA: deoxyribonucleic acid; dsDNA: double stranded DNA; EBV: Epstein-Barr Virus; GGT: Gamma-glutamyl transferase; HAV: Hepatitis A virus; HBc: Hepatitis B core; HBV: Hepatitis B virus; HCV: Hepatitis C virus; HEV: Hepatitis E virus; IgM: immunoglobulin M; IMP: investigational medicinal product; INR: international normalized ratio; lab: laboratory; LFT: liver function test; PT: prothrombin time; RNA: ribonucleic acid; LKM: liver kidney microsome; ULN: upper limit of normal.

Note: “Baseline” refers to ALT sampled at baseline visit; or if baseline value unavailable, to the latest ALT sampled before the baseline visit. The algorithm does not apply to the instances of increase in ALT during Screening.

TABLE 2G-A
Actions for cases of confirmed ALT elevation

In ANY CONFIRMED CASE, FOLLOW the instructions listed below:
INFORM the Site Monitor, who will forward the information to the Study Manager.
INVESTIGATE specifically for malaise with or without loss of consciousness,
dizziness, and/or hypotension and/or episode of arrhythmia in the previous 72
hours; rule out muscular injury.
PERFORM the following tests:
LFTs: AST, ALT, alkaline phosphatase, total and conjugated bilirubin and
prothrombin time/INR;
CPK, serum creatinine, complete blood count;
Anti-HAV IgM, anti-HBc IgM, (HBV-DNA if clinically indicated),
anti-HCV and HCV RNA, anti-CMV IgM and anti- HEV IgM
antibodies;
Depending on the clinical context, check for recent infection with EBV, herpes
viruses, and toxoplasma;
Consider iron, ferritin, transferrin saturation;
Consider hepatobiliary ultrasonography (or other imaging investigations if
needed);
CONSIDER auto-antibodies: antinuclear, anti-DNA, anti-smooth muscle, anti-LKM,
anti-mitochondrial;
CONSIDER DNA test for Gilbert's disease if clinically indicated;
CONSIDER consulting with a hepatologist;
CONSIDER patient hospitalization if INR >2 (or PT <50%) and/or central
nervous system disturbances suggesting hepatic encephalopathy;
MONITOR LFTs after permanent discontinuation of IMP:
Monitor as closely as possible (every 48 hours to every week)
until ALT is down-trending, then every 2 weeks until 1.5 ×
ULN, and then every scheduled visit;
Rechallenge Reinitiation of the study drug can only be considered after
discussion with the Sponsor's Medical Monitor once the ALT/AST decreases
below 1.5 × ULN, and there is no clinical contraindication. In case it is agreed
to re-start the study drug, it is recommended that ALT/AST be assessed
weekly for the first month and then monthly for the second and third months.
The occurrence of new elevation above 3 × ULN for the ALT/AST values will
lead to permanent discontinuation of the study drug; No rechallenge will be
considered for participants with >3 × ULN ALT and >2 × ULN bilirubin
increase
FREEZE serum sample (5 ml × 2).
COLLECT/STORE one PK sample following the instructions in the central
laboratory manual

Abbreviations in FIG. 5: ARF, acute renal failure; ULN, upper limit of normal; DIC, disseminated intravascular coagulation; CPK, creatine phosphokinase; ECG, electrocardiogram; PK, pharmacokinetic(s).

Increase in serum creatinine is to be recorded as an AE only if at least 1 of the criteria listed in the general guidelines for reporting AEs is met.

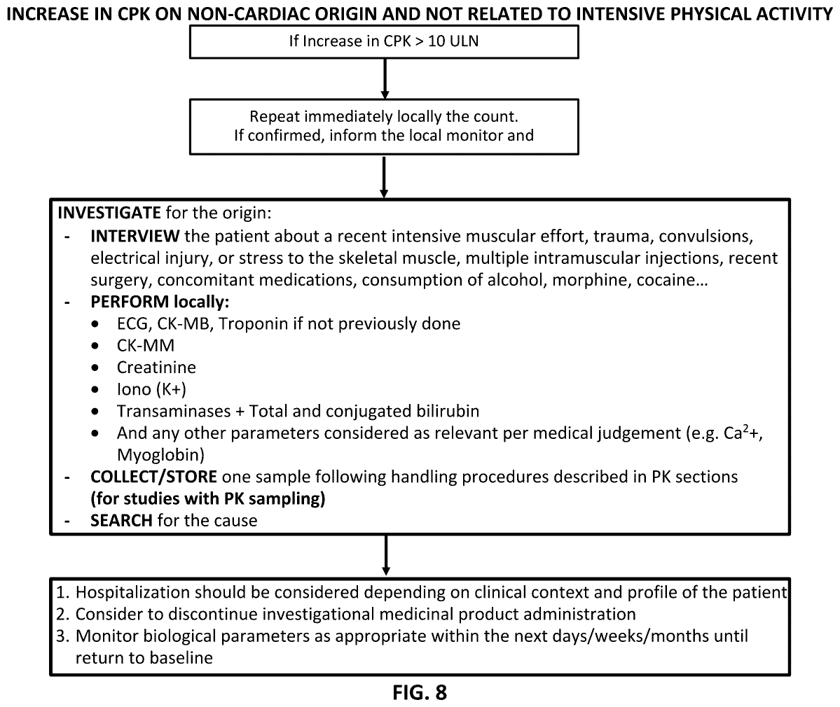

Abbreviations in FIG. 8: CK-MB, creatine kinase-MB; CK-MM, creatine kinase-MM; ECG, electrocardiogram; PK, pharmacokinetic(s); ULN, upper limit of normal.

Increase in CPK is to be recorded as an AE only if at least 1 of the criteria in the general guidelines for reporting AEs is met.

SUSPECTED PML: If either the clinical presentation or MRI features of a participant are suggestive of PML, the diagnostic and action algorithm described in FIG. 6 is recommended.

Abbreviations in FIG. 6: Abbreviations: CSF, cerebrospinal fluid; Gd, gadolinium; IMP, investigational medicinal product; JCV, John Cunningham virus; MRI, magnetic resonance imaging; PCR, polymerase chain reaction; PML, progressive multifocal leukoencephalopathy.

Clinical manifestations or MRI lesions features suspicious for PML are proposed in Table 2G-B (based on Berger et al. Neurology 2013, 80, 1430-1438 and Kappos et al. Lancet Neurol. 2007, 6, 431-441).

TABLE 2G-B
Clinical and MRI features suggestive of PML

| | |
|---|---|
| Clinical history | Subacute onset of weakness, sensory deficits, cognitive |
| | or behavioral abnormalities, gait dysfunction, speech/language |
| | difficulties or any other signs of cortical dysfunction, |
| | retrochiasmal visual defects or seizure |
| Brain MRI | ≥1 T2/FLAIR hyperintense and T1 hypointense lesions involving |
| | the subcortical and juxtacortical white matter, sparing the cortex, |
| | with no mass effect, with a continuous progression; new lesions |
| | with no enhancement (even when large) or with faint rim |
| | enhancement |
| | The detection of John Cunningham virus (JCV) DNA in the cerebrospinal fluid |
| | of a patient with clinical and MRI features suggestive of PML establishes the |
| | diagnosis of PML. |
| | If JCV DNA is not detected in cerebrospinal fluid and if clinical suspicion of |
| | PML remains high, another lumbar puncture should be performed. |
| | If diagnosis remains uncertain and suspicion of PML remains high, a brain |
| | biopsy may be considered to establish a definitive diagnosis |

Clinical or MRI features suggestive of PML should be recorded as an AE/AESI/SAE.

Examples of Drugs with a Potential to Change Tolebrutinib Metabolism

The following drugs should not be taken during the study concomitantly with the IMP due to their potential to change tolebrutinib kinetics due to interaction with P450-mediated metabolism, being potent/moderate inducers of CYP3A or potent inhibitors of CYP2C8 liver enzymes (per the lists of the Drug Interaction Database Program of the University of Washington).

Please note that the lists provided are not exhaustive and that the product information of drugs intended for concomitant use should be consulted.

TABLE 2H
CYP3A Inducers and CYP2C8 Inhibitors
Potent CYP3A Inducers:

| | |
|---|---|
| Rifampin | Carbamazepine |
| Phenobarbital | St John's wort extract |
| Avasimibe | Lumacaftor |
| Rifapentine | Rifabutin |
| Phenytoin | |

Potent CYP2C8 Inhibitors:

| | |
|---|---|
| Gemfibrozil | Deferasirox |
| Clopidogrel | |

Moderate CYP3A Inducers

| | |
|---|---|
| Semagacestat | Asunaprevir/beclabuvir/daclatasvir |
| Cenobamate | Nafcillin |
| Lesinurad | Modafinil |
| Bosentan | Telotristat ethyl |
| Thioridazine | Elagolix |
| Rifabutin | |

### Abbreviations

ADL: activities of daily living
AE: adverse event
AESI: adverse event of special interest
ALT: alanine aminotransferase
ARR: annualized adjudicated relapse rate
AUC: area under the curve
BCRP: breast cancer resistance protein
BTK: Bruton's tyrosine kinase
CDW: confirmed disability worsening
CFR: Code of Federal Regulations
Chi3L1: chitinase-3 like protein-1
CNS: central nervous system
CPK: creatine phosphokinase
CRF: case report form
CSR: clinical study report
C-SSRS: Columbia Suicide Severity Rating Scale
CYP: cytochrome P450
DILI: drug-induced liver injury
DMC: Data Monitoring Committee
DMT: disease-modifying therapy
DNA: deoxyribonucleic acid
ECG: electrocardiogram
eCRF: electronic case report form
EDSS: Expanded Disability Status Scale
EOS: end of study
EQ-5D-5L: EuroQol 5-dimension 5-level questionnaire
EU: European Union
FSH: follicle-stimulating hormone
GCP: Good Clinical Practice
Gd: gadolinium
HRT: hormone replacement therapy
ICF: informed consent form
ICH: International Council for Harmonisation
IEC: Independent Ethics Committee
IMP: investigational medicinal product
INR: international normalized ratio
IRB: Institutional Review Board
IRT: interactive response technology
ITT: intent to treat
IUD: intrauterine device
IUS: intrauterine hormone-releasing system
IV: intravenous
JCV: John Cunningham virus
LTS: long-term safety
MedDRA: Medical Dictionary for Regulatory Activities
MRI: magnetic resonance imaging
MS: multiple sclerosis
MSFC-3: Multiple Sclerosis Functional Composite-3
MSQol-54: Multiple Sclerosis Quality of Life-54
MTR: magnetization transfer ratio
NCI CTCAE: National Cancer Institute Common Terminology Criteria for Adverse
Events
NEDA: no evidence of disease activity-3
NfL: neurofilament light chain
NIMP: noninvestigational medicinal product
NSAID: nonsteroidal anti-inflammatory drug
OAT3: organic anion transporter3
PD: pharmacodynamic(s)
PK: pharmacokinetic(s)
PML: progressive multifocal leukoencephalopathy
PPMS: primary progressive multiple sclerosis
QTcF: QT interval corrected using Fridericia's formula
RMS: relapsing multiple sclerosis
SAE: serious adverse event
SAP: Statistical Analysis Plan
SEL: slowly evolving lesions
SmPC: summary of product characteristics
SoA: schedule of activities
SPMS: secondary progressive multiple sclerosis
Study Manual: Study Reference Manual
SWI: susceptibility-weighted imaging
TEAE: treatment-emergent adverse event
ULN: upper limit of normal
US: United States
USPI: United States prescribing information
WOCBP: woman of childbearing potential

### Example 3—A Phase 3, Randomized, Double-Blind, Efficacy and Safety Study Comparing SAR442168 to Placebo in Participants with Nonrelapsing Secondary Progressive Multiple Sclerosis (Hercules)

Overall design: This is a Phase 3, randomized, double-blind, 2-arm, placebo-controlled, parallel group, multicenter, event-driven (6-month CDP) trial with a variable treatment duration ranging from approximately 24 to 48 months in participants with NRSPMS. A graphical scheme of the study design is shown in FIG. 1C. Abbreviations used in FIG. 1C: CDP, confirmed disability progression; EOS, end of study; MRI, magnetic resonance imaging; R, randomization. “Month-1 (D-28-D-1)” refers to screening period as “Day-28 to Day-1”; “Month 0 (D1)” refers to randomization on Day 1.

Tables 3A1 and 3A2 shown below describe the schedule of activities during the course of study. Table 3B that follows describes the objective and endpoints of the overall study.

TABLE 3A1
Schedule of Activities (SOA)- Screening to Year 2

| | | Randomization/ | | | | | | | | | | | | | |
|---|---|---|---|---|---|---|---|---|---|---|---|---|---|---|---|
| | Screening^{a} | start of IMP | To Year 2 (M 24)^{b} | | | | | | | | | | | | |
| | Visit (a window of ±7 days is | | | | | | | | | | | | | | |
| | allowed for all visits after D 1) | | | | | | | | | | | | | | |
| | D-28 to | | | | | | | M 4, | | | | | | | |
| | D-1 | D 1 | M 1^{d} | M 1.5 | M 2^{d} | M 2.5 | M 3 | M 5^{d} | M 6 | M 9 | M 12 | M 15 | M 18 | M 21 | M 24 |
| | Visit Number | | | | | | | | | | | | | | |
| | | | | | | | | V6, | | | | | | | |
| Procedure | V1 | V2 | V3 | | V4 | | V5 | V7 | V8 | V9 | V10 | V11 | V12 | V13 | V14 |
| Informed | X | | | | | | | | | | | | | | |
| consent | | | | | | | | | | | | | | | |
| Demography | X | | | | | | | | | | | | | | |
| Inclusion/ | X | X | | | | | | | | | | | | | |
| exclusion | | | | | | | | | | | | | | | |
| criteria | | | | | | | | | | | | | | | |
| Medical/ | X | | | | | | | | | | | | | | |
| surgical | | | | | | | | | | | | | | | |
| history | | | | | | | | | | | | | | | |
| Prior/ | | <=========================================================================> | | | | | | | | | | | | | |
| concomitant | | | | | | | | | | | | | | | |
| medications^{g} | | | | | | | | | | | | | | | |
| Randomization | | X | | | | | | | | | | | | | |
| IRT contact | X | X | | | | | X | | X | X | X | X | X | X | X |

Study treatment administration

| | | | | | | | | | |
|---|---|---|---|---|---|---|---|---|---|
| IMP dispensation | X | X | X | X | X | X | X | X | X |
| IMP Compliance | | X | X | X | X | X | X | X | X |
| Paper diary | X | X | X | X | X | X | X | X | X |
| dispensation/ | | | | | | | | | |
| collection | | | | | | | | | |

Safety^{y}

| | | | | | | | | | | | | | | | |
|---|---|---|---|---|---|---|---|---|---|---|---|---|---|---|---|
| Physical | X | X | | | | | X | X | X | X | X | X | X | X | X |
| examination^{h} | | | | | | | | | | | | | | | |
| and vital signs | | | | | | | | | | | | | | | |
| Height | X | | | | | | | | | | | | | | |
| Body weight | X | X | | | | | X | | X | | X | | X | | X |
| Serology tests | X | | | | | | | | | | | | | | |
| for hepatitis | | | | | | | | | | | | | | | |
| B and C | | | | | | | | | | | | | | | |
| HIV and other | X | | | | | | | | | | | | | | |
| infectious | | | | | | | | | | | | | | | |
| diseases, if | | | | | | | | | | | | | | | |
| required locally | | | | | | | | | | | | | | | |
| TB/ | X | | | | | | | | | | | | | | |
| QuantiFERON ® | | | | | | | | | | | | | | | |
| TB Gold test or | | | | | | | | | | | | | | | |
| equivalentⁱ | | | | | | | | | | | | | | | |
| Body temperature | X | X | | | | | X | | X | X | X | X | X | X | X |
| 12-lead ECG^{j} | X | | | | | | X | | X | X | X | | | | X |
| Hematology, | X | X | X | | X | | X | X | X | X | X | X | X | X | X |
| biochemistry^{k} | | | | | | | | | | | | | | | |
| Liver function | | | | X | | X | | | | | | | | | |
| tests^{l} | | | | | | | | | | | | | | | |
| Iron panel | X | | | | | | | | | | | | | | |
| (serum): iron, | | | | | | | | | | | | | | | |
| ferritin, | | | | | | | | | | | | | | | |
| transferrin | | | | | | | | | | | | | | | |
| saturation, TIBC; | | | | | | | | | | | | | | | |
| to be repeated | | | | | | | | | | | | | | | |
| during the study | | | | | | | | | | | | | | | |
| if needed | | | | | | | | | | | | | | | |
| Coagulation: | X | | | | | | | | | | | | | | |
| PT/INR, aPTT | | | | | | | | | | | | | | | |
| (to be | | | | | | | | | | | | | | | |
| repeated | | | | | | | | | | | | | | | |
| during the | | | | | | | | | | | | | | | |
| study, if | | | | | | | | | | | | | | | |
| needed) | | | | | | | | | | | | | | | |
| Urinalysis | X | | | | | | X | | | | X | | X | | X |
| Pregnancy test (if | X | X | | | | | X | | X | X | X | X | X | X | X |
| applicable)^{m} | | | | | | | | | | | | | | | |
| Serum FSHⁿ | X | | | | | | | | | | | | | | |
| Suicidality | X | X | | | | | X | | X | X | X | X | X | X | X |
| assessment by | | | | | | | | | | | | | | | |
| C-SSRS | | | | | | | | | | | | | | | |

| | |
|---|---|
| Adverse event | <=================================================================================> |
| collection | |

Efficacy

| | | | | | | | | | | | |
|---|---|---|---|---|---|---|---|---|---|---|---|
| EDSS | X | X | X | X | X | X | X | X | X | X | X |
| Timed 25-foot | | X | X | | X | X | X | X | X | X | X |
| walk test | | | | | | | | | | | |
| 9-hole peg test | | X | X | | X | X | X | X | X | X | X |
| SDMT and | | X | X | | X | X | X | X | X | X | X |
| CVLT-II, | | | | | | | | | | | |
| where available^{o} | | | | | | | | | | | |
| Basic or expanded | X^{q} | | | | X | | X | | X | | X |
| MRI^{p} | | | | | | | | | | | |

| | |
|---|---|
| Actigraphy | X |
| (optional for | |
| subset of | |
| participants)^{r} | |

Clinical outcome assessments^{s}

| | | | | | |
|---|---|---|---|---|---|
| MSQoL-54 | X | X | X | X | X |
| EQ-5D-5L | X | X | X | X | X |

Pharmacokinetics^{s}

| | | | |
|---|---|---|---|
| SAR442168 and | X^{u} | X^{u} | X^{u} |
| relevant | | | |
| metabolite(s) | | | |
| pharmacokinetic | | | |
| plasma samples^{t} | | | |

Pharmacogenetics^{s}

| | |
|---|---|
| DNA sample | X |
| (optional)^{w} | |

Pharmacodynamics/biomarkers^{s}

| | | | | | | |
|---|---|---|---|---|---|---|
| Blood sample for | X | | | | | |
| archiving^{x} | | | | | | |
| Immunopheno- | | X | X | | X | |
| typing/RN A | | | | | | |
| sequencing | | | | | | |
| (ToleDYNAMIC/ | | | | | | |
| optional substudy | | | | | | |
| at selected | | | | | | |
| sites)^{aa} | | | | | | |
| Lymphocyte | | X | | | | |
| phenotyping by | | | | | | |
| flow cytometry | | | | | | |
| in whole blood | | | | | | |
| (subset of | | | | | | |
| participants)^{bb} | | | | | | |
| Plasma samples | | X | X | X | X | X |
| (NfL), serum | | | | | | |
| samples | | | | | | |
| (Chi3L1)^{v} | | | | | | |
| Serum samples | | X | | X | X | X |
| (Ig levels)^{v} | | | | | | |

TABLE 3A2
Schedule of Activities (SOA)- M27 to Follow-up Visit

| Procedure | | | | |
|---|---|---|---|---|
| | | Only for | | |
| | | participants who | | Only for participants who |
| | | prematurely | | completed treatment to EOS |
| From M27 to EOS^{b} | | discontinue IMP | For all participants | but do not enter LTS^{c} |
| Visit (a window of ±7 days is allowed for all visits after D 1) | | | | |
| Quarterly | | | | |
| visits | | | | |
| (M27, M30, | Semi-annual | | | |
| M33, M36, | visits | | | |
| M39, M42, | (M30, M36, | | EOS | |
| M45, | M42, | | “Common study end | Follow-up visit |
| M48 . . . ) | M48 . . . ) | pEOT^{e} | date” visit^{f} | (4 to 8 weeks) |
| Visit Number | | | | |
| V15, V16, | | | | |
| V17, V18, | | | | |
| V19, V20, | V16, V18, | | | |
| V21, V22 | V20, V22 | pEOT^{e} | EOS | FUV |

Informed consent
Demography
Inclusion/exclusion
criteria
Medical/surgical
history

| Prior/concomitant | <===========================================================================> | | | | |
|---|---|---|---|---|---|
| medications^{g} | | | | | |
| Randomization | | | | | |
| IRT contact | X | X | X | X | X |

Study treatment administration

| | | | | |
|---|---|---|---|---|
| IMP dispensation | X | X | | |
| IMP Compliance | X | X | X^{e} | X |
| Paper diary | X | X | X | X |
| dispensation/ | | | | |
| collection | | | | |

Safety^{y}

| Physical | X | X | X | X | X |
|---|---|---|---|---|---|
| examination^{h} | | | | | |
| and vital signs | | | | | |
| Height | | | | | |
| Body weight | | X | X | X | X |
| Serology tests | | | | | |
| for hepatitis B | | | | | |
| and C | | | | | |
| HIV and other | | | | | |
| infectious | | | | | |
| diseases, if | | | | | |
| required locally | | | | | |
| TB/QuantiFERON ® | | | | | |
| TB Gold test | | | | | |
| or equivalentⁱ | | | | | |
| Body temperature | X | X | X | X | X |
| 12-lead ECG^{j} | | yearly | X | X | |
| Hematology, | X | X | X | X | X |
| biochemistry^{k} | | | | | |
| Liver function | | | | | |
| tests^{l} | | | | | |
| Iron panel | | | | | |
| (serum): iron, | | | | | |
| ferritin, | | | | | |
| transferrin | | | | | |
| saturation, TIBC; | | | | | |
| to be repeated | | | | | |
| during the study | | | | | |
| if needed | | | | | |
| Coagulation: | | | | | |
| PT/INR, aPTT | | | | | |
| (to be repeated | | | | | |
| during the study, | | | | | |
| if needed) | | | | | |
| Urinalysis | | X | X | X | |
| Pregnancy test (if | X | X | X | X | X |
| applicable)^{m} | | | | | |
| Serum FSHⁿ | | | | | |
| Suicidality | X | X | X | X | X |
| assessment by | | | | | |
| C-SSRS | | | | | |
| Adverse event | <===========================================================================> | | | | |
| collection | | | | | |

Efficacy

| EDSS | X | X | X | X |
|---|---|---|---|---|
| Timed 25-foot | X | X | X | X |
| walk test | | | | |
| 9-hole peg test | X | X | X | X |
| SDMT and | X | X | X | X |
| CVLT-II, | | | | |
| where available^{o} | | | | |
| Basic or | | yearly | X | X |
| expanded MRI^{p} | | | | |
| Actigraphy | X | | | |
| (optional for | | | | |
| subset of | | | | |
| participants)^{r} | | | | |

Clinical outcome assessment^{s}

| | | | |
|---|---|---|---|
| MSQoL-54 | X | X | X |
| EQ-5D-5L | X | X | X |

Pharmacokinetics^{s}

| | |
|---|---|
| SAR442168 and | X^{e} |
| relevant | |
| metabolite(s) | |
| pharmacokinetic | |
| plasma samples^{t} | |

Pharmacogenetics^{s}

DNA sample
(optional)^{w}

Pharmacodynamics/biomarkers^{s}

| | | | |
|---|---|---|---|
| Blood sample for | | | |
| archiving^{x} | | | |
| Immunophenoty | | | |
| ping/RN A | | | |
| sequencing | | | |
| (ToleDYNAMIC/ | | | |
| optional | | | |
| substudy at | | | |
| selected sites)^{aa} | | | |
| Lymphocyte | | X | X |
| phenotyping by | | | |
| flow cytometry | | | |
| in whole blood | | | |
| (subset of | | | |
| participants)^{bb} | | | |
| Plasma samples | yearly | X | X |
| (NfL), serum | | | |
| samples | | | |
| (Chi3L1)^{v} | | | |
| Serum samples | yearly | X | X |
| (Ig levels)^{v} | | | |

aPTT, activated partial thromboplastin time; β-HCG, β-human chorionic gonadotropin; ALP, alkaline phosphatase; ALT, alanine aminotransferase; AST, aspartate aminotransferase; Chi3L1, chitinase-3-like protein 1; C-SSRS, Columbia Suicide Severity Rating Scale; D, day; DNA, deoxyribonucleic acid; ECG, electrocardiogram; EDSS, Expanded Disability Status Scale; EOS, end of study; EOT, end of treatment; EQ-5D-5L, EuroQol 5-dimension 5-level instrument; FSH, follicle stimulation hormone; FUV, follow-up visit; ICF, informed consent form; Ig, immunoglobulin; IRT, interactive response technology; HIV, human immunodeficiency virus; IMP, investigational medicinal product; INR, international normalized ratio; LTS, long term safety study; M, month; MCV, mean corpuscular volume; MCH, mean corpuscular hemoglobin; MRI: magnetic resonance imaging; MS: multiple sclerosis; SDMT, Symbol Digit Modalities Test; CVLT-II, California Verbal Learning Test-II; MSQoL-54: Multiple Sclerosis Quality of Life-54; NfL: neurofilament light chain; pEOT: premature end of treatment; PK: pharmacokinetic; PT: prothrombin time; RBC, red blood cell; SWI, susceptibility weighted imaging; TB, tuberculosis; TIBC: total iron-binding capacity; V: visit; WBC, white blood cell.
Note:
All assessments should be done as designated in this SoA unless not permitted according to local regulations. All visit assessments should be performed during the visit window unless otherwise specified in this protocol.
^{a}Screening period can range from D-28 to D-1; Randomization visit can be performed only once IMPs are available at site. The interval between screening and randomization visits can range from 11 days (minimum) to 28 days (maximum). However, if required, the randomization visit can be performed earlier than 11 days upon IMP receipt at the site, assuming the participant is eligible for randomization. In case of any delay to screening (MRI rescheduling, lab retests, etc), an additional period of up to 2 weeks is allowed.
^{b}From D 1 to EOS, unscheduled visits may be performed at any time by the Investigator (eg, for evaluation of an adverse event). Assessments may be done on as needed basis to evaluate the participant in accordance with the Investigator's best judgement and in-line with the study protocol. At a minimum, a physical examination should be performed, and body temperature and vital signs should be measured.
^{c}At the EOS, the participants who have completed treatment with IMP (double blind or open label if meeting CDP) will be offered participation in LTS study. Follow-up visit assessment only performed for those participants who completed treatment and are not willing to take part in the LTS study.
^{d}These visits may be done as home health visits (where applicable) or onsite visits (it is preferable that tests are performed at the central laboratory). In any situations where this is not possible (to be documented in source documents), the tests for these visits may be performed at a local laboratory.
^{e}If a participant prematurely permanently discontinues treatment with IMP, the participant will undergo pEOT visit as soon as possible. A PK sample should also be collected if the pEOT visit can be scheduled within a maximum 24 hours after the last IMP dose. Participants will then be asked to continue with the study visits as scheduled until the global EOS Visit is reached. During these visits, all study procedures/assessments will be performed except IMP administration and blood sampling for PK and biomarkers (NfL, Chi3L1, and Ig levels). MRI scans for these participants will only be performed annually (using the next annual visit as the starting point).
^{f}For participants continuing in the study, the common EOS visit will be done when the prespecified number of events for 6-month CDP is expected to be reached. The timing and window of this visit will be communicated to sites.
^{g}Any disease-modifying therapy for MS taken at any time prior to signing the informed consent needs to be reported in the eCRF; other prior medications will be reported for the period of 6 months prior to signing the ICF.
^{h}Complete physical examination due at screening, baseline, yearly (M12, M24, M36, M48) and at EOS; brief physical examination is sufficient for the rest of the visits (complete and brief physical examinations will include neurological examination and collection of the following vital signs: arterial blood pressure, heart rate, temperature).
ⁱTo be performed at screening for all participants. Tuberculosis examination will be repeated based on clinical judgment, borderline results, or clinical suspicion of TB infection. For further details, refer to E 01, Table 3D. Screening tests for TB are described in Table 2F.
^{j}ECG and 30 second rhythm strips will be obtained locally.
^{k}Hematology (platelet count, RBC count, hemoglobin, hematocrit, MCV, MCH, reticulocytes, WBC count with differential: neutrophils, lymphocytes, monocytes, eosinophils, basophils).
Biochemistry (blood urea nitrogen [BUN], creatinine, glucose, sodium, potassium, bicarbonate, calcium, liver function tests [AST, ALT, ALP, total and direct bilirubin], total protein; creatine phosphokinase. Lipase will be tested at the Screening Visit, then quarterly. Monthly visits (M1, M2, M4, and M5) will include hematology and full liver panel only. Additional safety assessments can be performed if required by local regulations. Such testing shall be performed at local laboratories.
Note:
a one-time retest at screening may be performed if laboratory test abnormality is considered temporary. Additional safety assessments can be performed if required by local regulations; such testing shall be performed locally whenever possible. Additional visits may be added if required by local regulations.
^{l}At intermediate timepoints M1.5, and M2.5, only liver function tests will be collected (AST, ALT, albumin, alkaline phosphatase, total and direct bilirubin, total protein, and creatine phosphokinase).
^{m}At screening, perform the serum β-hCG pregnancy test at the central laboratory. At randomization and other scheduled visits during the study, urine pregnancy tests should be performed. At randomization, a pregnancy test should be performed prior to the first dose of IMP. Additional serum or urine pregnancy tests may be performed, as determined necessary by the Investigator or required by local regulation, to establish the absence of pregnancy at any time during the participant's participation in the study. Communication by phone of the result of a pregnancy test performed at home to the site is allowed.
ⁿOnly in female participants, if needed to establish menopausal status.
^{o}The SDMT and CVLT-II will be performed in all participants. If for some reason, CVLT-II is not available at a given site due to reasons such as lack of translation, local certification, etc., then only the SDMT will be assessed for that participant.
^{p}A subset of sites that have 3T MRI capacity will perform additional sequences (eg, SWI). Further details will be defined in a central MRI manual.
^{q}A visit window of ±21 days is acceptable for MRIs performed after D 1. For systemic corticosteroids and adrenocorticotropic hormone, 1-month wash-out required prior to the MRI scans. The screening MRI scan should be performed as close as possible before the start of IMP. As much as possible, the MRI scan should be performed during the screening period only after it has been established that the participant meets all inclusion and no exclusion criteria.
^{r}A noninvasive activity monitor (actigraphy) may be optionally implemented by the Sponsor in a subset of participants during the course of the study if results from pilot assessment demonstrate feasibility.
Actigraphy baseline assessment will be done at Screening.
^{s}When available, clinical outcome assessments are to be completed by the participant prior to discussing their health status and prior to study treatment administration or other study related procedures. Where available per local regulations.
^{t}On days of PK sampling, the IMP needs to be taken at the study site after a “regular meal”. In case a participant forgot IMP at home or took IMP prior to arriving at the site on the day of visits with PK sampling, he/she will be asked to have a repeat assessment within 3 days of the missed PK sampling. A PK assessment shall be done as soon as possible after an overdose or if otherwise specified per protocol (eg, investigation of abnormal laboratory test values).
^{u}M6 and M12: Two samples: one sample between 30 to 90 minutes and one sample between 2.5 to 5 hours after IMP administration. M9: one sample 30-90 minutes after IMP
^{v}Pharmacodynamics and biomarkers samples will be collected only if permitted per local regulations.
^{w}DNA testing will be allowed at any time after signature of consent (in case it could not be done for some reason at Day 1). Participation is optional. Participants who do not wish to participate in the genetic research may still participate in the study. A separate consent is required for the genetic analysis component of the study.
^{x}This sample will be collected and stored for use if any unexpected safety issue occurs to ensure that a pre-dose baseline value is available for previously not assessed parameters (eg, serology) and for biomarkers research, if agreed.
^{y}Additional safety assessments can be performed if required by local regulations; such testing shall be performed locally whenever possible. Additional visits may be added if required by local regulations.
^{z}Samples for hematology and biochemistry tests on Day 1 (randomization) must be collected prior to administration of the first IMP dose.
^{aa}Samples must be shipped within 24 hours.
^{bb}Blood sample collection for lymphocyte phenotyping by flow cytometry will be performed in a subset of randomized participants. Participants who do did not have a baseline sample collected, will no longer have this test performed; participants with who had a baseline sample collected will have a second sample collected at EOT/pEOT.

Objectives and endpoints for the treatment are shown in Table 3B.

TABLE 3B
Objectives and endpoints

| Objectives | Endpoints |
|---|---|
| Primary | |
| To determine the efficacy of SAR442168 | Time to onset of 6-month CDP defined as |
| compared to placebo in delaying disability | follows: |
| progression in NRSPMS | Increase of ≥1.0 point from the |
| | baseline EDSS score when the |
| | baseline score is ≤5.0, OR |
| | Increase of ≥0.5 points when the |
| | baseline EDSS score is >5.0 |
| Secondary | |
| To evaluate efficacy of SAR442168 | Time to onset of sustained 20% |
| compared to placebo on clinical endpoints, | increase in the 9-hole peg test (9-HPT) |
| MRI lesions, cognitive performance, | confirmed over at least 3 months |
| physical function, and quality of life | Time to onset of sustained 20% |
| | increase in the timed 25-foot walk |
| | (T25-FW) test confirmed over at least 3 |
| | months |
| | Time to onset of 3-month CDP as |
| | assessed by the EDSS score |
| | Total number of new and/or enlarging |
| | T2-hyperintense lesions as detected by |
| | MRI, defined as the sum of the |
| | individual number of new and/or |
| | enlarging T2 lesions at all scheduled |
| | visits starting after baseline up to and |
| | including the end of study (EOS) visit |
| | Time to onset of CDI defined as ≥1.0 |
| | point decrease on the EDSS score from |
| | baseline confirmed over at least 6 |
| | months |
| | Percent change in brain volume loss |
| | (BVL) as detected by brain MRI scans |
| | at EOS compared to Month 6 |
| | Change in cognitive function at the |
| | EOS compared to baseline as assessed |
| | by SDMT |
| | Change in cognitive function at EOS |
| | compared to baseline as assessed by |
| | CVLT-II, where available |
| | Change in MSQoL-54 questionnaire |
| | score from baseline through the EOS |
| To evaluate safety and tolerability of | Adverse events (AEs), serious AEs, |
| SAR442168 | AEs leading to permanent study |
| | intervention discontinuation, AEs of |
| | special interest, and potentially |
| | clinically significant abnormalities in |
| | laboratory tests, safety scales, ECG, |
| | and vital signs during the study period |
| To evaluate population PK of SAR442168 | Plasma concentration of SAR442168 |
| and relevant metabolite(s) in NRSPMS and | (population PK assessment) at Months |
| its relationship to efficacy and safety | 6, 9, and 12 |
| To evaluate pharmacodynamics of | Change in plasma NfL levels at the |
| SAR442168 | EOS compared to baseline |
| | Change in lymphocyte phenotype |
| | subsets in whole blood at the |
| | EOS compared to baseline in a subset |
| | of participants |
| | Change in serum immunoglobulin level |
| | at the EOS compared to baseline |
| | Change in serum Chi3L1 levels at the |
| | EOS compared to baseline |
| Tertiary/exploratory | |
| To evaluate efficacy of SAR442168 on | Time to onset of sustained 20% increase |
| disease progression and activity in | in the 9-HPT for at least 6 months |
| NRSPMS, assessed by other clinical and | Time to onset of sustained 20% increase |
| imaging measures and by self reported | in the T25-FW for at least 6 months |
| assessment | Time to onset of a 4-point decrease in the |
| | SDMT, confirmed over at least 3 and 6 |
| | months |
| | The proportion of participants with CDI |
| | confirmed over at least 6 months |
| | The proportion of participants with CDI |
| | confirmed over at least 6 months and |
| | maintained until the EOS |
| | Change from baseline to Months 12, 18, |
| | and 24 and to the EOS in the EDSS score, |
| | T25-FW test, 9-HPT, SDMT, and CVLT- |
| | II |
| | Change from baseline to Months 12, 18, |
| | and 24 and to the EOS in modified |
| | MSFC-3, assessed as the composite of the |
| | T25-FW test, 9-HPT, and SDMT |
| | Proportion of participants with NEDA-3 |
| | at Months 18, 24, 30, 36, and the EOS |
| | The annualized adjudicated relapse rate |
| | (ARR) |
| | Actigraphic analysis of activity counts and |
| | indices of change from baseline to the |
| | EOS summarized over time (in a subset of |
| | participants) |
| | Change from baseline of total volume of |
| | T2-hyperintense lesions as detected by |
| | brain MRI at Months 18, 24, and the EOS |
| | Total number of new Gd-enhancing T1 |
| | hyperintense lesions as detected by MRI, |
| | defined as the sum of the individual |
| | number of new Gd-enhancing T1- |
| | hyperintense lesions at all scheduled visits |
| | starting after baseline up to and including |
| | the EOS visit |
| | Change from baseline by visit in the |
| | volume of T1-hypointense lesions and |
| | cumulative number of new T1 |
| | hypointense lesions |
| | MTR recovery at EOS in new MTR |
| | lesions detected at months 6 and 12 |
| To evaluate the treatment effect of | Change in number of phase rim lesions in |
| SAR442168 via changes in participants' | SWI MRI from baseline through the EOS |
| health-related quality of life (HRQoL), and | (subset of centers with capacity of 3T |
| working capacity | MRI) |
| | Number and volume of slowly evolving |
| | lesions (SELs) Normalized T1 (nT1) |
| | intensity evolution in SELs |
| | Change in EQ-5D-5L from baseline by |
| | visit over time |

Abbreviations: 9-HPT, 9-hole peg test; AE, adverse event; AESI, adverse event of special interest; CDI, confirmed disability improvement; CDP, confirmed disability progression; Chi3L1, chitinase-3-like protein 1; ECG, electrocardiogram; EDSS, Expanded Disability Status Scale; EOS, end of study; MRI, magnetic resonance imaging; MSQoL-54, Multiple Sclerosis Quality of Life-54 Questionnaire; NfL, neurofilament light chain; PD, pharmacodynamic; PK, pharmacokinetic; nrSPMS, non-relapsing secondary progressive multiple sclerosis; T25-FW, timed 25-foot walk.

### Number of Participants:

Approximately 1700 people will be screened to achieve 1290 participants randomly assigned to study intervention.

Enrolled participants will be randomly assigned at a ratio of 2:1 to 60 mg (established from dose-finding Study DRI15928) of oral, daily SAR442168 or daily matching placebo.

Randomization will be stratified by age at screening (>40 versus ≤40 years) and geographic region (US versus non-US).

Note: “Enrolled” means a participant's, or their legally acceptable representative's, agreement to participate in a clinical study following completion of the informed consent process. Potential participants who are screened for the purpose of determining eligibility for the study, but do not participate in the study, are not considered enrolled, unless otherwise specified by the protocol.

### Intervention Groups and Duration:

### Study Intervention(s)

### Investigational Medicinal Product(s)

Formulation: SAR442168 film coated tablet
Route(s) of administration: oral
Dose regimen: 60 mg once daily
Investigational medicinal product(s)
Formulation: placebo to match SAR442168 film coated tablet
Route(s) of administration: oral
Dose regimen: once daily

### Noninvestigational Medicinal Product(s)

Formulation: MRI contrast-enhancing preparations
Route(s) of administration: intravenous (IV)
Dose regimen: per respective label

### Temporary Investigational Medicinal Product (IMP) Interruption Due to Surgery

If surgery is needed during the study, consider the benefit-risk of withholding the IMP for at least 3 to 7 days pre- and post-surgery and the risk of bleeding

The duration of treatment will vary for individual participants, depending on the time of recruitment. With a planned recruitment period of approximately 24 months and an assumed event rate (discussed below), the duration of the study should be approximately 48 months. All recruited participants will be followed in the study until the common study end, which will be estimated and announced by the Sponsor to ensure that approximately 288 events of 6-month CDP have been observed before the study end.

### Statistical Considerations:

### Primary Endpoint:

The primary endpoint will be the treatment difference between SAR442168 and placebo in time to onset of 6 month-CDP regardless of completion of the treatment period. This endpoint corresponds to a “treatment policy strategy”. This endpoint will be considered primary for supporting regulatory decision making.

The time to onset of 6-month CDP will be analyzed by a Cox proportional hazards model with terms for treatment, age at screening (>40, ≤40 years) and geographic region (US, non-US). A log-rank test stratified by age at screening (>40, ≤40 years) and geographic region (US, non-US) to compare SAR442168 to placebo will also be examined.

In this Primary ITT Analysis:
For participants who complete the study without an initial disability progression or prematurely discontinue the study before 6-month confirmation of an onset of disability progression, the participant's event time will be censored at the date of last EDSS assessment.
For participants who have an initial onset of disability progression but reach the common study end date prior to 6-month confirmation, the event status of the participant will be determined by an imputation approach. Since in this setting, the partial missing data can reasonably be assumed to be missing at random, this approach leverages the partial information and follows the ITT principle. A logistic model with terms for age at screening (>40, ≤40 years) and geographic region (US, non-US) will be used to determine the event status as the imputation model within each treatment. A multiple imputation approach will be used to summarize the results.

Only EDSS assessments measured more than 90 days after the onset of an adjudicated relapse will be used to determine onset of disability progression. In addition, for the purpose of confirmation, only EDSS scores measured more than 90 days after the onset of an adjudicated relapse will be used. In case of such MS relapse, the next quarterly EDSS assessment will be used for CDP confirmation. The minimum increase in score required for progression must also be maintained for any non-confirmatory (ie, intervening) EDSS assessment(s) between the initial (onset) and confirmation EDSS scores.

### Main Secondary Endpoints:

For other time-to-event endpoints (time to onset of sustained 20% increase in the 9-HPT, of sustained 20% increase in the T25-FW, of 3-month CDP, and of CDI), similar analysis as for the primary analysis of the primary efficacy endpoint will be performed in the ITT population.

Continuous endpoints (percent change in brain volume loss, change in cognitive function, change in physical function, and change in MSQoL-54 at EOS) will be analyzed using a mixed-effect model with repeated measures (MMRM) approach. The model will include change/percent change values for the respective endpoint at each scheduled visit as response variables, and treatment, age at screening (>40, ≤40 years), geographic region (US, non-US), visit, treatment by-visit interaction, baseline value for the endpoint being assessed and baseline value-by-visit interaction as covariates.

Categorical efficacy endpoints with count data (new and/or enlarging T2 hyperintense over the study period after baseline) will be analyzed using a negative binomial regression model. The model will include the total count occurring during the observation period as the response variable, with treatment group, age at screening (>40, ≤40 years), and geographic region (US, non-US) as covariates. Log transformed number of scans will be the offset variable.

### Analysis of Safety Data:

All safety summaries will be descriptive; no statistical significance tests will be performed on safety data. This includes treatment-emergent adverse events (TEAEs) and other safety information (eg, clinical laboratory evaluations, electrocardiograms [ECGs], and vital signs). TEAEs are defined as adverse events (AEs) that developed or worsened or became serious during the treatment period. These analyses will be based on the safety population, defined as all participants randomly assigned and exposed to study intervention, regardless of the amount of exposure, analyzed according to the treatment actually received.
Two participants had treatment-emergent transient alanine aminotransferase (ALT) increase >3×ULN, 1 during the 30 mg SAR442168 treatment period (at Week 8, 105 U/L [normal range 6 to 34 U/L]) that returned to normal range within 4 days and 1 during the 60 mg SAR442168 treatment period (at Week 4, 107 U/L [normal range 6 to 34 U/L]). The participant in the 60 mg group had slightly elevated ALT at screening (48 U/L) and at baseline (50 U/L); ALT levels returned to the normal range in 8 weeks. Both participants continued study treatment during this period. All other liver enzyme levels for both participants were within normal ranges during the treatment period; one event was assessed as related and one as unrelated to the study drug by the Investigators. Both participants completed the DRI15928 study and successfully rolled over to the LTS follow-up study.
One event of mild petechia in a female participant (at Week 8 in the SAR442168 30 mg group) and 2 events of mild microscopic hematuria in 2 male participants (1 event at Week 16 in the SAR442168 30 mg group and 1 event on Day 1 in the SAR442168 60 mg group, with occult blood noted in urine) were reported during the treatment period in the SAR442168 Phase 2b trial. The hematology results were clinically insignificant for all 3 participants from the onset of the events. The participant with mild petechia had benign pigmentary lesions noted during screening, and the event was assessed as related to the study drug by the Investigator. The 2 events of mild microscopic hematuria were assessed as unrelated to the study drug. All 3 events resolved spontaneously.
No severe infections occurred. The most frequently reported (≥3 events total) in the SAR442168 treatment period were upper respiratory tract infection, nasopharyngitis, gastroenteritis, and respiratory tract infection.
No clinically significant cytopenia, including thrombocytopenia and neutropenia, were reported or detected based on hematologic laboratory results, and no clinically significant cardiac arrhythmia was observed via ECG monitoring during the study. An identified risk for tolebrutinib has been identified as follows:
Treatment-emergent SAEs of drug-induced liver injury (DILI) were reported in the ongoing Phase 3 trials; however, all cases occurred between Months 2 to 3 and appear reversible after treatment discontinuation, with potential confounders identified for some of the cases.

Drug-induced liver injury has been identified in the ongoing Phase 3 trials. The reported events occurred Months 2 to 3 after the start of the IMP, and the elevation of liver enzymes appears reversible after IMP discontinuation. Exclusion criteria and monitoring frequency have been updated in all actively recruiting protocols to mitigate risk of hepatic injury.

This is a Phase 3, randomized, double-blind, 2-arm, placebo-controlled, parallel group, multicenter, event-driven (6-month CDP) trial with a variable treatment duration ranging from approximately 24 to 48 months in participants with NRSPMS.

The study will consist of the following study periods:

Screening period: Day −28 to Day −1.

Randomization/start of IMP: Eligible participants will be randomly assigned at a 2:1 ratio to receive oral SAR442168 (60 mg) daily or matching placebo daily.

Intervention period: Double-blind treatment period for assessment of efficacy and safety up to the EOS as described in the End of Study Definition.

A month is defined as a period of 28 days by convention.

Safety follow-up period/EOS: 4 to 8 weeks after the last dose of study treatment (for participants completing IMP treatment [double blind or open-label, if rescued after 6-month CDP] and not entering the LTS study) to collect safety data.

EOS: A participant is considered to have completed the study if he/she has completed all periods of the study including the EOS Visit, whether remaining on IMP or not.

Participants with 6-month CDP are eligible for open-label active treatment as rescue (SAR442168) (under heading Rescue medicine).

The duration of the treatment period will vary for individual participants, depending on the time of recruitment and study end as described below. All recruited participants will be followed in the study until approximately 288 events of 6-month CDP are observed. With a planned recruitment period of approximately 24 months and an assumed event rate, the duration of the study should be approximately 48 months, with estimated mean treatment duration of 33 to 36 months.

Participants will be encouraged to remain in the study and comply with all study visits until the EOS in the case that they discontinue the study intervention early.

To minimize possible biases in the study outcome, the study is double blinded. The blind of initial treatment will be kept from participants, any Investigator site staff, and the Sponsor until the study end.

### Study Population

Prospective approval of protocol deviations to recruitment and enrollment criteria, also known as protocol waivers or exemptions, is not permitted.

### Inclusion Criteria

Participants are eligible to be included in the study only if all of the following criteria apply as shown in Table 3C:

TABLE 3C
Inclusion Criteria

| Category | Criteria | |
|---|---|---|
| Age | I 01. | Participant must be 18 to 60 years of age inclusive, at the |
| | | time of signing the informed consent. |
| Type of | I 02. | The participant must have a previous diagnosis of RRMS |
| participant | | in accordance with the 2017 revised McDonald criteria |
| and disease | | (Thompson et al. Lancet Neurol. 2018, 17, 162-173). |
| characteristics | I 03. | The participant must have a current diagnosis of SPMS in |
| | | accordance with the clinical course criteria (Lublin et al. |
| | | Neurology 1996 46, 907-911) revised in 2013 (Lublin et |
| | | al. Neurology 2014, 83, 278-286) and endorsed by an |
| | | Adjudication Committee. |
| | I 04. | The participant must have documented evidence of |
| | | disability progression observed during the 12 months |
| | | before screening. Eligibility will be analyzed by an |
| | | Adjudication Committee (to evaluate source data for |
| | | disability confirmation). |
| | I 05. | Absence of clinical relapses for at least 24 months. |
| | I 06. | The participant must have an EDSS score at screening |
| | | from 3.0 to 6.5 points, inclusive. |
| Weight | I 08. | Not applicable. |
| Sex | I 09. | Male and/or female |
| | | Contraceptive use should be consistent with local regulations |
| | | regarding the methods of contraception for those |
| | | participating in clinical studies. |
| | | A) Male participants Not applicable. |
| | | B) Female participants |
| | | A female participant is eligible to participate if she is not |
| | | pregnant or breastfeeding, and at least one of the |
| | | following conditions applies: |
| | | Is not a woman of childbearing potential (WOCBP) |
| | | OR |
| | | Is a WOCBP and agrees to use an acceptable |
| | | contraceptive method during the intervention period. |
| | | WOCBP must use reliable means of contraception at |
| | | a minimum. |
| | | A WOCBP must have a negative highly sensitive |
| | | pregnancy test (urine or serum as required by local |
| | | regulations) at screening and before the first dose of |
| | | study intervention. |
| | | If a urine test cannot be confirmed as negative (eg, an |
| | | ambiguous result), a serum pregnancy test is required. |
| | | In such cases, the participant must be excluded from |
| | | participation if the serum pregnancy result is positive. |
| | | Requirements for pregnancy testing during |
| | | and after study intervention are located in the |
| | | SoA (Tables 3A1 and 3A2). |
| | | Additional requirements for pregnancy |
| | | testing during and after study intervention are |
| | | located in Table 3E. |
| | | The Investigator is responsible for review of medical |
| | | history, menstrual history, and recent sexual activity to |
| | | decrease the risk for inclusion of a woman with an early |
| | | undetected pregnancy, if allowed by local regulations. |
| Informed | I 10. | The participant is capable of giving signed informed |
| Consent | | consent which includes compliance with the requirements and |
| | | restrictions listed in the informed consent form (ICF). A specific |
| | | ICF for legally minor participants must also be signed by the |
| | | participant's legally authorized representative. |

### Exclusion Criteria

Participants are excluded from the study if any of the following criteria apply as shown in Table 3D:

TABLE 3D
Exclusion Criteria

| Category | Criteria | |
|---|---|---|
| Medical | E 01. | The participant has a history of infection or may be at risk |
| conditions | | for infection: |
| | | A history of T-lymphocyte or T-lymphocyte-receptor |
| | | vaccination, transplantation (including solid organ, stem |
| | | cell, and bone marrow transplantation) and/or antirejection |
| | | therapy. |
| | | The participant has received any live (attenuated) vaccine |
| | | (including but not limited to varicella zoster, oral polio, and |
| | | nasal influenza) within 2 months before the first treatment |
| | | visit. |
| | | The participant has a lymphocyte count less than the lower |
| | | limit of normal (LLN) at the Screening Visit. |
| | | A history of diagnosis of progressive multifocal |
| | | leukoencephalopathy (PML) or evidence of findings |
| | | suggestive of PML on the screening MRI. |
| | | A history of infection with human immunodeficiency virus |
| | | (HIV). |
| | | A history of active or latent tuberculosis (TB); TB testing |
| | | should be performed at screening and again during the |
| | | study, if clinically indicated and maybe repeated based on |
| | | clinical judgment, borderline results, or clinical suspicion |
| | | of TB infection. In case of confirmed active or latent TB |
| | | the patient can be re-screened after full completion of anti- |
| | | tuberculosis treatment. |
| | | NOTE: The Investigator may consult with an infectious disease |
| | | expert if required, eg, test results are unclear or there is a |
| | | suspicion of false-positive test results. If the infectious disease |
| | | expert considers the test results as false-positive and not |
| | | clinically relevant and confirms that the participant can be |
| | | enrolled in the trial, the Investigator must document this in the |
| | | source data and may then randomize the participant. |
| | | Persistent chronic or active or recurring system infection |
| | | that may adversely affect participation or IMP |
| | | administration in this study, as judged by the Investigator. |
| | | Fever within 4 weeks of the Screening Visit (≥38° C.; |
| | | however, if due to brief and mild ear, nose, throat viral |
| | | infection participant may be included based on the |
| | | Investigator's judgment). |
| | | Participants at risk of developing or having reactivation of |
| | | hepatitis: results at screening for serological markers for |
| | | hepatitis B and C indicating acute or chronic infection. See |
| | | the Study Manual for further details. |
| | E 02. | The presence of psychiatric disturbance or substance abuse |
| | | as evidenced by: |
| | | A history of any psychiatric disease, behavioral condition, or |
| | | depression requiring hospitalization within 2 years prior to |
| | | the Screening Visit. |
| | | A documented history of attempted suicide or suicidal |
| | | ideation of category 4 or |
| | | 5 according to the Columbia Suicide Severity Rating Scale |
| | | (C-SSRS) baseline/screening version over the 6 months |
| | | prior to the Screening Visit, OR if in the Investigator's |
| | | judgment, the participant is at risk for a suicide attempt. |
| | | Active alcohol use disorder or a history of alcohol or drug |
| | | abuse within 1 year prior to the Screening Visit. |
| | | Current alcohol intake >2 drinks per day for men and >1 |
| | | drink per day for women |
| | | (1 drink = approximately 14 grams of alcohol = 350 mL |
| | | beer = 140 mL wine = 40 mL of spirits). |
| | E 03. | The following findings obtained during the Screening Visit |
| | | considered in the Investigator's judgment to be clinically |
| | | significant in the context of this trial: |
| | | Any screening laboratory values outside normal limits. |
| | | Abnormal ECG. |
| | E 04. | Conditions that may predispose the participant to excessive |
| | | bleeding: |
| | | A bleeding disorder or known platelet dysfunction at any |
| | | time prior to the Screening Visit. |
| | | A platelet count <150 000/μL at the Screening Visit. |
| | | The participant has had major surgery within 4 weeks prior |
| | | to the Screening Visit, which could affect the participant's |
| | | safety or affect immune response (as judged by the |
| | | Investigator) or has planned any elective major surgery |
| | | during the study. |
| | | A history of significant bleeding event within 6 months |
| | | prior to screening, according to the Investigator's judgment |
| | | such as, but not limited to cerebral or gastrointestinal |
| | | bleeding. |
| | E 05. | Conditions that would adversely affect participation in the |
| | | study or make the primary efficacy endpoint non-evaluable: |
| | | A short life expectancy due to pre-existing health |
| | | condition(s) as determined by their treating neurologist. |
| | | A history or presence of significant other concomitant |
| | | illness according to the Investigator's judgment such as, but |
| | | not limited to cardiovascular (including Stage III or IV |
| | | cardiac failure according to New York Heart Association |
| | | [NYHA] classification), or renal (ie, undergoing dialysis), |
| | | neurological, endocrine, gastrointestinal, metabolic, |
| | | pulmonary, or lymphatic disease that would adversely |
| | | affect participation in this study. |
| | | Acute liver disease, cirrhosis, chronic liver disease (unless |
| | | considered stable for >6 months). |
| | | Confirmed screening ALT >1.5 × upper limit of normal |
| | | (ULN) OR AST >1.5 × ULN OR alkaline phosphatase >2 × |
| | | ULN (unless caused by non-liver-related disorder or |
| | | explained by a stable chronic liver disorder) OR total |
| | | bilirubin >1.5 × ULN (unless due to Gilbert syndrome or |
| | | non-liver-related disorder). |
| | | At screening, elevated transferrin saturation (>50% in |
| | | males and >40% in females) and/or with elevated ferritin |
| | | levels >500 μg/L. |
| | | Any malignancy within 5 years prior to the Screening Visit |
| | | (except for effectively treated carcinoma in situ of the |
| | | cervix or adequately treated non-metastatic squamous or |
| | | basal cell carcinoma of the skin) will also be exclusionary. |
| | | Any other medical condition(s) or concomitant disease(s) |
| | | making them nonevaluable for the primary efficacy |
| | | endpoint or that would adversely affect participation in this |
| | | study, as judged by the Investigator. |
| Prior/ | E 06. | A requirement for concomitant treatment that could bias |
| concomitant | | the primary evaluation, such as any of the following |
| therapy | | medications/treatments within the specified time frame |
| | | before any randomization assessment (no wash out is |
| | | required for dimethyl fumarate, interferon beta or |
| | | glatiramer acetate treatments although use is not permitted |
| | | on or after Day 1): |

| | Exclusionary if |
|---|---|
| | used/used within |
| | required wash- |
| Medication | out period |
| Systemic corticosteroids, | 1 month prior to |
| adrenocorticotropic hormone | screening MRI |
| | scan |
| Siponimod, ponesimod | 1 week before |
| | randomization |
| | with MRI and |
| | clinical assessment |
| | for PML prior to |
| | randomization |
| Plasma exchange | 1 month prior to |
| | randomization |
| IV immunoglobulin | 2 months prior to |
| | randomization |
| Fingolimod, ozanimod | 6 weeks before |
| | randomization |
| | with MRI and |
| | clinical assessment |
| | for PML |
| Teriflunomide^{a} mildly to moderately | 3 months prior to |
| immunosuppressive/chemotherapeutic | randomization |
| medications such azathioprine, | |
| mycophenolate mofetil, and | |
| methotrexate | |
| Natalizumab | 2 months before |
| | randomization |
| | with MRI and |
| | clinical |
| | assessments for |
| | PML |
| B-cell depleting therapies such as | 6 months prior |
| ocrelizumab and rituximab | |
| to randomization Ofatumumab | 4 months |
| Highly | 2 years prior to |
| immunosuppressive/chemotherapeutic | randomization |
| medications: mitoxantrone up to 120 | |
| mg/m² body surface area, | |
| cyclophosphamide, cladribine, | |
| cyclosporine | |
| Alemtuzumab | 4 years prior to |
| | randomization |
| Other MS-disease-modifying | 5 half-lives or until |
| therapies | end of |
| | pharmacodynamics |
| | activity, whichever |
| | is longer |
| Lymphoid irradiation, bone marrow | No patient who has |
| transplantation, mitoxantrone (with | received any of |
| evidence of cardiotoxicity following | these treatments at |
| treatment, or cumulative lifetime | any time is |
| dose >120 mg/m²), other strongly | eligible. |
| immunosuppressive treatments with | |
| very long-lasting effects | |

| | | |
|---|---|---|
| | | IV: intravenous, MRI: magnetic resonance imaging, MS: multiple |
| | | sclerosis, PML: progressive multifocal leukoencephalopathy |
| | | ^{a}No time restriction if accelerated elimination procedure is done. |
| Prior/ | E 07. | The participant is receiving potent and moderate inducers of |
| concurrent | | CYP3A or potent inhibitors of CYP2C8 hepatic enzymes as |
| clinical study | | listed in Table 3I. |
| experience | E 08. | The participant is receiving anticoagulant/antiplatelet |
| | | therapies; those that are not permitted to be taken |
| | | concomitantly with the IMP, include the following: |
| | | Acetylsalicylic acid (aspirin) >81 mg/day. |
| | | Antiplatelet drugs (eg, clopidogrel). |
| | | Warfarin (vitamin K antagonist). |
| | | Heparin, including low molecular weight heparin |
| | | (antithrombin agents). |
| | | Dabigatran (direct thrombin inhibitor). |
| | | Apixaban, edoxaban, rivaroxaban (direct factor Xa |
| | | inhibitors). |
| | | Note: All the above-mentioned drugs must be stopped at |
| | | least 5 half-lives before study drug administration except |
| | | for aspirin, which must be stopped at least 8 days before. |
| | | These washout periods are only applicable in the case that |
| | | the Investigator deems it clinically appropriate to |
| | | discontinue the listed medications or there is a recent |
| | | history of use of these medications (as in the case when |
| | | short-term treatment with anticoagulants is clinically |
| | | recommended for certain thrombotic events), and |
| | | therefore these washout periods will need to be followed |
| | | prior to randomization. |
| | | If however the participant has a chronic underlying medical |
| | | condition (stroke, coronary or carotid artery disease, |
| | | valvular heart disease etc) requiring continued use of these |
| | | medications, the participant cannot be enrolled in the study. |
| | E 09. | The participant has sensitivity to any of the study |
| | | interventions, or components thereof, or has a drug or other |
| | | allergy that, in the opinion of the Investigator, |
| | | contraindicates participation in the study. |
| | E 10. | The participant was previously exposed to any BTK inhibitor, |
| | | including SAR442168. |
| | E 11. | The participant has taken other investigational drugs within |
| | | 3 months or 5 half-lives, whichever is longer, before the Screening |
| | | Visit. |
| Diagnostic | E 12. | The participant has a contraindication for MRI, ie, presence |
| assessments | | of pacemaker, metallic implants in high risk areas (ie, |
| | | artificial heart valves, aneurysm/vessel clips), presence of |
| | | metallic material (eg, shrapnel) in high risk areas, known |
| | | history of allergy to any contrast medium, or history of |
| | | claustrophobia that would prevent completion of all |
| | | protocol scheduled MRI. |
| | | Note: People with a contraindication to Gd can be enrolled |
| | | into the study but cannot receive Gd contrast dyes during |
| | | their MRI scan. |
| Other | E 13. | Individuals accommodated in an institution because of |
| exclusions | | regulatory or legal order; prisoners or participants who are |
| | | legally institutionalized. |
| | E 14. | Any country-related specific regulation that would prevent |
| | | the participant from entering the study. |
| | E 15. | The participant is not suitable for participation, whatever the |
| | | reason, as judged by the Investigator, including medical or |
| | | clinical conditions, or participants potentially at risk of |
| | | noncompliance to study procedures or not able to follow the |
| | | schedule of protocol assessments due to other reasons. |
| | E 16. | Participants are employees of the clinical study site or other |
| | | individuals directly involved in the conduct of the study, or |
| | | immediate family members of such individuals (in |
| | | conjunction with Section 1.61 of the International Council |
| | | for Harmonisation [ICH] - Good Clinical Practice [GCP] |
| | | Ordinance E6). |
| | | Any other situation during study implementation/course that |
| | | may raise ethics considerations. |
| | | Note: a one-time retest at screening may be |
| | | performed if an abnormal laboratory test value is |
| | | considered temporary. |

### Lifestyle Considerations

Meals and dietary restrictions: SAR442168 shall be taken with a regular meal. When possible, the meal with which SAR442168 is taken (eg, breakfast, lunch, or dinner) should be consistent throughout the study. The typical meal with which the IMP is taken will be recorded at each visit. In case the mealtime needs to be changed for IMP administration, a gap of a minimum of 12 hours between 2 doses should be maintained.

Caffeine, alcohol, and tobacco: For each visit with PK/PD assessment (refer to Tables 3A1 and 3A2), participants will abstain from ingesting caffeine- or xanthine-containing products (eg, coffee, tea, cola drinks, and chocolate) for 2 hours before the start of treatment until after collection of the final PK and/or PD sample later that day.

For each visit with PK/PD assessment (refer to Tables 3A1 and 3A2), participants will abstain from alcohol for 24 hours before the start of treatment until after collection of the final PK and/or PD sample later that day.

During the entire study, participants should be warned not to consume substantial quantities of alcohol, defined as >14 grams (1 standard drink) per day in female participants or >28 grams (2 standard drinks) per day in male participants on a regular basis.

### Study Intervention

Study intervention is defined as any investigational intervention(s), marketed product(s), or placebo intended to be administered to a study participant according to the study protocol.

### Criteria for Temporarily Delaying Enrollment and Administration of Study Intervention

During a regional or national emergency declared by a governmental agency, if the site is unable to adequately follow protocol mandated procedures, contingency measures should be considered for screening, enrollment, randomization, and administration of the study intervention.

TABLE 3E
Overview of study interventions administered

| | | |
|---|---|---|
| ARM name | SAR442168 | Placebo |
| Intervention name | SAR442168 | Placebo |
| Type | Drug | Drug |
| Dose formulation | Film coated tablet | Film coated tablet |
| Unit dose | 60 mg | 0 mg |
| strength(s) | | |
| Dosage level(s) | Once daily | Once daily |
| Route of | Oral | Oral |
| administration | | |
| Use | Experimental | Placebo |
| IMP and NIMP | IMP | IMP |
| Packaging and | Study intervention will be | Study intervention will be provided in |
| labeling | provided in wallet blister | wallet blister packaging. Each wallet |
| | packaging. Each wallet blister | blister packaging will be labeled as per |
| | packaging will be labeled as | country requirements. |
| | per country requirements. | |

IMP: investigational medicinal product; NIMP: noninvestigational medicinal product.

Between the protocol-scheduled on-site visits, interim visits may be required for IMP dispensing. As an alternative to these visits or to replace on-site IMP dispensation, if needed, SAR442168 may be supplied from the site to the participant via a Sponsor-approved courier company (direct-to-patient [DTP]) where allowed by local regulations and approved by the Sponsor.

### Noninvestigational Medicinal Product

### MRI Contrast-Enhancing Preparations

Route(s) of administration: IV
Dose regimen: as per respective label

### Concomitant Therapy

Any medication or vaccine (including over-the-counter or prescription medicines, vitamins, and/or herbal supplements) that the participant is receiving at the time of enrollment or receives during the study must be recorded along with:
Reason for use.
Dates of administration including start and end dates.
Dosage information including dose and frequency.

Any live (attenuated) vaccine within 2 months before the first treatment visit and during the intervention period is prohibited.

Therapies for MS noted in the exclusion criterion E 06 are not permitted after randomization while the participant is on study treatment. Short-term use (3 to 5 days) of glucocorticoids (eg, for MS relapse treatment or an acute illness) and local corticosteroids (eg, topical, nasal, ocular, otic, intra-articular) are allowed.

The Medical Monitor should be contacted if there are any questions regarding concomitant or prior therapy.

Participants must abstain from taking prescription or nonprescription drugs (including vitamins and dietary or herbal supplements) within 7 days (or 14 days if the drug is a potential enzyme inducer) or 5 half-lives (whichever is longer) before the start of study intervention until completion of the follow-up visit, unless, in the opinion of the Investigator and Sponsor, the medication will not interfere with the study.

Medications for treatment of MS symptoms (eg, walking impairment, fatigue, spasticity, incontinence, pain) should be maintained at a stable dose prior to screening and for the duration of the treatment period, if clinically feasible.

Anticoagulant/antiplatelet therapies are not permitted to be taken concomitantly with the IMP, including the following:
Acetylsalicylic acid (aspirin) >81 mg/day.
Antiplatelet drugs (eg, clopidogrel).
Warfarin (vitamin K antagonist).
Heparin, including low molecular weight heparin (antithrombin agents).
Dabigatran (direct thrombin inhibitor).
Apixaban, edoxaban, rivaroxaban (direct factor Xa inhibitors).

Paracetamol/acetaminophen, at doses of ≤3 grams/day, is permitted for use at any time during the study. Short courses (up to 5 days) of nonsteroidal anti-inflammatory drugs (NSAIDs) (other than acetylsalicylic acid), preferably selective cyclooxygenase-2 inhibitors at the lowest effective dose, may be given during the study if clinically necessary for the treatment of an existing medical condition or a new event. The Investigator must record the use of NSAIDs (and any other comedication) in the eCRF.

### CYP Inhibitors and Inducers:

Potent and moderate inducers of CYP3A or potent inhibitors of CYP2C8 hepatic enzymes are not permitted throughout the study (Table 3I).

Based on nonclinical drug metabolism studies, SAR442168 is a substrate of the CYP3A and CYP2C8 isoenzymes. In healthy participants, potent CYP3A4 inhibitor (itraconazole 200 mg once daily for 4 days) increased SAR442168 area under the curve AUC exposure 1.8-fold (Study INT16385) and potent CYP2C8 inhibitor (gemfibrozil 600 mg twice daily for 6 days) increased SAR442168 AUC exposure 8.4-fold (Study INT16726). Based on a satisfactory safety and tolerability profile and on the observed exposure in healthy participants who received SAR442168 at a dose of up to 240 mg SAR442168 once daily for 14 days under fed conditions (Study TDR16862), drugs that strongly inhibit CYP3A4 are allowed and drugs that strongly inhibit CYP2C8 are not permitted. In healthy participants, potent CYP3A4 and moderate CYP2C8 induction by rifampicin (600 mg once daily for 8 days) decreased SAR442168 exposure 6-fold (Study INT16726). Therefore, potent and also moderate (based on prediction) CYP3A inducers are not permitted due to their potential to decrease SAR442168 exposure and efficacy. See Table 3I for the list of drugs to be avoided.

The Sponsor should be contacted if there are any questions regarding concomitant or prior therapy.

### Rescue Medicine

If a participant achieves the primary endpoint (6-month CDP), they may, in conjunction with the Treating Investigator, choose to receive one of the following:
1. To switch to open-label SAR442168 treatment and continue regularly planned study visits;
2. To switch to a non-study treatment approved for NRSPMS in their respective country. The participant will be encouraged to remain in the study for planned clinical visits until common study end.

Should the participant and Investigator opt for the provision of rescue medicine, they will remain blinded to the original treatment assignment.

All individual blinded data will be reviewed and all queries resolved, if possible, before the switch to the rescue to ensure data integrity for primary assessment. Prior to initiation of rescue treatment, the Investigator shall confirm that there has been no adjudicated relapse within 90 days prior to the onset or confirmation of 6-month CDP. Based on individual symptoms and assessed risk of further progression, the Investigator and participant may choose for the participant to remain on the initial double-blind treatment after achieving 6-month CDP.

The initial treatment assignment will be kept blinded from participants, any Investigator site staff, and the Sponsor until the study end.

The supply of SAR442168 as rescue medication will be specified at the country level.

Multiple sclerosis relapses are not frequent in the NRSPMS population, but their occurrence during the study cannot be ruled out completely. In the case of MS relapse, treatments are allowed as per local routine practice (eg, high dose IV methylprednisolone for 3 to 5 days). The date and time of rescue medication administration as well as the name and dosage regimen of the rescue medication must be recorded.

### Dose Modification

Dose modification is not foreseen in this study. Treatment might need to be interrupted or permanently discontinued if deemed necessary due to an AE.

### Discontinuation of Study Intervention and Participant Discontinuation/Withdrawal

### Discontinuation of Study Intervention

### Definitive Discontinuation

The study intervention should be continued whenever possible.

Permanent intervention discontinuation is any treatment discontinuation associated with the definitive decision by the Investigator or the participant not to re-expose the participant to the study intervention at any time.

In rare instances, it may be necessary for a participant to permanently discontinue the study intervention. If study intervention is permanently discontinued, the participant will be asked to remain in the study to be evaluated until the EOS visit. For this set of participants (participants who discontinued the IMP and/or switched to other DMTs), no PK or biomarker samples will be collected after the pEOT visit, and MRI assessments will be performed only annually using the next annual visit as the starting point.

This will be important to continue to evaluate for safety and efficacy. See the SoA (Tables 3A1 and 3A2) for data to be collected at the time of discontinuation of the study intervention. In the case that the study intervention is permanently discontinued, the participant should be treated for MS according to local clinical practice and the best judgment of the Investigator.

The following may be justifiable reasons for the Investigator or Sponsor to discontinue a participant from treatment:
Adverse events that endanger the safety of the participant, or if discontinuation of study intervention is desired or considered necessary by the Investigator and/or participant.
If IMP discontinuation criteria are met as per the guidance for the follow up of laboratory abnormalities (Table 3F).
The participant is no longer deriving a therapeutic/clinical benefit in the opinion of the Investigator.
If a female participant becomes pregnant or wishes to become pregnant during the study.
At participant's request, ie, withdrawal of the consent for treatment.
Any serious opportunistic infections (eg, PML [see FIG. 6], HIV)
Continued need for/chronic use of prohibited a concomitant medication (see Table 3I).
Use of open-label SAR442168 or non-study disease modifying therapy approved for NRSPMS in their respective countries (eg, after 6-month CDP).
A brief physical examination will include, at a minimum, assessments of the skin, lungs, cardiovascular system, neurological examination, and abdomen (liver and spleen).
Investigators should pay special attention to clinical signs related to previous serious illnesses.
Any clinically significant new finding or worsening of previous finding should be reported as an AE, per Investigator's judgement.

### Safety Assessments

### Physical Examinations

A physical examination will be performed at the time points specified in the SoA (Tables 3A1 and 3A2).
A complete physical examination will include, at a minimum, assessments of the general appearance, head and neck, abdomen, lymph nodes, skin, cardiovascular system, respiratory system, musculoskeletal system, and neurological examination by the Treating Investigator. Height (at screening) and weight will also be measured and recorded. Further details will be provided in the Study Manual.
A brief physical examination will include, at a minimum, assessments of the skin, lungs, cardiovascular system, neurological examination, and abdomen (liver and spleen).
Investigators should pay special attention to clinical signs related to previous serious illnesses.
Any clinically significant new finding or worsening of previous finding should be reported as an AE, per Investigator's judgement.

The extent of the physical examination can be broadened at the discretion of the Treating Investigator in order to evaluate AEs or abnormal clinical laboratory test values.

### Vital Signs

Body temperature, heart rate, and blood pressure will be assessed.
Blood pressure and pulse measurements will be assessed with the participant in a supine or sitting position with a completely automated device. Manual techniques will be used only if an automated device is not available.
Blood pressure and pulse measurements should be preceded by at least 5 minutes of rest for the participant in a quiet setting without distractions (eg, television, cell phones).
Vital signs (to be taken before blood collection for laboratory tests) will consist of 1 heart rate and 3 blood pressure measurements (3 consecutive blood pressure readings will be recorded at intervals of at least 1 minute).

### Electrocardiograms

12-lead ECG will be obtained as outlined in the SoA (see Tables 3A1 and 3A2) using an ECG machine that automatically calculates the heart rate and measures PR, QRS, QT, and QTcF intervals. In case the ECG machine does not automatically calculate QTcF, manual calculation using nomogram or automatic website calculator (eg, https.//reference.medscape.com/calculator/48/ecg-corrected-qt) is acceptable
ECG and 30 second rhythm strips will be obtained locally. Further details will be included in the Study Manual.

### Clinical Safety Laboratory Assessments

See Table 3I for the list of clinical laboratory tests to be performed. Per the SoA, serology tests for hepatitis B and C will be performed during screening; testing for other infectious disease should be performed during screening if required locally.
The Treating Investigator may solicit emergency local laboratory data in case of emergent safety events to allow for appropriate treatment decisions. All clinically relevant solicited emergency local laboratory data will be recorded in the eCRF.
The Treating Investigator must review the laboratory report, document this review, and record any clinically relevant changes occurring during the study in the AE section of the eCRF. The laboratory reports must be filed with the source documents. Clinically significant abnormal laboratory findings are those which are not associated with the underlying disease, unless judged by the Investigator to be more severe than expected for the participant's condition.
All laboratory tests with values considered clinically significantly abnormal during participation in the study or within 30 days after the last dose of study intervention should be repeated until the values return to normal or baseline or are no longer considered clinically significant by the Investigator.
   If abnormal laboratory values do not return to normal/baseline within a period of time judged reasonable by the Investigator, the etiology should be identified and the Sponsor notified.
All protocol-required laboratory assessments, as defined in Table 3I, must be conducted in accordance with the laboratory manual and the SoA (see Tables 3A1 and 3A2). In the event the laboratory assessments in FIG. 2-8 indicate discontinuation of IMP, temporary discontinuation should be considered unless otherwise specified.
   If laboratory values from non-protocol specified laboratory assessments performed at the institution's local laboratory require a change in participant management or are considered clinically significant by the Investigator (eg, SAE or AE or dose modification), then the results must be recorded in the eCRF.

### Suicidal Ideation and Behavior Risk Monitoring

SAR442168 crosses the blood-brain barrier. Assessment of suicidal ideation and behavior as well as treatment-emergent suicidal ideation and behavior will be monitored during Study EFC16645 using the C-SSRS. For safety reasons, C-SSRS will be administered throughout the study by the Treating Investigator or delegated to an individual who is certified to administer the scale.

Study drug administration must be interrupted if a participant scores “yes” on items 4 or 5 of the Suicidal Ideation Section of the C-SSRS, or “yes” on any item of the Suicidal Behavior Section. A psychiatrist will be consulted and decide whether the study drug can be restarted and if any additional risk mitigation strategies are required (eg, increased monitoring, antidepressant administration).

### Adverse Event of Special Interest

An AESI is an AE (serious or nonserious) of scientific and medical concern specific to the Sponsor's product or program, for which ongoing monitoring and immediate notification by the Investigator to the Sponsor is required. Such events may require further investigation in order to characterize and understand them. Adverse events of special interest may be added, modified, or removed during a study by protocol amendment.
Pregnancy of a female participant entered in a study as well as pregnancy occurring in a female partner of a male participant entered in a study with IMP/NIMP
   It will be qualified as an SAE only if it fulfills one of the seriousness criteria (see Appendix 3 [Section 10.3]),
   In the event of pregnancy in a female participant, IMP should be discontinued,
   Follow-up of the pregnancy in a female participant or in a female partner of a male participant is mandatory until the outcome has been determined.
Symptomatic overdose (serious or nonserious) with IMP
   An overdose (accidental or intentional) with the IMP is an event suspected by the Investigator or spontaneously notified by the participant (not based on systematic pills count) and defined as at least twice the intended dose within the intended therapeutic interval (eg, >2 tablets of the IMP within a 12-hour interval).
Increase in alanine transaminase (ALT) >3×ULN
   ALT increase >3×ULN confirmed by retest within 72 hours or in the absence of a retest within 72 hours.
Other project specific AESI(s)
   ECG observation of atrial fibrillation or atrial flutter,
   Severe infection (NCI CTCAE Grade 3 or above) infection, that may or may not meet seriousness criteria (eg, a Grade 3 opportunistic infection),
   Moderate or severe hemorrhagic events (NCI CTCAE Grade 2 or above), including but not limited to symptomatic bleeding, bleeding in a critical area or organ such as the CNS, or intraocular bleeding,
   Thrombocytopenia, platelet count <75000/mm³ (see FIG. 3 for management flow chart).

### Clinical Laboratory Tests

The tests detailed in Table 3F will be performed by the central laboratory when feasible.

Local laboratory results are only required in the event that the central laboratory results are not available in time for either study intervention administration and/or response evaluation.

Additionally, if the local laboratory results are used to make either a study intervention decision or response evaluation, the results must be entered into the eCRF.

Protocol-specific requirements for inclusion or exclusion of participants are detailed in Tables 3C and 3D.

Additional tests may be performed at any time during the study as determined necessary by the Investigator or required by local regulations. Additional serum or urine pregnancy tests may be performed, as determined necessary by the Investigator or required by local regulation, to establish the absence of pregnancy at any time during the participant's participation in the study.

TABLE 3F
Protocol-required laboratory assessments

| Laboratory | | | |
|---|---|---|---|
| assessments | Parameters | | |
| Hematology | Platelet count | RBC indices: | WBC count with |
| | | | differential: |
| | RBC count | MCV | Neutrophils |
| | Hemoglobin | MCH | Lymphocytes |
| | Hematocrit | % Reticulocytes | Monocytes |
| | | | Eosinophils |
| | | | Basophils |
| Clinical | BUN | Sodium | AST/SGOT |
| chemistry | Creatinine | Calcium | ALT/SGPT |
| | Glucose | Total and direct bilirubin | Alkaline phosphatase |
| | Potassium | Total protein | Albumin |
| | | Chloride | Creatine phosphokinase |
| | | Bicarbonate | Lipase |

| | |
|---|---|
| Routine | Specific gravity |
| urinalysis | pH, glucose, protein, blood, ketones, bilirubin, urobilinogen, nitrite, leukocyte |
| | esterase by dipstick |
| | Microscopic examination (if blood or protein is abnormal and for signs of |
| | infection) |
| Other | FSH and estradiol (if needed, only in female participants to confirm |
| screening | postmenopausal state) |
| tests | Highly sensitive serum or urine β-hCG pregnancy test (as needed for |
| | women of childbearing potential) ^{b} |
| | Serology tests for hepatitis B (HBs Ag, anti-HBc IGM and total, anti-HBs) |
| | and C virus (anti-HCV); in case these results are inconclusive (eg anti-HBS |
| | negative and anti-HBc positive or anti-HC IgG positive), HBV-DNA or |
| | HCV-RNA testing, respectively, should be performed for confirmation. HIV |
| | and other infectious diseases, if locally required. |
| | Coagulation: PT/ INR, aPTT |
| | Tuberculosis test: Blood testing (eg, QuantiFERON ® TB Gold test) is |
| | preferred; skin testing (eg, tuberculin skin test) with ancillary testing will be |
| | allowed if blood testing is not available. T-SPOT can also be performed, if |
| | available. |
| | Iron panel (serum): iron, ferritin, trnasferrin saturation TIBC. |

ALT: alanine aminotransferase; anti-HBc; antibody to hepatitis B core antigen; anti-HBs: hepatitis B surface antibody; aPTT: activated partial thromboplastin time; AST: aspartate aminotransferase; BUN: blood urea nitrogen; β-hCG: human chorionic gonadotropin; FSH; follicle-stimulating hormone; IEC: independent ethics committee; INR: international normalized ratio; HBsAg: hepatitis B surface antigen; HBV: hepatitis B virus; HCV: hepatitis C virus; HIV: human immunodeficiency virus; Ig: immunoglobulin; IRB: institutional review board; MCH: mean corpuscular hemoglobin; MCV: mean corpuscular volume; PT: prothrombin time; RBC: red blood cell; SGOT: serum glutamic-oxaloacetic transaminase; SGPT: serum glutamic-pyruvic transaminase; TB: tuberculosis; ULN: upper limit of normal; WBC: white blood cell
^{a} Details of liver chemistry stopping criteria and required actions and follow-up assessments after observations of ALT >3 × ULN are given in FIG. 2-8. Clinical laboratory findings of ALT >3 × ULN and bilirubin ≥2 × ULN (>35% direct bilirubin), or ALT >3 × ULN and international normalized ratio (INR) >1.5, if INR measured, that may suggest severe liver injury and must be reported as an SAE.
^{b} Local urine testing for pregnancy will be standard for the protocol (except for the Screening Visit, when a serum pregnancy test is required) unless serum testing is required by local regulation or IRB/IEC.

Investigators must document their review of each laboratory safety report.

Laboratory/analyte results that could unblind the study will not be reported to investigative sites or other blinded personnel until the study has been unblinded. This includes PK assessments and any post-baseline biomarker or PD assessments.

### Liver and Other Safety: Actions and Follow-Up Assessments

These actions described in Table 3G and FIGS. 2-8 are required for ALT increase and thrombocytopenia events ONLY. For all other safety events described, these are suggested per the Investigator's medical judgement.

Neutropenia is to be recorded as an AE only if at least 1 of the criteria listed in the general guidelines for reporting AEs in Appendix 3 (Section 10.3) is met.

Thrombocytopenia is to be recorded as an AE only if at least 1 of the criteria listed in the general guidelines for reporting AEs in Appendix 3 (Section 10.3) is met.

Abbreviations in FIG. 3: aPTT: activated partial thromboplastin time; EDTA: Ethylenediaminetetraacetic acid; INR: international normalized ratio; PK: pharmacokinetic; PT: prothrombin time.

Abbreviations in FIG. 4A: ALT: alanine aminotransferase; ANCA: antineutrophil cytoplasmic antibodies; AST: aspartate aminotransferase; CMV: cytomegalovirus; CRF: case report form; DNA: deoxyribonucleic acid; dsDNA: double stranded DNA; EBV: Epstein-Barr Virus; GGT: Gamma-glutamyl transferase; HAV: Hepatitis A virus; HBc: Hepatitis B core; HBV: Hepatitis B virus; HCV: Hepatitis C virus; HEV: Hepatitis E virus; IgM: immunoglobulin M; IMP: investigational medicinal product; INR: international normalized ratio; lab: laboratory; LFT: liver function test; PT: prothrombin time; RNA: ribonucleic acid; LKM: liver kidney microsome; ULN: upper limit of normal.

Note: “Baseline” refers to ALT sampled at baseline visit; or if baseline value unavailable, to the latest ALT sampled before the baseline visit. The algorithm does not apply to the instances of increase in ALT during Screening.

TABLE 3G
Actions for cases of confirmed ALT elevation
In ANY CONFIRMED CASE, FOLLOW the instructions listed below:

INFORM the Site Monitor, who will forward the information to the Study Manager.
INVESTIGATE specifically for malaise with or without loss of consciousness,
dizziness, and/or hypotension and/or episode of arrhythmia in the previous 72
hours; rule out muscular injury.
PERFORM the following tests:
LFTs: AST, ALT, alkaline phosphatase, total and conjugated bilirubin and
prothrombin time/INR;
CPK, serum creatinine, complete blood count;
Anti-HAV IgM, anti-HBC IgM, (HBV-DNA if clinically indicated),
anti-HCV and HCV RNA, anti-CMV IgM and anti- HEV IgM
antibodies;
Depending on the clinical context, check for recent infection with EBV, herpes
viruses, and toxoplasma;
Consider iron, ferritin, transferrin saturation;
Consider hepatobiliary ultrasonography (or other imaging investigations if
needed);
CONSIDER auto-antibodies: antinuclear, anti-DNA, anti-smooth muscle, anti-LKM,
anti-mitochondrial;
CONSIDER DNA test for Gilbert's disease if clinically indicated;
CONSIDER consulting with a hepatologist;
CONSIDER patient hospitalization if INR >2 (or PT <50%) and/or central
nervous system disturbances suggesting hepatic encephalopathy;
MONITOR LFTs after permanent discontinuation of IMP:
Monitor as closely as possible (every 48 hours to every week)
until ALT is down-trending, then every 2 weeks until 1.5 ×
ULN, and then every scheduled visit;
Rechallenge Reinitiation of the study drug can only be considered after
discussion with the Sponsor's Medical Monitor once the ALT/AST decreases
below 1.5 × ULN, and there is no clinical contraindication. In case it is agreed
to re-start the study drug, it is recommended that ALT/AST be assessed
weekly for the first month and then monthly for the second and third months.
The occurrence of new elevation above 3 × ULN for the ALT/AST values will
lead to permanent discontinuation of the study drug; No rechallenge will be
considered for participants with >3 × ULN ALT and >2 × ULN bilirubin
increase
FREEZE serum sample (5 ml × 2).
COLLECT/STORE one PK sample following the instructions in the central
laboratory manual

Increase in serum creatinine is to be recorded as an AE only if at least 1 of the criteria listed in the general guidelines for reporting adverse events in Appendix 3 (Section 10.3) is met.

Increase in CPK is to be recorded as an AE only if at least 1 of the criteria in the general guidelines for reporting adverse events in Appendix 3 (Section 10.3) is met.

Abbreviations in FIG. 5: ARF, acute renal failure; ULN, upper limit of normal; DIC, disseminated intravascular coagulation; CPK, creatine phosphokinase; ECG, electrocardiogram; PK, pharmacokinetic(s).

Increase in serum creatinine is to be recorded as an AE only if at least 1 of the criteria listed in the general guidelines for reporting AEs in Appendix 3 (Section 10.3) is met.

Abbreviations in FIG. 8: CK-MB, creatine kinase-MB; CK-MM, creatine kinase-MM; ECG, electrocardiogram; PK, pharmacokinetic(s); ULN, upper limit of normal.

Increase in CPK is to be recorded as an AE only if at least 1 of the criteria in the general guidelines for reporting AEs in Appendix 3 (Section 10.3) is met.

SUSPECTED PML: If either the clinical presentation or MRI features of a participant are suggestive of PML, the diagnostic and action algorithm described in FIG. 6 is recommended.

Abbreviations in FIG. 6: Abbreviations: CSF, cerebrospinal fluid; Gd, gadolinium; IMP, investigational medicinal product; JCV, John Cunningham virus; MRI, magnetic resonance imaging; PCR, polymerase chain reaction; PML, progressive multifocal leukoencephalopathy

Clinical manifestations or MRI lesions features suspicious for PML are proposed in Table 3H (based on Berger et al. Neurology 2013, 80, 1430-1438 and Kappos et al. Lancet Neurol. 2007, 6, 431-441).

In the event that PML is suspected based on imaging results, the local radiologist will directly inform the Investigator and a central review of the MRI will not be required. The Investigator will obtain additional plasma, urine, and CSF samples for John Cunningham virus (JCV) analysis. Samples will be analyzed upon receipt and the results will be provided directly to the investigational site and to the Sponsor. Further management will be deferred to the Treating Investigator. However, next steps will include discontinuation of study treatment. Additional imaging will be at the discretion of the Investigator depending on the diagnostic workup and treatment plan.
The detection of John Cunningham virus (JCV) DNA in the cerebrospinal fluid of a patient with clinical and MRI features suggestive of PML establishes the diagnosis of PML.
If JCV DNA is not detected in cerebrospinal fluid and if clinical suspicion of PML remains high, another lumbar puncture should be performed.
If diagnosis remains uncertain and suspicion of PML remains high, a brain biopsy may be considered to establish a definitive diagnosis

TABLE 3H
Clinical and MRI features suggestive of PML

| | |
|---|---|
| Clinical | Subacute onset of weakness, sensory deficits, cognitive |
| history | or behavioral abnormalities, gait dysfunction, speech/ |
| | language difficulties or any other signs of cortical |
| | dysfunction, retrochiasmal visual defects or seizure |
| Brain | ≥1 T2/FLAIR hyperintense and T1 hypointense lesions |
| MRI | involving the subcortical and juxtacortical white matter, |
| | sparing the cortex, with no mass effect, with a continuous |
| | progression; new lesions with no enhancement (even when |
| | large) or with faint rim enhancement |

Clinical or MRI features suggestive of PML should be recorded as an AE/AESI/SAE following the definitions and procedures in Appendix 3 (Section 10.3).

Example of Drugs with a Potential to Change SAR442168 Metabolism or Absorption

The following drugs should not be taken during the study concomitantly with IMP due to their potential to change SAR442168 kinetics due to interaction with P450-mediated metabolism, being potent and moderate inducers of CYP3A or potent inhibitors of CYP2C8 liver enzymes (per the lists of the Drug Interaction Database Program of the University of Washington [www.druginteractioninfo.org]).

Please note that the lists provided are not exhaustive and that the product information of drugs intended for concomitant use should be consulted.

TABLE 3I
CYP3A Inducers and CYP2C8 Inhibitors
Potent CYP3A Inducers:

| | |
|---|---|
| Rifampin | Carbamazepine |
| St John's Wort extract | Phenobarbital |
| Avasimibe | Lumacaftor |
| Rifapentine | Rifabutin |
| Phenytoin | |

Potent CYP2C8 Inhibitors:

| | |
|---|---|
| Gemfibrozil | Clopidogrel |

Moderate CYP3A Inducers:

| | |
|---|---|
| Semagacestat | Asunaprevir/beclabuvir/daclatasvir |
| Cenobamate | Nafcillin |
| Lesinurad | Modafinil |
| Bosentan | Telotristat ethyl |
| Thioridazine | Elagolix |
| Rifabutin | |

### Abbreviations

ADL: activity of daily living
AE: adverse event
AESI: adverse event of special interest
ALT: alanine transaminase
aPTT: activated partial thromboplastin time
BTK: Bruton's tyrosine kinase
CD: cluster of differentiation
CFR: Code of Federal Regulation
Chi3L1: chitinase-3 like protein-1
CNS: central nervous system
COVID-19: Coronavirus Disease 2019
CPK: creatine phosphokinase
CSF: cerebrospinal fluid
CSR: clinical study report
C-SSRS: Columbia-suicide severity rating scale
DDI: drug-drug interaction
DILI: drug-induced liver injury
DMC: data monitoring committee
DNA: deoxyrebonucleic acid
DNAM-1: DNAX accessory molecule 1
DTP: direct-to-patient
EC: ethics committee
eCRF: electronic case report form
EDSS: expanded disability status scale
EU: European Union
FSH: follicle stimulating hormone
GCP: Good Clinical Practice
Gd: gadolinium
GM-CSF: granulocyte-macrophage-colony stimulating factor
GrA: human granzyme A
GrB: human granzyme B
GrK: human granzyme K
GrM: human granzyme M
HIV: human immunodeficiency virus
HR: hazard ratio
HRT: hormonal replacement therapy
ICF: informed consent form
ICH: International Council for Harmonisation
IEC: Independent Ethics Committees
IFNγ: Interferon γ
IL: interleukin
IP: investigational medicinal product
INR: international normalized ratio
IRB: Institutional Review Boards
IRT: interactive response technology
IUD: intrauterine device
IUS: intrauterine hormone-releasing system
IV: intravenous
IWRS: interactive web response system
JCV: John Cunningham virus
LTS: long-term safety
MCAM: melanoma cell adhesion molecule
MedDRA: Medical Dictionary for Regulatory Activities
MRI: magnetic resonance imaging
MS: multiple sclerosis
MSFC-3: multiple sclerosis functional composite 3
MTR: magnetization transfer ratio
NCI CTCAE: National Cancer Institute Common Terminology Criteria for Adverse
Event
NEDA: no evidence of disease activity-3
NfL: neurofilament light chain
NRSPMS: nonrelapsing secondary progressive multiple sclerosis
NSAIDs: nonsteroidal anti-inflammatory drugs
PD: pharmacodynamic(s)
PK: pharmacokinetics
PML: progressive multifocal leukoencephalopathy
PPMS: primary progressive multiple sclerosis
pTreg: peripheral regulatory T cells
RMS: relapsing multiple sclerosis
RTE: Recent thymic emigrant
SAP: statistical analysis plan
SEL: slowly evolving lesions
SoA: schedule of activities
SPMS: secondary progressive multiple sclerosis
SUSAR: suspected unexpected serious adverse reaction
SWI: susceptibility-weighted imaging
TB: tuberculosis
TEAE: treatment-emergent adverse event
Tfh: T follicular helper
Th 17: T helper 17 cells
Th1: T helper 1 cells
Th2: T helper 2 cells
TIGIT: T cell immunoglobulin and immunoreceptor tyrosine-based inhibitory motif (ITIM) domain
TNFα: tumor necrosis factor α
Treg: regulatory T cell
tTreg: Thymically derived Foxp3(+) regulatory T cells
ULN: upper limit of normal
WBC: white blood cells
WOCBP: women of childbearing potential

### Example 4—A Phase 3, Randomized, Double-Blind Efficacy and Safety Study Comparing SAR442168 to Placebo in Participants with Primary Progressive Multiple Sclerosis (Perseus)

The goal of this Phase 3 clinical trial is to demonstrate the efficacy and safety of SAR442168 compared to placebo in participants with PPMS. The primary endpoint is time to onset of 6-month confirmed disability progression (CDP) assessed via the Expanded Disability Status Scale (EDSS) score, which is shown in FIG. 7.

A graphical scheme of the study design is shown in FIG. 1D. Abbreviations used in FIG. 1D: CDP, confirmed disability progression; DMT, disease modifying therapy; EOS, end-of-study; MRI, magnetic resonance imaging.

Tables 4A1, 4A2, and 4A3 shown below describe the schedule of activities during the course of study. Table 4B that follows describes the objective and endpoints of the overall study.

TABLE 4A1
Schedule of Activities (SOA)- Screening to Month 5

| | | Randomization/ | | | | | | | |
|---|---|---|---|---|---|---|---|---|---|
| | Screening^{a} | start of IMP | To Year 2 (M 24)b | | | | | | |
| | Visit (a window of ±7 days is | | | | | | | | |
| | allowed for all visits after D 1) | | | | | | | | |
| | | | | | M 1.25 (W 5) | | M 2.25 (W 9) | | |
| | D-28 to | | M 0.5 | M 1 | M 1.5 (W 6) | M 2 | M 2.5 (W 10) | | M 4, |
| | D-1 | D 1 | (W 2) | (W 4)^{d} | M 1.75 (W 7) | (W 8)^{d} | M 2.75 (W 11) | M 3 | M 5^{d} |
| | Visit Number | | | | | | | | |
| | | | | | | | | | V6, |
| Procedure | V1 | V2 | | V3 | | V4 | | V5 | V7 |
| Informed consent | X | | | | | | | | |
| Demography | X | | | | | | | | |
| Inclusion/ | X | X | | | | | | | |
| exclusion | | | | | | | | | |
| criteria | | | | | | | | | |
| Lumbar | X | | | | | | | | |
| puncture for CSF | | | | | | | | | |
| analysis^{g} | | | | | | | | | |
| Medical/ | X | | | | | | | | |
| surgical history | | | | | | | | | |
| (including | | | | | | | | | |
| substance usage) | | | | | | | | | |
| Prior/ | <=======================================================================> | | | | | | | | |
| concomitant | | | | | | | | | |
| medications^{h} | | | | | | | | | |
| Randomization | | X | | | | | | | |
| IRT contact | X | X | | | | | | X | |
| IMP | | X | | | | | | X | |
| dispensationⁱ | | | | | | | | | |
| IMP compliance | | | | | | | | X | |
| Paper diary | | X | | | | | | X | |
| dispensation/ | | | | | | | | | |
| collection | | | | | | | | | |

Safety^{j}

| | | | | | | | | | |
|---|---|---|---|---|---|---|---|---|---|
| Physical | X | X | | | | | | X | |
| examination^{l} | | | | | | | | | |
| Height | X | | | | | | | | |
| Body weight | X | X | | | | | | X | |
| Serology tests | X | | | | | | | | |
| for hepatitis B | | | | | | | | | |
| and C HIV and other | | | | | | | | | |
| infectious | | | | | | | | | |
| diseases, if | | | | | | | | | |
| required locally | | | | | | | | | |
| TB/QuantiFERON ® | X | | | | | | | | |
| TB Gold test or | | | | | | | | | |
| equivalent^{m} | | | | | | | | | |
| Vital signs | X | X | | | | | | X | |
| Body temperature | X | X | | | | | | X | |
| 12-lead ECGⁿ | X | | | | | | | X | |
| Hematology, | X | X^{bb} | | X | | X | | X | X |
| biochemistry^{j} | | | | | | | | | |
| Liver function | | | X | | X | | X | | |
| tests^{k} | | | | | | | | | |
| Iron panel (serum): | X | | | | | | | | |
| iron, ferritin, | | | | | | | | | |
| transferrin saturation, | | | | | | | | | |
| TIBC; to be repeated | | | | | | | | | |
| during the study | | | | | | | | | |
| if needed | | | | | | | | | |
| Coagulation: | X | | | | | | | | |
| PT/INR, aPTT | | | | | | | | | |
| (to be repeated | | | | | | | | | |
| during the study, | | | | | | | | | |
| if needed) | | | | | | | | | |
| Urinalysis^{o} | X | | | | | | | X | |
| Pregnancy test (if | X | X | | | | | | X | |
| applicable)^{p} | | | | | | | | | |
| Serum FSH^{q} | X | | | | | | | | |
| Suicidality assessment | X | X | | | | | | X | |
| by C-SSRS | | | | | | | | | |

| | |
|---|---|
| Adverse event | <========================================================================> |
| collection | |

Efficacy

| | | | |
|---|---|---|---|
| EDSS | X | X | X |
| Timed 25-foot | | X | X |
| walk test | | | |
| 9-hole peg test | | X | X |
| SDMT and CVLT-II, | | X | X |
| where available^{r} | | | |
| Basic or expanded | X^{t} | | |
| MRI scan^{s} | | | |

| | |
|---|---|
| Actigraphy (option | X |
| for subset of | |
| participants)^{u} | |

Clinical outcome assessments^{V}

| | |
|---|---|
| MSQoL-54 | X |
| EQ-5D-5L | X |

Pharmacokinetics^{W}

SAR442168
pharmacokinetic
plasma samplesⁱ

Pharmacogenetics

| | |
|---|---|
| DNA sample | X |

Pharmacodynamics/biomarkers^{W}

| Blood sample | X | | |
|---|---|---|---|
| for archiving^{aa} | | | |
| Plasma samples | | X | X |
| (NfL) serum | | | |
| samples (Chi3L1)^{y} | | | |
| Immunophenotyping/ | | X | X |
| RNA sequencing | | | |
| (ToleDYNAMIC/ | | | |
| optional substudy | | | |
| at selected | | | |
| sites)^{dd} | | | |
| OCT substudy^{ee} | X | | |
| Lymphocyte phenotyping | | X | |
| by flow cytometry in | | | |
| whole blood (subset of | | | |
| participants)^{cc} | | | |
| Serum samples | | X | |
| (Ig levels)^{y} | | | |

TABLE 4A2
Schedule of Activities (SOA)- Month 6 to Month 24

| | To Year 2 (M 24)b | | | | | | | | |
|---|---|---|---|---|---|---|---|---|---|
| | Visit (a window of ±7 days is | | | | | | | | |
| | allowed for all visits after D 1) | | | | | | | | |
| | | M 7 | | M 10 | | | | | |
| | M 6 | M 8 | M 9 | M 11 | M 12 | M 15 | M 18 | M 21 | M 24 |
| | Visit Number | | | | | | | | |
| Procedure | V8 | | V9 | | V10 | V11 | V12 | V13 | V14 |
| Informed consent | | | | | | | | | |
| Demography | | | | | | | | | |
| Inclusion/ | | | | | | | | | |
| exclusion criteria | | | | | | | | | |
| Lumbar puncture | | | | | | | | | |
| for CSF analysis^{g} | | | | | | | | | |
| Medical/surgical | | | | | | | | | |
| history (including | | | | | | | | | |
| substance usage) | | | | | | | | | |
| Prior/ | <==============================================> | | | | | | | | |
| concomitant | | | | | | | | | |
| medications^{h} | | | | | | | | | |
| Randomization | | | | | | | | | |
| IRT contact | X | | X | | X | X | X | X | X |
| IMP dispensationⁱ | X | | X | | X | X | X | X | X |
| IMP compliance | X | | X | | X | X | X | X | X |
| Paper diary | X | | X | | X | X | X | X | X |
| dispensation/ | | | | | | | | | |
| collection | | | | | | | | | |

Safety^{j}

| | | | | | | | | | |
|---|---|---|---|---|---|---|---|---|---|
| Physical | X | | X | | X | X | X | X | X |
| examination^{l} | | | | | | | | | |
| Height | | | | | | | | | |
| Body weight | X | | | | X | | X | | X |
| Serology tests | | | | | | | | | |
| for hepatitis B | | | | | | | | | |
| and C HIV and other | | | | | | | | | |
| infectious diseases, | | | | | | | | | |
| if required locally | | | | | | | | | |
| TB/QuantiFERON ® | | | | | | | | | |
| TB Gold test or | | | | | | | | | |
| equivalent^{m} | | | | | | | | | |
| Vital signs | X | | X | | X | X | X | X | X |
| Body temperature | X | | X | | X | X | X | X | X |
| 12-lead ECGⁿ | X | | X | | X | | | | X |
| Hematology, | X | | X | | X | X | X | X | X |
| biochemistry^{j} | | | | | | | | | |
| Liver function | | X | | X | | | | | |
| tests^{k} | | | | | | | | | |
| Iron panel (serum): | | | | | | | | | |
| iron, ferritin, | | | | | | | | | |
| transferrin | | | | | | | | | |
| saturation, TIBC; | | | | | | | | | |
| to be repeated | | | | | | | | | |
| during the study | | | | | | | | | |
| if needed | | | | | | | | | |
| Coagulation: | | | | | | | | | |
| PT/INR, aPTT (to | | | | | | | | | |
| be repeated during | | | | | | | | | |
| the study, if needed) | | | | | | | | | |
| Urinalysis^{o} | | | | | X | | X | | X |
| Pregnancy test | X | | X | | X | X | X | X | X |
| (if applicable)^{p} | | | | | | | | | |
| Serum FSH^{q} | | | | | | | | | |
| Suicidality assessment | X | | X | | X | X | X | X | X |
| by C-SSRS | | | | | | | | | |

| | |
|---|---|
| Adverse event | <===============================================> |
| collection | |

Efficacy

| | | | | | | | |
|---|---|---|---|---|---|---|---|
| EDSS | X | X | X | X | X | X | X |
| Timed 25-foot | X | X | X | X | X | X | X |
| walk test | | | | | | | |
| 9-hole peg test | X | X | X | X | X | X | X |
| SDMT and CVLT-II, | X | X | X | X | X | X | X |
| where available^{r} | | | | | | | |
| Basic or expanded | X | | X | | X | | X |
| MRI scan^{s} | | | | | | | |
| Actigraphy (option | | | X | | | | |
| for subset of | | | | | | | |
| participants)^{u} | | | | | | | |

Clinical outcome assessments^{V}

| | | | | |
|---|---|---|---|---|
| MSQoL-54 | X | X | X | X |
| EQ-5D-5L | X | X | X | X |

Pharmacokinetics^{W}

| | | | |
|---|---|---|---|
| SAR442168 | X | X | X |
| pharmacokinetic | | | |
| plasma samplesⁱ | | | |

Pharmacogenetics

DNA sample

Pharmacodynamics/biomarkers^{W}

| | | | | |
|---|---|---|---|---|
| Blood sample | | | | |
| for archiving^{aa} | | | | |
| Plasma samples | X | | X | X |
| (NfL) serum samples | | | | |
| (Chi3L1)^{y} | | | | |
| Immunophenotyping/ | X | | X | |
| RNA sequencing | | | | |
| (ToleDYNAMIC/optional | | | | |
| substudy at selected | | | | |
| sites)^{dd} | | | | |
| OCT substudy^{ee} | | X | | |
| Lymphocyte phenotyping | | | | |
| by flow cytometry in | | | | |
| whole blood (subset | | | | |
| of participants)^{c} | | | | |
| Serum samples | X | | X | X |
| (Ig levels)^{y} | | | | |

TABLE 4A3
Schedule of Activities (SOA)- Month 27 to Follow-up

| | | | Only for | | Only for participants |
|---|---|---|---|---|---|
| | | | participants | | who completed |
| | | | who prematurely | For all | treatment to EOS but |
| | From M27 to EOS^{b} | | discontinue IMP | participants | do not enter LTS^{c} |
| | Visit (a window of ±7 days is allowed for all visits after D 1) | | | | |
| | Quarterly visits | Semi-annual | | EOS | Follow-up |
| | (M 27, M 30, M 33, | visits (M 30, | | “Common | visit (4 to |
| | M 36, M 39, M 42, | M 36, M 42, | | study end | 8 weeks |
| | M 45, M 48 . . .) | M 48 . . .) | pEOT^{e} | date” visit^{f} | after EOS) |
| | Visit Number | | | | |
| | V15, V16, V17, | | | | |
| | V18, V19, V20, | V16, V18, | | | |
| Procedure | V21, V22 | V20, V22 | pEOT^{e} | EOS | FUV |

Informed consent
Demography
Inclusion/exclusion
criteria
Lumbar puncture for
CSF analysis^{g}
Medical/surgical
history (including
substance usage)

| Prior/concomitant | <========================================================================> | | | | |
|---|---|---|---|---|---|
| medications^{h} | | | | | |
| Randomization | | | | | |
| IRT contact | X | X | X | X | X |
| IMP dispensationⁱ | X | X | | | |
| IMP compliance | X | X | X | X | |
| Paper diary | X | X | X | X | |
| dispensation/ | | | | | |
| collection | | | | | |

Safety^{j}

| Physical | X | X | X | X | X |
|---|---|---|---|---|---|
| examination^{l} | | | | | |
| Height | | | | | |
| Body weight | | X | X | X | X |
| Serology tests for | | | | | |
| hepatitis B and C | | | | | |
| HIV and other | | | | | |
| infectious | | | | | |
| diseases, if | | | | | |
| required locally | | | | | |
| TB/QuantiFERON ® | | | | | |
| TB Gold test or | | | | | |
| equivalent^{m} | | | | | |
| Vital signs | X | X | X | X | X |
| Body temperature | X | X | X | X | X |
| 12-lead ECGⁿ | | yearly | X | X | |
| Hematology, | X | X | X | X | X |
| biochemistry^{j} | | | | | |
| Liver function tests^{k} | | | | | |
| Iron panel (serum): | | | | | |
| iron, ferritin, | | | | | |
| transferrin | | | | | |
| saturation, TIBC; to | | | | | |
| be repeated during | | | | | |
| the study if needed | | | | | |
| Coagulation: | | | | | |
| PT/INR, aPTT (to be | | | | | |
| repeated during the | | | | | |
| study, if needed) | | | | | |
| Urinalysis^{o} | | X | X | X | |
| Pregnancy test (if | X | X | X | X | X |
| applicable)^{p} | | | | | |
| Serum FSH^{q} | | | | | |
| Suicidality | X | X | X | X | X |
| assessment by | | | | | |
| C-SSRS | | | | | |
| Adverse event | <=========================================================================> | | | | |
| collection | | | | | |

Efficacy

| EDSS | X | X | X | X |
|---|---|---|---|---|
| Timed 25-foot walk | X | X | X | X |
| test | | | | |
| 9-hole peg test | X | X | X | X |
| SDMT and CVLT-II, | X | X | X | X |
| where available^{r} | | | | |
| Basic or expanded | | yearly | X | X |
| MRI^{s} | | | | |
| Actigraphy (optional | X | | | |
| for subset of | | | | |
| participants)^{u} | | | | |

Clinical outcome assessments^{v}

| | | | |
|---|---|---|---|
| MSQoL-54 | X | X | X |
| EQ-5D-5L | X | X | X |

Pharmacokinetics^{W}

| | |
|---|---|
| SAR442168 | X^{e} |
| pharmacokinetic | |
| plasma samplesⁱ | |

Pharmacogenetics^{s}

DNA sample^{z}

Pharmacodynamics/biomarkers^{W}

| Blood sample for | | | | |
|---|---|---|---|---|
| archiving^{aa} | | | | |
| Plasma samples | | Yearly | X | X |
| (Nfl) serum samples | | | | |
| (Chi3L1)^{y} | | | | |
| Immunophenotyping/ | | | | |
| RNA sequencing | | | | |
| (ToleDYNAMIC/ | | | | |
| optional substudy at | | | | |
| selected sites)^{dd} | | | | |
| OCT substudy^{ee} | X | | | |
| Lymphocyte | | | X | |
| phenotyping by | | | | |
| flow cytometry in | | | | |
| whole blood | | | | |
| (subset of | | | | |
| participants)^{cc} | | | | |
| Serum samples (Ig | | yearly | X | X |
| levels)^{y} | | | | |

aPTT: activated partial thromboplastin time; β-HCG: β-human chorionic gonadotropin; AST: aspartate aminotransferase; ALT: alanine aminotransferase; Chi3L1: chitinase-3-like protein 1; C-SSRS: Columbia Suicide Severity Rating Scale; D: day; DNA: deoxyribonucleic acid; ECG: electrocardiogram; EDSS: Expanded Disability Status Scale; EOS: end of study; EOT: end of treatment; EQ 5D 5L: EuroQol 5-dimension 5-level questionnaire; FSH: follicle stimulating hormone; HIV: human immunodeficiency virus; Ig: immunoglobulin; IMP: investigational medicinal product; INR: international normalized ratio; IRT: interactive response technology; LTS: long term safety; M: month (28 days); MCV: mean corpuscular volume; MCH: mean corpuscular hemoglobin; MRI: magnetic resonance imaging; MS: multiple sclerosis; MSQoL-54: Multiple Sclerosis Quality of Life-54; NfL: neurofilament light chain; OCT: optical coherence tomography; pEOT: premature end of treatment; PK: pharmacokinetics; PT: prothrombin time; RBC: red blood cell; SWI: susceptibility weighted imaging; TB: tuberculosis; TIBC: total iron-binding capacity; V: visit; WBC: white blood cell.
Note:
All assessments shall be done as designated in this SoA unless not permitted according to local regulations. All visit assessments should be performed during the visit window, unless otherwise specified in this protocol.
^{a}Screening period can range from D-28 to D-1; Randomization visit can be performed only once IMPs are available at site. The interval between screening and randomization visits can range from 11 days (minimum) to 28 days (maximum). However, if required, randomization visit can be performed earlier than 11 days upon IMP receipt at the site, assuming the participant is eligible for randomization. In case of any delay to screening (MRI rescheduling, lab retests, etc), an additional period of up to 2 weeks is allowed. For other situations where a follow-up visit is needed, please see the Study Manual.
^{b}From D 1 to EOS, unscheduled visits may be performed at any time by the Investigator (eg, for evaluation of an adverse event). Assessments may be done as needed to evaluate the participant in accordance with the Investigator's best judgement and in-line with the study protocol. At a minimum, a physical examination should be performed, and body temperature and vital signs should be measured.
^{c}At the EOS, all the participants who have completed the study and remain on treatment with IMP (double blind or open label if meeting 6-month CDP) will be offered participation in a LTS study. Follow-up Visit assessment only performed for those participants who completed treatment and are not willing to take part in the LTS study. For other situations where a follow-up visit is needed, please see the Study Manual.
^{d}These visits may be done as home health visits (where applicable) or on-site visits (it is preferable that tests are performed at the central laboratory). In any situation where this is not possible (to be documented in source documents), the tests for these visits may be performed at a local laboratory.
^{e}If a participant prematurely permanently discontinues treatment with IMP, the participant will undergo a pEOT visit as soon as possible. A PK sample should also be collected if the pEOT visit can be scheduled within a maximum 24 hours after the last IMP dose. Participants will then be asked to continue with the study visits as scheduled until the global EOS Visit is reached. During these visits, all study procedures/assessments will be performed except IMP administration and blood sampling for PK and biomarkers (NfL, Chi3L1, and Ig levels). MRI scans for these participants will only be performed annually (using the next annual visit as the starting point).
^{f}Common EOS Visit will be done when the prespecified number of events for 6-month CDP is expected to be reached. The timing and window of this visit will be communicated to sites.
^{g}If no historical documentation, cerebrospinal fluid draw will be performed locally during the screening period.
^{h}Any disease modifying therapy (DMT) for MS taken at any time prior to signing the informed consent needs to be reported in the case report form (CRF); other prior medications will be reported for the period of 6 months prior to signing the informed consent form (ICF).
ⁱOn days of PK sampling the IMP needs to be taken at the study site after a “regular meal”. In case a participant forgot IMP at home or took IMP prior to arriving at the site on the day of visits with PK sampling, he/she will be asked to have a PK assessment within 3 days of the missed PK sampling. A PK assessment shall be done as soon as possible after an overdose or if otherwise specified per protocol (eg, investigation of abnormal laboratory test values).
^{j}Hematology (platelet count, RBC count, hemoglobin, hematocrit, MCV, MCH, reticulocytes, WBC count with differential: neutrophils, lymphocytes, monocytes, eosinophils, basophils). Biochemistry (blood urea nitrogen [BUN], creatinine, glucose, potassium, sodium, chloride, bicarbonate, calcium, liver function tests [AST, ALT, albumin, alkaline phosphatase, total and direct bilirubin, total protein; creatine phosphokinase]) will be tested. Lipase will be tested at Screening only. Monthly visits (M 1, M 2, M 4, and M 5) will include hematology and full liver panel only. Additional safety assessments can be performed if required by local regulations; such testing shall be performed at local laboratories. Additional visits may be added if required by local regulations.
Note:
a one-time retest at screening may be performed if an abnormal laboratory test value is considered temporary.
^{k}At intermediate timepoints (W 2, W 5, W 6, W 7, W 9, W 10, W 11, M 7, M 8, M 10, and M 11), only liver function tests will be collected (AST, ALT, albumin, alkaline phosphatase, total and direct bilirubin, total protein) and creatine phosphokinase; these can be performed at central laboratory (preferred, as on-site visits or home nursing as applicable for the site) or at local laboratory. For participants switching to open-label treatment, please refer to Rescue medicine section.
^{l}Complete physical examination due at screening, baseline, yearly, and EOS; brief physical examination is sufficient for the rest of the visits (complete and brief physical examinations will include neurological examination and collection of the following vital signs: arterial blood pressure, heart rate, and temperature).
^{m}To be performed at screening for all participants. To be repeated based on clinical judgment, borderline results or clinical suspicion of TB infection. See E 01, Table 4D for details. Screening tests for TB are described in Table 4F.
ⁿECG and 30-second rhythm strips will be obtained locally.
^{o}Urinalysis will be done as per Table 4F.
^{p}At screening, perform the serum β-HCG pregnancy test at the central laboratory. At randomization and other scheduled visits during the study, urine pregnancy tests should be performed. At randomization, a pregnancy test should be performed prior to the first dose of IMP. Additional serum or urine pregnancy tests may be performed, as determined necessary by the Investigator or required by local regulation, to establish the absence of pregnancy at any time during participant's participation in the study.
^{q}Only in female participants, if needed to establish menopausal status. The SDMT and CVLT-II will be performed in all participants. If for some reason, CVLT-II is not available at a given site due to reasons such as lack of translation, local certification, etc., then only the SDMT will be assessed for that participant.
^{s}A subset of sites that have 3T MRI capacity will perform additional sequences (eg, SWI). Further details will be defined in a separate manual. A visit window of ±21 days is acceptable for MRIs performed after D 1.
^{t}A visit window of ±21 days is acceptable for MRIs performed after D 1. The MRI scan should be performed as close as possible before the start of IMP. As much as possible, the screening MRI scan should be performed during the screening period only after it has been established that the participant meets all inclusion and no exclusion criteria.
^{u}A noninvasive activity monitor (actigraphy) may be optionally implemented by the Sponsor in a subset of participants during the course of the study if results from pilot assessment demonstrate feasibility. Actigraphy baseline assessment will be done at screening.
^{v}When available, clinical outcome assessments are to be completed by the participant prior to discussing their health status and prior to study treatment administration or other study related procedures.
^{W}Where available per local regulations.
^{x}M 6 and M 12: Two samples: one sample between 30 to 90 minutes and one sample between 2.5 to 5 hours after IMP administration. M 9: one sample 30-90 minutes after IMP administration.
^{y}Pharmacodynamics and biomarkers untimed samples will be collected if permitted per local regulations.
^{z}The DNA testing may be done at any time after signature of consent (in case it could not be done for some reason at Day 1). Participation is optional.
^{aa}This sample will be collected and stored for use if any unexpected safety issue occurs to ensure that a pre-dose baseline value is available for previously not assessed parameters (eg, serology) and for biomarkers research, if agreed.
^{bb}Samples for hematology and biochemistry tests on Day 1 (randomization) will be collected prior to administration of the first IMP dose.
^{cc}Blood sample collection for lymphocyte phenotyping by flow cytometry will be performed in a subset of randomized participants. Participants who did not have a baseline sample collected will no longer have this test performed. Participants who had a baseline sample collected will have a second sample collected at EOT/pEOT.
^{dd}Samples must be shipped within 24 hours.
^{ee}The substudy will be conducted at selected sites.

Objectives and endpoints for the treatment are shown in Table 4B.

TABLE 4B
Objectives and endpoints

| Objectives | Endpoints |
|---|---|
| Primary | |
| To determine the efficacy of SAR442168 | Time to onset of 6-month CDP defined as |
| compared to placebo in delaying disability | follows: |
| progression in PPMS | Increase of ≥1.0 point from the |
| | baseline EDSS score when the |
| | baseline score is ≤5.5, OR |
| | Increase of ≥0.5 points when the |
| | baseline EDSS score is >5.5 |
| Secondary | |
| To evaluate efficacy of SAR442168 | Time to onset of 3-month CDP as |
| compared to placebo on clinical endpoints, | assessed by the EDSS score |
| MRI lesions, cognitive performance, | Time to onset of sustained 20% |
| physical function, and quality of life | increase in the 9-hole peg test (9-HPT) |
| | confirmed over at least 3 months |
| | Time to onset of sustained 20% |
| | increase in the timed 25-foot walk |
| | (T25-FW) test confirmed over at least 3 |
| | months |
| | Total number of new and/or enlarging |
| | T2-hyperintense lesions as detected by |
| | MRI, defined as the sum of the |
| | individual number of new and/or |
| | enlarging T2 lesions at all scheduled |
| | visits starting after baseline up to and |
| | including the end of study (EOS) visit |
| | Time to onset of CDI defined as ≥1.0 |
| | point decrease on the EDSS score from |
| | baseline confirmed over at least 6 |
| | months |
| | Percent change in brain volume loss |
| | (BVL) as detected by brain MRI scans |
| | at EOS compared to Month 6 |
| | Change in cognitive function at the |
| | EOS compared to baseline as assessed |
| | by SDMT |
| | Change in cognitive function at EOS |
| | compared to baseline as assessed by |
| | CVLT-II, where available |
| To evaluate safety and tolerability of | Change in MSQoL-54 questionnaire |
| SAR442168 | score from baseline through the EOS |
| | Adverse events (AEs), serious AEs, |
| | AEs leading to permanent study |
| | intervention discontinuation, AEs of |
| | special interest, and potentially |
| To evaluate population PK of SAR442168 | clinically significant abnormalities in |
| in PPMS and its relationship to efficacy and | laboratory tests, safety scales, ECG, |
| Safety | and vital signs during the study period |
| To evaluate pharmacodynamics of | Plasma concentration of SAR442168 |
| SAR442168 | (population PK assessment) at Months |
| | 6, 9, and 12 |
| | Change in plasma NfL levels at the |
| | EOS compared to baseline |
| | Change in lymphocyte phenotype |
| | subsets in whole blood at the |
| | EOS compared to baseline in a subset |
| | of participants |
| | Change in serum immunoglobulin level |
| | at the EOS compared to baseline |
| | Change in serum Chi3L1 levels at the |
| | EOS compared to baseline |

Abbreviations: 9-HPT, 9-hole peg test; AE, adverse event; BVL, brain volume loss; CDI,
confirmed disability improvement; CDP, confirmed disability progression; Chi3L1, chitinase-3-like protein 1; CVLT-II, California Verbal Learning Test-II; ECG, electrocardiogram; EDSS, Expanded Disability Status Scale; EOS, end-of-study; MRI, magnetic resonance imaging; MSQoL-54, Multiple Sclerosis Quality of Life-54 Questionnaire; NfL, neurofilament light chain; PK, pharmacokinetic; PPMS, primary progressive multiple sclerosis; SDMT, Symbol Digit Modalities test; T25-FW, timed 25 foot walk

### Dose Regimen

The choice of the dose of 60 mg of the BTK inhibitor taken with food is based on the results of a Phase 2b dose-finding trial for tolebrutinib in participants with relapsing multiple sclerosis (DRI15928). See for example, Reich, D. S., et al., Safety and efficacy of tolebrutinib, an oral brain-penetrant BTK inhibitor, in relapsing multiple sclerosis: a phase 2b, randomised, double-blind, placebo-controlled trial. Lancet Neurol, 2021. 20(9): p. 729-738.

Analysis of the PK data and effect of fed status on tolebrutinib exposure showed a positive food effect with an increase in AUC₀₋₂₄ of approximately 2-fold. Moreover, the correlation between the treatment response and the exposure to tolebrutinib showed that higher exposure was associated with low numbers of new gadolinium-enhancing T1-hyperintense lesions after 12 weeks of treatment. Taken together, these data support the recommendation to take tolebrutinib with food.

There was no correlation between the dose of tolebrutinib administered and the number of TEAEs. The most common events (preferred terms) observed in participants in the tolebrutinib treatment arms were headache, upper respiratory tract infection, and nasopharyngitis. There were low numbers of AESIs and PCSAs observed. Overall, no new risks were identified in this trial.

### Overall Design:

This is a Phase 3, randomized, double-blind, 2-arm, placebo-controlled, parallel-group, multicenter, event-driven (6-month CDP) trial in participants with PPMS with a variable treatment duration ranging from approximately 24 to 48 months.

### Disclosure Statement:

This is a Parallel Treatment study with 2 arms that is blinded/masked for participants, any Investigator, site staff, and the Sponsor.

### Number of Participants:

Approximately 1320 people will be screened to achieve 990 participants randomly assigned to study intervention.

### Intervention Groups and Duration:

Participants will be randomly assigned at a 2:1 ratio to receive 60 mg of oral, daily SAR442168 or daily matching placebo. Randomization will be stratified by age at screening (>40 versus ≤40 years), geographic region (United States [US] versus non-US) and PPMS McDonald diagnosis criteria (2005 [Polman et al. Ann Neurol. 2005, 58, 840] and 2017 version versus 2017 version [Thompson et al. Lancet Neurol. 2018, 17, 162]).

### Study Intervention(s):

### Investigational Medicinal Product(s)

Formulation: SAR442168 film coated tablet
Route(s) of administration: oral
Dose regimen: 60 mg once daily

### Investigational Medicinal Product(s)

Formulation: placebo to match SAR442168 film coated tablet
Route of administration: oral
Dose regimen: once daily

### Noninvestigational Medicinal Products(s)

Formulation: MRI contrast-enhancing preparations
Route(s) of administration: Intravenous (IV)
Dose regimen: per respective label

### Temporary IMP Interruption Due to Surgery

If surgery is needed during the study, consider the benefit-risk of withholding the IMP for at least 3 to 7 days pre- and post-surgery, depending upon the type of surgery and the risk of bleeding.

The goal of this Phase 3 clinical trial is to demonstrate the efficacy and safety of SAR442168 compared to placebo in participants with PPMS. Six-month CDP was selected as the primary endpoint in this trial. It is widely used in clinical trials and is considered clinically more meaningful than 3-month CDP. Confirmed disability improvement (CDI) over at least 6 months measured by the EDSS score will also be used as a secondary endpoint due to its clinical importance.

Magnetic resonance imaging outcomes will include change in brain volume, which is considered as a marker of the CNS degenerative process and is therefore recommended for use in progressive MS trials (EMA Guideline on clinical investigation of medicinal products for the treatment of multiple sclerosis. London, 26 Mar. 2015. EMA/CHMIP/771815/2011, Rev. 2 [Cited 2020 Feb. 13]).

Population pharmacokinetics (PK) will be performed in order to obtain a larger and more diverse population to evaluate the PK of SAR442168 in the PPMS population, to assess sources of PK variability (ethnicity, special populations), and to establish exposure correlation to clinical efficacy, biomarkers, and safety endpoints. Analysis of the population PK data may be performed as soon as all participants have finished the Month 12 visit (a separate unblinded team will be used to preserve blinding integrity).

In the ongoing Phase 3 and LTS studies, tolebrutinib has been generally well tolerated to date. An identified risk for tolebrutinib has been identified as follows:
Treatment-emergent SAEs of drug-induced liver injury (DILI) were reported in the ongoing Phase 3 trials; however, all cases occurred between Months 2 to 3 and appear reversible after treatment discontinuation, with potential confounders identified for some of the cases.

### End of Study Definition

A participant is considered to have completed the study if he/she has completed all periods of the study including the EOS Visit, whether remaining on IMP or not.

This study will end when approximately 290 primary endpoint events of 6-month CDP have been observed. With a planned recruitment period of approximately 24 months, the duration of the study is estimated to be approximately 48 months.

The event rates based on the blinded data will be regularly monitored to predict the event timelines and timely estimate the possible study end date.

### Study Population

### Inclusion Criteria

Participants are eligible to be included in the study only if all of the following criteria apply as shown in Table 4C.

TABLE 4C
Inclusion Criteria

| Category | Criteria | |
|---|---|---|
| Age | I 01. | The participant must be 18 to 85 years of age inclusive, at the time |
| | | of signing the informed consent. |
| Type of | I 02. | The participant must have a current diagnosis of PPMS in |
| participant | | accordance with the 2017 revision of the McDonald criteria |
| and disease | | (Thompson et al. Lancet Neurol. 2018, 17, 162). |
| characteristics | I 03. | The participant must have an EDSS score at screening from 2.0 to |
| | | 6.5 points, inclusive. |
| | I 04. | The participant must have positive CSF (isoelectric focusing |
| | | evidence of oligoclonal bands and/or elevated IgG index) either |
| | | during screening or previous historical assessment. Supportive |
| | | source documentation must be available. |
| Weight | I 05. | Not Applicable. |
| Sex | I 06. | Male or Female. |
| | Contraceptive use should be consistent with local regulations regarding | |
| | the methods of contraception for those participating in clinical studies. | |
| | c) Male participants | |
| | Not applicable. | |
| | d) Female participants | |
| | A female participant is eligible to participate if she is not | |
| | pregnant or breastfeeding, and at least one of the following | |
| | conditions applies: | |
| | Is not a woman of childbearing potential (WOCBP); or | |
| | Is a WOCBP and agrees to use an acceptable contraceptive | |
| | method during the intervention period (at a minimum until | |
| | after the last dose of study intervention). | |
| | A WOCBP must have a negative highly sensitive pregnancy | |
| | test (urine or serum as required by local regulations) at | |
| | screening before the first dose of study intervention. If a urine | |
| | test cannot be confirmed as negative (eg, an ambiguous result), | |
| | a serum pregnancy test is required. In such cases, the | |
| | participant must be excluded from participation if the serum | |
| | pregnancy result is positive. | |
| | Requirements for pregnancy testing during and after study | |
| | intervention are located in the schedule of activities (SoA); | |
| | Tables 4A1 and 4A2). | |
| | I 07. | The Investigator is responsible for review of medical history, |
| | | menstrual history, and recent sexual activity to decrease the risk for |
| | | inclusion of a woman with an early undetected pregnancy, if |
| | | allowed by local regulations. |
| Informed | I 08. | The participant must have given written informed consent prior to |
| Consent | | undertaking any study related procedure. This includes consent to |
| | | comply with the requirements and restrictions listed in the |
| | | informed consent form (ICF) and in this protocol. In countries |
| | | where the legal age of maturity is greater than 18 years, a specific |
| | | ICF for such legally minor participants must also be signed by the |
| | | participant's legally authorized representative. |

### Exclusion Criteria

Participants are excluded from the study if any of the following criteria apply as shown in Table 4D.

TABLE 4D
Exclusion Criteria

| Category | Criteria | |
|---|---|---|
| Medical conditions | E 01. | The participanthas a history of infection or may be at risk for |
| | | infection: |
| | | A history of T-lymphocyte or T-lymphocyte-receptor |
| | | vaccination, transplantation (including solid organ, |
| | | stem cell, and bone marrow transplantation) and/or |
| | | antirejection therapy. |
| | | The participant has received any live (attenuated) |
| | | vaccine (including but not limited to varicella zoster, |
| | | oral polio, and nasal influenza) within 2 months |
| | | before the first treatment visit. |
| | | The participant has a lymphocyte count less than the |
| | | lower limit of normal (LLN) at the Screening Visit. |
| | | A history of diagnosis of progressive multifocal |
| | | leukoencephalopathy (PML) or evidence of findings |
| | | suggestive of PML on the screening MRI. |
| | | A history of infection with human immunodeficiency |
| | | virus (HIV) (eg, any known positive HIV test or |
| | | information from participant interview). |
| | | A history of active or latent tuberculosis (TB); TB |
| | | testing should be performed at screening and again |
| | | during the study, if clinically indicated, and may be |
| | | repeated based on clinical judgment, borderline |
| | | results, or clinical suspicion of TB infection. |
| | | Screening tests for TB are described in Table 4F. |
| | | NOTE: The Investigator may consult with an |
| | | infectious disease expert if required, eg, test results |
| | | are unclear or there is a suspicion of false positive test |
| | | results. If the infectious disease expert considers the |
| | | test results as false positive and not clinically relevant |
| | | and confirms that the participant can be enrolled in |
| | | the trial, the Investigator must document this in |
| | | source data and may then randomize the participant. |
| | | Persistent chronic or active recurring infection |
| | | requiring treatment with antibiotics, antivirals, or |
| | | antifungals. |
| | | Fever within 4 weeks of the Screening Visit (≥38° C.; |
| | | however, if due to brief and mild ear, nose, throat |
| | | viral infection participant may be included based on |
| | | the Investigator's judgment). |
| | | Participants at risk of developing or having |
| | | reactivation of hepatitis, ie, results at screening for |
| | | serological markers for hepatitis B and C viruses |
| | | indicating acute or chronic infection. See the Study |
| | | Manual for further details. |
| | E 02. | The presence of psychiatric disturbance or substance abuse as |
| | | evidenced by: |
| | | A history of any psychiatric disease, behavioral |
| | | condition, or depression requiring hospitalization |
| | | within 2 years prior to the Screening Visit. |
| | | A documented history of attempted suicide or suicidal |
| | | ideation of category 4 or 5 according to the Columbia |
| | | Suicide Severity Rating Scale (C-SSRS) |
| | | baseline/screening version over the 6 months prior to |
| | | the Screening Visit, OR if in the Investigator's |
| | | judgment, the participant is at risk for a suicide |
| | | attempt. |
| | | Active alcohol use disorder or a history of alcohol or |
| | | drug abuse within 1 year prior to the Screening Visit. |
| | | Current alcohol intake >2 drinks per day for men and >1 |
| | | drink per day for women (1 drink = approximately |
| | | 14 grams of alcohol = 350 mL beer = 140 mL wine = |
| | | 40 mL of spirits). |
| | E 03. | The following findings obtained during the Screening Visit |
| | | considered in the Investigator's judgment to be clinically |
| | | significant in the context of this clinical trial: |
| | | Any screening laboratory values outside normal limits. |
| | | Abnormal ECG. |
| | E 04. | Conditions that may predispose the participant to excessive |
| | | bleeding: |
| | | A bleeding disorder or known platelet dysfunction at |
| | | any time prior to the screening visit. |
| | | A platelet count <150 000/μL at the screening visit. |
| | | The participant has had major surgery within 4 weeks |
| | | prior to the screening visit, which could affect the |
| | | participant's safety (as judged by the Investigator) or |
| | | has planned any elective major surgery during the study. |
| | | A history of significant bleeding event within 6 |
| | | months prior to screening, according to the |
| | | Investigator's judgment such as, but not limited to |
| | | cerebral or gastrointestinal bleeding. |
| | E 05. | Conditions that would adversely affect participation in the |
| | | study or make the primary efficacy endpoint non-evaluable: |
| | | A short life expectancy due to pre-existing health condition(s) |
| | | as determined by their treating neurologist. |
| | | A history or presence of significant other concomitant illness |
| | | according to the Investigator's judgment such as, but not |
| | | limited to cardiovascular (including Stage III or IV cardiac |
| | | failure according to New York Heart Association [NYHA] |
| | | classification), or renal (ie, undergoing dialysis), neurological, |
| | | endocrine, gastrointestinal, metabolic, pulmonary (eg, |
| | | interstitial pneumonia or pulmonary fibrosis), or lymphatic |
| | | disease that would adversely affect participation in this study. |
| | | Acute liver disease, cirrhosis, chronic liver |
| | | disease (unless considered stable for >6 months). |
| | | Confirmed screening ALT >1.5 × ULN OR AST >1.5 × ULN |
| | | OR alkaline phosphatase >2 × ULN (unless caused by |
| | | non-liver-related disorder or explained by a stable |
| | | chronic liver disorder) OR total bilirubin >1.5 × ULN |
| | | (unless due to Gilbert syndrome or non-liver-related disorder). |
| | | At screening, elevated transferrin saturation (>50% in |
| | | males and >40% in females) and/or with elevated |
| | | ferritin levels >500 μg/L. |
| | | Any malignancy within 5 years prior to the |
| | | Screening Visit (except for effectively |
| | | treated carcinoma in situ of the cervix or |
| | | adequately treated non-metastatic squamous |
| | | or basal cell carcinoma of the skin) will also |
| | | be exclusionary. |
| | | Any other medical condition(s) or |
| | | concomitant disease(s) making them non- |
| | | evaluable for the primary efficacy endpoint |
| | | or that would adversely affect participation |
| | | in this study, as judged by the Investigator. |
| Prior/concomitant | E 19. | The participant has received any of the following therapy |
| | | medications/treatments within the specified time frame before |
| | | any randomization assessment (no washout is required for |
| | | dimethyl fumarate, interferon beta, or glatiramer acetate |
| | | treatments although use is not permitted on or after Day 1): |

| | Exclusionary if used/used |
|---|---|
| Medication | within required wash out period |
| Systemic corticosteroids, | 1 month prior to |
| adrenocorticotropic hormone | screening MRI scan |
| Siponimod, ponesimod | 1 week before randomization |
| | with MRI and clinical |
| | assessment for PML |
| Plasma exchange | 1 month prior to randomization |
| IV immunoglobulin | 2 months prior to randomization |
| Fingolimod, ozanimod | 6 weeks before randomization |
| | with MRI and clinical |
| | assessment for PML |
| Natalizumab | 2 months before randomization |
| | with MRI and clinical |
| | assessments for PML |
| Teriflunomide^{a}, mildly to moderately | 3 months prior to randomization |
| immunosuppressive/chemotherapeutic | |
| medications such as azathioprine and | |
| methotrexate, mycophenolate | |
| Lymphoid irradiation, bone marrow | A participant who has received |
| transplantation, mitoxantrone (with | any of these treatments at any |
| evidence of cardiotoxicity following | time is not eligible. |
| treatment, or cumulative lifetime dose >120 | |
| mg/m²), other strongly | |
| immunosuppressive treatments with | |
| very long-lasting effects | |
| Ofatumumab | 4 months prior |
| B-cell-depleting therapies such as | 6 months prior to randomization |
| ocrelizumab ^{b} and rituximab | |
| Highly | 2 years prior to randomization |
| immunosuppressive/chemotherapeutic | |
| medications: mitoxantrone up to | |
| 120 mg/m2 body surface area, | |
| cyclophosphamide, cladribine, | |
| cyclosporine | |
| Alemtuzumab | 4 years prior to randomization |
| Other MS-disease modifying | 5 half-lives or until end of |
| treatments | pharmacodynamics activity, |
| | whichever is longer |

Abbreviations: IV: intravenous MRI: magnetic resonance imaging, MS:
multiple sclerosis, PML: progressive multifocal leukoencephalopathy
^{a}No time restriction if accelerated elimination procedure is done
^{b} Ocrelizumab therapy: see country specific requirements

| Category | Criteria | |
|---|---|---|
| | E 07. | The participant is receiving potent and moderate inducers of |
| | | cytochrome P450 3A (CYP3A) or potent inhibitors of |
| | | CYP2C8 hepatic enzymes. |
| | E 08. | The participant is receiving anticoagulant/antiplatelet |
| | | therapies, including: |
| | | Acetylsalicylic acid (aspirin) >81 mg/day, |
| | | Antiplatelet drugs (eg, clopidogrel), |
| | | Warfarin (vitamin K antagonist), |
| | | Heparin, including low molecular weight heparin |
| | | (antithrombin agents), |
| | | Dabigatran (direct thrombin inhibitor), |
| | | Apixaban, edoxaban, rivaroxaban (direct factor Xa |
| | | inhibitors). |
| | | Note: All the above-mentioned drugs must be stopped at least 5 half- |
| | | lives before study drug administration except for aspirin, which must |
| | | be stopped at least 8 days before. If this is not clinically appropriate, |
| | | the participant cannot be included. These washout periods are only |
| | | applicable in the case that the Investigator deems it clinically |
| | | appropriate to discontinue the listed medications or there is a recent |
| | | history of use of these medications (as in the case when short term |
| | | treatment with anticoagulants is clinically recommended for certain |
| | | thrombotic events), and therefore these washout periods will need to |
| | | be followed prior to randomization. |
| | | If the participant has a chronic underlying medical condition (stroke, |
| | | coronary or carotid artery disease, heart valvular disease etc.) |
| | | requiring continued use of these medications, the participant cannot |
| | | be enrolled in the study. |
| | E 09. | The participant has sensitivity to any of the study |
| | | interventions, or components thereof, or has a drug or other |
| | | allergy that, in the opinion of the Investigator, contraindicates |
| | | participation in the study. |
| Prior/concurrent | E 10. | The participant was previously exposed to any BTK inhibitor, |
| clinical study | | including tolebrutinib. |
| experience | E 11. | The participant has taken other investigational drugs within 3 |
| | | months or 5 half-lives, whichever is longer, before the |
| | | screening visit. |
| Diagnostic | E 12. | The participant has a contraindication for MRI, ie, presence of |
| assessments | | pacemaker, metallic implants in high-risk areas (ie, artificial |
| | | heart valves, aneurysm/vessel clips), presence of metallic |
| | | material (eg, shrapnel) in high risk areas, known history of |
| | | allergy to any contrast medium, or history of claustrophobia |
| | | that would prevent completion of all protocol scheduled MRI. |
| | | Note: People with a contraindication to Gd can be enrolled |
| | | into the study but cannot receive Gd contrast dyes during their |
| | | MRI scan. |
| Other exclusions | E 13. | Individuals accommodated in an institution because of |
| | | regulatory or legal order; prisoners or participants who are |
| | | legally institutionalized. |
| | E 14. | Participant not suitable for participation, whatever the reason, |
| | | as judged by the Investigator, including medical or clinical |
| | | conditions, or participants potentially at risk of noncompliance |
| | | to study procedures or not able to follow the schedule of |
| | | protocol assessments due to other reasons. |
| | E 15. | Participants are employees of the clinical study site or other |
| | | individuals directly involved in the conduct of the study, or |
| | | immediate family members of such individuals. |
| | E 16. | Any other situation during study implementation/course that |
| | | may raise ethics considerations. |
| | | Note: a one-time retest at screening may be performed if an |
| | | abnormal laboratory test value is considered temporary |

### Lifestyle Considerations

Meals and dietary restrictions: tolebrutinib (IMP) shall be taken with a regular meal. When possible, the meal with which IMP is taken (eg, breakfast, lunch, or dinner) should be consistent throughout the study. The typical meal with which the IMP is taken will be recorded at each visit. In case the mealtime for IMP administration needs to be changed, a gap of a minimum of 12 hours between 2 doses should be maintained.

### Caffeine, Alcohol, and Tobacco:

For each visit with PK/PD assessment, participants will abstain from ingesting caffeine- or xanthine-containing products (eg, coffee, tea, cola drinks, and chocolate) for 2 hours before the start of treatment until after collection of the final PK and/or PD sample later that day.

For each visit with PK/PD assessment, participants will abstain from alcohol for 24 hours before the start of treatment until after collection of the final PK and/or PD sample later that day.

During the entire study, participants should be warned not to consume substantial quantities of alcohol, defined as >14 grams (1 standard drink) per day in female participants or >28 grams (2 standard drinks) per day in male participants on a regular basis.

### Study Intervention(s) and Concomitant Therapy

Study intervention is defined as any investigational intervention(s), marketed product(s), placebo, or medical device(s) intended to be administered to a study participant according to the study protocol.

TABLE 4E
Overview of study interventions administered

| | | |
|---|---|---|
| ARM name | tolebrutinib | Placebo |
| Intervention name | tolebrutinib 60 mg | Placebo |
| Type | Drug | Drug |
| Dose formulation | film-coated tablet | film-coated tablet |
| Unit dose strength(s) | 60 mg | 0 mg |
| Dosage level(s) | Once daily | Once daily |
| Route of administration | Oral | Oral |
| IMP and NIMP | IMP | IMP |
| Packaging and labeling | Study intervention will be | Study intervention will be |
| | provided in wallet blister | provided in wallet blister |
| | packaging. The content of | packaging. The content of |
| | the labeling is in accordance | the labeling is in accordance |
| | with the local regulatory | with the local regulatory |
| | specifications and requirements. | specifications and requirements. |
| Current name | SAR442168-Tolebrutininb | Not applicable |

### Dose Modification

Dose reduction is not foreseen in this study. Treatment may need to be interrupted or permanently discontinued if deemed necessary due to an AE.

### Concomitant Therapy

Any medication or vaccine (including over-the-counter or prescription medicines, vitamins, and/or herbal supplements) that the participant is receiving at the time of enrollment or receives during the study must be recorded along with:
Reason for use.
Dates of administration including start and end dates.
Dosage information including dose and frequency.

Live (attenuated) vaccines should not be administered during the intervention period. Therapies for MS noted in the exclusion criterion E 06 are not permitted after randomization while the participant is on study treatment. Short-term use (3 to 5 days) of glucocorticoids (eg, for MS relapse treatment or an acute illness) and local corticosteroids (eg, topical, nasal, ocular, otic, intra-articular) are allowed.

The Medical Monitor should be contacted if there are any questions regarding concomitant or prior therapy.

Participants must abstain from taking prescription or nonprescription drugs (including vitamins and dietary or herbal supplements) within 7 days (or 14 days if the drug is a potential enzyme inducer) or 5 half-lives (whichever is longer) before the start of study intervention until completion of the follow-up visit, unless, in the opinion of the Investigator and Sponsor, the medication will not interfere with the study. Medications for MS treatment of MS symptoms (eg, walking impairment, fatigue, spasticity, incontinence, pain) should be maintained at a stable dose prior to screening and for the duration of the treatment period, if clinically feasible.

Anticoagulant/antiplatelet therapies are not permitted to be taken concomitantly with the IMP, including:
Acetylsalicylic acid (aspirin) >81 mg/day.
Antiplatelet drugs (eg, clopidogrel).
Warfarin (vitamin K antagonist).
Heparin, including low molecular weight heparin (antithrombin agents).
Dabigatran (direct thrombin inhibitor).
Apixaban, edoxaban, rivaroxaban (direct factor Xa inhibitors).

Paracetamol/acetaminophen, at doses of ≤3 grams/day, is permitted for use at any time during the study. Short courses (up to 5 days) of nonsteroidal anti-inflammatory drugs (NSAIDs) (other than acetylsalicylic acid), preferably selective cyclooxygenase-2 inhibitors at the lowest effective dose, may be given during the study if clinically necessary for the treatment of an existing medical condition or a new event. The Investigator must record the use of NSAIDs (and any other comedication) in the eCRF.

### CYP Inhibitor/Inducer:

Potent and moderate inducers of CYP3A or potent inhibitors of CYP2C8 hepatic enzymes are not allowed throughout the study (Appendix 8 [Section 10.8]). Based on nonclinical drug metabolism studies, SAR442168 is a substrate of the CYP3A4 and CYP2C8 isoenzymes. In healthy participants, potent CYP3A4 inhibitor (itraconazole 200 mg once daily for 4 days) increased SAR442168 area under the curve (AUC) exposure 1.8-fold (INT16385 study) and potent CYP2C8 inhibitor (gemfibrozil 600 mg twice daily for 6 days) increased SAR442168 AUC exposure 8.4-fold (INT16726 study). Based on a satisfactory safety and tolerability profile and on the observed exposure in healthy participants who received SAR442168 at a dose of up to 240 mg SAR442168 once daily for 14 days under fed conditions (TDR16862 study), drugs that strongly inhibit CYP3A4 are allowed and drugs that strongly inhibit CYP2C8 are not permitted. In healthy participants, potent CYP3A4 and moderate CYP2C8 induction by rifampicin (600 mg once daily for 8 days) decreased SAR442168 exposure 6-fold (INT16726 study). Therefore, potent and also moderate (based on prediction) CYP3A inducers are not permitted due to their potential to decrease SAR442168 exposure and efficacy. See Appendix 8 (Section 10.8) for the list of drugs to be avoided.

The Sponsor should be contacted if there are any questions regarding concomitant or prior therapy.

### Rescue Medicine

If a participant achieves the primary endpoint (6-month CDP), they may, in conjunction with the Treating Investigator, choose to receive one of the following:
1. To switch to open-label SAR442168 treatment; OR
2. To switch to a non-study treatment approved for PPMS in their respective country. In this case, the participant will permanently discontinue the IMP and will be encouraged to remain in the study for planned clinical visits until common study end.

In case the participant switches to open-label SAR442168 treatment, he/she will need to be monitored for liver function tests after the first open-label dose at Weeks 2, 4, 5, 6, 7, 8, 9, 10, 11, and 12, and then monthly for the next 9 months. After that, the schedule visits timepoints per SoA (Tables 4A1 and 4A2) will be resumed (ie, every 3 months), until the common study end. Whenever a timepoint will coincide with a scheduled visit as per SoA (Section 1.3), the full scheduled visit assessments will be performed instead of the liver monitoring testing alone.

Should the participant and Investigator opt for the provision of rescue medicine, they will remain blinded to the original treatment assignment.

All individual blinded data will be reviewed and all queries resolved, if possible, before the switch to the chosen rescue approach to ensure data integrity for primary assessment. Prior to initiation of rescue treatment, the Investigator shall confirm that there has been no adjudicated relapse within 90 days prior to the onset or confirmation of 6-month CDP. Based on individual symptoms and assessed risk of further progression, the Investigator and participant may choose for the participant to remain on the initial double-blind treatment after achieving 6-month CDP.

### Discontinuation of Study Intervention and Participant Discontinuation/Withdrawal

### Discontinuation of Study Intervention

### Definitive Discontinuation

The study intervention should be continued whenever possible.

Permanent intervention discontinuation is any treatment discontinuation associated with the definitive decision by the Investigator or the participant not to re-expose the participant to study intervention at any time. In rare instances, it may be necessary for a participant to permanently discontinue study intervention. If study intervention is permanently discontinued, the participant shall be asked to remain in the study to be evaluated until the follow-up/EOS Visit. For this set of participants (participants who discontinued the IMP and/or switched to another DMT), no PK or biomarker samples will be collected after the pEOT visit, and MRI assessments will be performed once annually using the next annual visit as the starting point. This will be important to continue to evaluate for safety and efficacy. See the SoA (Tables 4A1 and 4A2) for data to be collected at the time of discontinuation of study intervention. In the case that the study intervention is permanently discontinued, the participant should be treated for MS according to local clinical practice and the best judgment of the Treating Investigator.

The following may be justifiable reasons for the Investigator or Sponsor to discontinue a participant from treatment:
Adverse events that endanger the safety of the participant, or if discontinuation of study intervention is desired or considered necessary by the Investigator and/or participant.
If IMP discontinuation criteria are met as per the guidance for the follow up of laboratory abnormalities (Table 4F).
The participant is no longer deriving a therapeutic/clinical benefit in the opinion of the Investigator.
If a female participant becomes pregnant or wishes to become pregnant during the study.
At participant's request, ie, withdrawal of the consent for treatment.
Any serious opportunistic infections (eg, PML [see FIG. 6], HIV)
Continued need for/chronic use of prohibited a concomitant medication (see Table 4I).
Use of open-label SAR442168 or non-study disease modifying therapy approved for PPMS in their respective countries (eg, after 6-month CDP).
A brief physical examination will include, at a minimum, assessments of the skin, lungs, cardiovascular system, neurological examination, and abdomen (liver and spleen).
Investigators should pay special attention to clinical signs related to previous serious illnesses.
Any clinically significant new finding or worsening of previous finding should be reported as an AE, per Investigator's judgement.

Discontinuation of study intervention for abnormal liver function should be considered by the Investigator when a participant meets one of the conditions outlined in the algorithm (Table 4B) or if the Investigator believes that it is in the best interest of the participant.

### Clinical Laboratory Tests

The tests detailed in Table 4F will be performed by the central laboratory, when feasible. Local laboratory results are only required in the event that the central laboratory results are not available in time for either study intervention administration and/or response evaluation. Additionally, if the local laboratory results are used to make either a study intervention decision or response evaluation, the results must be entered into the eCRF.

Protocol-specific requirements for inclusion or exclusion of participants are detailed in Tables 4C and 4D of the protocol.

Additional tests may be performed at any time during the study as determined necessary by the Investigator or required by local regulations. Additional serum or urine pregnancy tests may be performed, as determined necessary by the Investigator or required by local regulation, to establish the absence of pregnancy at any time during the subject's participation in the study.

TABLE 4F
Protocol-required laboratory assessments

| Laboratory | | | |
|---|---|---|---|
| assessments | Parameters | | |
| Hematology | Platelet count | RBC indices: | WBC count with |
| | | | differential: |
| | RBC count | MCV | Neutrophils |
| | Hemoglobin | MCH | Lymphocytes |
| | Hematocrit | % Reticulocytes | Monocytes |
| | | | Eosinophils |
| | | | Basophils |
| Clinical | BUN | Sodium | AST |
| chemistry | Creatinine | Calcium | ALT |
| | Glucose | Total and direct bilirubin | Alkaline phosphatase |
| | Potassium | Total protein | Albumin |
| | | Chloride | Creatine phosphokinase |
| | | Bicarbonate | Lipase |

| | |
|---|---|
| Routine | Specific gravity |
| urinalysis | pH, glucose, protein, blood, ketones, bilirubin, urobilinogen, nitrite, |
| | leukocyte esterase by dipstick |
| | Microscopic examination (if blood or protein is abnormal and for signs of |
| | infection) |
| Other | FSH and estradiol (if needed, only in female participants to confirm |
| screening | postmenopausal state) |
| tests | Highly sensitive serum or urine β-hCG pregnancy test (as needed for |
| | women of childbearing potential) ^{b} |
| | Coagulation: PT/ INR, aPTT |
| | Serology tests for hepatitis B (HBs Ag, anti-HBC IGM and total, anti-HBs) |
| | and C virus (anti-HCV); in case these results are inconclusive (eg anti-HBS |
| | negative and anti-HBc positive or anti-HC IgG positive), HBV-DNA or |
| | HCV-RNA testing, respectively, should be performed for confirmation. HIV |
| | and other infectious diseases, if locally required. |
| | Tuberculosis test: Blood testing (eg, QuantiFERON ® TB Gold test) is |
| | preferred; skin testing (eg, tuberculin skin test) with ancillary testing will be |
| | allowed if blood testing is not available. T-SPOT can also be performed, if |
| | available. |
| | Iron panel (serum): iron, ferritin, transferrin saturation TIBC. |

ALT: alanine aminotransferase; anti-HBc; antibody to hepatitis B core antigen; anti-HBs: hepatitis B surface antibody; aPTT: activated partial thromboplastin time; AST: aspartate aminotransferase; BUN: blood urea nitrogen; β-hCG: human chorionic gonadotropin; FSH; follicle stimulating hormone; IEC: independent ethics committee; INR: international normalized ratio; HBsAg: hepatitis B surface antigen; HBV: hepatitis B virus; HCV: hepatitis C virus; HIV: human immunodeficiency virus; Ig: immunoglobulin; IRB: institutional review board; MCH: mean corpuscular hemoglobin; MCV: mean corpuscular volume; PT: prothrombin time; RBC: red blood cell; SGOT: serum glutamic-oxaloacetic transaminase; SGPT: serum glutamic-pyruvic transaminase; TB: tuberculosis; TIBC: total iron-binding capacity; ULN: upper limit of normal; WBC: white blood cell.
a Details of liver chemistry stopping criteria and required actions and follow-up assessments after observations of ALT >3 × ULN are given in FIG. 2-8. Clinical laboratory findings of ALT >3 × ULN and bilirubin ≥2 × ULN (>35% direct bilirubin), or ALT >3 × ULN and international normalized ratio (INR) >1.5, if INR measured, that may suggest severe liver injury and must be reported as an SAE.
^{b} Local urine testing for pregnancy will be standard for the protocol (except for the Screening Visit when a serum pregnancy test is required) unless serum testing is required by local regulation or IRB/IEC.
c Further details are provided in E 01, Table 4D.

Investigators must document their review of each laboratory safety report.

Laboratory/analyte results that could unblind the study will not be reported to investigative sites or other blinded personnel until the study has been unblinded. This includes PK assessments and any post-baseline biomarker or PD assessments.

### Liver and Other Safety: Actions and Follow-Up Assessments

These actions described in Table 4G and FIGS. 2-8 are required for ALT increase and thrombocytopenia events ONLY. For all other safety events described, these are suggested per the Investigator's medical judgement.

Neutropenia is to be recorded as an AE only if at least 1 of the criteria listed in the general guidelines for reporting AEs in Appendix 3 (Section 10.3) is met.

Thrombocytopenia is to be recorded as an AE only if at least 1 of the criteria listed in the general guidelines for reporting AEs in Appendix 3 (Section 10.3) is met.

Abbreviations in FIG. 3: aPTT: activated partial thromboplastin time; EDTA: Ethylenediaminetetraacetic acid; INR: international normalized ratio; PK: pharmacokinetic; PT: prothrombin time.

Note: “Baseline” refers to ALT sampled at baseline visit; or if baseline value unavailable, to the latest ALT sampled before the baseline visit. The algorithm does not apply to the instances of increase in ALT during Screening.

TABLE 4G
Actions for cases of confirmed ALT elevation

In ANY CONFIRMED CASE of ALT >5 × ULN, the following steps are REQUIRED
(recommended for ALT >3 × ULN but ALT <5 × ULN, as clinically indicated):
INFORM the Site Monitor, who will forward the information to the Study Manager.
INVESTIGATE specifically for malaise with or without loss of consciousness,
dizziness, and/or hypotension and/or episode of arrhythmia since the last visit,
particularly in the previous 72 hours; rule out muscular injury.
PERFORM the following tests/actions:
LFTs: AST, ALT, alkaline phosphatase, total and conjugated
bilirubin and prothrombin time/INR (mandatory assessments for ALT >3 × ULN);
CPK, serum creatinine, complete blood count;
Anti-HAV IgM, anti-HBc IgM, (HBV-DNA if clinically indicated),
anti-HCV and HCV RNA, anti-CMV IgM and anti- HEV IgM antibodies;
Iron, ferritin, transferrin saturation;
Auto-antibodies: antinuclear, anti-DNA, anti-smooth muscle, anti-LKM, anti-mitochondrial;
Evaluate recent infection with EBV, herpes viruses. Depending on the clinical
context, consider testing for toxoplasma;
Collect and freeze serum sample (5 mL × 2);
Collect and store one PK sample following the instructions in the central
laboratory manual;
Consider hepatobiliary ultrasonography (or other imaging investigations if needed);
Consider DNA test for Gilbert's disease if clinically indicated;
Consider consulting with a hepatologist (mandatory if ALT >8 × ULN or is
associated with elevated bilirubin);
Discuss clinical indication for potential liver biopsy with the hepatologist;
Consider patient hospitalization if INR >2 (or PT <50%) and/or central nervous
system disturbances suggesting hepatic encephalopathy
MONITOR LFTs after discontinuation of IMP:
Monitor closely (every 2-3 days) until ALT is down-trending, then weekly
until <1.5 × ULN, and then at everyscheduled visit;
This frequent LFT monitoring may be done through central or local lab, or via
home visit (depending on the Investigator's assessment and/or local regulatory
requirements).
Rechallenge: Re-initiation of the study drug can only be considered after
discussion with the Sponsor's Medical Monitor once the ALT/AST decreases
to <1.5 × ULN, and there is no clinical contraindication. No rechallenge will
be considered for participants with >3 × ULN ALT and >2 × ULN bilirubin increase.
In case it is agreed to re-start the study drug, it is recommended that
ALT/AST be assessed per protocol schedule of assessments for the
first 6 months of the treatment period.
The occurrence of new elevation >3 × ULN for the ALT/AST values will
lead to permanent discontinuation of the study drug.

Increase in serum creatinine is to be recorded as an AE only if at least 1 of the criteria listed in the general guidelines for reporting adverse events in Appendix 3 (Section 10.3) is met.

Increase in CPK is to be recorded as an AE only if at least 1 of the criteria in the general guidelines for reporting adverse events in Appendix 3 (Section 10.3) is met.

Abbreviations in FIG. 5: ARF, acute renal failure; ULN, upper limit of normal; DIC, disseminated intravascular coagulation; CPK, creatine phosphokinase; ECG, electrocardiogram; PK, pharmacokinetic(s).

Increase in serum creatinine is to be recorded as an AE only if at least 1 of the criteria listed in the general guidelines for reporting AEs in Appendix 3 (Section 10.3) is met.

Abbreviations in FIG. 8: CK-MB, creatine kinase-MB; CK-MM, creatine kinase-MM; ECG, electrocardiogram; PK, pharmacokinetic(s); ULN, upper limit of normal.

Increase in CPK is to be recorded as an AE only if at least 1 of the criteria in the general guidelines for reporting AEs in Appendix 3 (Section 10.3) is met.

SUSPECTED PML: If either the clinical presentation or MRI features of a participant are suggestive of PML, the diagnostic and action algorithm described in FIG. 6 is recommended.

Abbreviations in FIG. 6: Abbreviations: CSF, cerebrospinal fluid; Gd, gadolinium; IMP, investigational medicinal product; JCV, John Cunningham virus; MRI, magnetic resonance imaging; PCR, polymerase chain reaction; PML, progressive multifocal leukoencephalopathy

Clinical manifestations or MRI lesions features suspicious for PML are proposed in Table 3H (based on Berger et al. Neurology 2013, 80, 1430-1438 and Kappos et al. Lancet Neurol. 2007, 6, 431-441).

In the event that PML is suspected based on imaging results, the local radiologist will directly inform the Investigator and a central review of the MRI will not be required. The Investigator will obtain additional plasma, urine, and CSF samples for John Cunningham virus (JCV) analysis. Samples will be analyzed upon receipt and the results will be provided directly to the investigational site and to the Sponsor. Further management will be deferred to the Treating Investigator. However, next steps will include discontinuation of study treatment. Additional imaging will be at the discretion of the Investigator depending on the diagnostic workup and treatment plan.
The detection of John Cunningham virus (JCV) DNA in the cerebrospinal fluid of a patient with clinical and MRI features suggestive of PML establishes the diagnosis of PML.
If JCV DNA is not detected in cerebrospinal fluid and if clinical suspicion of PML remains high, another lumbar puncture should be performed.
If diagnosis remains uncertain and suspicion of PML remains high, a brain biopsy may be considered to establish a definitive diagnosis

TABLE 4H
Clinical and MRI features suggestive of PML

| | |
|---|---|
| Clinical | Subacute onset of weakness, sensory deficits, cognitive |
| history | or behavioral abnormalities, gait dysfunction, |
| | speech/language difficulties or any other signs of |
| | cortical dysfunction, retrochiasmal visual defects or seizure |
| Brain MRI | ≥1 T2/FLAIR hyperintense and T1 hypointense lesions |
| | involving the subcortical and juxtacortical white matter, |
| | sparing the cortex, with no mass effect, with a |
| | continuous progression; new lesions with no enhancement |
| | (even when large) or with faint rim enhancement |

Clinical or MRI features suggestive of PML should be recorded as an AE/AESI/SAE following the definitions and procedures in Appendix 3 (Section 10.3).

Example of Drugs with a Potential to Change SAR442168 Metabolism or Absorption

The following drugs should not be taken during the study concomitantly with IMP due to their potential to change SAR442168 kinetics due to interaction with P450-mediated metabolism, being potent and moderate inducers of CYP3A or potent inhibitors of CYP2C8 liver enzymes (per the lists of the Drug Interaction Database Program of the University of Washington [www.druginteractioninfo.org]).

Please note that the lists provided are not exhaustive and that the product information of drugs intended for concomitant use should be consulted.

TABLE 4I
CYP3A Inducers and CYP2C8 Inhibitors
Potent CYP3A Inducers:

| | |
|---|---|
| Rifampin | Carbamazepine |
| St John's Wort extract | Phenobarbital |
| Avasimibe | Lumacaftor |
| Rifapentine | Rifabutin |
| Phenytoin | |

Potent CYP2C8 Inhibitors:

| | |
|---|---|
| Gemfibrozil | Clopidogrel |

Moderate CYP3A Inducers:

| | |
|---|---|
| Semagacestat | Asunaprevir/beclabuvir/daclatasvir |
| Cenobamate | Nafcillin |
| Lesinurad | Modafinil |
| Bosentan | Telotristat ethyl |
| Thioridazine | Elagolix |
| Rifabutin | |

### Abbreviations

9-HPT: 9-hole peg test
ADL: activities of daily living
AE: adverse event
AESI: adverse event of special interest
ALT: alanine aminotransferase
aPTT: activated partial thromboplastin time
ARR: annualized relapse rate
BTK: Bruton's tyrosine kinase
BVL: brain volume loss
CD: cluster of differentiation
CDI confirmed disability improvement
CDP: confirmed disability progression
CFR: code of federal regulation
Chi3L1: chitinase-3-like protein 1
CNS: central nervous system
CPK: creatinine phosphokinase
CRF: case report form
CSF: cerebrospinal fluid
CSR: clinical study report
C-SSRS: Columbia Suicide Severity Rating Scale
CVLT-II California Verbal Learning Test-II
CYP: cytochrome P450
DILI: drug-induced liver injury
DMC: Data Monitoring Committee
DMT: disease-modifying therapies
DNA: deoxyribonucleic acid
DNAM-1: DNAX accessory molecule 1
DTP: direct to patient
ECG: electrocardiogram, electrocardiography
eCRF: electronic case report form
EDSS: expanded disability status scaleNumbered Embodiments
eGFR: estimated glomerular filtration rate
EOS: end of study
EOT: end of treatment
EQ-5D-5L: EuroQol 5-dimension 5-level questionnaire
FSH: follicle stimulating hormone
GCIPL: ganglion cell-inner plexiform layer
GCP: good clinical practice
Gd: gadolinium
GM-CSF: granulocyte-macrophage-colony stimulating factor
GrA: human granzyme A
GrB: human granzyme B
GrK: human granzyme K
GrM: human granzyme M
HIV: human immunodeficiency virus
HR: hazard ratio
HRT: hormone replacement therapy
IB: Investigators Brochure
ICF: informed consent form
ICH: International Council for Harmonisation
IEC: independent ethics committees
IFNγ: Interferon γ
Ig: immunoglobulin
IL: interleukin
IMP: investigational medicinal product
INR: international normalized ratio
IRB: institutional review board
IRT: interactive response technology
ITT: intent-to-treat
IUD: intrauterine device
IUS: intrauterine hormone-releasing system
IV: intravenous
IWRS: interactive web response system
JCV: John Cunningham virus
KM: Kaplan-Meier
LLN: lower limit of normal
MCAM: melanoma cell adhesion molecule
MedDRA: Medical Dictionary for Regulatory Activities
MMRM: mixed-effect model with repeated measures
MRI: magnetic resonance imaging
MS: multiple sclerosis
MSFC-3: Multiple Sclerosis Functional Composite-3
MSQoL-54: multiple sclerosis quality of life-54 questionnaire
MTR: magnetization transfer ratio
NCI CTCAE: National Cancer Institute Common Terminology Criteria for Adverse
Event
NEDA: no evidence of disease activity-3
NfL: neurofilament light chain
NIMP: noninvestigational medicinal product
NSAIDs: nonsteroidal anti-inflammatory drugs
NYHA: New York Heart Association
PD: pharmacodynamic
PK: pharmacokinetic(s)
PML: progressive multifocal leukoencephalopathy
PPMS: primary progressive multiple sclerosis
pRNFL: peripapillary retinal nerve fiber layer
pTreg: peripheral regulatory T cells
QTcF: QT interval corrected using Fridericia's formula
RMS: relapsing multiple sclerosis
RTE: Recent thymic emigrant
SAE: serious adverse event
SAP: statistical analysis plan
SDMT: symbol digit modalities test
SEL: slowly evolving lesions
SoA: schedule of activities
SPMS: secondary progressive multiple sclerosis
SUSAR: suspected unexpected serious adverse reaction
SWI susceptibility-weighted imaging
T25-FW: timed 25 foot walk
TB: tuberculosis
TEAE: treatment-emergent adverse event
Tfh: T follicular helper
Th 17: T helper 17 cells
Th 17: T helper 17
Th1: T helper 1 cells, T helper 1
Th2: T helper 2 cells
Th2: T helper 2
TIGIT: T cell immunoglobulin and immunoreceptor tyrosine-based inhibitory
motif (ITIM) domain
TNFα: tumor necrosis factor α
Treg: regulatory T cell
tTreg: Thymically derived Foxp3(+) regulatory T cells
ULN: upper limit of normal
US: United States
WOCBP: woman of childbearing potential

Embodiment #1: A method of treating MS in a patient in need thereof comprising determining whether the patient has elevated transferrin or elevated ferritin levels and when the patient is found to not have elevated transferrin or elevated ferritin levels, administering to a patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #2: A method of treating MG in a patient in need thereof comprising determining whether the patient has elevated transferrin or elevated ferritin levels and when the patient is found to not have elevated transferrin or elevated ferritin levels, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #3: A method of treating MS in a patient in need thereof comprising determining the patient's iron panel, and when the patient is found to have a suitable iron panel, administering to a patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #4: A method of treating MG in a patient in need thereof comprising determining the patient's iron panel, and when the patient is found to have a suitable iron panel, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #5: A method of treating MS in a patient in need thereof comprising determining the patient's alcohol consumption, and when the patient is found to have a suitable alcohol consumption, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #6: A method of treating MG in a patient in need thereof comprising determining the patient's alcohol consumption, and when the patient is found to have a suitable alcohol consumption, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #7: A method of treating MS in a patient in need thereof comprising determining if the patient has inducers of CYP3A or inhibitor of CYP3C8 in the patient's system, and when the patient is found to not have an inducer of CYP3A or an inhibitor of CYP3C8, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #8: A method of treating MG in a patient in need thereof comprising determining if the patient has inducers of CYP3A or inhibitor of CYP3C8 in the patient's system, and when the patient is found to not have an inducer of CYP3A or an inhibitor of CYP3C8, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #9: A method of treating MS in a patient in need thereof comprising determining if the patient has elevated ALT enzymes, and when the patient is found to not have elevated ALT enzymes, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #10: A method of treating MG in a patient in need thereof comprising determining if the patient has elevated ALT enzymes, and when the patient is found to not have elevated ALT enzymes, administering to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #11: A method of treating MS in a patient in need thereof comprising determining whether the patient has elevated transferrin or elevated ferritin levels and when the patient is found to have elevated transferrin or elevated ferritin levels, discontinue the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #12: A method of treating MG in a patient in need thereof comprising determining whether the patient has elevated transferrin or elevated ferritin levels and when the patient is found to have elevated transferrin or elevated ferritin levels, discontinue the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #13: A method of treating MS in a patient in need thereof comprising determining the patient's iron panel, and when the patient is found to not have a suitable iron panel, discontinue the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #14: A method of treating MG in a patient in need thereof comprising determining the patient's iron panel, and when the patient is found to not have a suitable iron panel, discontinue the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #15: A method of treating MS in a patient in need thereof comprising determining the patient's alcohol consumption, and when the patient is found to not have a suitable alcohol consumption, discontinue the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #16: A method of treating MG in a patient in need thereof comprising determining the patient's alcohol consumption, and when the patient is found to not have a suitable alcohol consumption, discontinue the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #17: A method of treating MS in a patient in need thereof comprising determining if the patient has inducers of CYP3A or inhibitor of CYP3C8 in the patient's systems, and when the patient is found to have an inducer of CYP3A or an inhibitor of CYP3C8, discontinue the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #18 A method of treating MG in a patient in need thereof comprising determining if the patient has inducers of CYP3A or inhibitor of CYP3C8 in the patient's systems, and when the patient is found to have an inducer of CYP3A or an inhibitor of CYP3C8, discontinue the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #19: A method of treating MS in a patient in need thereof comprising determining if the patient has elevated ALT enzymes, and when the patient is found to have elevated ALT enzymes, discontinue the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #20: A method of treating MG in a patient in need thereof comprising determining if the patient has elevated ALT enzymes, and when the patient is found to have elevated ALT enzymes, discontinue the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #21: A method of treating MS in a patient in need thereof comprising determining if the patient has elevated ALT >1.5×ULN OR AST >1.5×ULN OR alkaline phosphatase >2×ULN (unless caused by non-liver-related disorder or explained by a stable chronic liver disorder) OR total bilirubin

>1.5×ULN (unless due to Gilbert syndrome or non-liver-related disorder), and when the patient is found to have any of these conditions, discontinue the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #22: A method of treating MG in a patient in need thereof comprising determining if the patient has elevated ALT >1.5×ULN OR AST >1.5×ULN OR alkaline phosphatase >2×ULN (unless caused by non-liver-related disorder or explained by a stable chronic liver disorder) OR total bilirubin >1.5×ULN (unless due to Gilbert syndrome or non-liver-related disorder), and when the patient is found to have any of these conditions, discontinue the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #23: A method of treating MS in a patient in need thereof comprising determining if the patient has elevated ALT >1.5×ULN OR AST >1.5×ULN OR alkaline phosphatase >2×ULN (unless caused by non-liver-related disorder or explained by a stable chronic liver disorder) OR total bilirubin

>1.5×ULN (unless due to Gilbert syndrome or non-liver-related disorder), and when the patient is found to not have any of these conditions, administer to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #24: A method of treating MG in a patient in need thereof comprising determining if the patient has elevated ALT >1.5×ULN OR AST >1.5×ULN OR alkaline phosphatase >2×ULN (unless caused by non-liver-related disorder or explained by a stable chronic liver disorder) OR total bilirubin

>1.5×ULN (unless due to Gilbert syndrome or non-liver-related disorder), and when the patient is found to not have any of these conditions, administer to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #25: A method of treating MS in a patient in need thereof comprising determining if the patient has mild, moderate or severe hepatic impairment, and when the patient is found to not have any of these conditions, administer to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #26: A method of treating MG in a patient in need thereof comprising determining if the patient has mild, moderate or severe hepatic impairment, and when the patient is found to not have any of these conditions, administer to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #27: A method of treating MS in a patient in need thereof comprising determining if the patient has mild, moderate or severe hepatic impairment, and when the patient is found to have any of these conditions, discontinuing the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #28: A method of treating MG in a patient in need thereof comprising determining if the patient has mild, moderate or severe hepatic impairment, and when the patient is found to have any of these conditions, discontinuing the administration to the patient a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one.

Embodiment #29: A method of treating MS comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have elevated transferrin or elevated ferritin levels.

Embodiment #30: A method of treating MG comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have elevated transferrin or elevated ferritin levels.

Embodiment #31: A method of treating MS comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient has a suitable iron panel.

Embodiment #32: A method of treating MG comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient has a suitable iron panel.

Embodiment #33: A method of treating MS comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient has a suitable alcohol consumption.

Embodiment #34: A method of treating MG comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient has a suitable alcohol consumption.

Embodiment #35: A method of treating MS comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have an inducer of CYP3A or an inhibitor of CYP3C8 in the patient's system.

Embodiment #36: A method of treating MG comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have an inducer of CYP3A or an inhibitor of CYP3C8 in the patient's system.

Embodiment #37: A method of treating MS comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have elevated ALT enzymes.

Embodiment #38: A method of treating MG comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have elevated ALT enzymes.

Embodiment #39: A method of treating MS comprising administrating to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have elevated ALT >1.5×ULN OR AST >1.5×ULN OR alkaline phosphatase >2×ULN (unless caused by non-liver-related disorder or explained by a stable chronic liver disorder) OR total bilirubin >1.5×ULN (unless due to Gilbert syndrome or non-liver-related disorder).

Embodiment #40: A method of treating MG comprising to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have elevated ALT

>1.5×ULN OR AST >1.5×ULN OR alkaline phosphatase >2×ULN (unless caused by non-liver-related disorder or explained by a stable chronic liver disorder) OR total bilirubin >1.5×ULN (unless due to Gilbert syndrome or non-liver-related disorder).

Embodiment #41: A method of treating MS comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have mild hepatic impairment, moderate hepatic impairment, or severe hepatic impairment.

Embodiment #42: A method of treating MG comprising administering to a patient in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the patient does not have mild hepatic impairment, moderate hepatic impairment, or severe hepatic impairment.

1.-35. (canceled)

36. A method of administering (R)-1-(1-acryloylpiperidin-3-yl)-4-amino-3-(4-phenoxyphenyl)-1H-imidazo[4,5-c]pyridin-2(3H)-one to a patient with a suitable iron panel, comprising the steps of:
(a) performing an iron panel test in a patient's blood or serum;
(b) detecting levels of the iron panel test that are within normal ranges; and
(c) administering a therapeutically effective amount of (R)-1-(1-acryloylpiperidin-3-yl)-4-amino-3-(4-phenoxyphenyl)-1H-imidazo[4,5-c]pyridin-2(3H)-one to the patient,
wherein the iron panel test measures any one or more of levels of iron, ferritin, transferrin saturation, and total iron-binding capacity (TIBC) in a patient's blood or serum and wherein the normal ranges of the iron panel test include one or more of (i) an iron level of 60 to 170 μg/dL, (ii) a ferritin level of ≤500 μg/L (iii) a transferrin saturation level ≤50% in a male patient or ≤40% in a female patient, and (iv) a TIBC of 240 to 450 μg/dL;
or comprising the steps of:
(a) detecting a level of transferrin saturation in a patient's blood or serum that is within normal range; and
(b) administering a therapeutically effective amount of (R)-1-(1-acryloylpiperidin-3-yl)-4-amino-3-(4-phenoxyphenyl)-1H-imidazo[4,5-c]pyridin-2(3H)-one to the patient,
wherein the transferrin saturation level that is within normal range in the blood or serum of a male patient is a transferrin saturation of ≤50%, and the transferrin saturation level that is within normal range in the blood or serum of a female patient is a transferrin saturation of ≤40%;
or comprising the steps of:
(a) detecting a level of ferritin in a patient's blood or serum that is within normal range; and
(b) administering a therapeutically effective amount of (R)-1-(1-acryloylpiperidin-3-yl)-4-amino-3-(4-phenoxyphenyl)-1H-imidazo[4,5-c]pyridin-2(3H)-one to the patient,
wherein the ferritin level that is within normal range in the blood or serum of the patient is ≤500 μg/L.

37. The method of claim 36, wherein the method comprises the steps of:
(a) performing an iron panel test in a patient's blood or serum;
(b) detecting levels of the iron panel test that are within normal ranges; and
(c) administering a therapeutically effective amount of (R)-1-(1-acryloylpiperidin-3-yl)-4-amino-3-(4-phenoxyphenyl)-1H-imidazo[4,5-c]pyridin-2(3H)-one to the patient,
wherein the iron panel test measures any one or more of levels of iron, ferritin, transferrin saturation, and total iron-binding capacity (TIBC) in a patient's blood or serum and wherein the normal ranges of the iron panel test include one or more of (i) an iron level of 60 to 170 μg/dL, (ii) a ferritin level of ≤500 μg/L (iii) a transferrin saturation level ≤50% in a male patient or ≤40% in a female patient, and (iv) a TIBC of 240 to 450 μg/dL.

38. The method of claim 36, wherein the method comprises the steps of:
(a) detecting a level of transferrin saturation in a patient's blood or serum that is within normal range; and
(b) administering a therapeutically effective amount of (R)-1-(1-acryloylpiperidin-3-yl)-4-amino-3-(4-phenoxyphenyl)-1H-imidazo[4,5-c]pyridin-2(3H)-one to the patient,
wherein the transferrin saturation level that is within normal range in the blood or serum of a male patient is a transferrin saturation of ≤50%, and the transferrin saturation level that is within normal range in the blood or serum of a female patient is a transferrin saturation of ≤40%.

39. The method of claim 36, wherein the method comprises the steps of:
(a) detecting a level of ferritin in a patient's blood or serum that is within normal range; and
(b) administering a therapeutically effective amount of (R)-1-(1-acryloylpiperidin-3-yl)-4-amino-3-(4-phenoxyphenyl)-1H-imidazo[4,5-c]pyridin-2(3H)-one to the patient,
wherein the ferritin level that is within normal range in the blood or serum of the patient is ≤500 μg/L.

40. A method of administering (R)-1-(1-acryloylpiperidin-3-yl)-4-amino-3-(4-phenoxyphenyl)-1H-imidazo[4,5-c]pyridin-2(3H)-one to a patient, comprising the steps of:
(a) administering a therapeutically effective amount of (R)-1-(1-acryloylpiperidin-3-yl)-4-amino-3-(4-phenoxyphenyl)-1H-imidazo[4,5-c]pyridin-2(3H)-one (Compound) to the patient;
(b) measuring the level of alanine aminotransferase (ALT) and bilirubin in the patient;
(c) detecting a level of ALT of >8× upper limit of normal (ULN);
(d) ceasing administration of the Compound to the patient and monitoring the level of ALT in the patient every 2-3 days until the level of ALT is down-trending and then monitoring weekly; and optionally
(e) resuming administration of a therapeutically effective amount of the Compound to the patient when the patient's level of ALT is determined to be <1.5×ULN;
or comprising the steps of:
(a) administering a therapeutically effective amount of (R)-1-(1-acryloylpiperidin-3-yl)-4-amino-3-(4-phenoxyphenyl)-1H-imidazo[4,5-c]pyridin-2(3H)-one (Compound) to the patient;
(b) measuring the level of alanine aminotransferase (ALT) and bilirubin in the patient;
(c) detecting a level of ALT of >5× upper limit of normal (ULN);
(d) ceasing administration of the Compound to the patient and monitoring the level of ALT in the patient every 2-3 days until the level of ALT is down-trending and then monitoring weekly; and optionally
(e) resuming administration of a therapeutically effective amount of the Compound to the patient when the patient's level of ALT is determined to be <1.5×ULN;
or comprising the steps of:
(a) administering a therapeutically effective amount of (R)-1-(1-acryloylpiperidin-3-yl)-4-amino-3-(4-phenoxyphenyl)-1H-imidazo[4,5-c]pyridin-2(3H)-one (Compound) to the patient;
(b) measuring the level of alanine aminotransferase (ALT) and bilirubin in the patient;
(c) detecting a level of ALT of >3× upper limit of normal (ULN);
(d) detecting one or more of total bilirubin >2×ULN;
(e) ceasing administration of the Compound to the patient and monitoring the level of ALT in the patient every 2-3 days until the level of ALT is down-trending and then monitoring weekly; and optionally
(f) resuming administration of a therapeutically effective amount of the Compound to the patient when the patient's level of ALT is determined to be <1.5×ULN;
or comprising the steps of:
(a) administering a therapeutically effective amount of (R)-1-(1-acryloylpiperidin-3-yl)-4-amino-3-(4-phenoxyphenyl)-1H-imidazo[4,5-c]pyridin-2(3H)-one (Compound) to the patient;
(b) measuring the level of alanine aminotransferase (ALT) and bilirubin in the patient;
(c) detecting a level of ALT of >3× upper limit of normal (ULN);
(d) ceasing administration of the Compound to the patient if the patient experiences one or more of fatigue, nausea, vomiting, right upper quadrant pain or tenderness, fever, rash, and eosinophilia >5% and monitoring the level of ALT in the patient 2-3 times weekly until the level of ALT is down-trending and then monitoring weekly; and optionally
(e) resuming administration of a therapeutically effective amount of the Compound to the patient when the patient's level of ALT is determined to be <1.5×ULN.

41. The method of claim 40, wherein the method comprises the steps of:
(a) administering a therapeutically effective amount of (R)-1-(1-acryloylpiperidin-3-yl)-4-amino-3-(4-phenoxyphenyl)-1H-imidazo[4,5-c]pyridin-2(3H)-one (Compound) to the patient;
(b) measuring the level of alanine aminotransferase (ALT) and bilirubin in the patient;
(c) detecting a level of ALT of >8× upper limit of normal (ULN);
(d) ceasing administration of the Compound to the patient and monitoring the level of ALT in the patient every 2-3 days until the level of ALT is down-trending and then monitoring weekly; and optionally
(e) resuming administration of a therapeutically effective amount of the Compound to the patient when the patient's level of ALT is determined to be <1.5×ULN.

42. The method of claim 40, wherein the method comprises the steps of:
(a) administering a therapeutically effective amount of (R)-1-(1-acryloylpiperidin-3-yl)-4-amino-3-(4-phenoxyphenyl)-1H-imidazo[4,5-c]pyridin-2(3H)-one (Compound) to the patient;
(b) measuring the level of alanine aminotransferase (ALT) and bilirubin in the patient;
(c) detecting a level of ALT of >5× upper limit of normal (ULN);
(d) ceasing administration of the Compound to the patient and monitoring the level of ALT in the patient every 2-3 days until the level of ALT is down-trending and then monitoring weekly; and optionally
(e) resuming administration of a therapeutically effective amount of the Compound to the patient when the patient's level of ALT is determined to be <1.5×ULN.

43. The method of claim 40, wherein the method comprises the steps of:
(a) administering a therapeutically effective amount of (R)-1-(1-acryloylpiperidin-3-yl)-4-amino-3-(4-phenoxyphenyl)-1H-imidazo[4,5-c]pyridin-2(3H)-one (Compound) to the patient;
(b) measuring the level of alanine aminotransferase (ALT) and bilirubin in the patient;
(c) detecting a level of ALT of >3× upper limit of normal (ULN);
(d) detecting one or more of total bilirubin >2×ULN;
(e) ceasing administration of the Compound to the patient and monitoring the level of ALT in the patient every 2-3 days until the level of ALT is down-trending and then monitoring weekly; and optionally
(f) resuming administration of a therapeutically effective amount of the Compound to the patient when the patient's level of ALT is determined to be <1.5×ULN.

44. The method of claim 40, wherein the method comprises the steps of:
(a) administering a therapeutically effective amount of (R)-1-(1-acryloylpiperidin-3-yl)-4-amino-3-(4-phenoxyphenyl)-1H-imidazo[4,5-c]pyridin-2(3H)-one (Compound) to the patient;
(b) measuring the level of alanine aminotransferase (ALT) and bilirubin in the patient;
(c) detecting a level of ALT of >3× upper limit of normal (ULN);
(d) ceasing administration of the Compound to the patient if the patient experiences one or more of fatigue, nausea, vomiting, right upper quadrant pain or tenderness, fever, rash, and eosinophilia >5% and monitoring the level of ALT in the patient 2-3 times weekly until the level of ALT is down-trending and then monitoring weekly; and optionally
(e) resuming administration of a therapeutically effective amount of the Compound to the patient when the patient's level of ALT is determined to be <1.5×ULN.

45. The method of claim 40, wherein the level of ALT in any one of steps (b) is measured at least monthly, at least every two weeks, or at least weekly.

46. The method of claim 45, wherein the level of ALT in any one of steps (b) is measured at least monthly for at least the first three months of administration, at least the first six months of administration, or at least the first twelve months of administration.

47. The method of claim 45, wherein the level of ALT in any one of steps (b) is measured at least every two weeks for at least the first three months of administration or at least the first six months of administration.

48. The method of claim 45, wherein the level of ALT in any one of steps (b) is measured at least weekly for at least the first three months of administration.

49. The method of claim 40, wherein the detected level of ALT of >3×ULN is <5×ULN.

50. The method of claim 40, wherein the administration of a therapeutically effective amount of the Compound is not resumed if:
(i) the detected level of ALT in any one of steps (c) was >8×ULN;
(ii) the detected level of ALT in any one of steps (c) was >5×ULN for greater than 2 weeks; or
(iii) the detected level of ALT in any of steps (c) was >3×ULN and the detected level of bilirubin was greater than 2×ULN.

51. The method of claim 40, wherein following the resuming administration of a therapeutically effective amount of the Compound, the method further comprises the steps of:
(a) measuring the level of alanine aminotransferase (ALT) in the patient at least monthly, at least every two weeks, or at least weekly for at least the first three months following the resumption of administration;
(b) detecting a level of ALT of >3× upper limit of normal (ULN);
(c) ceasing administration of the Compound to the patient.

52. The method of claim 36, wherein the therapeutically effective amount is 60 mg of (R)-1-(1-acryloylpiperidin-3-yl)-4-amino-3-(4-phenoxyphenyl)-1H-imidazo[4,5-c]pyridin-2(3H)-one taken once daily.

53. The method of claim 40, wherein the therapeutically effective amount is 60 mg of (R)-1-(1-acryloylpiperidin-3-yl)-4-amino-3-(4-phenoxyphenyl)-1H-imidazo[4,5-c]pyridin-2(3H)-one taken once daily.
